SEQUENCE LISTING (40 sequences)

SEQ ID NO: 1 - DNA, 18301 bp, synthetic construct
  source 1..18301: mol_type = other DNA
  misc_feature 1..18301: note = Sequence human GAA including c.-32-13T>G mutation
cgaccccggagtctccgcgggcggccagggcgcgcgtgcgcggaggtgagccgggccggggctgcggggcttccctgagcgcgggccgggtcggtggggcggtcggctgcccgcgcggcctctcagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgcgcgtgccgcagctgacggggaaactgaggcacggagcgggtgagacacctgacgtctgccccgcgctgccggcggtaacatcccagaagcgggtttgaacgtgcctagccgtgcccccagcctcttcccctgagcggagcttgagccccagacctctagtcctcccggtctttatctgagttcagcttagagatgaacggggagccgccctcctgtgctgggcttggggctggaggctgcatcttcccgtttctagggtttcctttccccttttgatcgacgcagtgctcagtcctggccgggacccgagccacctctcctgctcctgcaggacgcacatggctgggtctgaatccctggggtgaggagcaccgtggcctgagagggggcccctgggccagctctgaaatctgaatgtctcaatcacaaagacccccttaggccaggccaggggtgactgtctctggtctttgtccctggttgctggcacatagcacccgaaacccttggaaaccgagtgatgagagagccttttgctcatgaggtgactgatgaccggggacaccaggtggcttcaggatggaagcagatggccagaaagaccaaggcctgatgacgggttgggatggaaaaggggtgaggggctggagattgagtgaatcaccagtggcttagtcaaccatgcctgcacaatggaaccccgtaagaaaccacagggatcagagggcttcccgccgggttgtggaacacaccaaggcactggagggtggtgcgagcagagagcacagcatcactgcccccacctcacaccaggccctacgcatctcttccatacggctgtctgagttttatcctttgtaataaaccagcaactgtaagaaacgcactttcctgagttctgtgaccctgaagagggagtcctgggaacctctgaatttataactagttgatcgaaagtacaagtgacaacctgggatttgccattggcctctgaagtgaaggcagtgttgtgggactgagcccttaacctgtggagtctgtgctgactccaggtagtgtcaagattgaattgaattgtaggacacccagccgtgtccagaaagttgcagaattgatgggtgtgagaaaaaccctacacatttaatgtcagaagtgtgggtaaaatgtttcaccctccagcccagagagccctaatttaccagtggcccacggtggaacaccacgtccggccgggggcagagcgttcccagccaagccttctgtaacatgacatgacaggtcagactccctcgggccctgagttcacttcttcctggtatgtgaccagctcccagtaccagagaaggttgcacagtcctctgctccaaggagcttcactggccaggggctgctttctgaaatccttgcctgcctctgctccaaggcccgttcctcagagacgcagacccctctgatggctgactttggtttgaggacctctctgcatccctcccccatggccttgctcctaggacaccttcttcctcctttccctggggtcagacttgcctaggtgcggtggctctcccagccttccccacgccctccccatggtgtattacacacaccaaagggactcccctattgaaatccatgcatattgaatcgcatgtgggttccggctgctcctgggaggagccaggctaatagaatgtttgccataaaatattaatgtacagagaagcgaaacaaaggtcgttggtacttgttaaccttaccagcagaataatgaaagcgaacccccatatctcatctgcacgcgacatccttgttgtgtctgtacccgaggctccaggtgcagccactgttacagagactgtgtttcttccccatgtacctcgggggccgggaggggttctgatctgcaaagtcgccagaggttaagtcctttctctcttgtggctttgccacccctggagtgtcaccctcagctgcggtgcccaggattccccactgtggtatgtccgtgcaccagtcaataggaaagggagcaaggaaaggtactgggtccccctaaggacatacgagttgccagaatcacttccgctgacacccagtggaccaagccgcacctttatgcagaagtggggctcccagccaggcgtggtcactcctgaaatcccagcacttcggaaggccaaggggggtggatcacttgagctcaggagttcgagaccagcctgggtaacatggcaaaatcccgtctctacaaaaatacagaaaattagctgggtgcggtggtgtgtgcctacagtcccagctactcaggaggctgaagtgggaggattgcttgagtctgggaggtggaggttgcagtgagccaggatctcaccacagcactctggcccaggcgacagctgtttggcctgtttcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccccacctgcctgggtgctgcagtgccagccgcggttgatgtctcagagctgctttgagagccccgtgagtgccgcccctcccgcctccctgctgagcccgcttgcttctcccgcaggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacggtgggcagggcaggggcgggggcggcggccagggcagagggtgcgcgtggacatcgacacccacgcacctcacaagggtggggtgcatgttgcaccactgtgtgctgggcccttgctgggagcggaggtgtgagcagacaatggcagcgcccctcggggagcagtggggacaccacggtgacaggtactccagaaggcagggctcggggctcattcatctttatgaaaaggtgggtcaggtagagtagggctgccagaggttgcgaatgaaaacaggatgcccagtaaacccgaattgcagataccccaggcatgactttgtttttttgtgtaaggatgcaaaatttgggatgtatttatactagaaaagctgcttgttgtttatctgaaattcagagttatcaggtgttctgtattttacctccatcctgggggaggcgtcctcctcctggctctgcagatgagggagccgaggctcagagaggctgaatgtgctgcccatggtcccacatccatgtgtggctgcaccaggacctgacctgtccttggcgtgcgggttgttctctggagagtaaggtggctgtggggaacatcaataaacccccatctcttctagatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgtgagttctgggctctgtgccagcatgatggggagggcgacgcgcatttctcacacggcagggagggccacacgcgtttgtttctcacacgatgggcagggcgacacatgtttgtttctcacacggcggggagggcgacgggcatttctcacagggcgctccctgggtcttttactcacataggtctaaatcccatgtaaacacgtgttcaggactcaccaagcccctgcttgtcatttaactcaggaaaactctcaggaacgacagcacttggatttgccttaatcttaagagaagttgccttcggaaatgcgtttttctttttttgctcattcatttactcagtgtccacgcactgaccctccgtgccgggtggtttggatcctgctcccggggacagacacacagtgaggggaagccataagcaagtccatgcagacacagcgtcagggagtggtcatgcagagagcacgctagaagccagctgtgcagacacggggcagggaggtcccctctagaagccagctgtgcagacgcaggggacagggatggcctctctggaagccagctgtgcagatgttgggggcaggggtggcctctctggaagccagctgtgcaggagtggggggtggggaggccactctggaaaccagctgtgcagatgcaggggacaggggtggcctctctgagctgacctctgagtagagagacccaagagaagtttctcaaagcatcttatcaagctaggtatggtggttcatgtctgcaattccagcactttgggaggccaaggcgagagggtcacttgagcccaggagttcaagaccatcctgggcaacatagcaagaccccatctcttaaaaaataaaaataaaaaattagctgggaattgtggcacatgcctgtggtcccagctactcaggaggctgaggcaagaggatcccttgagcccaggggttcgaggttgcagtgaaccatgattttgccactgcacttcagccttgctgaagaccccgtctcaaaaaacaaacaacaaacaggcatcttatcagatctcggtcttgaaagcactcagcgtagtcttgcccaggggagggtgggtgcggtgtgagcccgtcctgcgaaattagctgtgctgtgttaacagaggacgcgtcttcctgtggaccgggtttatctgcggctttcatttctcggaggtgctgtttgccttgcacttgacccccagcaaacctcaggggtccttctcaggcatggctgggctgggatctgggaggactttggccacaagctcctaggcctggaaaggttctgttcagcccctgcccagccttgcttggggtcatgggacaggcatgtgtgccagttccggtaccagccagttcctggaggtcagccccttgggggcccctcaggggtggtgtgggcccagccaggcggtgcgcctcttctgatatgccctgagagttgatcacgctggtgccagggtgccaagggctgcagggctcggcacggccgcctgtcccagggtcagtgtgctgcagggctggccaggccactccgccctcccagggcaccagggcccgggggtgctctctgggtgctctcaggctcgtgtggccccttgggtgtgagcaagcctggctggcctctgtcccgcaggctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacggtacagcggcgggcggcgggcgggggcactgagctggggagcgcaggtgctgaagcgccgtctcctgcatgtcccagcccggtgcgaacctctacgggtctcaccctttctacctggcgctggaggacggcgggtcggcacacggggtgttcctgctaaacagcaatgccatgggtaagctgcccgccgcccagcgcccgggccggggtctcctccgtgctgcctgccctggagactggaggtccgcatgaggggccctgggcacggtgctgggccttgtgttttctgggaaatgagtcctatgggctgatgcctctcccaactctggccttctgtgctcctaaggagggttctggggccctgcctggaggtgggctggcaccacatatctttccgtcccatgccaggttcctcctgagtcaggcttagcacggcttccccaggccactctgagctcctcgtggggagagagcctcaactctccgcctgtgattggcccatctgtggggtgcagagccctccaagtgaagaatctgtcccccaaccccagagctgcttcccttccagatgtggtcctgcagccgagccctgcccttagctggaggtcgacaggtgggatcctggatgtctacatcttcctgggcccagagcccaagagcgtggtgcagcagtacctggacgttgtgggtagggcctgctccctggccgcggcccccgccccaaggctccctcctccctccctcatgaagtcggcgttggcctgcaggatacccgttcatgccgccatactggggcctgggcttccacctgtgccgctggggctactcctccaccgctatcacccgccaggtggtggagaacatgaccagggcccacttccccctggtgagttggggtggtggcaggggaggcaaggggctggccgggacgcgtctcctcaggccccagcagacggtcccgtgttgtggctgcaggacgtccagtggaacgacctggactacatggactcccggagggacttcacgttcaacaaggatggcttccgggacttcccggccatggtgcaggagctgcaccagggcggccggcgctacatgatgatcgtggtgtgtgcccccacactgtgggtctttgggaagggggccgcccggtgcccagtggctccttctctgtgcagcgtcatcctcgtgcctgtgtggtcgccgaggatgttttctgagggtctttgtgatatcgagggaatatcaagaagtttgcaggcttggccccagctgtccagggaggtcgggtttgagggtccccagaaatggccgggtgctactcagggttctgtcagatgtaggttacttgaactgccttaaagcaaaaggccaggggcatgataaactgatgtcacctggtcctggaaagtggagggcccggtgggcctgggcatgggtatcgctggaactgtggaggctccgtgtgccttctggccgtgcctctccttctggccggctctgaatccctggaaaggacggcgtgagtgagggcagcttccagccctcatgctggcaccacagagcggagacttcttcccatcagctcccatagaaaagtcccaaagcaggactcttgagtcacccagcacaaagaggcccttccctgagccagtcccacagccagaaggatgcagtttgggggctggtccagcccgagtctggtgtccggcacgatggccagaggaggaggtgggaggcagggcgagctgaaaagatccaacagttcctgcccggaagatccacttcagcagaggaagcacagatgagatgtggggctgtgctgatgctgcctgtttccatccctgccttctgcaggcagcaaacagtagtagcccttaagagcaggagtggaaacacagacttttttctttctcacatttttttaattataaaagaaaagtgattactgtagaacacttgggaaactctagaggtttaaagaaaaggtaaaggtaaagcttccattccggcgcgcccctcatcagccagctggtcctgactcgcccggccctggctcctctccaggcaggcgtgtgcaggcatgtgcaggtacacaggcaggcatgctgtacacacgcatgatgtcatccccagcctcatcctctcactgtctcagttttccccgtggctggcgccagggctctgggccaccctcaccttgacaggtttccctcttcccaggatcctgccatcagcagctcgggccctgccgggagctacaggccctacgacgagggtctgcggaggggggttttcatcaccaacgagaccggccagccgctgattgggaaggtagggcgagggtccaggggacgggggttagaaagcagaggcctccagccagggggagccggcagctgctcaggaagacggtgggatttgaggagccatcacgcccagtgggacagctgagaggaatgggccacagtggcccgtgacgatggtggctcctacaaggaatggccccgtgagttcttccatcagcaggcctttgacttcatgggcagctgggcctggcccaggcacaagccctgcagaccctcagtgaggccttagggtcctccttgtcctcccagccccccaggggcctccaggcagggcccccgctgagggagcagctagggagggtctggtgcggatgtgaggctgcctggcagggcttgcacggggccgtctccgctgcccttctccctgacgctctctggttctgcagcccagcccctgggtggacgtgttgggggtgacccctcgttttcccagggttgaggccccttggccccgcatcagtgccttgtggagaaagagctgctcattgacctccagggtgcaggtctctcagatttgcaaatgtgggcgtccactaagagtgaggctgcccctctgctcaggctgaggctcagtggggcttccatgcaggccctgggtggggccgggtctccccactgcagcctctcgttgtccaggtatggcccgggtccactgccttccccgacttcaccaaccccacagccctggcctggtgggaggacatggtggctgagttccatgaccaggtgcccttcgacggcatgtggattgtaagtgtggccccctcctgagcatccccaaggcctctggggactaccccaccctcctcactctgggcagagtcacctaccagcagcgcttctcttgcaggacatgaacgagccttccaacttcatcaggggctctgaggacggctgccccaacaatgagctggagaacccaccctacgtgcctggtcagctcgccccccacctaccctggggacttaatcaaatcagagactcccttgtctggcctgggagacttagcaccctcatctctgagaagcagatgggccagcggggaaaggggcgggggggggatccccaggagaaaggctcaggctgggagactcagccaagcagtgcagacagggtgggtgcagaggcacaggccctgccggaggagacgccgctcacaggtgcttgccagagcacagtgaggccgactcgactcagagccgtctcgataggcgcagggaccatgcagcggagacctacccacccgtggggagaggtcaggcccaactcgaatgcagcacgggcaagtggatttctagccagggagcagggtgggctcagaggcaggaattaccaagaagaagcatgggggtcagggggattctggctgaactgacccagcaggattcttgctgaaggcaggccagggtgaccagacatcgcctgaggggtggtggaggttggggcttctcgccaaactgtcttagcaggaatggcagaaactgggttttacaaggaagtacaaggatgggcctgggagaaggtttgggggcctgaggctatagtttggcccagcaaagaatcagtgagaggatggggttttgggcttaggtaaacaggcaggggagtgcttgaaatgggccaagagacggtggatgtgaagtctgggggtctgcagagcccaggctccagcacccgcccagccctgtcttagaagcagtggagatgattacccaggttcccgggtaacgccagccccacagaggcgtggggagcggctgcaggtgcacctccagggccagcctgaagaggcagcgacctgcacaggggctcctgggaggtggggggcagggagggcaccttggagcctgccgggaggaagctccctggaaaccagcccccgcctcttccaggggtggttggggggaccctccaggcggccaccatctgtgcctccagccaccagtttctctccacacactacaacctgcacaacctctacggcctgaccgaagccatcgcctcccacaggtgagggccacgtcccgccccactgggctctgccctcacagcctgtcctacaaggttggggcctctgcagggcctcagggaggaggaaaagcggaggcccagaccacccggggcccgctggcggcccgagtgctctccccacccgctgcctgcaccccagcctgaagctggagcgctccttcccacttcatgcctggggcttggagaggaaggaccctggatgctgacaggagtctgcatcagcggggacctcatgactcctgtgaggctggggggggtcctggctcacctacaggcatcaggtggcccagacagaggcaactgtgcccgcagacatgggcagtagcctcgccgtcctcctccccagcctctgcctcatcccagaaagctccttgctcccagctctgccctgctggtgacagggttcccgagtgaccccgctccacacagccctcacggtgtcccccaccaccccagggcgctggtgaaggctcgggggacacgcccatttgtgatctcccgctcgacctttgctggccacggccgatacgccggccactggacgggggacgtgtggagctcctgggagcagctcgcctcctccgtgccaggtgagctcctaccaggaggggctgctcagcagagtagagccgggggcctctatgggaggcttgccggggccccccacccacttagcaggtggggctctgggtcacttggcctgagctggctctgctgcagcagcctgaggaccagcctgactctgccctcccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctggtagggtgggggtggcggcatggcaggtgggcgatcccacccacccaagactctcccctgggaatcccacccctgctggagaagcaccccatgctgggtggctgagaagtgcagctctcccgaggcggggactccaggggaccgcggccccagcacccaagtgcttcctttgcccccgcctgccctgcagccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagtgagtgacctaggcaggggcggtggcccatgtgtgccctgggggaggggcacgtaactcccaggcagccctgtcctgctgtgggctgtgttccccaggacccagcaggttgccgctgagtgagacaacatttgggcctggcttaagggggaagggcagcaagaaaacccagtaatatcccccagacaggccgtagtacacacgaggagttcctaacaacagccctgcacatcagtgtgttgagggaggattcccagagagtgaggtgattagttaactatttgcagaagtcaatttatttttcttgcataccatagaaacataagttccagataaattaaatagttaatgtcaaaaatcaagctgtggaggccaggcacggtggctaacgcctgtaatcccagaactttgggaggctgaggcgagtggatcacctgaggtcaagagttcgaggccagcctggccaacatggtgaaacccatctctactaaaaatacaaaaattagccgtgcatggtggtgggcgcctgtagtccctgctactcaggaggctgaggccagagaatcccttgaacctgggaggaggagattgcagtgagccgagatcgcgccactgtactccagcctgtgtgactccatctcaaaaaaaaaaaaccaagctgtgaaagactccacaggaaaagataagtgaatgtgtatctctggggaaaggtttcatttagagaaaaaaaaaaaaaaaaggtgaactttatttgaccatagcagaaaaaaaaatgtaaatctctgaatataaacacaaaaagattaaaactgctctgaacatggactgtaacagcagagaaggcattgtcaataaaacagcaaatagctactcttcctaataggtagagaactcatacgttggtaagaccaagactaagaaccaaattggaggaacactgtttgcaaaacaggagatacgaatggtatccacacattcttcacccggaaatccaagaaacgcaatttgtgttttaattactattttaatgaccttgttgtttctgtcactacacttttttttttttttttttagtggttgccctagggattacaattaacatcttaattttagcctcgttggaactgatgccaacttgctttcaatagcataaaaaagctttgcttctatgtggtttccattgcctctcgttcctctgtgctgttacacgtctgtatccatgatgagcccatccacgcagatttataatgaccgccttattcaattatcttttaagtcaaataggagggaaaaatgggttacaaacaaaaagtacatacacactgtctgccttttatatttactgatgtagttacctttacccatggttttatttcttcatgtggctttgacttcttgtctaacatctttcatttacagcagaaggactccctttagtatttattgtagggcagttctgctagtgatgaattctctcagtttttgttgacttcagagtctcttaatttctccttcattttttgaaagttctgctcaacgtagggttcttgactggcggtcgtttctctgaacactttgaacttgtcagcccatggcctccgtggtttctgctgagtagcttctgtcttgcctcttcccagattctctgtctttggcctttgaagtcccgatggtgtgtctaggtgtagatctctgagtttatcttactgggagtttgttgagcttcttggacgtatgcattgatgtttttcatcaaacttgggcggtttttcagccattacttcataaaatattttttctgcccctttctgtcttctactctgggacttccgttccacacattggtatgcttgacggtgcccaggggtctttgcagctctgttgactccttcctcgttgtgttttctttctgttcttcagactgcttagtctgactgccctgtcttcaaggtcactgattcttttttccaccagttctcatctgctgttgagcccctctagtgaacttctcattttagttgctctcttttcaactccagaatttctgtttcattcctttacgtttctctcgttaaggatattccctatctggtgagtcatgttctcacactttcctttagttcttcagacacggtttcgctgagctctttgaacacatttaaagtagctgacgtaaactctttgtctagtaagtccaatgtctgggcttctctagggacaatttttattaactactgtccccccatctgtgggccatactttcttgtttctttgcgtgtctcatacatttttgtttcagactggacattttaaacgcagctgctgtggtcatcagattccccatccccctcaggtctcgttgctattgctgattgttttgtgactttcttgagctaattccataaggtctgtgttcttcattgtgtgtggccaccaaagtctctgcttgactagcttagtggacagccaataattggtcagatatccttcacagatggcatccgtgagtctcccagcctttgctggggcgaggtggggagcaccgtcaacacttagctaggccaaggattcttgctgttcttacaaagattcagccattttgttaaatacatgctccccagatggctgcaaacccttggttagtttcaagagtcctaaaaatgtcaactctgaccatttttgccgatgttcctgttttcacagaggagaggatcttgagaggggcttactccaccatcttcactgacatcactccaagaaatgtattttgcaagaaatattttgaagcagaaagacaccatttattttgcccatgaattagaatacattagagaaaataagactatcccttgctggcaagaacacagtgacacagtagggtggaatataaattggcacatttgtggaaagcaacagtacatatcagtcaatgtttttgagattcacattgatgcgttaattttacaaatagaaatagggcctaaagaagtcacctcaaaaggcatgaacgctccatgaatgtatccatgcagggatcatagctgagcactggatgccccctgcacgtccgggggcaggaaacaggacagggcagagctgcgtcacagggcaggacagtctccgttagacggagaatcctccgtagagctgcttgcacatgtacattcatctttttgtcagatgttaattcaagttgcctttggttgtgggactgggaggatcttttctctttgttgatactttttcgtactttccaaatacttgactgatgagcacatgctgccttggttaccggaggataagtgagcgagcaaagtgaggccagtgctgtgtccatcctggtgcctcaagcacaagcccctattcctgccctgagcccagctgccggcatgtccggggagaaggcttctcccagctccggcattgacttctatctgctggaatcatccctgcccgtctgacctgagtcctccaagtcctccggcaccttgagctccagagagcagaattcagcctcttcctgtgcctccccagggtgggcatatgagccagccccatcccattcatcacccgtatgcctgtgtgcccatcccccttgcaggttccccaaggactctagcacctggactgtggaccaccagctcctgtggggggaggccctgctcatcaccccagtgctccaggccgggaaggccgaagtgactggctacttccccttgggcacatggtacgacctgcagacggtgagtctggggaccctaagccctggggagacgggagaccagagcagccctcccacctgccccctccacccagttggtgtgaccaggtggcggaaagaggaacgtatgtgttgagtcccggccatgtgccaggcccccacccggctgctccgcacccatcagcctctccgctcctcacaccatccccatttcccagatgagcagactgaggcctgcttgcagaacctggccaagtcccacggccatcacaggctgtgcctgtgctgagctggcatacccaggcctctcaggcactgtccccactcagtagccaggagggtccctacctacagtgagccctgagtctgcgcctgaagtcacagttcagcccgtctgtgccaggcctcctaggcctccacgtggagccccgggagatggagagcgtggttcctgaggacagcatgggggcctcggcacggcccagaatcctcaaagcaacatctccctccaggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcaggtacctgggccaggcggctatggtgggggtgtggacagcacactgcagagctgggggaggcacagggagatggtgggggagaggcccaggtggggcttctgaggggccgccccccgcagtgtaggttatcaaggagccagccaggccagtgaggtggggagggcacagccccacaaaggcgtggagcatggccggcaggagctcagtggtctgcatggtggaggttctgccgggcccggcctcgggcagccgtgggatagcacttgaggtggggaaggtcttgggtcatcaccacggggttccagcccctgcggccgcaggtgttcctgcagatcctagttactggcagcctggtgctgtaccagcctagcattcccgggccctggaggcctccacctccaccagggtggggatgatgacatcacgtgtccttccctttccagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaatgtgagtcctggggctgctcaggctggtgggcaggggccggctcggggttgagaaggggtgaggggacctgggcttgggggtcccacgatggctacctgccactaggacactctagcaggtggcctggggtcctagagtgagcagtggggccgtgcactctgccctttcgtgtacacagagggaggtcacctccctgatgccatcatgagtccctgttctcatgggtgttcctgccccagctgtctgctgacacctccacattctctgccttttcatctctctctgctcggcccagaacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggcaagagggcccagagtggcacagggatcgcgtcccccagccgtggtgcagggggcagaaggtgctgggcgtcctggtgaccgatgccaggaacagaggatgctgggacctcccaagggggtctttggggaggagtgggaagggtcaggccacacaggctgtgcctttcctcctcctgtgtctacacgtgggtgatggggccacaatgacgacctctgagccgtgttgaagcagcaccgcgtttctggcgtgcgttaaggtgacccgcactgagagccggggtccccctgcgcctgccggggaggaaccgggtgcgaagcatcccagggccagacggagctgccccctgagcgccgggcctcgctgctgctgggatctcggggccagatggagccgccttctgagcgctggggtctcactgctgctgggatctcgggctgctccatttgtgctctctcttttccaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggccctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgctgcccggctgcccagagggctggatgcctgccggtccccgagcaagcctgggaactcaggaaaattcacaggacttgggagattctaaatcttaagtgcaattatttttaataaaaggggcatttggaatcag

SEQ ID NO: 2 - DNA, 17751 bp, synthetic construct
  source 1..17751: mol_type = other DNA
  misc_feature 1..17751: note = KnockIn
ctaacaccagctgacgagaaactgctctcccatcaacagcgagacacagatgtccaggacctggaaaagagagagcacaaatggagcagcccgagatcccagcagcagtgagaccccagcgctcagaaggcggctccatctggccccgagatcccagcagcagcgaggcccggcgctcagggggcagctccgtctggccctgggatgcttcgcacccggttcctccccggcaggcgcagggggaccccggctctcagtgcgggtcaccttaacgcacgccagaaacgcggtgctgcttcaacacggctcagaggtcgtcattgtggccccatcacccacgtgtagacacaggaggaggaaaggcacagcctgtgtggcctgacccttcccactcctccccaaagacccccttgggaggtcccagcatcctctgttcctggcatcggtcaccaggacgcccagcaccttctgccccctgcaccacggctgggggacgcgatccctgtgccactctgggccctcttgccttggtgtcggggctgtaggtgaagttggagacagggacaccgttggagaggacctgctggggcgccgtggccacgcccaggacagtcaccttctgcagctgcaggccagctccctcactggtcacacgtaccagctcattcacgatcgtgttctgggccgagcagagagagatgaaaaggcagagaatgtggaggtgtcagcagacagctggggcaggaacacccatgagaacagggactcatgatggcatcagggaggtgacctccctctgtgtacacgaaagggcagagtgcacggccccactgctcactctaggaccccaggccacctgctagagtgtcctagtggcaggtagccatcgtgggacccccaagcccaggtcccctcaccccttctcaaccccgagccggcccctgcccaccagcctgagcagccccaggactcacattcctggccaggaagatgacctgtgtgtaggcccctcgctccagcacttccaggctctctccatcgtcccagaacagctcccctcgggcctccccacccttggtcagggccacagccagggccatgggctgctggcgggactctgtggttgtgaggccagggccctggaaagggaaggacacgtgatgtcatcatccccaccctggtggaggtggaggcctccagggcccgggaatgctaggctggtacagcaccaggctgccagtaactaggatctgcaggaacacctgcggccgcaggggctggaaccccgtggtgatgacccaagaccttccccacctcaagtgctatcccacggctgcccgaggccgggcccggcagaacctccaccatgcagaccactgagctcctgccggccatgctccacgcctttgtggggctgtgccctccccacctcactggcctggctggctccttgataacctacactgcggggggcggcccctcagaagccccacctgggcctctcccccaccatctccctgtgcctcccccagctctgcagtgtgctgtccacacccccaccatagccgcctggcccaggtacctgcagggggatgatgtacccagcccggaggtggacgttgatggtgtccaggggggccggcagcgtcacccactgcccctcgctgtggatggctggctcacggggagctgcaggtgggggtgggaggctgccaagggcctctactggcacctggagggagatgttgctttgaggattctgggccgtgccgaggcccccatgctgtcctcaggaaccacgctctccatctcccggggctccacgtggaggcctaggaggcctggcacagacgggctgaactgtgacttcaggcgcagactcagggctcactgtaggtagggaccctcctggctactgagtggggacagtgcctgagaggcctgggtatgccagctcagcacaggcacagcctgtgatggccgtgggacttggccaggttctgcaagcaggcctcagtctgctcatctgggaaatggggatggtgtgaggagcggagaggctgatgggtgcggagcagccgggtgggggcctggcacatggccgggactcaacacatacgttcctctttccgccacctggtcacaccaactgggtggagggggcaggtgggagggctgctctggtctcccgtctccccagggcttagggtccccagactcaccgtctgcaggtcgtaccatgtgcccaaggggaagtagccagtcacttcggccttcccggcctggagcactggggtgatgagcagggcctccccccacaggagctggtggtccacagtccaggtgctagagtccttggggaacctgcaagggggatgggcacacaggcatacgggtgatgaatgggatggggctggctcatatgcccaccctggggaggcacaggaagaggctgaattctgctctctggagctcaaggtgccggaggacttggaggactcaggtcagacgggcagggatgattccagcagatagaagtcaatgccggagctgggagaagccttctccccggacatgccggcagctgggctcagggcaggaataggggcttgtgcttgaggcaccaggatggacacagcactggcctcactttgctcgctcacttatcctccggtaaccaaggcagcatgtgctcatcagtcaagtatttggaaagtacgaaaaagtatcaacaaagagaaaagatcctcccagtcccacaaccaaaggcaacttgaattaacatctgacaaaaagatgaatgtacatgtgcaagcagctctacggaggattctccgtctaacggagactgtcctgccctgtgacgcagctctgccctgtcctgtttcctgcccccggacgtgcagggggcatccagtgctcagctatgatccctgcatggatacattcatggagcgttcatgccttttgaggtgacttctttaggccctatttctatttgtaaaattaacgcatcaatgtgaatctcaaaaacattgactgatatgtactgttgctttccacaaatgtgccaatttatattccaccctactgtgtcactgtgttcttgccagcaagggatagtcttattttctctaatgtattctaattcatgggcaaaataaatggtgtctttctgcttcaaaatatttcttgcaaaatacatttcttggagtgatgtcagtgaagatggtggagtaagcccctctcaagatcctctcctctgtgaaaacaggaacatcggcaaaaatggtcagagttgacatttttaggactcttgaaactaaccaagggtttgcagccatctggggagcatgtatttaacaaaatggctgaatctttgtaagaacagcaagaatccttggcctagctaagtgttgacggtgctccccacctcgccccagcaaaggctgggagactcacggatgccatctgtgaaggatatctgaccaattattggctgtccactaagctagtcaagcagagactttggtggccacacacaatgaagaacacagaccttatggaattagctcaagaaagtcacaaaacaatcagcaatagcaacgagacctgagggggatggggaatctgatgaccacagcagctgcgtttaaaatgtccagtctgaaacaaaaatgtatgagacacgcaaagaaacaagaaagtatggcccacagatggggggacagtagttaataaaaattgtccctagagaagcccagacattggacttactagacaaagagtttacgtcagctactttaaatgtgttcaaagagctcagcgaaaccgtgtctgaagaactaaaggaaagtgtgagaacatgactcaccagatagggaatatccttaacgagagaaacgtaaaggaatgaaacagaaattctggagttgaaaagagagcaactaaaatgagaagttcactagaggggctcaacagcagatgagaactggtggaaaaaagaatcagtgaccttgaagacagggcagtcagactaagcagtctgaagaacagaaagaaaacacaacgaggaaggagtcaacagagctgcaaagacccctgggcaccgtcaagcataccaatgtgtggaacggaagtcccagagtagaagacagaaaggggcagaaaaaatattttatgaagtaatggctgaaaaaccgcccaagtttgatgaaaaacatcaatgcatacgtccaagaagctcaacaaactcccagtaagataaactcagagatctacacctagacacaccatcgggacttcaaaggccaaagacagagaatctgggaagaggcaagacagaagctactcagcagaaaccacggaggccatgggctgacaagttcaaagtgttcagagaaacgaccgccagtcaagaaccctacgttgagcagaactttcaaaaaatgaaggagaaattaagagactctgaagtcaacaaaaactgagagaattcatcactagcagaactgccctacaataaatactaaagggagtccttctgctgtaaatgaaagatgttagacaagaagtcaaagccacatgaagaaataaaaccatgggtaaaggtaactacatcagtaaatataaaaggcagacagtgtgtatgtactttttgtttgtaacccatttttccctcctatttgacttaaaagataattgaataaggcggtcattataaatctgcgtggatgggctcatcatggatacagacgtgtaacagcacagaggaacgagaggcaatggaaaccacatagaagcaaagcttttttatgctattgaaagcaagttggcatcagttccaacgaggctaaaattaagatgttaattgtaatccctagggcaaccactaaaaaaaaaaaaaaaaaagtgtagtgacagaaacaacaaggtcattaaaatagtaattaaaacacaaattgcgtttcttggatttccgggtgaagaatgtgtggataccattcgtatctcctgttttgcaaacagtgttcctccaatttggttcttagtcttggtcttaccaacgtatgagttctctacctattaggaagagtagctatttgctgttttattgacaatgccttctctgctgttacagtccatgttcagagcagttttaatctttttgtgtttatattcagagatttacatttttttttctgctatggtcaaataaagttcaccttttttttttttttttctctaaatgaaacctttccccagagatacacattcacttatcttttcctgtggagtctttcacagcttggttttttttttttgagatggagtcacacaggctggagtacagtggcgcgatctcggctcactgcaatctcctcctcccaggttcaagggattctctggcctcagcctcctgagtagcagggactacaggcgcccaccaccatgcacggctaatttttgtatttttagtagagatgggtttcaccatgttggccaggctggcctcgaactcttgacctcaggtgatccactcgcctcagcctcccaaagttctgggattacaggcgttagccaccgtgcctggcctccacagcttgatttttgacattaactatttaatttatctggaacttatgtttctatggtatgcaagaaaaataaattgacttctgcaaatagttaactaatcacctcactctctgggaatcctccctcaacacactgatgtgcagggctgttgttaggaactcctcgtgtgtactacggcctgtctgggggatattactgggttttcttgctgcccttcccccttaagccaggcccaaatgttgtctcactcagcggcaacctgctgggtcctggggaacacagcccacagcaggacagggctgcctgggagttacgtgcccctcccccagggcacacatgggccaccgcccctgcctaggtcactcactccaggaagaggggccgggccacggtctcccccgcgacgtgggcctggtggaacagtgtgtagaggtgggggaggagtgcgtagcgcagggtgagggccttcctcatggcctgctgggccggctcgctgaagctgtacggctcctggggctgcagggcaggcgggggcaaaggaagcacttgggtgctggggccgcggtcccctggagtccccgcctcgggagagctgcacttctcagccacccagcatggggtgcttctccagcaggggtgggattcccaggggagagtcttgggtgggtgggatcgcccacctgccatgccgccacccccaccctaccagactgagcaggctgttgtggttccgcatgaaggggtagaaggcccccagctgggtccagcgcacacacagctcctctgaggtgttgcccaggaagccgcagacgtcggccccgaccagaggcacccccagcaggttaaactgcaggatttctgggagggcagagtcaggctggtcctcaggctgctgcagcagagccagctcaggccaagtgacccagagccccacctgctaagtgggtggggggccccggcaagcctcccatagaggcccccggctctactctgctgagcagcccctcctggtaggagctcacctggcacggaggaggcgagctgctcccaggagctccacacgtcccccgtccagtggccggcgtatcggccgtggccagcaaaggtcgagcgggagatcacaaatgggcgtgtcccccgagccttcaccagcgccctggggtggtgggggacaccgtgagggctgtgtggagcggggtcactcgggaaccctgtcaccagcagggcagagctgggagcaaggagctttctgggatgaggcagaggctggggaggaggacggcgaggctactgcccatgtctgcgggcacagttgcctctgtctgggccacctgatgcctgtaggtgagccaggaccccccccagcctcacaggagtcatgaggtccccgctgatgcagactcctgtcagcatccagggtccttcctctccaagccccaggcatgaagtgggaaggagcgctccagcttcaggctggggtgcaggcagcgggtggggagagcactcgggccgccagcgggccccgggtggtctgggcctccgcttttcctcctccctgaggccctgcagaggccccaaccttgtaggacaggctgtgagggcagagcccagtggggcgggacgtggccctcacctgtgggaggcgatggcttcggtcaggccgtagaggttgtgcaggttgtagtgtgtggagagaaactggtggctggaggcacagatggtggccgcctggagggtccccccaaccacccctggaagaggcgggggctggtttccagggagcttcctcccggcaggctccaaggtgccctccctgccccccacctcccaggagcccctgtgcaggtcgctgcctcttcaggctggccctggaggtgcacctgcagccgctccccacgcctctgtggggctggcgttacccgggaacctgggtaatcatctccactgcttctaagacagggctgggcgggtgctggagcctgggctctgcagacccccagacttcacatccaccgtctcttggcccatttcaagcactcccctgcctgtttacctaagcccaaaaccccatcctctcactgattctttgctgggccaaactatagcctcaggcccccaaaccttctcccaggcccatccttgtacttccttgtaaaacccagtttctgccattcctgctaagacagtttggcgagaagccccaacctccaccacccctcaggcgatgtctggtcaccctggcctgccttcagcaagaatcctgctgggtcagttcagccagaatccccctgacccccatgcttcttcttggtaattcctgcctctgagcccaccctgctccctggctagaaatccacttgcccgtgctgcattcgagttgggcctgacctctccccacgggtgggtaggtctccgctgcatggtccctgcgcctatcgagacggctctgagtcgagtcggcctcactgtgctctggcaagcacctgtgagcggcgtctcctccggcagggcctgtgcctctgcacccaccctgtctgcactgcttggctgagtctcccagcctgagcctttctcctggggatcccccccccgcccctttccccgctggcccatctgcttctcagagatgagggtgctaagtctcccaggccagacaagggagtctctgatttgattaagtccccagggtaggtggggggcgagctgaccaggcacgtagggtgggttctccagctcattgttggggcagccgtcctcagagcccctgatgaagttggaaggctcgttcatgtcctgcaagagaagcgctgctggtaggtgactctgcccagagtgaggagggtggggtagtccccagaggccttggggatgctcaggagggggccacacttacaatccacatgccgtcgaagggcacctggtcatggaactcagccaccatgtcctcccaccaggccagggctgtggggttggtgaagtcggggaaggcagtggacccgggccatacctggacaacgagaggctgcagtggggagacccggccccacccagggcctgcatggaagccccactgagcctcagcctgagcagaggggcagcctcactcttagtggacgcccacatttgcaaatctgagagacctgcaccctggaggtcaatgagcagctctttctccacaaggcactgatgcggggccaaggggcctcaaccctgggaaaacgaggggtcacccccaacacgtccacccaggggctgggctgcagaaccagagagcgtcagggagaagggcagcggagacggccccgtgcaagccctgccaggcagcctcacatccgcaccagaccctccctagctgctccctcagcgggggccctgcctggaggcccctggggggctgggaggacaaggaggaccctaaggcctcactgagggtctgcagggcttgtgcctgggccaggcccagctgcccatgaagtcaaaggcctgctgatggaagaactcacggggccattccttgtaggagccaccatcgtcacgggccactgtggcccattcctctcagctgtcccactgggcgtgatggctcctcaaatcccaccgtcttcctgagcagctgccggctccccctggctggaggcctctgctttctaacccccgtcccctggaccctcgccctaccttcccaatcagcggctggccggtctcgttggtgatgaaaacccccctccgcagaccctcgtcgtagggcctgtagctcccggcagggcccgagctgctgatggcaggatcctgggaagagggaaacctgtcaaggtgagggtggcccagagccctggcgccagccacggggaaaactgagacagtgagaggatgaggctggggatgacatcatgcgtgtgtacagcatgcctgcctgtgtacctgcacatgcctgcacacgcctgcctggagaggagccagggccgggcgagtcaggaccagctggctgatgaggggcgcgccggaatggaagctttacctttaccttttctttaaacctctagagtttcccaagtgttctacagtaatcacttttcttttataattaaaaaaatgtgagaaagaaaaaagtctgtgtttccactcctgctcttaagggctactactgtttgctgcctgcagaaggcagggatggaaacaggcagcatcagcacagccccacatctcatctgtgcttcctctgctgaagtggatcttccgggcaggaactgttggatcttttcagctcgccctgcctcccacctcctcctctggccatcgtgccggacaccagactcgggctggaccagcccccaaactgcatccttctggctgtgggactggctcagggaagggcctctttgtgctgggtgactcaagagtcctgctttgggacttttctatgggagctgatgggaagaagtctccgctctgtggtgccagcatgagggctggaagctgccctcactcacgccgtcctttccagggattcagagccggccagaaggagaggcacggccagaaggcacacggagcctccacagttccagcgatacccatgcccaggcccaccgggccctccactttccaggaccaggtgacatcagtttatcatgcccctggccttttgctttaaggcagttcaagtaacctacatctgacagaaccctgagtagcacccggccatttctggggaccctcaaacccgacctccctggacagctggggccaagcctgcaaacttcttgatattccctcgatatcacaaagaccctcagaaaacatcctcggcgaccacacaggcacgaggatgacgctgcacagagaaggagccactgggcaccgggcggcccccttcccaaagacccacagtgtgggggcacacaccacgatcatcatgtagcgccggccgccctggtgcagctcctgcaccatggccgggaagtcccggaagccatccttgttgaacgtgaagtccctccgggagtccatgtagtccaggtcgttccactggacgtcctgcagccacaacacgggaccgtctgctggggcctgaggagacgcgtcccggccagccccttgcctcccctgccaccaccccaactcaccagggggaagtgggccctggtcatgttctccaccacctggcgggtgatagcggtggaggagtagccccagcggcacaggtggaagcccaggccccagtatggcggcatgaacgggtatcctgcaggccaacgccgacttcatgagggagggaggagggagccttggggcgggggccgcggccagggagcaggccctacccacaacgtccaggtactgctgcaccacgctcttgggctctgggcccaggaagatgtagacatccaggatcccacctgtcgacctccagctaagggcagggctcggctgcaggaccacatctggaagggaagcagctctggggttgggggacagattcttcacttggagggctctgcaccccacagatgggccaatcacaggcggagagttgaggctctctccccacgaggagctcagagtggcctggggaagccgtgctaagcctgactcaggaggaacctggcatgggacggaaagatatgtggtgccagcccacctccaggcagggccccagaaccctccttaggagcacagaaggccagagttgggagaggcatcagcccataggactcatttcccagaaaacacaaggcccagcaccgtgcccagggcccctcatgcggacctccagtctccagggcaggcagcacggaggagaccccggcccgggcgctgggcggcgggcagcttacccatggcattgctgtttagcaggaacaccccgtgtgccgacccgccgtcctccagcgccaggtagaaagggtgagacccgtagaggttcgcaccgggctgggacatgcaggagacggcgcttcagcacctgcgctccccagctcagtgcccccgcccgccgcccgccgctgtaccgtgggcgcaaggtcccggttccacagggtgatcctggtccagctggtgctgagcatcaggggactgaggtgctcggcgaggcctgtgatatactgcgagggcagcgaggtggacagctgaaggaactggtccgcaaagaacaggggcgccaccgtcgtgttcagcctgcgggacagaggccagccaggcttgctcacacccaaggggccacacgagcctgagagcacccagagagcacccccgggccctggtgccctgggagggcggagtggcctggccagccctgcagcacactgaccctgggacaggcggccgtgccgagccctgcagcccttggcaccctggcaccagcgtgatcaactctcagggcatatcagaagaggcgcaccgcctggctgggcccacaccacccctgaggggcccccaaggggctgacctccaggaactggctggtaccggaactggcacacatgcctgtcccatgaccccaagcaaggctgggcaggggctgaacagaacctttccaggcctaggagcttgtggccaaagtcctcccagatcccagcccagccatgcctgagaaggacccctgaggtttgctgggggtcaagtgcaaggcaaacagcacctccgagaaatgaaagccgcagataaacccggtccacaggaagacgcgtcctctgttaacacagcacagctaatttcgcaggacgggctcacaccgcacccaccctcccctgggcaagactacgctgagtgctttcaagaccgagatctgataagatgcctgtttgttgtttgttttttgagacggggtcttcagcaaggctgaagtgcagtggcaaaatcatggttcactgcaacctcgaacccctgggctcaagggatcctcttgcctcagcctcctgagtagctgggaccacaggcatgtgccacaattcccagctaattttttatttttattttttaagagatggggtcttgctatgttgcccaggatggtcttgaactcctgggctcaagtgaccctctcgccttggcctcccaaagtgctggaattgcagacatgaaccaccatacctagcttgataagatgctttgagaaacttctcttgggtctctctactcagaggtcagctcagagaggccacccctgtcccctgcatctgcacagctggtttccagagtggcctccccaccccccactcctgcacagctggcttccagagaggccacccctgcccccaacatctgcacagctggcttccagagaggccatccctgtcccctgcgtctgcacagctggcttctagaggggacctccctgccccgtgtctgcacagctggcttctagcgtgctctctgcatgaccactccctgacgctgtgtctgcatggacttgcttatggcttcccctcactgtgtgtctgtccccgggagcaggatccaaaccacccggcacggagggtcagtgcgtggacactgagtaaatgaatgagcaaaaaaagaaaaacgcatttccgaaggcaacttctcttaagattaaggcaaatccaagtgctgtcgttcctgagagttttcctgagttaaatgacaagcaggggcttggtgagtcctgaacacgtgtttacatgggatttagacctatgtgagtaaaagacccagggagcgccctgtgagaaatgcccgtcgccctccccgccgtgtgagaaacaaacatgtgtcgccctgcccatcgtgtgagaaacaaacgcgtgtggccctccctgccgtgtgagaaatgcgcgtcgccctccccatcatgctggcacagagcccagaactcacagcacgcggccgtccagctgccggcgcacgatcaccccgaagggctcctcggagaactccacgctgtagagtggggacggtgcccggctgtggacatgcggggtctccaagggcacctcgtagcgcctgttagctggatctttgatctagaagagatgggggtttattgatgttccccacagccaccttactctccagagaacaacccgcacgccaaggacaggtcaggtcctggtgcagccacacatggatgtgggaccatgggcagcacattcagcctctctgagcctcggctccctcatctgcagagccaggaggaggacgcctcccccaggatggaggtaaaatacagaacacctgataactctgaatttcagataaacaacaagcagcttttctagtataaatacatcccaaattttgcatccttacacaaaaaaacaaagtcatgcctggggtatctgcaattcgggtttactgggcatcctgttttcattcgcaacctctggcagccctactctacctgacccaccttttcataaagatgaatgagccccgagccctgccttctggagtacctgtcaccgtggtgtccccactgctccccgaggggcgctgccattgtctgctcacacctccgctcccagcaagggcccagcacacagtggtgcaacatgcaccccacccttgtgaggtgcgtgggtgtcgatgtccacgcgcaccctctgccctggccgccgcccccgcccctgccctgcccaccgtgaagtggaggcggttctcagtctccatcatcacgtccagccgcagggtcaggatgtccttggggaagaaggtgggggtggtacgggtcagggtggccgtgtagcccatttcagaggagctcaggttctccagcttgtagctggggtagctgggtgggaagaagcaccagggctgccccatctgggctccctgcagcccctgctttgcagggatgtagcaacagccgcgggcctcgcactgttcctgggtgatggccttgtcaggggcgcaatcgaagcggctgttgggggggacgtcgcactgtgtgggcactgctctgggacggccggggtgtgcctgggcatcccggggccctggtctgctggctccctgctggtgagctgggtgagtctcctccaggactggggaggagccactcagctctcggggaaccagcaggaaatcatggagtaggatgtgccccaggagtgcagcggttgccaaggacacgagggcgcagacggccaggagccggtgggagcagggcgggtgcctcactcccatggttggagatggcctggacagctcctacaggcctgcgggagaagcaagcgggctcagcagggaggcgggaggggcggcactcacggggctctcaaagcagctctgagacatcaaccgcggctggcactgcagcacccaggcaggtggggtaaggtggccagggtgggtgttgccctgctgtctagactggggagagggccagaaggaagggcgagaaaagctccagcaggggagtgcagagcacttgcacagtctgctaaaatgttacaaatcaaacacgcttagaatgtccccaggaagaccagcaaggcaggtagacacttgaaacaggccaaacagctgtcgcctgggccagagtgctgtggtgagatcctggctcactgcaacctccacctcccagactcaagcaatcctcccacttcagcctcctgagtagctgggactgtaggcacacaccaccgcacccagctaattttctgtatttttgtagagacgggattttgccatgttacccaggctggtctcgaactcctgagctcaagtgatccaccccccttggccttccgaagtgctgggatttcaggagtgaccacgcctggctgggagccccacttctgcataaaggtgcggcttggtccactgggtgtcagcggaagtgattctggcaactcgtatgtccttagggggacccagtacctttccttgctccctttcctattgactggtgcacggacataccacagtggggaatcctgggcaccgcagctgagggtgacactccaggggtggcaaagccacaagagagaaaggacttaacctctggcgactttgcagatcagaacccctcccggcccccgaggtacatggggaagaaacacagtctctgtaacagtggctgcacctggagcctcgggtacagacacaacaaggatgtcgcgtgcagatgagatatgggggttcgctttcattattctgctggtaaggttaacaagtaccaacgacctttgtttcgcttctctgtacattaatattttatggcaaacattctattagcctggctcctcccaggagcagccggaacccacatgcgattcaatatgcatggatttcaataggggagtccctttggtgtgtgtaatacaccatggggagggcgtggggaaggctgggagagccaccgcacctaggcaagtctgaccccagggaaaggaggaagaaggtgtcctaggagcaaggccatgggggagggatgcagagaggtcctcaaaccaaagtcagccatcagaggggtctgcgtctctgaggaacgggccttggagcagaggcaggcaaggatttcagaaagcagcccctggccagtgaagctccttggagcagaggactgtgcaaccttctctggtactgggagctggtcacataccaggaagaagtgaactcagggcccgagggagtctgacctgtcatgtcatgttacagaaggcttggctgggaacgctctgcccccggccggacgtggtgttccaccgtgggccactggtaaattagggctctctgggctggagggtgaaacattttacccacacttctgacattaaatgtgtagggtttttctcacacccatcaattctgcaactttctggacacggctgggtgtcctacaattcaattcaatcttgacactacctggagtcagcacagactccacaggttaagggctcagtcccacaacactgccttcacttcagaggccaatggcaaatcccaggttgtcacttgtactttcgatcaactagttataaattcagaggttcccaggactccctcttcagggtcacagaactcaggaaagtgcgtttcttacagttgctggtttattacaaaggataaaactcagacagccgtatggaagagatgcgtagggcctggtgtgaggtgggggcagtgatgctgtgctctctgctcgcaccaccctccagtgccttggtgtgttccacaacccggcgggaagccctctgatccctgtggtttcttacggggttccattgtgcaggcatggttgactaagccactggtgattcactcaatctccagcccctcaccccttttccatcccaacccgtcatcaggccttggtctttctggccatctgcttccatcctgaagccacctggtgtccccggtcatcagtcacctcatgagcaaaaggctctctcatcactcggtttccaagggtttcgggtgctatgtgccagcaaccagggacaaagaccagagacagtcacccctggcctggcctaagggggtctttgtgattgagacattcagatttcagagctggcccaggggccccctctcaggccacggtgctcctcaccccagggattcagacccagccatgtgcgtcctgcaggagcaggagaggtggctcgggtcccggccaggactgagcactgcgtcgatcaaaaggggaaaggaaaccctagaaacgggaagatgcagcctccagccccaagcccagcacaggagggcggctccccgttcatctctaagctgaactcagataaagaccgggaggactagaggtctggggctcaagctccgctcaggggaagaggctgggggcacggctaggcacgttcaaacccgcttctgggatgttaccgccggcagcgcggggcagacgtcaggtgtctcacccgctccgtgcctcagtttccccgtcagctgcggcacgcgcagagcctccctcgctgaacaacgggcggacgaggagaacctagaggtggccggggtccttcgcgtcaccgaggtcactgtcctacctgctgcctcatccccgacctccacccgcggccccggcgacaacctcagctttcccaactgagaggccgcgcgggcagccgaccgccccaccgacccggcccgcgctcagggaagccccgcagccccggcccggctcacctccgcgcacgcgcgccctggccgcccgcggagactccggggtcg

SEQ ID NO: 3 - DNA, 1726 bp, synthetic construct
  source 1..1726: mol_type = other DNA
  misc_feature 1..1726: note = CAG promoter
aattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaat

SEQ ID NO: 4 - DNA, 522 bp, synthetic construct
  source 1..522: mol_type = other DNA
  misc_feature 1..522: note = rBG pA
gatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccacaccagccaccaccttctgataggcagcctgcacctgagga

SEQ ID NO: 5 - DNA, 21 bp, synthetic construct
  source 1..21: mol_type = other DNA
  misc_feature 1..21: note = GAA TV1 Fwd
aaggaccccggccacctctag

SEQ ID NO: 6 - DNA, 19 bp, synthetic construct
  source 1..19: mol_type = other DNA
  misc_feature 1..19: note = GAA TV1 Rev
cctacaggcccgctccgtg

SEQ ID NO: 7 - DNA, 19 bp, synthetic construct
  source 1..19: mol_type = other DNA
  misc_feature 1..19: note = GAA TV2 Fwd
cgcggaggttctcctcgtc

SEQ ID NO: 8 - DNA, 17 bp, synthetic construct
  source 1..17: mol_type = other DNA
  misc_feature 1..17: note = GAA TV2 Rev
agctcctacaggcccgc

SEQ ID NO: 9 - DNA, 17 bp, synthetic construct
  source 1..17: mol_type = other DNA
  misc_feature 1..17: note = GAA TV3 Fwd
cgcggaggcctgtagga

SEQ ID NO: 10 - DNA, 16 bp, synthetic construct
  source 1..16: mol_type = other DNA
  misc_feature 1..16: note = GAA TV3 Rev
aggacacgagggcgca

SEQ ID NO: 11 - DNA, 20 bp, synthetic construct
  source 1..20: mol_type = other DNA
  misc_feature 1..20: note = GAA Ex1-5 Fwd
ggaaactgaggcacggagcg

SEQ ID NO: 12 - DNA, 21 bp, synthetic construct
  source 1..21: mol_type = other DNA
  misc_feature 1..21: note = GAA Ex1-5 Rev
ggaccacatccatggcattgc

SEQ ID NO: 13 - DNA, 28396 bp, synthetic construct
  source 1..28396: mol_type = other DNA
  misc_feature 1..28396: note = Sequence of the final targeting vector
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacgcggccgcattctggtaccacgagcgaccagagttgtcacaaggccgcaagaacaggggaggtggggggctcagggacagaaaaaaaagtatgtgtattttgagagcagggttgggaggcctctcctgaaaagggtataaacgtggagtaggcaatacccaggcaaaaaggggagaccagagtagggggaggggaagagtcctgacccagggaagacattaaaaaggtagtggggtcgactagatgaaggagagcctttctctctgggcaagagcggtgcaatggtgtgtaaaggtagctgagaagacgaaaagggcaagcatcttcctgctaccaggctggggaggcccaggcccacgaccccgaggagagggaacgcagggagactgaggtgacccttctttcccccggggcccggtcgtgtggttcggtgtctcttttctgttggacccttaccttgacccaggcgctgccggggcctgggcccgggctgcggcgcacggcactcccgggaggcagcgagactcgagttaggcccaacgcggcgccacggcgtttcctggccgggaatggcccgtacccgtgaggtgggggtggggggcagaaaaggcggagcgagcccgaggcggggagggggagggccaggggcggagggggccggcactactgtgttggcggactggcgggactagggctgcgtgagtctctgagcgcaggcgggcggcggccgcccctcccccggcggcggcagcggcggcagcggcggcagctcactcagcccgctgcccgagcggaaacgccactgaccgcacggggattcccagtgccggcgccaggggcacgcgggacacgccccctcccgccgcgccattggcctctccgcccaccgccccacacttattggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatgggcggccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttcatgcatgatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccacaccagccaccaccttctgataggcagcctgcacctgaggacaattttaattaactaacaccagctgacgagaaactgctctcccatcaacagcgagacacagatgtccaggacctggaaaagagagagcacaaatggagcagcccgagatcccagcagcagtgagaccccagcgctcagaaggcggctccatctggccccgagatcccagcagcagcgaggcccggcgctcagggggcagctccgtctggccctgggatgcttcgcacccggttcctccccggcaggcgcagggggaccccggctctcagtgcgggtcaccttaacgcacgccagaaacgcggtgctgcttcaacacggctcagaggtcgtcattgtggccccatcacccacgtgtagacacaggaggaggaaaggcacagcctgtgtggcctgacccttcccactcctccccaaagacccccttgggaggtcccagcatcctctgttcctggcatcggtcaccaggacgcccagcaccttctgccccctgcaccacggctgggggacgcgatccctgtgccactctgggccctcttgccttggtgtcggggctgtaggtgaagttggagacagggacaccgttggagaggacctgctggggcgccgtggccacgcccaggacagtcaccttctgcagctgcaggccagctccctcactggtcacacgtaccagctcattcacgatcgtgttctgggccgagcagagagagatgaaaaggcagagaatgtggaggtgtcagcagacagctggggcaggaacacccatgagaacagggactcatgatggcatcagggaggtgacctccctctgtgtacacgaaagggcagagtgcacggccccactgctcactctaggaccccaggccacctgctagagtgtcctagtggcaggtagccatcgtgggacccccaagcccaggtcccctcaccccttctcaaccccgagccggcccctgcccaccagcctgagcagccccaggactcacattcctggccaggaagatgacctgtgtgtaggcccctcgctccagcacttccaggctctctccatcgtcccagaacagctcccctcgggcctccccacccttggtcagggccacagccagggccatgggctgctggcgggactctgtggttgtgaggccagggccctggaaagggaaggacacgtgatgtcatcatccccaccctggtggaggtggaggcctccagggcccgggaatgctaggctggtacagcaccaggctgccagtaactaggatctgcaggaacacctgcggccgcaggggctggaaccccgtggtgatgacccaagaccttccccacctcaagtgctatcccacggctgcccgaggccgggcccggcagaacctccaccatgcagaccactgagctcctgccggccatgctccacgcctttgtggggctgtgccctccccacctcactggcctggctggctccttgataacctacactgcggggggcggcccctcagaagccccacctgggcctctcccccaccatctccctgtgcctcccccagctctgcagtgtgctgtccacacccccaccatagccgcctggcccaggtacctgcagggggatgatgtacccagcccggaggtggacgttgatggtgtccaggggggccggcagcgtcacccactgcccctcgctgtggatggctggctcacggggagctgcaggtgggggtgggaggctgccaagggcctctactggcacctggagggagatgttgctttgaggattctgggccgtgccgaggcccccatgctgtcctcaggaaccacgctctccatctcccggggctccacgtggaggcctaggaggcctggcacagacgggctgaactgtgacttcaggcgcagactcagggctcactgtaggtagggaccctcctggctactgagtggggacagtgcctgagaggcctgggtatgccagctcagcacaggcacagcctgtgatggccgtgggacttggccaggttctgcaagcaggcctcagtctgctcatctgggaaatggggatggtgtgaggagcggagaggctgatgggtgcggagcagccgggtgggggcctggcacatggccgggactcaacacatacgttcctctttccgccacctggtcacaccaactgggtggagggggcaggtgggagggctgctctggtctcccgtctccccagggcttagggtccccagactcaccgtctgcaggtcgtaccatgtgcccaaggggaagtagccagtcacttcggccttcccggcctggagcactggggtgatgagcagggcctccccccacaggagctggtggtccacagtccaggtgctagagtccttggggaacctgcaagggggatgggcacacaggcatacgggtgatgaatgggatggggctggctcatatgcccaccctggggaggcacaggaagaggctgaattctgctctctggagctcaaggtgccggaggacttggaggactcaggtcagacgggcagggatgattccagcagatagaagtcaatgccggagctgggagaagccttctccccggacatgccggcagctgggctcagggcaggaataggggcttgtgcttgaggcaccaggatggacacagcactggcctcactttgctcgctcacttatcctccggtaaccaaggcagcatgtgctcatcagtcaagtatttggaaagtacgaaaaagtatcaacaaagagaaaagatcctcccagtcccacaaccaaaggcaacttgaattaacatctgacaaaaagatgaatgtacatgtgcaagcagctctacggaggattctccgtctaacggagactgtcctgccctgtgacgcagctctgccctgtcctgtttcctgcccccggacgtgcagggggcatccagtgctcagctatgatccctgcatggatacattcatggagcgttcatgccttttgaggtgacttctttaggccctatttctatttgtaaaattaacgcatcaatgtgaatctcaaaaacattgactgatatgtactgttgctttccacaaatgtgccaatttatattccaccctactgtgtcactgtgttcttgccagcaagggatagtcttattttctctaatgtattctaattcatgggcaaaataaatggtgtctttctgcttcaaaatatttcttgcaaaatacatttcttggagtgatgtcagtgaagatggtggagtaagcccctctcaagatcctctcctctgtgaaaacaggaacatcggcaaaaatggtcagagttgacatttttaggactcttgaaactaaccaagggtttgcagccatctggggagcatgtatttaacaaaatggctgaatctttgtaagaacagcaagaatccttggcctagctaagtgttgacggtgctccccacctcgccccagcaaaggctgggagactcacggatgccatctgtgaaggatatctgaccaattattggctgtccactaagctagtcaagcagagactttggtggccacacacaatgaagaacacagaccttatggaattagctcaagaaagtcacaaaacaatcagcaatagcaacgagacctgagggggatggggaatctgatgaccacagcagctgcgtttaaaatgtccagtctgaaacaaaaatgtatgagacacgcaaagaaacaagaaagtatggcccacagatggggggacagtagttaataaaaattgtccctagagaagcccagacattggacttactagacaaagagtttacgtcagctactttaaatgtgttcaaagagctcagcgaaaccgtgtctgaagaactaaaggaaagtgtgagaacatgactcaccagatagggaatatccttaacgagagaaacgtaaaggaatgaaacagaaattctggagttgaaaagagagcaactaaaatgagaagttcactagaggggctcaacagcagatgagaactggtggaaaaaagaatcagtgaccttgaagacagggcagtcagactaagcagtctgaagaacagaaagaaaacacaacgaggaaggagtcaacagagctgcaaagacccctgggcaccgtcaagcataccaatgtgtggaacggaagtcccagagtagaagacagaaaggggcagaaaaaatattttatgaagtaatggctgaaaaaccgcccaagtttgatgaaaaacatcaatgcatacgtccaagaagctcaacaaactcccagtaagataaactcagagatctacacctagacacaccatcgggacttcaaaggccaaagacagagaatctgggaagaggcaagacagaagctactcagcagaaaccacggaggccatgggctgacaagttcaaagtgttcagagaaacgaccgccagtcaagaaccctacgttgagcagaactttcaaaaaatgaaggagaaattaagagactctgaagtcaacaaaaactgagagaattcatcactagcagaactgccctacaataaatactaaagggagtccttctgctgtaaatgaaagatgttagacaagaagtcaaagccacatgaagaaataaaaccatgggtaaaggtaactacatcagtaaatataaaaggcagacagtgtgtatgtactttttgtttgtaacccatttttccctcctatttgacttaaaagataattgaataaggcggtcattataaatctgcgtggatgggctcatcatggatacagacgtgtaacagcacagaggaacgagaggcaatggaaaccacatagaagcaaagcttttttatgctattgaaagcaagttggcatcagttccaacgaggctaaaattaagatgttaattgtaatccctagggcaaccactaaaaaaaaaaaaaaaaaagtgtagtgacagaaacaacaaggtcattaaaatagtaattaaaacacaaattgcgtttcttggatttccgggtgaagaatgtgtggataccattcgtatctcctgttttgcaaacagtgttcctccaatttggttcttagtcttggtcttaccaacgtatgagttctctacctattaggaagagtagctatttgctgttttattgacaatgccttctctgctgttacagtccatgttcagagcagttttaatctttttgtgtttatattcagagatttacatttttttttctgctatggtcaaataaagttcaccttttttttttttttttctctaaatgaaacctttccccagagatacacattcacttatcttttcctgtggagtctttcacagcttggttttttttttttgagatggagtcacacaggctggagtacagtggcgcgatctcggctcactgcaatctcctcctcccaggttcaagggattctctggcctcagcctcctgagtagcagggactacaggcgcccaccaccatgcacggctaatttttgtatttttagtagagatgggtttcaccatgttggccaggctggcctcgaactcttgacctcaggtgatccactcgcctcagcctcccaaagttctgggattacaggcgttagccaccgtgcctggcctccacagcttgatttttgacattaactatttaatttatctggaacttatgtttctatggtatgcaagaaaaataaattgacttctgcaaatagttaactaatcacctcactctctgggaatcctccctcaacacactgatgtgcagggctgttgttaggaactcctcgtgtgtactacggcctgtctgggggatattactgggttttcttgctgcccttcccccttaagccaggcccaaatgttgtctcactcagcggcaacctgctgggtcctggggaacacagcccacagcaggacagggctgcctgggagttacgtgcccctcccccagggcacacatgggccaccgcccctgcctaggtcactcactccaggaagaggggccgggccacggtctcccccgcgacgtgggcctggtggaacagtgtgtagaggtgggggaggagtgcgtagcgcagggtgagggccttcctcatggcctgctgggccggctcgctgaagctgtacggctcctggggctgcagggcaggcgggggcaaaggaagcacttgggtgctggggccgcggtcccctggagtccccgcctcgggagagctgcacttctcagccacccagcatggggtgcttctccagcaggggtgggattcccaggggagagtcttgggtgggtgggatcgcccacctgccatgccgccacccccaccctaccagactgagcaggctgttgtggttccgcatgaaggggtagaaggcccccagctgggtccagcgcacacacagctcctctgaggtgttgcccaggaagccgcagacgtcggccccgaccagaggcacccccagcaggttaaactgcaggatttctgggagggcagagtcaggctggtcctcaggctgctgcagcagagccagctcaggccaagtgacccagagccccacctgctaagtgggtggggggccccggcaagcctcccatagaggcccccggctctactctgctgagcagcccctcctggtaggagctcacctggcacggaggaggcgagctgctcccaggagctccacacgtcccccgtccagtggccggcgtatcggccgtggccagcaaaggtcgagcgggagatcacaaatgggcgtgtcccccgagccttcaccagcgccctggggtggtgggggacaccgtgagggctgtgtggagcggggtcactcgggaaccctgtcaccagcagggcagagctgggagcaaggagctttctgggatgaggcagaggctggggaggaggacggcgaggctactgcccatgtctgcgggcacagttgcctctgtctgggccacctgatgcctgtaggtgagccaggaccccccccagcctcacaggagtcatgaggtccccgctgatgcagactcctgtcagcatccagggtccttcctctccaagccccaggcatgaagtgggaaggagcgctccagcttcaggctggggtgcaggcagcgggtggggagagcactcgggccgccagcgggccccgggtggtctgggcctccgcttttcctcctccctgaggccctgcagaggccccaaccttgtaggacaggctgtgagggcagagcccagtggggcgggacgtggccctcacctgtgggaggcgatggcttcggtcaggccgtagaggttgtgcaggttgtagtgtgtggagagaaactggtggctggaggcacagatggtggccgcctggagggtccccccaaccacccctggaagaggcgggggctggtttccagggagcttcctcccggcaggctccaaggtgccctccctgccccccacctcccaggagcccctgtgcaggtcgctgcctcttcaggctggccctggaggtgcacctgcagccgctccccacgcctctgtggggctggcgttacccgggaacctgggtaatcatctccactgcttctaagacagggctgggcgggtgctggagcctgggctctgcagacccccagacttcacatccaccgtctcttggcccatttcaagcactcccctgcctgtttacctaagcccaaaaccccatcctctcactgattctttgctgggccaaactatagcctcaggcccccaaaccttctcccaggcccatccttgtacttccttgtaaaacccagtttctgccattcctgctaagacagtttggcgagaagccccaacctccaccacccctcaggcgatgtctggtcaccctggcctgccttcagcaagaatcctgctgggtcagttcagccagaatccccctgacccccatgcttcttcttggtaattcctgcctctgagcccaccctgctccctggctagaaatccacttgcccgtgctgcattcgagttgggcctgacctctccccacgggtgggtaggtctccgctgcatggtccctgcgcctatcgagacggctctgagtcgagtcggcctcactgtgctctggcaagcacctgtgagcggcgtctcctccggcagggcctgtgcctctgcacccaccctgtctgcactgcttggctgagtctcccagcctgagcctttctcctggggatcccccccccgcccctttccccgctggcccatctgcttctcagagatgagggtgctaagtctcccaggccagacaagggagtctctgatttgattaagtccccagggtaggtggggggcgagctgaccaggcacgtagggtgggttctccagctcattgttggggcagccgtcctcagagcccctgatgaagttggaaggctcgttcatgtcctgcaagagaagcgctgctggtaggtgactctgcccagagtgaggagggtggggtagtccccagaggccttggggatgctcaggagggggccacacttacaatccacatgccgtcgaagggcacctggtcatggaactcagccaccatgtcctcccaccaggccagggctgtggggttggtgaagtcggggaaggcagtggacccgggccatacctggacaacgagaggctgcagtggggagacccggccccacccagggcctgcatggaagccccactgagcctcagcctgagcagaggggcagcctcactcttagtggacgcccacatttgcaaatctgagagacctgcaccctggaggtcaatgagcagctctttctccacaaggcactgatgcggggccaaggggcctcaaccctgggaaaacgaggggtcacccccaacacgtccacccaggggctgggctgcagaaccagagagcgtcagggagaagggcagcggagacggccccgtgcaagccctgccaggcagcctcacatccgcaccagaccctccctagctgctccctcagcgggggccctgcctggaggcccctggggggctgggaggacaaggaggaccctaaggcctcactgagggtctgcagggcttgtgcctgggccaggcccagctgcccatgaagtcaaaggcctgctgatggaagaactcacggggccattccttgtaggagccaccatcgtcacgggccactgtggcccattcctctcagctgtcccactgggcgtgatggctcctcaaatcccaccgtcttcctgagcagctgccggctccccctggctggaggcctctgctttctaacccccgtcccctggaccctcgccctaccttcccaatcagcggctggccggtctcgttggtgatgaaaacccccctccgcagaccctcgtcgtagggcctgtagctcccggcagggcccgagctgctgatggcaggatcctgggaagagggaaacctgtcaaggtgagggtggcccagagccctggcgccagccacggggaaaactgagacagtgagaggatgaggctggggatgacatcatgcgtgtgtacagcatgcctgcctgtgtacctgcacatgcctgcacacgcctgcctggagaggagccagggccgggcgagtcaggaccagctggctgatgaggggcgcgccggaatggaagctttacctttaccttttctttaaacctctagagtttcccaagtgttctacagtaatcacttttcttttataattaaaaaaatgtgagaaagaaaaaagtctgtgtttccactcctgctcttaagggctactactgtttgctgcctgcagaaggcagggatggaaacaggcagcatcagcacagccccacatctcatctgtgcttcctctgctgaagtggatcttccgggcaggaactgttggatcttttcagctcgccctgcctcccacctcctcctctggccatcgtgccggacaccagactcgggctggaccagcccccaaactgcatccttctggctgtgggactggctcagggaagggcctctttgtgctgggtgactcaagagtcctgctttgggacttttctatgggagctgatgggaagaagtctccgctctgtggtgccagcatgagggctggaagctgccctcactcacgccgtcctttccagggattcagagccggccagaaggagaggcacggccagaaggcacacggagcctccacagttccagcgatacccatgcccaggcccaccgggccctccactttccaggaccaggtgacatcagtttatcatgcccctggccttttgctttaaggcagttcaagtaacctacatctgacagaaccctgagtagcacccggccatttctggggaccctcaaacccgacctccctggacagctggggccaagcctgcaaacttcttgatattccctcgatatcacaaagaccctcagaaaacatcctcggcgaccacacaggcacgaggatgacgctgcacagagaaggagccactgggcaccgggcggcccccttcccaaagacccacagtgtgggggcacacaccacgatcatcatgtagcgccggccgccctggtgcagctcctgcaccatggccgggaagtcccggaagccatccttgttgaacgtgaagtccctccgggagtccatgtagtccaggtcgttccactggacgtcctgcagccacaacacgggaccgtctgctggggcctgaggagacgcgtcccggccagccccttgcctcccctgccaccaccccaactcaccagggggaagtgggccctggtcatgttctccaccacctggcgggtgatagcggtggaggagtagccccagcggcacaggtggaagcccaggccccagtatggcggcatgaacgggtatcctgcaggccaacgccgacttcatgagggagggaggagggagccttggggcgggggccgcggccagggagcaggccctacccacaacgtccaggtactgctgcaccacgctcttgggctctgggcccaggaagatgtagacatccaggatcccacctgtcgacctccagctaagggcagggctcggctgcaggaccacatctggaagggaagcagctctggggttgggggacagattcttcacttggagggctctgcaccccacagatgggccaatcacaggcggagagttgaggctctctccccacgaggagctcagagtggcctggggaagccgtgctaagcctgactcaggaggaacctggcatgggacggaaagatatgtggtgccagcccacctccaggcagggccccagaaccctccttaggagcacagaaggccagagttgggagaggcatcagcccataggactcatttcccagaaaacacaaggcccagcaccgtgcccagggcccctcatgcggacctccagtctccagggcaggcagcacggaggagaccccggcccgggcgctgggcggcgggcagcttacccatggcattgctgtttagcaggaacaccccgtgtgccgacccgccgtcctccagcgccaggtagaaagggtgagacccgtagaggttcgcaccgggctgggacatgcaggagacggcgcttcagcacctgcgctccccagctcagtgcccccgcccgccgcccgccgctgtaccgtgggcgcaaggtcccggttccacagggtgatcctggtccagctggtgctgagcatcaggggactgaggtgctcggcgaggcctgtgatatactgcgagggcagcgaggtggacagctgaaggaactggtccgcaaagaacaggggcgccaccgtcgtgttcagcctgcgggacagaggccagccaggcttgctcacacccaaggggccacacgagcctgagagcacccagagagcacccccgggccctggtgccctgggagggcggagtggcctggccagccctgcagcacactgaccctgggacaggcggccgtgccgagccctgcagcccttggcaccctggcaccagcgtgatcaactctcagggcatatcagaagaggcgcaccgcctggctgggcccacaccacccctgaggggcccccaaggggctgacctccaggaactggctggtaccggaactggcacacatgcctgtcccatgaccccaagcaaggctgggcaggggctgaacagaacctttccaggcctaggagcttgtggccaaagtcctcccagatcccagcccagccatgcctgagaaggacccctgaggtttgctgggggtcaagtgcaaggcaaacagcacctccgagaaatgaaagccgcagataaacccggtccacaggaagacgcgtcctctgttaacacagcacagctaatttcgcaggacgggctcacaccgcacccaccctcccctgggcaagactacgctgagtgctttcaagaccgagatctgataagatgcctgtttgttgtttgttttttgagacggggtcttcagcaaggctgaagtgcagtggcaaaatcatggttcactgcaacctcgaacccctgggctcaagggatcctcttgcctcagcctcctgagtagctgggaccacaggcatgtgccacaattcccagctaattttttatttttattttttaagagatggggtcttgctatgttgcccaggatggtcttgaactcctgggctcaagtgaccctctcgccttggcctcccaaagtgctggaattgcagacatgaaccaccatacctagcttgataagatgctttgagaaacttctcttgggtctctctactcagaggtcagctcagagaggccacccctgtcccctgcatctgcacagctggtttccagagtggcctccccaccccccactcctgcacagctggcttccagagaggccacccctgcccccaacatctgcacagctggcttccagagaggccatccctgtcccctgcgtctgcacagctggcttctagaggggacctccctgccccgtgtctgcacagctggcttctagcgtgctctctgcatgaccactccctgacgctgtgtctgcatggacttgcttatggcttcccctcactgtgtgtctgtccccgggagcaggatccaaaccacccggcacggagggtcagtgcgtggacactgagtaaatgaatgagcaaaaaaagaaaaacgcatttccgaaggcaacttctcttaagattaaggcaaatccaagtgctgtcgttcctgagagttttcctgagttaaatgacaagcaggggcttggtgagtcctgaacacgtgtttacatgggatttagacctatgtgagtaaaagacccagggagcgccctgtgagaaatgcccgtcgccctccccgccgtgtgagaaacaaacatgtgtcgccctgcccatcgtgtgagaaacaaacgcgtgtggccctccctgccgtgtgagaaatgcgcgtcgccctccccatcatgctggcacagagcccagaactcacagcacgcggccgtccagctgccggcgcacgatcaccccgaagggctcctcggagaactccacgctgtagagtggggacggtgcccggctgtggacatgcggggtctccaagggcacctcgtagcgcctgttagctggatctttgatctagaagagatgggggtttattgatgttccccacagccaccttactctccagagaacaacccgcacgccaaggacaggtcaggtcctggtgcagccacacatggatgtgggaccatgggcagcacattcagcctctctgagcctcggctccctcatctgcagagccaggaggaggacgcctcccccaggatggaggtaaaatacagaacacctgataactctgaatttcagataaacaacaagcagcttttctagtataaatacatcccaaattttgcatccttacacaaaaaaacaaagtcatgcctggggtatctgcaattcgggtttactgggcatcctgttttcattcgcaacctctggcagccctactctacctgacccaccttttcataaagatgaatgagccccgagccctgccttctggagtacctgtcaccgtggtgtccccactgctccccgaggggcgctgccattgtctgctcacacctccgctcccagcaagggcccagcacacagtggtgcaacatgcaccccacccttgtgaggtgcgtgggtgtcgatgtccacgcgcaccctctgccctggccgccgcccccgcccctgccctgcccaccgtgaagtggaggcggttctcagtctccatcatcacgtccagccgcagggtcaggatgtccttggggaagaaggtgggggtggtacgggtcagggtggccgtgtagcccatttcagaggagctcaggttctccagcttgtagctggggtagctgggtgggaagaagcaccagggctgccccatctgggctccctgcagcccctgctttgcagggatgtagcaacagccgcgggcctcgcactgttcctgggtgatggccttgtcaggggcgcaatcgaagcggctgttgggggggacgtcgcactgtgtgggcactgctctgggacggccggggtgtgcctgggcatcccggggccctggtctgctggctccctgctggtgagctgggtgagtctcctccaggactggggaggagccactcagctctcggggaaccagcaggaaatcatggagtaggatgtgccccaggagtgcagcggttgccaaggacacgagggcgcagacggccaggagccggtgggagcagggcgggtgcctcactcccatggttggagatggcctggacagctcctacaggcctgcgggagaagcaagcgggctcagcagggaggcgggaggggcggcactcacggggctctcaaagcagctctgagacatcaaccgcggctggcactgcagcacccaggcaggtggggtaaggtggccagggtgggtgttgccctgctgtctagactggggagagggccagaaggaagggcgagaaaagctccagcaggggagtgcagagcacttgcacagtctgctaaaatgttacaaatcaaacacgcttagaatgtccccaggaagaccagcaaggcaggtagacacttgaaacaggccaaacagctgtcgcctgggccagagtgctgtggtgagatcctggctcactgcaacctccacctcccagactcaagcaatcctcccacttcagcctcctgagtagctgggactgtaggcacacaccaccgcacccagctaattttctgtatttttgtagagacgggattttgccatgttacccaggctggtctcgaactcctgagctcaagtgatccaccccccttggccttccgaagtgctgggatttcaggagtgaccacgcctggctgggagccccacttctgcataaaggtgcggcttggtccactgggtgtcagcggaagtgattctggcaactcgtatgtccttagggggacccagtacctttccttgctccctttcctattgactggtgcacggacataccacagtggggaatcctgggcaccgcagctgagggtgacactccaggggtggcaaagccacaagagagaaaggacttaacctctggcgactttgcagatcagaacccctcccggcccccgaggtacatggggaagaaacacagtctctgtaacagtggctgcacctggagcctcgggtacagacacaacaaggatgtcgcgtgcagatgagatatgggggttcgctttcattattctgctggtaaggttaacaagtaccaacgacctttgtttcgcttctctgtacattaatattttatggcaaacattctattagcctggctcctcccaggagcagccggaacccacatgcgattcaatatgcatggatttcaataggggagtccctttggtgtgtgtaatacaccatggggagggcgtggggaaggctgggagagccaccgcacctaggcaagtctgaccccagggaaaggaggaagaaggtgtcctaggagcaaggccatgggggagggatgcagagaggtcctcaaaccaaagtcagccatcagaggggtctgcgtctctgaggaacgggccttggagcagaggcaggcaaggatttcagaaagcagcccctggccagtgaagctccttggagcagaggactgtgcaaccttctctggtactgggagctggtcacataccaggaagaagtgaactcagggcccgagggagtctgacctgtcatgtcatgttacagaaggcttggctgggaacgctctgcccccggccggacgtggtgttccaccgtgggccactggtaaattagggctctctgggctggagggtgaaacattttacccacacttctgacattaaatgtgtagggtttttctcacacccatcaattctgcaactttctggacacggctgggtgtcctacaattcaattcaatcttgacactacctggagtcagcacagactccacaggttaagggctcagtcccacaacactgccttcacttcagaggccaatggcaaatcccaggttgtcacttgtactttcgatcaactagttataaattcagaggttcccaggactccctcttcagggtcacagaactcaggaaagtgcgtttcttacagttgctggtttattacaaaggataaaactcagacagccgtatggaagagatgcgtagggcctggtgtgaggtgggggcagtgatgctgtgctctctgctcgcaccaccctccagtgccttggtgtgttccacaacccggcgggaagccctctgatccctgtggtttcttacggggttccattgtgcaggcatggttgactaagccactggtgattcactcaatctccagcccctcaccccttttccatcccaacccgtcatcaggccttggtctttctggccatctgcttccatcctgaagccacctggtgtccccggtcatcagtcacctcatgagcaaaaggctctctcatcactcggtttccaagggtttcgggtgctatgtgccagcaaccagggacaaagaccagagacagtcacccctggcctggcctaagggggtctttgtgattgagacattcagatttcagagctggcccaggggccccctctcaggccacggtgctcctcaccccagggattcagacccagccatgtgcgtcctgcaggagcaggagaggtggctcgggtcccggccaggactgagcactgcgtcgatcaaaaggggaaaggaaaccctagaaacgggaagatgcagcctccagccccaagcccagcacaggagggcggctccccgttcatctctaagctgaactcagataaagaccgggaggactagaggtctggggctcaagctccgctcaggggaagaggctgggggcacggctaggcacgttcaaacccgcttctgggatgttaccgccggcagcgcggggcagacgtcaggtgtctcacccgctccgtgcctcagtttccccgtcagctgcggcacgcgcagagcctccctcgctgaacaacgggcggacgaggagaacctagaggtggccggggtccttcgcgtcaccgaggtcactgtcctacctgctgcctcatccccgacctccacccgcggccccggcgacaacctcagctttcccaactgagaggccgcgcgggcagccgaccgccccaccgacccggcccgcgctcagggaagccccgcagccccggcccggctcacctccgcgcacgcgcgccctggccgcccgcggagactccggggtcggaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcgacggataccacgtgggcgcgcctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagctattaaccattaatggtttccaggtggatgtctcctcccccaatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttggggatatccacccctaggaggtatcgcgacggatggatccaagaaccagcccgggcggtggagctcgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggacagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 14 - DNA, 2115 bp, synthetic construct
  source 1..2115: mol_type = other DNA
  misc_feature 1..2115: note = Homology arm 1
acgagcgaccagagttgtcacaaggccgcaagaacaggggaggtggggggctcagggacagaaaaaaaagtatgtgtattttgagagcagggttgggaggcctctcctgaaaagggtataaacgtggagtaggcaatacccaggcaaaaaggggagaccagagtagggggaggggaagagtcctgacccagggaagacattaaaaaggtagtggggtcgactagatgaaggagagcctttctctctgggcaagagcggtgcaatggtgtgtaaaggtagctgagaagacgaaaagggcaagcatcttcctgctaccaggctggggaggcccaggcccacgaccccgaggagagggaacgcagggagactgaggtgacccttctttcccccggggcccggtcgtgtggttcggtgtctcttttctgttggacccttaccttgacccaggcgctgccggggcctgggcccgggctgcggcgcacggcactcccgggaggcagcgagactcgagttaggcccaacgcggcgccacggcgtttcctggccgggaatggcccgtacccgtgaggtgggggtggggggcagaaaaggcggagcgagcccgaggcggggagggggagggccaggggcggagggggccggcactactgtgttggcggactggcgggactagggctgcgtgagtctctgagcgcaggcgggcggcggccgcccctcccccggcggcggcagcggcggcagcggcggcagctcactcagcccgctgcccgagcggaaacgccactgaccgcacggggattcccagtgccggcgccaggggcacgcgggacacgccccctcccgccgcgccattggcctctccgcccaccgccccacacttattggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatgggcggccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttc

SEQ ID NO: 15 - DNA, 2153 bp, synthetic construct
  source 1..2153: mol_type = other DNA
  misc_feature 1..2153: note = Homology arm 2
tagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagctattaaccattaatggtttccaggtggatgtctcctcccccaatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttggg

SEQ ID NO: 16 - DNA, 24 bp, synthetic construct
  source 1..24: mol_type = other DNA
  misc_feature 1..24: note = 5'arm forward primer (F2)
tacgccacagggagtccaagaatg

SEQ ID NO: 17 - DNA, 23 bp, synthetic construct
  source 1..23: mol_type = other DNA
  misc_feature 1..23: note = 3'arm reverse primer (R1)
ctggaaatcaggctgcaaatctc

SEQ ID NO: 18 - DNA, 23 bp, synthetic construct
  source 1..23: mol_type = other DNA
  misc_feature 21..23: note = PAM
ctccagtctttctagaagatggg

SEQ ID NO: 19 - DNA, 24 bp, synthetic construct
  source 1..24: mol_type = other DNA
  misc_feature 1..24: note = 3'KI reverse primer (R2)
gcatctgacttctggctaataaag

SEQ ID NO: 20 - DNA, 23 bp, synthetic construct
  source 1..23: mol_type = other DNA
  misc_feature 1..23: note = 5'KI forward primer (F1)
gatggggagagtgaagcagaacg

SEQ ID NO: 21 - DNA, 21 bp, synthetic construct
  source 1..21: mol_type = other DNA
  misc_feature 1..21: note = Forward primer (F5)
attcaggctgcgcaactgttg

SEQ ID NO: 22 - DNA, 23 bp, synthetic construct
  source 1..23: mol_type = other DNA
  misc_feature 1..23: note = Reverse primer (R5)
ctttttgcctgggtattgcctac

SEQ ID NO: 23 - DNA, 24 bp, synthetic construct
  source 1..24: mol_type = other DNA
  misc_feature 1..24: note = Internal control PCR primer F1
gcagaagaggacagatacattcat

SEQ ID NO: 24 - DNA, 24 bp, synthetic construct
  source 1..24: mol_type = other DNA
  misc_feature 1..24: note = Internal control PCR primer R1
cctactgaagaatctatcccacag

SEQ ID NO: 25 - DNA, 26 bp, synthetic construct
  source 1..26: mol_type = other DNA
  misc_feature 1..26: note = Forward primer (F6)
tggtgcttgcctttatgcctttaatc

SEQ ID NO: 26 - DNA, 21 bp, synthetic construct
  source 1..21: mol_type = other DNA
  misc_feature 1..21: note = Reverse primer (R6)
gcatcagagcagccgattgtc

SEQ ID NO: 27 - DNA, 24 bp, synthetic construct
  source 1..24: mol_type = other DNA
  misc_feature 1..24: note = Internal control PCR primer F2
catgccaatggttcactctaaggt

SEQ ID NO: 28 - DNA, 25 bp, synthetic construct
  source 1..25: mol_type = other DNA
  misc_feature 1..25: note = Internal control PCR primer R2
tctctatgtcccaaagtgcagacac

SEQ ID NO: 29 - DNA, 23 bp, synthetic construct
  source 1..23: mol_type = other DNA
  misc_feature 1..23: note = LOPD seq 1
cacttgctctcccaaagtcgctc

SEQ ID NO: 30 - DNA, 21 bp, synthetic construct
  source 1..21: mol_type = other DNA
  misc_feature 1..21: note = LOPD seq 2
atactccgaggcggatcacaa

SEQ ID NO: 31 - DNA, 22 bp, synthetic construct
  source 1..22: mol_type = other DNA
  misc_feature 1..22: note = LOPD seq 3
tcctggggacattctaagcgtg

SEQ ID NO: 32 - DNA, 22 bp, synthetic construct
  source 1..22: mol_type = other DNA; note = PPMO 1
ccagaaggaaggcgagaaaagc

SEQ ID NO: 33 - DNA, 23 bp, synthetic construct
  source 1..23: mol_type = other DNA; note = PPMO 2
  modified_base 11: mod_base = OTHER; note = abasic subunit
ccagaaggaanggcgagaaaagc

SEQ ID NO: 34 - DNA, 23 bp, synthetic construct
  source 1..23: mol_type = other DNA; note = PPMO 3
  modified_base 13: mod_base = OTHER; note = abasic subunit
ccagaaggaaggncgagaaaagc

SEQ ID NO: 35 - DNA, 25 bp, synthetic construct
  source 1..25: mol_type = other DNA; note = PPMO 4
  modified_base 11: mod_base = OTHER; note = abasic subunit
gcactcacggngctctcaaagcagc

SEQ ID NO: 36 - DNA, 25 bp, synthetic construct
  source 1..25: mol_type = other DNA; note = PPMO 5
  modified_base 10..11: mod_base = OTHER; note = abasic subunit
gcactcacgnngctctcaaagcagc

SEQ ID NO: 37 - DNA, 18 bp, synthetic construct
  source 1..18: mol_type = other DNA; note = PPMO 6
gctattaccttaacccag

SEQ ID NO: 38 - DNA, 20 bp, synthetic construct
  source 1..20: mol_type = other DNA; note = Fully Humanized Mice - Wildtype Forward
tcctgagcccaaacacttct

SEQ ID NO: 39 - DNA, 20 bp, synthetic construct
  source 1..20: mol_type = other DNA; note = Fully Humanized Mice - Common
attgttgcacaacgctcttg

SEQ ID NO: 40 - DNA, 20 bp, synthetic construct
  source 1..20: mol_type = other DNA; note = Fully Humanized Mice - Mutant Forward
cgttggctacccgtgatatt

(19) United States
(12) Patent Application Publication
(10) Pub. No.: US 2026/0123610 A1
(43) Pub. Date: May 7, 2026

(54) POMPE DISEASE MOUSE MODEL GENERATION, CHARACTERIZATION AND METHODS OF USE

(71) Applicant: Sarepta Therapeutics, Inc., Cambridge, MA (US)

(72) Inventors: Ryan OLIVER, Cambridge, MA (US); Kevin KIM, Cambridge, MA (US)

(73) Assignee: Sarepta Therapeutics, Inc., Cambridge, MA (US)

(21) Appl. No.: 19/114,326

(22) PCT Filed: Sep. 26, 2023
(86) PCT No.: PCT/US2023/075111

§ 371 Date: Mar. 21, 2025

Related U.S. Application Data
(60) Provisional application No. 63/377,516, filed on Sep. 28, 2022.

(51) Int. Cl. A01K 67/0278; C12N 9/22; C12N 9/26; C12N 15/10; C12N 15/11; C12N 15/113; C12N 15/85; C12Q 1/6806; C12Q 1/686; C12Q 1/6883; G01N 33/573

(52) U.S. Cl. CPC A01K 67/0278; C12N 9/226; C12N 9/2408; C12N 15/1096; C12N 15/111; C12N 15/113; C12N 15/8509; C12Q 1/6806; C12Q 1/686; C12Q 1/6883; G01N 33/573; A01K 2207/15; A01K 2217/072; A01K 2227/105; A01K 2267/0306; C12N 2015/8527; C12N 2310/11; C12N 2310/20; C12N 2830/50; C12Y 302/0102; G01N 2333/924

(57) ABSTRACT

Disclosed herein are transgenic non-human animal models of Pompe disease, methods of making, and methods of using the same. Disclosed herein are also nucleic acid molecules useful for making the non-human animal models of Pompe disease.

### CROSS-REFERENCE TO RELATED APPLICATIONS

The present application claims the priority benefit of U.S. Provisional Application No. 63/377,516 filed Sep. 28, 2022, which is incorporated by reference herein in its entirety.

### RELATED INFORMATION

The contents of any patents, patent applications, and references cited throughout this specification are hereby incorporated by reference in their entireties.

### REFERENCE TO SEQUENCE LISTING SUBMITTED ELECTRONICALLY VIA EFS-WEB

The content of the electronically submitted sequence listing (Name: 4140_0610001_SequenceListing_ST26.xml: Size: 122,920 bytes; and Date of Creation: Sep. 5, 2023), filed with the application, is incorporated herein by reference in its entirety.

### FIELD OF DISCLOSURE

The present disclosure pertains to the medical field including inherited genetic disorders and diseases, more specifically to the generation and methods of use of non-human animal models for the investigation of the etiology of, and for the investigation of therapy for, inherited genetic disorders and diseases.

### BACKGROUND

Pompe disease (glycogen storage disease type II, OMIM #232300) is an inherited autosomal metabolic disorder due to deficiency of acid alpha-glucosidase (GAA) that acts within lysosomes and is responsible of glycogen breakdown to glucose (Hirschhorn R, Reuser A J J. Glycogen storage disease type II: Acid alpha-glucosidase (acid maltase) deficiency. In: Scriver C R, Beaudet A L, Sly W S, et al. (Eds.) The Metabolic & Molecular Bases of Inherited Disease, McGraw-Hill, New York, 2001:3389-420: van der Ploeg A T. Reuser A J. Pompe's disease. Lancet 2008; 372:1342-53; Toscano A, Musumeci O. Pathophysiological mechanisms in Glycogenosis type II. In: Filosto M, Toscano A, Padovani A, editors. Advances in Diagnosis and Management of Glycogenosis II. New York: Nova Science Publisher Inc; 2012:17-21).

Glycogen accumulates in the lysosome as well as in the cytoplasm leading to tissue damage both directly and by affecting different downstream metabolic pathways including the autophagic process. Although cardiac and skeletal muscles are the main tissues involved, GAA deficiency is ubiquitous, and Pompe disease is considered a multisystem disorder (Chan J, et al., The emerging phenotype of late-onset Pompe disease: A systematic literature review. Mol Genet Metab 2017:120:163-72: Montagnese F, et al. Clinical and molecular aspects of 30 patients with late-onset Pompe disease (LOPD): unusual features and response to treatment. J Neurol 2015; 262:968-78; van Capelle C I, et al. Childhood Pompe disease: clinical spectrum and genotype in 31 patients. Orphanet J Rare Dis 2016; 11:65).

Pompe disease is a progressive disorder and based on the age at onset, Pompe disease can manifest as a severe infantile form (IOPD), presenting with cardiac hypertrophy, respiratory dysfunction and floppiness, and as a late onset form (LOPD), which is more benign and more heterogeneous with respiratory and skeletal muscles involvement (van der Ploeg A T, Reuser A J. Pompe's disease. Lancet 2008:372:1342-53).

In LOPD, the first clinical manifestation can be either proximal muscle weakness or other complaints such as exercise intolerance, muscle pain or even isolated hyperCKemia. The clinical presentations are similar to those in other hereditary or acquired muscle disorders such as, for example, limb-girdle muscular dystrophies (LGMD), other muscle glycogenosis, and inflammatory mvopathies (Preisler N. et al. Late-onset Pompe disease is prevalent in unclassified limb-girdle muscular dystrophies. Mol Genet Metab 2013; 110:287-9; Savarese M, et al. The genetic basis of undiagnosed muscular dystrophies and myopathies: Results from 504 patients. Neurology 2016; 87:71-6).

Multiple mutations in the acid maltase gene have been shown to cause Pompe disease. In LOPD, the most common mutation is the IVS1 splice site mutation (IVS1-13T-G; 606800.0006), which may be present in heterozvgosity or homozvgosity (e.g., Montalvo, A. L. E, et al., Mutation profile of the GAA gene in 40 Italian patients with late onset glycogen storage disease type II. Hum. Mutat. 27: 999-1006, 2006; Herbert, M. et al., Early-onset of symptoms and clinical course of Pompe disease associated with the c.-32-13T-G variant. Molec. Genet. Metab. 126: 106-116, 2019).

Several animal models exist which mimic the phenotypical manifestations of Pompe disease. For example, the acid maltase-deficient Japanese quails exhibit progressive myopathy and cannot lift their wings, fly, or right themselves from the supine position in the flip test (Kikuchi, T, et al., Clinical and metabolic correction of Pompe disease by enzyme therapy in acid maltase-deficient quail. J. Clin. Invest. 101: 827-833, 1998). In mice in whom the GAA gene was disrupted by gene targeting in embryonic stem cells, homozygosity for the knock-out was associated with lack of enzyme activity and accumulation of glycogen in cardiac and skeletal muscle lvsosomes by 3 weeks of age, with a progressive increase thereafter (Raben, N, et al., Targeted disruption of the acid alpha-glucosidase gene in mice causes an illness with critical features of both infantile and adult human glycogen storage disease type II. J. Biol. Chem. 273: 19086-19092, 1998). Gaa-null mice, also showed increased glycogen levels in cervical spinal cord motor neurons and larger soma size of phrenic neurons, had decreased ventilation during quiet breathing and hypercapnic challenge compared to wild type mice, indicating respiratory insufficiency (DeRuisseau, L. R., et al., Neural deficits contribute to respiratory insufficiency in Pompe disease. Proc. Nat. Acad. Sci. 106: 9419-9424, 2009).

All the currently available Pompe disease muse models carry null mutations for the GAA gene (Gaa^{tm1Rabn}/Gaa^{tm1Rabn}, Bijvoet A G; van de Kamp E H. et al., Generalized glycogen storage and cardiomegaly in a knockout mouse model of Pompe disease, Hum Mol Genet, 1998, 7 (1) 53-62; Gaa^{tm1Rabn}/Gaa^{tm1Rabn}, Raben N, et al., Targeted disruption of the acid alpha-glucosidase gene in mice causes an illness with critical features of both infantile and adult human glycogen storage disease type IL J Biol Chem 1998 273 (30) 19086-92, and Raben N, et al., Modulation of disease severity in mice with targeted disruption of the acid alpha-glucosidase gene, Neuromuscul Disord 2000 10 (4-5) 283-91; Gaa^{tm1Rabn}/Gaa^{tm1Rabn} Tg(CMV-GAA*P545L) #Kjv/0; Khanna R, et al., The pharmacological chaperone AT2220 increases the specific activity and lysosomal delivery of mutant acid alpha-glucosidase, and promotes glycogen reduction in a transgenic mouse model of Pompe disease. PLoS One. 2014; 9(7): e102092; Gaa^{tm2Rabn}/Gaa^{tm2Rabn}, Raben N, et al., Modulation of disease severity in mice with targeted disruption of the acid alpha-glucosidase gene. Neuromuscul Disord. 2000 June; 10(4-5):283-91; Gaa^{tm1Rabn}/Gaa^{tm1Rabn} Raben N, et al., Targeted disruption of the acid alpha-glucosidase gene in mice causes an illness with critical features of both infantile and adult human glycogen storage disease type II. J Biol Chem. 1998 July 24:273(30):19086-92), and none of them harbor the most common IVS1-13T-G mutation.

While these models are useful for investigating a number of possible therapies for Pompe Disease, none of them is suitable for investigating potential therapies targeting specifically the highly prevalent IVS1-13T-G mutation, such as, for example, gene therapies targeting the specific mutation.

For over a decade, enzyme replacement therapy (ERT) with recombinant human acid α-glucosidase (rhGAA) has been the only specific therapy for the disease.

Several studies demonstrated the efficacy of ERT, mainly in IOPD, but it became evident that early initiation of ERT is essential to avoid irreversible muscle damage (Angelini C, et al., Observational clinical study in juvenile-adult glycogenosis type 2 patients undergoing enzyme replacement therapy for up to 4 years. J Neurol 2012; 259:952-8; Chien Y H, et al., Pompe disease: early diagnosis and early treatment make a difference. Pediatr Neonatol 2013; 54:219-27). Therefore, there is a need for an animal model of Pompe disease, which is suitable for the investigation of alternative therapies, including genetic therapies specifically targeting the highly prevalent IVS1-13T-G mutation.

### BRIEF SUMMARY

In some aspects, provide herein are transgenic non-human animal models, comprising a nucleic acid sequence of an acid alpha-glucosidase (GAA) gene, or fragment thereof, comprising a mutation, wherein the mutation causes a defective splicing of the pre-mRNA transcribed from the nucleic acid sequence, and wherein the nucleic acid sequence comprising the mutation would have encoded a polypeptide having a GAA activity, if the nucleic acid sequence would have not comprised the mutation In some aspects, the mutation is a T-G mutation. In some aspects, the mutation is an IVS1-13T-G mutation.

In some aspects, the GAA gene, or fragment thereof, comprising the mutation is transcribed into pre-mRNA. In some aspects, the pre-mRNA transcribed from the GAA gene, or fragment thereof, comprising the mutation is processed by splicing into mature mRNA.

In some aspects, the mature mRNA derived from the pre-mRNA transcribed from the GAA gene, or fragment thereof, comprising the mutation is different from the mature mRNA derived from a pre-mRNA transcribed from a GAA gene, or fragment thereof, not comprising the mutation. In some aspects, the mutation weakens the splice acceptor of GAA exon 2. In some aspects, the mutation leads to skipping of exon 2. In some aspects, the mature mRNA derived from the pre-mRNA transcribed from the GAA gene, or fragment thereof, comprising the mutation does not comprise exon 2.

In some aspects, the mature mRNA derived from the pre-mRNA transcribed from the GAA gene, or fragment thereof, comprising the mutation is translated into a polypeptide having reduced GAA activity compared to a polypeptide translated from the mature mRNA transcribed from the a GAA gene, or fragment thereof, not comprising the mutation. In some aspects, the mature mRNA derived from the pre-mRNA transcribed from the GAA gene, or fragment thereof, comprising the mutation is translated into a polypeptide not having GAA activity.

In some aspects, the non-human animal model is a model of Pompe disease. In some aspects, the non-human animal model is a model of late onset Pompe disease.

In some aspects, the nucleic acid sequence of the GAA gene, or fragment thereof, comprising the mutation comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1 or 2.

In some aspects, the nucleic acid sequence of the GAA gene, or fragment thereof, comprising the mutation is inserted in a Rosa26 locus. In some aspects, the nucleic acid sequence of the GAA gene, or fragment thereof, comprising the mutation is inserted in an endogenous GAA locus.

In some aspects, the nucleic acid sequence of the GAA gene, or fragment thereof, comprising the mutation is operably linked to a heterologous promoter. In some aspects, the heterologous promoter is selected from the group consisting of a CMV early enhancer/chicken R actin (CBA) promoter, CAG promoter, CMV, EF1α, EF1 with a CMV enhancer, a CMV promoter with a CMV enhancer (CMVe/p), a CMV promoter with a SV40 intron. In some aspects, the heterologous promoter is a CAG promoter. In some aspects, the heterologous promoter comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 3.

In some aspects, the nucleic acid sequence of the GAA gene, or fragment thereof, comprising the mutation is operably linked to a heterologous polyadenylation signal. In some aspects, the heterologous polyadenylation signal is an rGB-pA polyadenylation signal. In some aspects the polyadenylation signal comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 4.

In some aspects, the transgenic non-human animal model is generated by RNA-guided CRISPR-Cas nuclease system.

In some aspects, the transgenic non-human animal model is a mouse. In some aspects, the mouse is a C57BL/6 mouse.

In some aspects, the acid alpha-glucosidase (GAA) gene is a human acid alpha-glucosidase (GAA) gene.

In some aspects, at least one copy of an acid alpha-glucosidase gene endogenous to the non-human animal model is present in the genome of the transgenic non-human animal model. In some aspects, all copies of the acid alpha-glucosidase gene endogenous to the non-human animal model are present in the genome of the transgenic non-human animal model. In some aspects, at least one copy of an acid alpha-glucosidase gene endogenous to the non-human animal model is absent from the genome of the transgenic non-human animal model. In some aspects, all copies of the acid alpha-glucosidase gene endogenous to the non-human animal model are absent from the genome of the transgenic non-human animal model.

In some aspects, provide herein are recombinant nucleic acid molecules, comprising a 5′ homology arm, a polyadenylation signal, a nucleic acid sequence of an acid alpha-glucosidase (GAA) gene, or fragment thereof, comprising a mutation, wherein the mutation causes a defective splicing of the pre-mRNA transcribed from the nucleic acid sequence, and wherein the nucleic acid sequence comprising the mutation would have encoded a polypeptide having a GAA activity, if the nucleic acid sequence would have not comprised the mutation, a promoter, a 3′ homology arm.

In some aspects, the recombinant nucleic acid molecules further comprise a Neo (neomycin) resistance gene, an Amp (ampicillin) resistance gene.

In some aspects, the nucleic acid sequence of an acid alpha-glucosidase (GAA) gene, or fragment thereof, comprising the mutation, comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1 or 2.

In some aspects, the promoter is selected from the group consisting of a CMV early enhancer/chicken β actin (CBA) promoter, CAG promoter, CMV, EF1α, EF1α with a CMV enhancer, a CMV promoter with a CMV enhancer (CMVe/p), a CMV promoter with a SV40 intron. In some aspects, the promoter is a CAG promoter. In some aspects, the promoter comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 3.

In some aspects, the polyadenylation signal is an rGB-pA polyadenylation signal. In some aspects, the polyadenylation signal comprises a nucleic acid sequence which is at least 70%, at least 80%, at least 85%, at least 90%, at least 910%, at least 92%, at least 93%, at least 94%, at least 95%, at least 96%, at least 97%, at least 98%, at least 99%, or 100% identical to SEQ ID NO: 4.

In some aspects, the homology arms comprise a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to a region of the Rosa26 locus in a mouse genome. In some aspects, the homology arms comprise a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to a region of the GAA locus in a mouse genome.

In some aspects, the acid alpha-glucosidase (GAA) gene is a human acid alpha-glucosidase (GAA) gene.

In some aspects, provide herein are methods of generating a transgenic mouse, comprising delivering to a cell the recombinant nucleic acid molecule of the disclosure.

In some aspects, the cell is a mouse embryonic stem cell or a one-cell mouse embryo. In some aspects, the methods of generating a transgenic mouse, comprising delivering to a cell the recombinant nucleic acid molecule of the disclosure further comprise delivering to the cell a sgRNA and a Cas9 nuclease. In some aspects, the delivered sgRNA target a locus in the mouse cell genome.

In some aspects, the polyadenylation signal, the nucleic acid sequence of an acid alpha-glucosidase (GAA) gene, or fragment thereof, comprising the mutation, and the promoter, comprised in the recombinant nucleic acid molecule of the disclosure, are stably integrated in a locus in the mouse genome.

In some aspects, the polyadenylation signal, the nucleic acid sequence of an acid alpha-glucosidase (GAA) gene, or fragment thereof, comprising the mutation, the promoter, the Neo (neomycin) resistance gene, and the Amp (ampicillin) resistance gene, comprised in the recombinant nucleic acid molecule of the disclosure, are stably integrated in a locus in the mouse genome.

In some aspects, the locus is a Rosa26 locus or GAA locus.

In some aspects, the cell comprises at least one copy of an acid alpha-glucosidase gene endogenous to the cell. In some aspects, the cell comprises all copies of the acid alpha-glucosidase gene endogenous to the cell. In some aspects, the cell lacks at least one copy of an acid alpha-glucosidase gene endogenous to the cell. In some aspects, the cell lacks all copies of the acid alpha-glucosidase gene endogenous to the cell.

In some aspects, provide herein are methods of testing a splice modulating agent comprising (a) administering a splice modulating agent to the transgenic non-human animal model of the disclosure, (b) obtaining a testing sample from the non-human animal model (c) and assaying for the presence of (i) mature mRNA derived from pre-mRNA transcribed from the GAA gene, or fragment thereof, comprising the mutation, and/or (ii) mature mRNA derived from pre-mRNA transcribed from a GAA gene, or fragment thereof, not comprising the mutation.

In some aspects, the splice modulating agent is a small molecule, or an antisense oligonucleotide. In some aspects, the splice modulating agent is administered to the transgenic non-human animal model. In some aspects, the splice modulating agent is administered to cells, tissues, or organs derived from the transgenic non-human animal model.

In some aspects, the mature mRNA derived from the pre-mRNA transcribed from the GAA gene, or fragment thereof, comprising the mutation, is not different from the mature mRNA derived from a pre-mRNA transcribed from a GAA gene, or fragment thereof, not comprising the mutation, after administration of the splice modulating agent. In some aspects, the mature mRNA derived from the pre-mRNA transcribed from the GAA gene, or fragment thereof, comprising the mutation, comprises exon 2, after administration of the splice modulating agent. In some aspects, the mature mRNA derived from the pre-mRNA transcribed from the GAA gene, or fragment thereof, comprising the mutation is translated into a polypeptide having a GAA activity of a polypeptide translated from the mature mRNA transcribed from a GAA gene, or fragment thereof, not comprising the mutation, after administration of the splice modulating agent.

In some aspects, the mature mRNA derived from the pre-mRNA transcribed from the GAA gene, or fragment thereof, comprising the mutation is different from the mature mRNA derived from a pre-mRNA transcribed from a GAA gene, or fragment thereof, not comprising the mutation, after administration of the splice modulating agent. In some aspects, the mature mRNA derived from the pre-mRNA transcribed from the GAA gene, or fragment thereof, comprising the mutation does not comprise exon 2, after administration of the splice modulating agent. In some aspects, wherein the mature mRNA derived from the pre-mRNA transcribed from the GAA gene, or fragment thereof, comprising the mutation is translated into a polypeptide having reduced GAA activity compared to a polypeptide translated from the mature mRNA transcribed from a GAA gene, or fragment thereof, not comprising the mutation, after administration of the splice modulating agent.

In some aspects, the method of the disclosure comprises extracting the mRNA from the cells, the tissues, or the organs derived from the transgenic non-human animal model prior to administering the splice modulating agent, and after administering the splice modulating agent. In some aspects, the method of the disclosure comprises retrotranscribing the extracted mRNA into cDNA. In some aspects, the cDNA is amplified by a PCR comprising a first pair of primers capable of amplifying an exon junction which is not affected by the mutation, and a second pair of primers capable of amplifying an exon junction which is affected by the mutation. In some aspects, the cDNA is amplified by a PCR comprising a pair of primers capable of amplifying cDNA derived from mRNA comprising exon junctions not affected by the mutation, and cDNA derived from mRNA comprising exon junctions affected by the mutation.

In some aspects, the primers comprise a nucleotide sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NOs: 11-12.

In some aspects, provide herein are methods of generating a transgenic mouse, comprising mating a first transgenic mouse generated by the methods disclosed herein with a second transgenic mouse lacking all copies of a mouse GAA gene.

In some aspects, provide herein are methods of testing a splice modulating agent comprising (a) administering a splice modulating agent to a transgenic non-human animal model disclosed herein, wherein all copies of the acid alpha-glucosidase gene endogenous to the non-human animal model are absent from the genome of the transgenic non-human animal model, (b) obtaining a testing sample from the non-human animal model (c) and assaying for the presence of (i) a protein product translated from a mature mRNA derived from a pre-mRNA transcribed from the GAA gene, or fragment thereof, comprising the mutation, and/or (ii) a protein product translated from a mature mRNA derived from a pre-mRNA transcribed from a GAA gene, or fragment thereof, not comprising the mutation. In some aspects, the splice modulating agent is a small molecule. In some aspects, the splice modulating agent is an antisense oligonucleotide. In some aspects, the splice modulating agent is administered to the transgenic non-human animal model. In some aspects, the splice modulating agent is administered to cells, tissues, or organs derived from the transgenic non-human animal model.

In some aspects, the protein product translated from a mature mRNA derived from a pre-mRNA transcribed from the GAA gene, or fragment thereof, comprising the mutation, is not different from a protein product translated from a mature mRNA derived from a pre-mRNA transcribed from a GAA gene, or fragment thereof, not comprising the mutation, after administration of the splice modulating agent. In some aspects, the protein product translated from a mature mRNA derived from a pre-mRNA transcribed from the GAA gene, or fragment thereof, comprising the mutation, comprises an amino acid sequence encoded by exon 2, after administration of the splice modulating agent. In some aspects, the protein product translated from a mature mRNA derived from a pre-mRNA transcribed from the GAA gene, or fragment thereof, comprising the mutation, has a GAA activity of a polypeptide translated from a mature mRNA transcribed from a GAA gene, or fragment thereof, not comprising the mutation, after administration of the splice modulating agent.

In some aspects, the protein product translated from a mature mRNA derived from a pre-mRNA transcribed from the GAA gene, or fragment thereof, comprising the mutation, is different from a protein product translated from a mature mRNA derived from a pre-mRNA transcribed from a GAA gene, or fragment thereof, not comprising the mutation, after administration of the splice modulating agent. In some aspects, the protein product translated from a mature mRNA derived from a pre-mRNA transcribed from the GAA gene, or fragment thereof, comprising the mutation, does not comprise an amino acid sequence encoded by exon 2, after administration of the splice modulating agent. In some aspects, the protein product translated from a mature mRNA derived from a pre-mRNA transcribed from the GAA gene, or fragment thereof, comprising the mutation, has a reduced GAA activity compared to a polypeptide translated from a mature mRNA transcribed from a GAA gene, or fragment thereof, not comprising the mutation, after administration of the splice modulating agent.

In some aspects, the protein content from the cells, the tissues, or the organs derived from the transgenic non-human animal model prior to administering the splice modulating agent, and after administering the splice modulating agent. In some aspects, the protein content is analyzed by a Western blot assay. In some aspects, the Western blot assay comprises an anti-GAA antibody.

### BRIEF DESCRIPTION OF DRAWINGS

FIG. 1 shows a schematic of the strategy used for the generation of the LOPD mouse of the disclosure. The entire genomic sequence of the human acid alpha-glucosidase (GAA) gene harboring the IVS1 mutation was inserted into the mouse Rosa26 genetic locus by CRISPR/Cas9 genome engineering. The inserted sequence further comprises a CAG promoter and an rBG pA polyadenylation signal.

FIG. 2 shows a schematic of the restriction map of the cloning vector, including, in the 5′ to 3′ orientation: the 5′ Rosa26 homology arm, the rGB pA, the entire genomic sequence of the human acid alpha-glucosidase (GAA) gene harboring the IVS1 mutation, the CAG promoter, the 3′ Rosa26 homology arm, the Neo (neomycin) cassette, the Amp (ampicillin) resistance gene.

FIG. 3 shows the gel electrophoresis of the indicated restriction enzyme digestions, showing the correct insertion of the insert into the cloning vector.

FIG. 4A shows the genotyping strategy for detection of the human GAA gene into the Rosa26 locus into the genome of F1 mice originating from a single F0 founder.

FIG. 4B shows the gel electrophoresis of the PCR reactions performed for the genotyping for detection of the human GAA gene into the Rosa26 locus into the genome of founder F0 knock-in mice. The genotyping analysis showed integration of the human GAA gene into the Rosa26 locus into the genome of F1 mice originating from a single F0 founder.

FIG. 5A shows the genotyping strategy for detection of the cloning vector into the genome of founder F0 knock-in mice (random integration).

FIG. 5B shows the gel electrophoresis of the PCR reactions performed for the genotyping for detection of the cloning vector into the genome of founder F0 knock-in mice (random integration). The genotyping analysis showed the absence of random integration of the cloning vector into the genome of founder F0 knock-in mice.

FIG. 6A-C show the results of the Sanger sequencing of the human GAA gene amplified by PCR from the genomic DNA of F0 knock-in mice, which confirmed both the correct orientation with respect to the 5′ and the 3′ Rosa26 homology arms, and the presence of the IVS1 mutation.

FIG. 7A shows the results of the Multiplex qPCR analysis performed on genomic DNA of F2 mice to determine the genomic copy number of the human GAA gene. All three analyzed mice showed insertion of one single copy of the GAA into the genome.

FIG. 7B shows the results of the qPCR analysis performed on genomic DNA of F2 mice to determine the relative expression levels of the three GAA alternative splicing forms (TV1, TV2, and TV3).

FIG. 8A shows the results of the qPCR analysis performed on genomic DNA of F2 mice to determine the relative expression levels of the human and the mouse GAA gene in different tissues.

FIG. 8B shows the results of the qPCR analysis performed on genomic cDNA derived from RNA extracted from LOPD mouse quadriceps muscles of F2 mice, to determine the relative amount of GAA RNA correctly spliced (exon 1-2 junctions) and total GAA RNA (exon 6-7 junctions). The results are consistent with mis-splicing at the exon 1-2 junction.

FIG. 9 shows the results of the endpoint RT-PCR targeting the exon 1-5 region performed on RNA extracted from LOPD mice (LOPD mouse), indicating a mis-splicing pattern similar to that observed in LOPD patient cells. The endpoint RT-PCR targeting the exon 1-5 region performed on RNA extracted from healthy myotubes (Healthy mvotubes) and of LOPD patient cells (LOPD myotubes) are also shown.

FIG. 10A shows the MiSeq analysis of the exon 1-5 RT-PCR amplification pool, showing several major GAA slice variants in LOPD mouse quadriceps muscle.

FIG. 10B shows a schematic representative of the similarity between major GAA splice variants observed in the LOPD mouse quadriceps muscle and those observed in LOPD patient cells.

FIG. 11A-F show an histo-cytological analysis (PAS, FIGS. 11A-C; and H&E, FIG. 11D-F) of quadriceps (FIGS. 11D-F) and diaphragm (FIG. 11A-C) muscles dissected from GAA^{LOPD(IVS1)+/-} Gaa^{+/+} (FIG. 11B-E), Gaa^{-/-} (JAX 004154) (FIGS. 11A-D), and wild type (FIG. 11C-F) mice.

FIG. 12 shows a MiSeq analysis of an exon 1-5 RT-PCR amplification pool of GAA^{LOPD(IVS1)+/-} Gaa^{+/+} mice treated with PPMO1 or with sterile saline.

FIG. 13A-B show results of a qPCR analysis of the amplification pool obtained from GAA^{LOPD(IVS1)+/-} Ga^{+/+} mice treated with PPMO2, PPMO3, NTC, or saline at the indicated doses.

FIG. 13A shows the % of correctly spliced GAA. FIG. 13B shows the % of mis-spliced SV1,2; SV5; and SV3,7 splice variants as indicated. Data represent mean+/−SE. FIG. 13B shows a schematic of SV1, SV2. SV53, SV5, and SV7.

FIG. 14A-B show the results of a qPCR analysis for GAA expression in RNA pools derived from GAA^{LOPD(IVS1)+/-} Gaa^{+/+} mice treated with PPMO1-5 (FIG. 14A) at the indicated doses or with PPMO in a dose-response experiment for PPMO2 (FIG. 14B) as indicated. Data represent mean+/−SE.

FIG. 15 shows GAA protein expression quantification by Western blot in fully humanized GAA^{LOPD(IVS1)+/-} Gaa^{-/-} mice treated with PPMO2, and in control (NTC and sterile saline-treated) animals. Data represent mean+/−SE.

### DETAILED DESCRIPTION OF THE DISCLOSURE

### 1. Definitions

In order that the present disclosure can be more readily understood, certain terms are first defined. As used in this application, except as otherwise expressly provided herein, each of the following terms shall have the meaning set forth below. Additional definitions are set forth throughout the application.

It is to be noted that, as used herein, the indefinite articles “a” or “an” should be understood to refer to “one or more” of any recited or enumerated component: for example, “a nucleic acid sequence,” is understood to represent one or more nucleic acid sequences, unless stated otherwise. As such, the terms “a” (or “an”), “one or more,” and “at least one” can be used interchangeably herein. The use of the alternative (e.g., “or”) should be understood to mean either one, both, or any combination thereof of the alternatives.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C: A, B, or C: A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” refers to a value that is within an acceptable error range for the particular value as determined by one of ordinary skill in the art, which will depend in part on how the value is measured or determined, i.e., the limitations of the measurement system. For example, “about” can mean within 1 or more than 1 standard deviation per the practice in the art. Alternatively, “about” can mean a range of up to 10% or 20% (i.e., +10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values are provided in the application and claims, unless otherwise stated, the meaning of “about” should be assumed to be within an acceptable error range for that particular value.

The term “at least” prior to a value or series of values is understood to include the values adjacent to the term “at least,” and all subsequent values (numbers, integers, or fractions) that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values (numbers, integers, or fractions), as logical from context, to zero. When “no more than” is present before a series of values or a range, it is understood that “no more than” can modify each of the value in the series or range.

As described herein, any concentration range, percentage range, ratio range or integer range is to be understood to include the value of any integer within the recited range and, when appropriate, fractions thereof (such as one-tenth and one-hundredth of an integer), unless otherwise indicated.

The term “derived from,” as used herein, refers to a component that is isolated from or made using a specified molecule or organism, or information (e.g., amino acid or nucleic acid sequence) from the specified molecule or organism.

As used herein, the term “testing sample” refers to a whole non-human animal model or any portion derived therefrom (e.g., an organ, a tissue, a cell, or any combination thereofl.

“Nucleic acid,” “polynucleotide,” and “oligonucleotide,” are used interchangeably in the present application. These terms refer only to the primary structure of the molecule. Thus, these terms include double- and single-stranded DNA, as well as double- and single-stranded RNA (e.g., messenger RNA (mRNA), plasmid DNA (pDNA), or complementary DNA (cDNA)). The terms “nucleic acid,” “polynucleotide,” and “oligonucleotide,” as used herein, are defined as it is generally understood by the person skilled in the art as a molecule comprising two or more covalently linked nucleosides. Such covalently bound nucleosides can also be referred to as nucleic acid molecules or oligomers. Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides. By “isolated” nucleic acid or polynucleotide is intended a nucleic acid molecule, DNA or RNA, which has been removed from its native environment. An isolated polynucleotide includes recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotides or a nucleic acid can be or can include a regulatory element such as a promoter, ribosome binding site, or a transcription terminator (e.g., polyadenylation signal). Nucleic acids may be comprised in a vector.

As used herein, the terms “ASO,” and “antisense oligomer” are used interchangeably and refer to a polynucleotide comprising nucleotides, that hybridizes to a target nucleic acid molecule (e.g., a pre-mRNA) sequence by Watson-Crick base pairing or wobble base pairing (G-U).

As used herein, the term “splice-switching oligonucleotide,” or “SSO,” refer to antisense reagents (e.g., and antisense oligomers) that modulates splicing by binding splice-sites and/or splicing regulatory sequences and competing or interacting with cis- and trans-acting factors for their targets. SSOs can restore aberrant splicing, modify the relative expression of existing mRNAs or produce novel splice variants that are not normally expressed.

As used herein, the term “specifically hybridizes” refers to the ability of a molecule (e.g., an antisense oligomer, such as an SSO) to hybridize to one nucleic acid sequence (e.g., splice-sites and/or splicing regulatory sequences) with greater affinity than it hvbridizes to another nucleic acid sequence. An antisense oligonucleotide can specifically hybridizes to more than one target sequence.

As used herein, the term “modulate,” or “modulation” refers to a change of amount or quality of a function or activity when compared to the function or activity prior to modulation. For example, modulation includes the change, either an increase (stimulation or induction) or a decrease (inhibition or reduction) in gene expression. As further example, modulation of expression can include perturbing splice site selection of pre-mRNA processing, resulting in a change in the amount of a particular splice-variant present compared to conditions that were not perturbed.

As used herein, “variant” refers to an alternative molecule (e.g., an alternative RNA transcript) that can be encoded and/or produced from a single genomic region of DNA. Variants include, but are not limited to “pre-mRNA variants” which are transcripts produced from the same genomic DNA that differ from other transcripts produced from the same genomic DNA in either their start or stop position and contain both intronic and exonic sequence. Variants also include, but are not limited to “mature mRNA variants,” mature mRNA variants are mRNA molecules which are derived from the same genomic region of DNA, and may be derived from the same or a different pre-mRNA, and are characterized by comprising different splice junctions, or alternate initiation and termination codons from one another.

As used herein, the term “nucleotide” refers to monomeric units of nucleic acid polymers (e.g., deoxyribonucleic acid (DNA) and ribonucleic acid (RNA)). Naturally occurring nucleotides are composed of three subunit molecules: a nucleobase, a five-carbon sugar (ribose or deoxyribose), and a phosphate group consisting of one to three phosphates. As used herein, the term “nucleobases”, also known as “nitrogenous bases” or “bases”, refers to biological compounds that form nucleosides, which, in turn, are components of nucleotides. Naturally occurring “nucleoside” comprise a nucleobase and a five-carbon sugar. Nucleotides, nucleosides, nucleobases, sugar moieties, and phosphate groups may be naturally occurring or modified. The term “naturally occurring nucleotides” includes deoxyribonucleotides and ribonucleotides. The term “modified nucleotides” includes nucleotides with modified or substituted sugar groups, modified nucleobases, modified phosphate groups, and/or having modified backbones.

A nucleobase may be any naturally occurring, such as adenine, guanine, cytosine, thymine and uracil, or any synthetic or modified nucleobase that is capable of hydrogen bonding with a nucleobase present on a target pre-mRNA. Examples of modified nucleobases include, without limitation, hypoxanthine, xanthine, 7-methylguanine, 5,6-dihydrouracil, 5-methylcytosine, and 5-hydroxymethoylcytosine.

As used herein, the term “backbone,” and “backbone structure”, refer to the connection between monomers of a nucleic acid. In naturally occurring oligonucleotides, the backbone comprises a 3′-5′ phosphodiester linkage connecting sugar moieties of the oligomer. The backbone structure may include, for example, phosphorothioate, phosphorodithioate, phosphoroselenoate, phosphorodiselenoate, phosphoroanilothioate, phosphoraniladate, phosphoramidate, and the like. See e.g., LaPlanche et al. Nucleic Acids Res. 14:9081 (1986); Stec et al. J. Am. Chem. Soc. 106:6077 (1984), Stein et al. Nucleic Acids Res. 16:3209 (1988). Zon et al. Anti Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). A backbone structure may not contain phosphorous but rather peptide bonds, for example in a peptide nucleic acid (PNA), or linking groups including carbamate, amides, and linear and cyclic hydrocarbon groups. A backbone modification can be a phosphothioate linkage, or a phosphoramidate linkage.

A sugar moiety may comprise ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-0-Me), 2′-O-methoxyethyl (2′MOE), 2′-O-aminoethyl, 2′F; N3′->P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. A sugar moiety modification may an extra bridge bond, such as in a locked nucleic acid (LNA). A sugar analog may contain a morpholine ring, such as phosphorodiamidate morpholino (PMO). A sugar moiety may comprise a ribofuransyl or 2′deoxyribofuransyl modification. A sugar moiety may comprise 2′4′-constrained 2′O-methyloxyethyl (cMOE) modifications. A sugar moiety may comprise cEt 2′,4′ constrained 2′-0 ethyl BNA modifications. A sugar moiety may comprise tricycloDNA (tcDNA) modifications. A sugar moiety may comprise ethylene nucleic acid (ENA) modifications. A sugar moiety may comprise MCE modifications. Modifications are described in the literature, e.g., by Jarver, et al., 2014, “A Chemical View of Oligonucleotides for Exon Skipping and Related Drug Applications,” Nucleic Acid Therapeutics 24(1): 37-47, incorporated by reference for this purpose herein.

The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may comprise specific functional sequences needed for insertion and/or expression of the desired DNA fragments. A “vector” can be any vehicle for the cloning of and/or transfer of a nucleic acid into a host cell, such as a plasmid, phage, transposon, cosmid, chromosome, artificial chromosome, virus, virion, etc. The term “vector” includes both viral and nonviral vehicles for introducing the nucleic acid into a cell in vitro, ex vivo or in vivo. In some aspects, insertion of a polynucleotide into a suitable vector can be accomplished by ligating the appropriate polynucleotide fragments into a chosen vector that may or not have complementary cohesive termini. Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes described in the literature include: genes providing resistance to neomycin, ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters described in the literature include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

As used herein, the term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a b-D-ribofuranosvl group. RNA encompasses without limitation, double stranded RNA, single stranded RNA, isolated RNA such as partially purified RNA, essentially pure RNA, synthetic RNA_recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA. The term “mRNA,” as used herein, refers to a single stranded RNA that encodes the amino acid sequence of one or more peptide (e.g., oligopeptide, or polypeptide) or protein. The term “mRNA,” as used herein includes in vitro transcribed RNA (IVT RNA) or synthetic RNA. An mRNA molecule may also contain a 5′ untranslated region (5′-UTR), and/or a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

The term “expression” as used herein refers to a process by which a gene produces a biochemical, for example, a polypeptide. The process includes any manifestation of the functional presence of the gene within the cell including, without limitation, gene knock-in, as well as both transient expression and stable expression. It may include, without limitation, transcription of the gene into messenger RNA (mRNA), and the translation of such mRNA into polypeptide(s). Expression of a gene produces a “gene product.” As used herein, a gene product can be either a nucleic acid, e.g., a messenger RNA, or a non-coding RNA, produced by transcription of a gene, or a peptide (e.g., a polypeptide) which is translated from an mRNA transcript. Gene products described herein further include nucleic acids with post transcriptional modifications, e.g., mRNAs which are processed, for example, by capping, splicing, and/or polyadenylation, or peptides with post translational modifications, e.g., methylation, glycosylation, the addition of lipids, association with other protein subunits, proteolytic cleavage, and the like.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain.” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. It can be generated in any manner, including by chemical synthesis.

A polypeptide as disclosed herein can be of a size of about 3 or more, 5 or more. 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

As used herein, the term “coding sequence” or a sequence “encoding” refers to a particular molecule which is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. Although a “stop codon” (e.g., TAG, TGA, or TAA) is not translated into an amino acid, it can be considered to be part of a coding region, but any flanking sequences, for example promoters, ribosome binding sites, transcriptional terminators, introns, and the like, are not part of a coding region. A coding sequence can include, but is not limited to, cDNA from prokarvotic or eukaryotic mRNA, genomic DNA sequences from prokaryotic or eukaryotic DNA, and synthetic DNA sequences. A transcription termination sequence will usually be located 3′ to the coding sequence.

As used herein, the term “exon” refers to coding sections of a DNA molecule, or of an RNA molecule which is transcribed from a DNA molecule that are translated into protein. Exons can be separated by intervening sections of DNA that do not code for proteins, known as “introns”. Therefore, the term “intron”, as used herein, refers to a segment of nucleic acid that is transcribed and is present in the “pre-mRNA” but excised by the splicing machinery and therefore not present in the mature mRNA transcript. Following transcription, new, immature strands of messenger RNA, called “pre-mRNA”, may contain both introns and exons. These pre-mRNA molecules go through a modification process in the nucleus called splicing during which the noncoding introns are cut out and only the coding exons remain in the “mature mRNA’. Splicing produces a mature messenger RNA molecule that is then translated into a protein. The term “first exon” refers to a coding sequence or sequence of nucleic acid that encodes a polypeptide or polypeptide region and the term “second exon” refers to a different second coding sequence or sequence of nucleic acid that encodes a second polypeptide region. Where the two exons are separated by an intervening intron in the pre-mRNA, the splicing machinery operates to remove the intervening intron and join the two exons in the mature mRNA.

The term “polyadenylation signal” refers to a nucleic acid sequence present in the RNA transcript that allows for the transcript, when in the presence of the enzyme polyadenyl transferase, to be polyadenylated.

The term “promoter” refers to a minimal sequence sufficient to direct transcription, preferably in a eukarvotic cell. A promoter is intended as a DNA region which binds RNA polymerase and directs the enzyme to transcribe an operably linked DNA sequence. A DNA sequence is operably linked to a promoter if the promoter is capable of effecting transcription of that DNA sequence. Promoters for use in the invention include viral, mammalian and yeast promoters that provide for high levels of expression, e.g., the CMV early enhancer/chicken β actin (CAG) promoter, or the mammalian cytomegalovirus or CMV promoter. The term “constitutive” promoter refers to a nucleotide sequence that, when operably linked to a polynucleotide encoding or specifying a gene product, results in the production of a gene product in the cell under most or all physiological conditions of the cell. The term “inducible” promoter means that when operably linked to a polynucleotide encoding a specified gene product, it basically results in the production of a gene in the cell only when the inducer corresponding to the promoter is present in the cell. As used herein, the term “regulatory sequence” refers to a nucleic acid sequence capable of regulating the expression of a gene operably linked to said regulatory sequence, non-limiting examples of regulatory sequences are enhancers (a DNA sequence that increases the level of transcription of an operably linked gene), and silencers enhancers (a DNA sequence that decreases the level of transcription of an operably linked gene).

As used herein the term “splicing” refers to the process by which introns are removed from primary transcripts (pre-mRNA) and exons are joined to form the mature mRNA. Introns are removed by the pre-mRNA by cleavage at conserved sequences called “splice sites”, or “splicing sites”. These sites are located at the 5′ and 3′ ends of introns. Most commonly, the RNA sequence that is removed begins with the dinucleotide GU at its 5′ end, and ends with AG at its 3′ end. These consensus sequences are known to be critical, because changing one of the conserved nucleotides may result in the inhibition of splicing. Another important sequence occurs at what is called the branch point, located anywhere from 18 to 40 nucleotides upstream from the 3′ end of an intron. The branch point always contains an adenine, but it is otherwise loosely conserved. A typical sequence is YNYYRAY, where Y indicates a pyrimidine, N denotes any nucleotide, R denotes any purine, and A denotes adenine. Rarely, splice site sequences are found that begin with the dinucleotide AU and end with AC, these are spliced through a similar mechanism.

Splicing occurs in several steps and is catalyzed by small nuclear ribonucleoproteins (snRNPs, commonly pronounced “snurps”). First, the pre-mRNA is cleaved at the 5′ end of the intron following the attachment of a snRNP called U1 to its complementary sequence within the intron. The cut end then attaches to the conserved branch point region downstream through pairing of guanine and adenine nucleotides from the 5′ end and the branch point, respectively, to form a looped structure known as a lariat. The bonding of the guanine and adenine bases takes place via a chemical reaction known as transesterification, in which a hydroxyl (OH) group on a carbon atom of the adenine attacks the bond of the guanine nucleotide at the splice site. The guanine residue is thus cleaved from the RNA strand and forms a new bond with the adenine.

Next, the snRNPs U2 and U4/U6 appear to contribute to positioning of the 5′ end and the branch point in proximity. With the participation of U5, the 3′ end of the intron is brought into proximity, cut, and joined to the 5′ end. This step occurs by transesterification; in this case, an OH group at the 3′ end of the exon attacks the phosphodiester bond at the 3′ splice site. The adjoining exons are covalently bound, and the resulting lariat is released with U2. U5, and U6 bound to it. In addition to consensus sequences at their splice sites, eukaryotic genes with long introns also contain exonic splicing enhancers (ESEs). These sequences, which help position the splicing apparatus, are found in the exons of genes and bind proteins that help recruit splicing machinery to the correct site. Most splicing occurs between exons on a single RNA transcript, but occasionally trans-splicing occurs, in which exons on different pre-mRNAs are ligated together.

The splicing process occurs in cellular machines called spliceosomes, in which the snRNPs are found along with additional proteins. The primary variety of spliceosome is one of the most plentiful structures in the cell, and recently, a secondary type of spliceosome has been identified that processes a minor category of introns. These introns are referred to as U12-type introns because they depend upon the action of a snRNP called U12 (the common introns described above are called U2-tvpe introns). The role of U12-type introns is not yet defined, but their persistence throughout evolution and conservation between homologous genes of widely divergent species suggests an important functional basis.

As used herein, the term “alternative splicing” refers to a deviation from the constitutive splicing in which introns are removed and exon are in the order in which they appear in a gene. In alternative splicing certain exons are skipped resulting in various forms of mature mRNA from a single pre-RNA transcript. Weaker splicing signals at alternative splice sites, shorter exon length or higher sequence conservation surrounding orthologous alternative exons influence the exons that are ultimately included in the mature mRNA. Three possible mechanisms: exon shuffling, exonization of transposable elements and constitutively spliced exons, have been proposed for the origin of alternative splicing. Alternative splicing is the mechanism that accounts for the discrepancy between the number of protein-coding genes (˜25,000) in humans and the >90,000 different proteins that are actually generated.

The terms “operatively linked,” “operatively inserted,” “operatively positioned,” “under control” or “under transcriptional control” means that the promoter is in the correct location and orientation in relation to the nucleic acid to control RNA polymerase initiation and expression of the gene. In some aspects, the term “operably linked” means that a DNA sequence and a regulatory sequence(s) are connected in such a way as to permit gene expression when the appropriate molecules (e.g., transcriptional activator proteins) are bound to the regulatory sequence(s). In some aspects, the term “operably inserted” means that the DNA of interest introduced into the cell is positioned adjacent a DNA sequence which directs transcription and translation of the introduced DNA (i.e., facilitates the production of, e.g., a polypeptide encoded by a DNA of interest).

As used herein, the term “recombinant DNA/RNA technology” refers to the manipulation of nucleic acid sequences outside of an organism. This technology comprises, but is not limited to, combining nucleic acid sequences (e.g., coding sequences, regulatory elements (e.g., promoters, enhancers, silencers, termination sequences), linkers (e.g., spacers, internal ribosome entry sites, cleavage sites)) derived from a variety of sources, inserting nucleic acid sequences from a variety of sources in appropriate vectors (e.g., delivery vectors, expression vectors, integrating vectors), modifying or altering nucleotide sequences (e.g., by mutagenesis, insertion of modified nucleotides, 5′-capping, polyadenylation), synthesizing artificial nucleotide sequence. A variety of techniques described in the literature (e.g., molecular cloning, polymerase chain reaction (PCR), digestion with restriction enzymes, in vitro ligation, mutagenesis, site-directed mutagenesis, prokaryotic and eukarvotic cell transformation or transduction, in vitro DNA/RNA synthesis, in vitro RNA-5′-capping, in vitro RNA-polyadenvlation, complementary DNA (cDNA) synthesis, nucleic acid isolation, and the like) can be used to manipulate nucleic acid sequences outside an organism (see for example Green & Sambrook Molecular Cloning: A Laboratory Manual, volumes 1-3,4^{th} edition).

As used herein, the term “recombinant”, refers to any nucleic acid (e.g., DNA, or RNA), peptide (e.g., oligopeptide, polypeptide, or protein), cell, or organism, which is made by combining genetic material from two or more different sources. For example, “recombinant DNA” molecules are DNA molecules derived from one organism and inserted in a host organism to produce new genetic combinations. For example, “recombinant RNA” molecule (e.g., recombinant mRNA molecules) are RNA molecules derived from one organism and inserted in a host organism to produce the expression of a desired genetic product in the host organism.

As used herein, the term “transgene” or “Tg” refers to the genetic material (e.g., gene) which has been or is about to be artificially inserted into the genome of an animal. The source from which the transgene is derived can be any source, for example, the transgene can derive from any living organism, for example an animal, or the transgene can be artificially synthesized by any of the techniques described in the literature, and the transgene can be manipulated or modified via any of the variety of techniques described in the literature which can be used to manipulate nucleic acid sequences outside of an organism. For example, a transgene can be isolated from the genome of an organism, manipulated outside of the organism to introduce a desired mutation, and then introduced into the genome of another organism. The coding region of the transgene can be operably linked to a promoter or to one or more regulatory sequences, which is capable of directing/modulating the expression of the transgene in the transgenic organism. The transgene can be present as an extrachromosomal element in a cell of the transgenic organism, or can be stably integrated into the genome of a cell of the transgenic organism. A transgene comprised in the genome of a germ cell of a transgenic organism can be transmitted to the offspring of the transgenic organism. A transgene comprised in the genome of a somatic cell of a transgenic organism cannot be transmitted to the offspring of the transgenic organism. A transgene can either integrate randomly, or in a specific genetic locus of the transgenic organism's genome. As used herein, the term “genetic locus”, refers to the physical site or location within a genome of a specific DNA sequence, for example a gene.

A non-human organism (e.g., prokaryotic or eukaryotic organism), which comprises a transgene (e.g., one or more transgenes), is defined as a non-human “transgenic organism”.

A “transgenic organism” (e.g., a mouse), as used herein, refers to any organism who has been genetically modified. For example, a transgenic organism is an organism whose genome has been genetically modified to comprise one or more transgenes, and/or to eliminate or inactivate (totally or partially) one or more specific genes. A transgenic organisms whose genome has been genetically modified to comprise a transgene (e.g., at a specific genetic locus, “targeted mutant”), is also referred to as a knock-in organism (e.g., knock-in mouse). A transgenic organisms whose genome has been genetically modified to achieve complete loss or inactivation of a gene is also referred to as knock-out organisms, or null-organism (e.g., knock-out mouse, or null-mouse). A transgenic organisms whose genome has been genetically modified to achieve partial loss or partial inactivation of a gene is also referred to as knock-dovn organisms (e.g., knock-down mouse).

The term “transgenic organism” also encompasses “conditional transgenic organisms”, where the genetic alteration can occur upon satisfaction of certain conditions, such as, exposure of the animal to a substance that promotes the genetic alteration, introduction of an enzyme that promotes the genetic alteration (e.g., Cre in the Cre-lox system), or other conditions that direct the genetic alteration at any time post-fertilization, or post-natally. A “transgenic organism”, can, for example, be a non-human animal, for example a non-human mammal, such as a mouse.

A transgenic organism can be also generated by replication of a parental transgenic organism, for example, by breeding parental transgenic organisms carrying one or more transgenes in their germ line cells genome, lacking (totally or partially) one or more genes in their germ line cells genome, or having one or more (totally or partially) inactivated genes in their germ line cells genome.

An organism can be genetically manipulated to obtain a transgenic organism by any of the techniques described in the literature. For example, an organism, such as a mouse, can be genetically manipulated, by retroviral infection of mouse embryos, by microinjection of foreign DNA into one-cell mouse embryos, or by genetic manipulation of mouse embryonic stem cells. An organism can be genetically manipulated, for example, by a number of genome editing technologies, including zinc-finger nucleases (ZFNs), transcription activator-like effector nucleases (TALENs), or the RNA-guided CRISPR-Cas nuclease system. ZFNs and TALENs use a strategy of tethering endonuclease catalytic domains to modular DNA-binding proteins for inducing targeted DNA double-stranded breaks (DSBs) at specific genomic loci. The CRISPR-Cas nuclease system is based on the use of the Cas9 nuclease which is guided by small RNAs through Watson-Crick base pairing to the target DNA.

As used herein, the term “mutation”, refers to any change in the DNA sequence of an organism. A mutation can indicate the substitution of one or more bases in the DNA sequence with one or more different bases (bases substitution, e.g., a T-G mutation). A mutation can also indicate the deletion or the insertion of one or more bases in the DNA sequence. Mutations can result from errors in DNA replication during cell division, exposure to mutagens, viral infection, or can be artificially introduced in a gene by any of the different techniques described in the literature (e.g., homologous recombination, or site directed mutagenesis). Germline mutations can be transmitted on to the offspring, while somatic mutations cannot.

CRISPR-Cas is a microbial adaptive immune system that uses RNA-guided nucleases to cleave foreign genetic elements. Three types (I-III) of CRISPR systems have been identified across a wide range of bacterial and archaeal hosts, wherein each system comprises a cluster of CRISPR-associated (Cas) genes, noncoding RNAs and a distinctive array of repetitive elements (direct repeats). These repeats are interspaced by short variable sequences derived from exogenous DNA targets known as protospacers, and together they constitute the CRISPR RNA (crRNA) array. Within the DNA target, each protospacer is always associated with a protospacer adjacent motif (PAM), which can vary depending on the specific CRISPR system.

The Type II CRISPR system is one of the best characterized, consisting of the nuclease Cas9, the crRNA array that encodes the guide RNAs and a required auxiliary trans-activating crRNA (tracrRNA) that facilitates the processing of the crRNA array into discrete units. Each crRNA unit then contains a 20-nt guide sequence and a partial direct repeat, where the former directs Cas9 to a 20-bp DNA target via Watson-Crick base pairing.

The RNA-guided nuclease function of CRISPR-Cas is reconstituted in mammalian cells through the heterologous expression of human codon-optimized Cas9 and the requisite RNA components. Furthermore, the crRNA and tracrRNA can be fused together to create a chimeric, single-guide RNA (sgRNA). Cas9 can thus be re-directed toward almost any target of interest in immediate vicinity of the PAM sequence by altering the 20-nt guide sequence within the sgRNA.

Cas9 then generates sequence-specific nuclease-induced DNA nicking or double-strand breaks (DSBs). Upon cleavage by Cas9, the target locus typically undergoes one of two major pathways for DNA damage repair: the error-prone nonhomologous end-joining (NHEJ) or the high-fidelity homology-directed repair (HDR) pathway, both of which can be used to achieve a desired editing outcome. In the absence of a repair template, DSBs are re-ligated through the NHEJ process, which leaves scars in the form of insertion/deletion (indel) mutations. NHEJ can be harnessed to mediate gene knockouts, as indels occurring within a coding exon can lead to frameshift mutations and premature stop codons. Multiple DSBs can additionally be exploited to mediate larger deletions in the genome. HDR is an alternative major DNA repair pathway. Although HDR typically occurs at lower and substantially more variable frequencies than NHEJ, it can be leveraged to generate precise, defined modifications at a target locus in the presence of an exogenously introduced repair template. The repair template can either be in the form of conventional double-stranded DNA targeting constructs with homology arms flanking the insertion sequence, or single-stranded DNA oligonucleotides (ssODNs). The latter provides an effective and simple method for making small edits in the genome, such as the introduction of single-nucleotide mutations for probing causal genetic variation. Unlike NHEJ. HDR is generally active only in dividing cells, and its efficiency can vary widely depending on the cell type and state, as well as the genomic locus and repair template.

As used herein, the term “cell” or “cells” refers not only to the particular subject cell, but also to the progeny or to the potential progeny of such cell(s). The scope of the term as used herein also encompasses the progeny that may or may not in fact be identical to the parent cell because certain modifications may occur in succeeding generations due to either mutation or environmental influences.

As used herein the terms “acid alpha-glucosidase,” “alpha-glucosidase,” “acid alpha-1,4-glucosidase.” or “acid maltase” (GAA), refers to a polypeptide having an enzymatic activity involved in the degradation of glycogen. GAA can hydrolyze glycogen to glucose (i.e., GAA activity). As used herein, the term “GGA gene” refers to any nucleic acid sequence encoding a polypeptide (e.g., an enzyme) having GAA activity (i.e., capable of being involved in the degradation (hydrolysis) of glycogen to glucose). For example, in humans a GAA gene is found on the long arm of chromosome 17 (17q25.2-q25.3) and consists of 20 exons. The GAA pre-RNA is subject to alternative splicing, which generates mature RNAs comprising different exons combination. The IVS1-13T-G (c.-32-13T>G) mutation weakens the splice acceptor of GAA exon 2 and leads to a splicing defect and skipping of exon 2. The a splicing defect and skipping of exon 2 leads to low level of active enzyme (12% of normal) generated from the leakage of normally spliced mRNA.

As used herein, the term “GAA splicing mutant”, refers to any nucleic acid sequence comprising a mutation (in respect to the wild type GAA gene), wherein the mutation causes a defective splicing of the pre-mRNA transcribed from the nucleic acid sequence, and wherein the nucleic acid sequence comprising the mutation would have encoded a polypeptide (e.g., an enzyme) having GAA activity (i.e., capable of being involved in the degradation (hydrolysis) of glycogen to glucose), if the nucleic acid sequence would not have comprised the mutation. A nucleic acid sequence comprising the sequence of the human GAA gene, comprising the IVS1-13T-G (c.-32-13T>G) mutation is an example of “GAA splicing mutant”. As used herein, the term “defective splicing”, refers to any form of splicing which is not physiological. For example, any mature mRNA which presents a combination of exons and introns, which is not found in physiological conditions is considered to be derived from a defective splicing.

As used herein, the term “administration” refers to the administration of a composition or substance to a subject or system. Administration to an animal subject (e.g., to a human) can be by any appropriate route. “Administering” refers to the physical introduction of a composition or substance, which may comprising a therapeutic agent, to a subject, using any of the various methods and delivery systems known to those skilled in the art. Examples of routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Administration can also be via a non-parenteral route, for example, orally. Other non-parenteral routes include a topical, epidermal or mucosal route of administration, for example, intranasally, vaginally, rectally, aborally, sublingually or topically. Administering can also be performed, for example, once, a plurality of times, and/or over one or more extended periods.

As used herein, the terms “treat,” “treated,” and “treating” mean both therapeutic and prophylactic treatment or preventative measures wherein the object is to reverse, alleviate, ameliorate, lessen, inhibit, slow down progression, development, severity or recurrence of an undesired symptom, complication, condition, biochemical indicia of a disorder, or disease, or obtain beneficial or desired clinical results. Beneficial or desired clinical results include, but are not limited to, alleviation of symptoms: diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years. As used herein, the term “prophylactic” (e.g., “prophylactic agent”, “prophylactic treatment”. “prophylactically effective amount”), refers to any complete or partial prevention of a disease or symptom thereof and/or can be therapeutic in terms of a partial or complete cure for a disease and/or adverse effect and/or symptom attributable to the disease.

As used herein, the term “gene therapy” is the administration of nucleic acid sequences (e.g., a polynucleotide comprising a promoter operably linked to a nucleic acid encoding an immunomodulatory protein (e.g., a cytokine or subunit thereof) or functional fragment thereof as disclosed herein) into an individual's cells and/or tissues to treat, reduce the symptoms of, or reduce the likelihood of a disease. Gene therapy also includes administration of splice-switching oligonucleotides (SSOs). As used herein, the term “splice-switching oligonucleotides”, refers to short, synthetic, antisense, modified nucleic acids that base-pair with a pre-mRNA and interfere with the splicing of a transcript by blocking/promoting the RNA-RNA base-pairing or protein-RNA binding interactions that occur between components of the splicing machinery and the pre-mRNA. Splice-switching oligonucleotides can be used to prevent aberrant splicing and enhance normal processing of a transcript. An exogenous molecule or sequence is understood to be molecule or sequence not normally occurring in the cell, tissue and/or individual to be treated. Both acquired and congenital diseases are amenable to gene therapy.

As used herein, the term “subject” refers to any organism to which a composition or a substance (e.g., a nucleotide molecule) can be administered, e.g., for experimental, diagnostic, prophylactic, and/or therapeutic purposes. Typical subjects include any animal (e.g., mammals such as mice, rats, rabbits, non-human primates, and humans). A subject can seek or be in need of treatment, require treatment, be receiving treatment, be receiving treatment in the future, or be a human or animal who is under care by a trained professional for a particular disease or condition.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2006, Oxford University Press, provide one of skill with a general dictionary of many of the terms used in this disclosure.

Units, prefixes, and symbols are denoted in their Systeme Intemational de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

Various aspects of the invention are described in further detail in the following subsections.

### 2. Genetically Engineered, Non-Human, Animal Models of Pompe Disease

The invention provides a method for generating a Pompe disease (PD) non-human animal model, by introducing into the non-human animal model's genome a nucleic acid sequence of GAA splicing mutant (here also referred to as mutGAA gene), or a fragment thereof. In some embodiments, the GAA splicing mutant, or fragment thereof, comprises a T-G mutation. In some embodiments, the GAA splicing mutant, or fragment thereof, comprises an IVS1-13T-G mutation (.i.e. “GAA-IVS1-13T-G”) which is associated with Pompe disease. In some embodiments, the nucleic acid sequence of a GAA splicing mutant, or a fragment thereof is comprised in a transgene. In some embodiments, the nucleic acid sequence of a GAA splicing mutant, or a fragment thereof is comprised in a transgene comprised in the non-human animal model's genome. In some embodiments, the non-human animal model of Pompe disease, is a transgenic non-human animal model comprising a transgene comprising a “GAA splicing mutant”. In some embodiments, the non-human animal model of Pompe disease, is a transgenic non-human animal model comprising a transgene comprising a T-G mutation. In some embodiments, the non-human animal model of Pompe disease, is a transgenic non-human animal model comprising a transgene comprising an IVS1-13T-G mutation. In some embodiments, the GAA gene is a human GAA gene, and the “GAA splicing mutant” is a human GAA splicing mutant. In some embodiments the “GAA splicing mutant”, or fragment thereof, comprises a nucleotide sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1 or 2.

In some embodiments, the genome of the transgenic Pompe disease non-human animal model comprises a single copy of the GAA splicing mutant, or a fragment thereof. In some embodiments, the genome of the transgenic Pompe disease non-human animal model comprises more than one copy of the GAA splicing mutant, or fragment thereof. In some embodiments, the genome of the transgenic Pompe disease non-human animal model comprises about 1, about 2, about 3, about 4, about 5, about 6, about 7, about 8, about 9, about 10, about 11, about 12, about 13, about 14, or about 15 copies of the GAA splicing mutant, or fragment thereof. In some embodiments, the GAA splicing mutant, or fragment thereof, comprises a T-G mutation. In some embodiments, the GAA splicing mutant, or fragment thereof, comprises an IVS1-13T-G mutation. In some embodiments, the GAA splicing mutant, or fragment thereof, exists as an extrachromosomal element (e.g., minichromosome, bacterial artificial chromosome (BAC), yeast artificial chromosomes (YAC)) in the transgenic Pompe disease non-human animal model. In some embodiments, the exogenous nucleic acid sequence is stably integrated in the genome of the transgenic Pompe disease non-human animal model. In some embodiments, the GAA splicing mutant, or fragment thereof, is stably integrated into the genome of the transgenic Pompe disease non-human animal model. In some embodiments, the GAA splicing mutant, or fragment thereof, is stably integrated into one chromosome of the transgenic Pompe disease non-human animal model. In some embodiments, the GAA splicing mutant, or fragment thereof, is stably integrated into more than one chromosome of the transgenic Pompe disease non-human animal model. In some embodiments, the GAA splicing mutant, or fragment thereof, is stably integrated into an autosome, or into a sex chromosome of the transgenic Pompe disease non-human animal model. In some embodiments, the GAA splicing mutant, or fragment thereof, is stably integrated into one homologous chromosome of the transgenic Pompe disease non-human animal model. In some embodiments, the GAA splicing mutant, or fragment thereof, is stably integrated into both homologous chromosomes of the transgenic Pompe disease non-human animal model.

In some embodiments, the GAA splicing mutant, or fragment thereof, is randomly integrated into the Pompe disease non-human animal model genome. In some embodiments GAA splicing mutant, or fragment thereof, is integrated into a specific locus of the Pompe disease non-human animal model genome. In some embodiments, the GAA splicing mutant, or fragment thereof, is integrated into the Rosa26 genetic locus of the Pompe disease non-human animal model genome. In some embodiments, the GAA splicing mutant, or fragment thereof, integrated into the Rosa26 genetic locus of the Pompe disease non-human animal model genome does not disrupt the expression of the endogenous GAA gene. In some embodiments, the GAA splicing mutant, or fragment thereof, is integrated into the endogenous GAA genetic locus of the Pompe disease non-human animal model genome. In some embodiments, the GAA splicing mutant, or fragment thereof, integrated into the endogenous GAA genetic locus of the Pompe disease non-human animal model genome disrupts the expression of the endogenous GAA gene. In some embodiments, the GAA splicing mutant, or fragment thereof, integrated into the endogenous GAA genetic locus of the Pompe disease non-human animal model genome does not disrupt the expression of the endogenous GAA gene.

As used herein, the term “Rosa26 locus”, or “Gt(ROSA)26Sor” refers to a specific genetic site (i.e., locus) that is located on mouse chromosome 6, encoding along non-coding RNA (lncRNA) under the control of a constitutive promoter. The Rosa26 locus is ubiquitously expressed in mouse embryos, and it is a widely used site for the integration of transgenes and reporter constructs, and is considered to be one of the ideal locations where knock-ins of interest can be targeted. In some embodiments, the GAA splicing mutant is integrated in a genomic safe harbor locus other than Rosa 26. As used herein, the term genomic safe harbors (GSHs) locus refers to a site in the genome able to accommodate the integration of new genetic material in a manner that ensures that the newly inserted genetic elements: (i) function predictably and (ii) do not cause alterations of the host genome posing a risk to the host cell or organism. Non-limiting example of GSHs loci are Rosa26 locus, Polr2a locus, MYH9 locus, and Hipp 11 intergenic region.

In some embodiments, the genome of the non-human animal model (e.g., the genome of a mouse model) comprises at least one copy of an acid alpha-glucosidase gene endogenous to the non-human animal model (i.e., at least one copy of an acid alpha-glucosidase gene endogenous to the non-human animal model is present in the genome of the transgenic non-human animal model). In some embodiments, the genome of the non-human animal model (e.g., the genome of a mouse model) comprises all the copies (e.g., two copies) of an acid alpha-glucosidase gene endogenous to the non-human animal model (i.e., all copies of an acid alpha-glucosidase gene endogenous to the non-human animal model are present in the genome of the transgenic non-human animal model). In some embodiments, the genome of the non-human animal model (e.g., the genome of a mouse model) lacks at least one of the copies of an acid alpha-glucosidase gene endogenous to the non-human animal model (i.e., at least one copy of an acid alpha-glucosidase gene endogenous to the non-human animal model is absent from the genome of the transgenic non-human animal model). In some embodiments, the genome of the non-human animal model (e.g., the genome of a mouse model) lacks all copies (e.g., two copies) of the acid alpha-glucosidase gene endogenous to the non-human animal model (i.e., all copies of the acid alpha-glucosidase gene endogenous to the non-human animal model are absent from the genome of the transgenic non-human animal model).

In some embodiments, the GAA splicing mutant, or fragment thereof, is comprised in the genome of a somatic cell of the Pompe disease non-human animal model. In some embodiments, the GAA splicing mutant, or fragment thereof, is comprised in the genome of a germ cell of the Pompe disease non-human animal model. In some embodiments GAA splicing mutant, or fragment thereof, is comprised in the genome of somatic and germ cells of the Pompe disease non-human animal model. In some embodiments, the GAA splicing mutant, or fragment thereof, is not transmitted on to the offspring. In some embodiments, the GAA splicing mutant, or fragment thereof, is transmitted on to the offspring.

In some embodiments, the non-human animal model is vertebrate, such as a mammal. The present embodiments are not limited to any one species of animal, but provides for any appropriate non-human species. For example, in certain embodiments, the animal is a non-human mammals, e.g., cow, pig, goat, horse, rodent (such as, rat, mouse, or hamster), etc. In some embodiments, the animal is a rodent, e.g., rat, mouse, hamster, etc. In specific embodiments, the animal is a mouse. For example, as described and exemplified herein, transgenic mice can be produced. Mouse strains that can be used for generating transgenic mice include, but are not limited to, CD-1@Nude mice, CD-1 mice, NU/NU mice, BALB/C Nude mice, BALB/C mice, NIH-Ill mice. SCID™ mice, outbred SCID™ mice, SCID™ Beige mice, C3H mice. C57BL/6 mice, DBA/2 mice, FVB mice, CB17 mice, 129 mice, SJL mice, B6C3F1 mice, BDF1 mice, CDFI mice, CB6F1 mice, CF-1 mice, Swiss Webster mice, SKH1 mice, PGP mice, and B6SJL mice, various substrains (e.g., J or N substrain) within each mouse strain can also be used. Additionally, mice derived from any breeding (e.g., in-breeding, inter-cross-breeding, cross-breeding, or back-cross-breeding) of any mouse strain can be used. As used herein, the term “inbreeding” refers to the mating of closely related individuals or of individuals having closely similar genetic constitutions. As used herein, the term “inter-cross-breeding” refers to breeding from parents of different varieties or species. As used herein, the term “cross-breeding” refers to the mating of purebred parents of two different breeds, varieties, or populations, often with the intention to create offspring that share the traits of both parent lineages. As used herein, the term “back-cross-breeding” refers to mating the crossbred offspring of a two-way cross back to one of the parent breeds. In some embodiments, the non-human animal model is C57BL/6 mouse.

During the initial construction of non-human transgenic animals (i.e., non-human animal models), “chimeras” or “chimeric animals” are generated, in which only a subset of cells have the altered genome (e.g., a genome comprising a transgene). Chimeras are primarily used for breeding to generate transgenic animals with germline transmission, i.e., transgenic animals with an exogenous nucleic acid sequence stably integrated in the genome of germ cells. Animals having a germline heterozygous alteration are produced by breeding of chimeras. Male and female heterozygotes with germline transmission are then bred to produce homozygous transgenic animals. Transgenic animals can also be bred with animals of different genetic backgrounds (e.g., xenograft animal model, various disease animal models, or transgenic animals with different transgenes) to produce transgenic animals with the particular genetic backgrounds. Thus, in some embodiments, the transgenic animals are chimeric transgenic animals. In certain embodiments, the transgenic animals are heterozygous transgenic animals. In other embodiments, the transgenic animals are homozygous transgenic animals. In yet other embodiments, the transgenic animals are homozygous or heterozygous transgenic animals with particular genetic backgrounds (e.g., xenograft animal model, various disease animal models, or transgenic animals with different transgenes).

In some embodiments, the non-human transgenic animal model of Pompe disease is a transgenic mouse, comprising a single copy of a GAA splicing mutant. In some embodiments, the GAA splicing mutant is stably integrated into the Rosa26 locus of the transgenic mouse genome. In some embodiments, the transgenic mouse is a C57BL/6 mouse. In some embodiments, the GAA splicing mutant, comprises an IVS1-13T-G mutation.

In some embodiments, the GAA splicing mutant is expressed in a similar expression pattern in the transgenic mice as mouse GAA is expressed in mice. In some embodiments, the GAA splicing mutant G is expressed in a similar expression pattern in the transgenic mice as human GAA is expressed in humans. In some embodiments, the level of expression of GAA splicing mutant in the transgenic mice is similar to the level of expression of mouse GAA in mice. In some embodiments, the level of expression of GAA splicing mutant G in the transgenic mice is similar to the level of expression of human GAA in humans. In some embodiments, the level of expression of GAA splicing mutant in the transgenic mice is different from the level of expression of mouse GAA in mice. In some embodiments, the level of expression of GAA splicing mutant in the transgenic mice is different from the level of expression of human GAA in humans.

In some embodiments, the levels of GAA splicing mutant expression are directly or indirectly determined by the copy number of GAA splicing mutant, the genomic site where the GAA splicing mutant is integrated, and/or the promoter and/or regulatory regions operably linked to the GAA splicing mutant

The patterns of expression of GAA splicing mutant or mouse GAA (e.g., in transgenic PD mice), as well patterns of expression of GAA in isolated human cells, can be assayed by methods described in the literature, including but not limited to, in situ hybridization, or immunohistochemical staining (HM), etc. The levels of expression of GAA splicing mutant G or mouse GAA (e.g., in transgenic PD mice), as well levels of expression of GAA in isolated human cells, can be measured by methods described in the literature, including but not limited to, Northem blot, Westem blot, RT-PCR, or quantitative RT-PCR. The expression levels of various housekeeping genes (e.g., Hprt. GADPH, β-actin, ubiquitin, or hsp 90) can be measured using similar methods. Relative gene expression levels normalized based on the expression levels of housekeeping genes from the same samples can be compared.

In some embodiments, the GAA splicing mutant gene is expressed in the cells of the non-human Pompe disease animal model. In some embodiments, the GAA splicing mutant gene is transcribed into pre-mRNA in the cells of the non-human Pompe disease animal model. In some embodiments, the pre-mRNA is processed into mature mRNA in the cells of the non-human Pompe disease animal model. In some embodiments, the mature mRNA is translated into a polypeptide in the cells of the non-human animal model.

In some embodiments, the mutation comprised in the GAA splicing mutant gene causes defective splicing. In some embodiments, the pre-mRNA transcribed from the GAA splicing mutant is processed by splicing in a different way compared to the way pre-mRNA transcribed from a GAA gene not comprising the mutation is processed. In some embodiments the polypeptide translated from the mature mRNA transcribed from the GAA splicing mutant gene has reduced GAA activity compared to the polypeptide translated from the mature mRNA transcribed from a GAA gene not comprising the mutation. In some embodiments, the polypeptide translated from the mature mRNA transcribed from the GAA splicing mutant gene has no GAA activity.

### 3. Generation of Genetically Engineered, Non-Human, Animal Models of Pompe Disease

In certain embodiments, the transgenic non-human animal models of Pompe disease of the disclosure are produced by introducing a GAA splicing mutant, or a fragment thereof, into the germline of the non-human animal (e.g., mouse). In some embodiments, the transgenic animals are transgenic mice, produced by introducing a GAA splicing mutant, or a fragment thereof into the germline of the mice.

In some embodiments, the GAA splicing mutant is derived from a wild type GAA gene (i.e., wt-GAA). In some embodiments, the mutation (e.g., a T-G mutation, such as a IVS1-13T-G mutation) may be introduced into a wild type GAA gene by any one of the techniques described in the literature. For example, the mutation can be introduced into the wild type GAA gene by site-directed mutagenesis (SDM), or by homologous recombination (HR). The desired mutation can be introduced into the wt-GAA nucleotide sequence as to obtain the GAA splicing mutant at any time, before, simultaneously, or after the introduction of the transgene into the non-human animal model. In some embodiments, the GAA splicing mutant is not derived from a wild type GAA gene.

The wt-GAA, or the GAA splicing mutant (e.g., comprising a T-G mutation, such as an IVST-13T-G mutation), may be of natural or artificial origin. In some embodiments, the wt-GAA, or the GAA splicing mutant is artificially synthesized by any of the techniques described in the literature. In some embodiments, the wt-GAA, or the GAA splicing mutant, is derived from a non-human animal, examples of non-human animals which can be used in the present embodiments are not limited to any one species of animal, but provides for any appropriate non-human species. For example, in certain embodiments, the animal is a non-human mammals, e.g., cow, pig, goat, horse, rodent (such as, rat, mouse, or hamster), etc. In some embodiments, the wt-GAA, or the GAA splicing mutant, is derived from a human. In some embodiments, the wt-GAA, or the GAA splicing mutant, is derived from an organ derived from a non-human animal, or from a human. In some embodiments, the wt-GAA, or the GAA splicing mutant, is derived from a tissue derived from a non-human animal, or from a human. In some embodiments, the wt-GAA, or the GAA splicing mutant, is derived from a cell derived from a non-human animal, or from a human. In some embodiments, the non-human animal, or the human, do not harbor a mutation in the GAA locus. In some embodiments, the non-human animal, or the human, harbors a mutation in the GAA locus. In some embodiments, the non-human animal, or the human, harbors a T-G mutation in the GAA locus. In some embodiments, the non-human animal, or the human, harbors an IVS1-13T-G mutation in the GAA locus.

In some embodiments, the wt-GAA, or the GAA splicing mutant may be genomic DNA, complementary DNA (cDNA), hybrid sequences, synthetic sequences, or semi-synthetic sequences.

In some embodiments, the wt-GAA, or the GAA splicing mutant can be derived from a genomic library. As used herein, the term “genomic library” refers to a collection of the total genomic DNA from a single organism. The DNA is stored in a population of identical vectors, each containing a different insert of DNA. The vectors of a genomic library can be any type of vectors, non-limiting examples of vectors, which can be used in a genomic library are: plasmids, phage lamba, cosmids, bacteriophage P1 vectors, P1 artificial chromosomes, bacterial artificial chromosomes (BAC), yeast artificial chromosomes (YAC), and the like. A genomic library can be screened to select the vector comprising the nucleic acid of interest, by any of the methods described in the literature.

In some embodiments, a wild type GAA gene comprised in a vector (e.g., a BAC derived from a genomic library) can be modified as to comprise a mutation (e.g., a T-G mutation, such as an IVS1-13T-G mutation), by any one of the described in the literature (e.g., SDM, or HR).

Any nucleotide sequence, which is desired to be operatively linked to GAA splicing mutant, can be operatively linked to the GAA splicing mutant by any one of the techniques described in the literature. Alternatively, any nucleotide sequence which is desired to be operatively linked to GAA splicing mutant, can be operatively linked to the wt-GAA, and the mutation, as to obtain the GAA splicing mutant, may be subsequently introduced into the wt-GAA nucleotide sequence, at any time, before, simultaneously, or after the introduction of the transgene into the non-human animal model.

In some embodiments, the GAA splicing mutant comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1 or 2.

In some embodiments, the GAA splicing mutant, or the wt-GAA, can be cloned (or sub-cloned, if comprised in a vector, for example in a genomic library vector), into any vector comprising any sequence which is desired to be operably linked to the GAA splicing mutant, or to the wt-GAA. For example, the GAA splicing mutant, or the wt-GAA may be cloned (or sub-cloned) in a vector comprising a specific promoter, a specific regulatory element (e.g., an enhancer), a specific polyadenylation signal, and/or any other specific nucleic acid sequence, which is desired to be operably linked to the GAA splicing mutant, or to the wt-GAA.

Molecular cloning techniques are described in the literature (See, e.g., Sambrook, Fritsch and Maniatis, Molecular Cloning: Cold Spring Harbor Laboratory Press (1989).

In some embodiments, the GAA splicing mutant, or the wt-GAA, is operably linked to a promoter, and/or regulatory regions (e.g., in a recombinant nucleic acid molecule). In some embodiments, the GAA splicing mutant, or the wt-GAA, is operably linked to an endogenous promoter, and/or regulatory regions, or to an exogenous promoter and/or regulatory regions. In some embodiments the promoter and/or regulatory regions are homologous (e.g., mouse promoter and/or regulatory regions for a transgenic mouse). In some embodiments, the promoter is homologous (e.g., mouse promoter for a transgenic mouse). In some embodiments, the regulatory regions are homologous (e.g., mouse regulatory regions for a transgenic mouse). In some embodiments, the promoter and the regulatory regions are homologous (e.g., mouse promoter and/or regulatory regions for a transgenic mouse). In some embodiments, the promoter is homologous mouse GAA, or Rosa26 promoter. In some embodiments, the regulatory regions are homologous mouse GAA, or Rosa26 regulatory regions. In some embodiments, the promoter and the regulatory regions are homologous mouse GAA, or Rosa26 promoter and regulatory regions. In some embodiments, the promoter and/or regulatory regions are heterologous (e.g., non-mouse eukaryotic (e.g. human), bacterial, or viral promoter and/or regulatory regions in a transgenic mouse). In some embodiments, the promoter is heterologous (e.g., non-mouse eukaryotic (e.g. human), bacterial, or viral promoter in a transgenic mouse). In some embodiments, the regulatory regions are heterologous (e.g., non-mouse eukaryotic (e.g. human), bacterial, or viral regulatory regions in a transgenic mouse). In some embodiments, the promoter and the regulatory regions are heterologous (e.g., non-mouse eukaryotic (e.g. human), bacterial, or viral promoter and/or regulatory regions in a transgenic mouse). In some embodiments, the promoter is a heterologous human promoter. In some embodiments, the heterologous human promoter is a GAA human promoter. In some embodiments, the regulatory regions are heterologous human regulatory regions. In some embodiments, the heterologous human regulatory regions are a GAA human regulatory regions.

Additional, non-limiting examples of heterologous promoters which can be used in the present invention are: CMV early enhancer/chicken β actin (CBA) promoter, CAG promoter, CMV, EF1α, EF1α with a CMV enhancer, a CMV promoter with a CMV enhancer (CMVe/p), a CMV promoter with a SV40 intron, and the like. Promoters can be constitutive or inducible (e.g., induced or repressed). Promoters can also be tissue-specific, or stage-specific promoters which designate the expression of the GAA splicing mutant, or the wt-GAA, to specific tissues or to certain stages of development. In a preferred embodiment, the GAA splicing mutant, or the wt-GAA, is operably linked to a heterologous promoter. In a more preferred embodiment, the heterologous promoter is a CAG promoter. In an even more preferred embodiment, the heterologous promoter is a CAG promoter, comprising a nucleotide sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 3.

Regulatory regions may be used to regulate (e.g., increase or decrease) the expression level of GAA splicing mutant or to designate the expression of the GAA splicing mutant to specific tissues or to certain stages of development. In some embodiments, the regulatory region increases expression of the GAA splicing mutant. In some embodiments, the regulatory region decreases expression of the GAA splicing mutant. In some embodiments, the regulatory region increases or decreases the expression of the GAA splicing mutant in specific tissues or to certain stages of development.

In some embodiments, the GAA splicing mutant, or the wt-GAA, is operably linked to a polyadenylation signal (e.g., in a recombinant nucleic acid molecule). The polyadenylation signal sequence can be selected from any of a variety of polyadenylation signal sequences described in the literature. In some embodiments, the polyadenylation signal is a homologous polyadenylation signal. In a more preferred embodiment, the polyadenylation signal sequence is an rBG pA. In an even more preferred embodiment, the polyadenylation signal sequence is an rBG pA, comprising a nucleotide sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 4.

In a preferred embodiment, the GAA splicing mutant, or the wt-GAA, is cloned in a vector comprising a CAG promoter and an rBG pA. In an even more preferred embodiment, the GAA splicing mutant, or the wt-GAA, is cloned in a vector comprising a CAG promoter comprising a nucleotide sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 3, and an rBG pA comprising a nucleotide sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 4.

In a preferred embodiment, a GAA-IVS1-13T-G is cloned into a PUC57 vector using seamless cloning enzyme C115 (Vazyme) (PUC57-hGAA-IVS1-13T-G). In an even more preferred embodiment the PUC57-hGAA-IVS1-13T-G comprises a nucleotide sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 13.

In some embodiment, the GAA splicing mutant, or the wt-GAA, optionally operably linked to any promoter, and/or regulatory element, and/or polyadenylation signal, is linked to a nucleic acid sequence comprising homology arms (e.g., in a recombinant nucleic acid molecule). As used herein, the term “homology arm” refers to a nucleic acid sequence which is (completely or partially) identical to a nucleic acid sequence comprised in the genome of the organism into which a transgene is desired to be inserted, and which is designed to enable the specific alignment of the sequence comprising the transgene to the desired genomic sequence (locus) of the organism genome.

In a preferred embodiment, the GAA-IVS1-13T-G, operably linked to a CAG promoter and an rBG pA, is cloned into the Rosa26 locus. In an even more preferred embodiment, the GAA-IVS1-13T-G, operably linked to a CAG promoter and an rBG pA is cloned into intron 1 of the Rosa26 locus in reverse orientation. The sequences of the Rosa26 locus flanking the GAA-IVS1-13T-G, operably linked to a CAG promoter and an rBG pA, can be used as homology arms.

In some embodiment, the GAA splicing mutant, or the wt-GAA, optionally operably linked to any promoter, and/or regulatory element, and/or polyadenylation signal, and linked to a nucleic acid sequence comprising homology arms, is amplified by any one of the techniques described in the literature (e.g., PCR), to obtain an amount of genetic material which is sufficient to genetically engineer an organism.

In a preferred embodiment the GAA-IVS1-13T-G, operably linked to a CAG promoter and an rBG pA, and cloned into intron 1 of the Rosa26 locus in reverse orientation, is amplified by PCR. In an even more preferred embodiment, the PCR amplification is performed by using primers annealing with the Rosa26 homology arms. In some embodiments the Rosa26 homology arms comprise a nucleotide sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NOs: 14 or 15 In some embodiments, the primers annealing with the Rosa26 homology arms comprise a nucleotide sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NOs: 16-17.

In some embodiments, the GAA splicing mutant, or the wt-GAA, optionally operably linked to any promoter, and/or regulatory element, and/or polyadenylation signal, and/or to a nucleic acid sequence comprising homology arms, is used for producing transgenic animals by any one of the techniques described in the literature.

In some embodiments, the GAA splicing mutant, or the wt-GAA, optionally operably linked to any promoter, and/or regulatory element (e.g., enhancer), and/or polyadenylation signal, and/or to a nucleic acid sequence comprising homology arms, are comprised in a recombinant nucleic acid molecule disclosed herein. In some aspects, the recombinant nucleic acid molecules disclosed herein are used for producing the transgenic animals disclosed herein by any one of the techniques described in the literature.

In some embodiments, the recombinant nucleic acid molecules disclosed herein comprise (i) a 5′ homology arm, (ii) a GAA splicing mutant or a fragment thereof, and (iii) a 3′ homology arm. In some embodiments, the GAA splicing mutant or a fragment thereof is a human GAA splicing mutant or a fragment thereof. In some embodiments, the GAA splicing mutant or a fragment thereof comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1 or 2.

In some embodiments, the homology arms comprise a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to a region of the Rosa26 locus in a mouse genome. In some embodiments, the homology arms comprise a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to a region of the GAA locus in a mouse genome.

In some embodiments, a recombinant nucleic acid molecule disclosed herein comprising (i) a 5′ homology arm, (ii) a GAA splicing mutant or a fragment thereof, and (iii) a 3′ homology arm is used for producing the transgenic animals disclosed herein by any one of the techniques described in the literature. In some embodiments, the genome of the non-human animal model (e.g., the genome of a mouse model) comprises a GAA splicing mutant or a fragment thereof operably linked to an endogenous homologous (e.g., mouse promoter, polyadenylation signal, or regulatory region (e.g., and enhancer) for a transgenic mouse) promoter, polyadenylation signal, regulatory region (e.g., and enhancer), or a combination thereof. In some embodiments, the endogenous homologous promoter, polyadenylation signal, or regulatory region (e.g., and enhancer), is a mouse GAA, or Rosa26 promoter, polyadenylation signal, regulatory region (e.g., and enhancer). In some embodiments, the endogenous homologous promoter is a mouse GAA, or Rosa26 promoter. In some embodiments, the endogenous homologous poly adenylation signal is a mouse GAA, or Rosa26 polyadenylation signal. In some embodiments, the endogenous homologous regulatory region is a mouse GAA, or Rosa26 regulatory region.

In some embodiments, the recombinant nucleic acid molecules disclosed herein further comprise a promoter, a polyadenylation signal, a regulatory region (e.g., and enhancer), or a combination thereof. For example, the recombinant nucleic acid molecules disclosed herein can comprise (i) a 5′ homology arm, (ii) a polyadenylation signal, (iii) a GAA splicing mutant or a fragment thereof, (iv) a promoter, and (v) a 3′ homology arm.

In some embodiments, the promoter, polyadenylation signal, or regulatory region (e.g., and enhancer) is a homologous (e.g., mouse promoter, polyadenylation signal, or regulatory region) promoter, polyadenylation signal, or regulatory region (e.g., and enhancer). In some embodiments, the homologous promoter, polyadenylation signal, or regulatory region (e.g., and enhancer), is a mouse GAA, or Rosa26 promoter, polyadenylation signal, or regulatory region (e.g., and enhancer). In some embodiments, the promoter, polyadenylation signal, or regulatory region (e.g., and enhancer) is a heterologous (e.g., non-mouse eukarvotic (e.g. human), bacterial, or viral promoter and/or regulatory regions in a transgenic mouse) promoter, polyadenylation signal, or regulatory region (e.g., and enhancer). In some embodiments, the heterologous promoter, polyadenylation signal, or regulatory region (e.g., and enhancer) is a human promoter, polyadenylation signal, or regulatory region (e.g., and enhancer). In some embodiments, the human promoter, polyadenylation signal, or regulatory region (e.g., and enhancer) is a GAA human promoter, polyadenylation signal, or regulatory region (e.g., and enhancer). In some embodiments, the promoter is a GAA human promoter. In some embodiments, the polyadenylation signal is a GAA human polyadenylation signal. In some embodiments, the regulatory region (e.g., and enhancer) is a GAA human regulatory region (e.g., and enhancer).

In some embodiments, the promoter is selected from the group consisting of a CMV early enhancer/chicken R actin (CBA) promoter, CAG promoter, CMV, EF1α, EFcl with a CMV enhancer, a CMV promoter with a CMV enhancer (CMVe/p), a CMV promoter with a SV40 intron. In some embodiments, the promoter is a CAG promoter. In some embodiments, the promoter comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 3. In some embodiments, the polyadenylation signal is an rGB-pA polyadenylation signal. In some embodiments, the polyadenylation signal comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 4.

In some embodiments, a recombinant nucleic acid molecule comprising (i) a 5′ homology arm, (ii) a GAA splicing mutant or a fragment thereof, (iii) a 3′ homology arm, and further comprising a promoter, a polyadenylation signal, a regulatory region (e.g., and enhancer), or a combination thereof is used for producing the transgenic animals disclosed herein by any one of the techniques described in the literature.

In some embodiments, the genome of the non-human animal model (e.g., the genome of a mouse model) comprises a GAA splicing mutant or a fragment thereof operably linked to an exogenous promoter, polyadenylation signal, regulatory region (e.g., and enhancer), or combination thereof. In some embodiments, the exogenous promoter, polyadenylation signal, or regulatory region (e.g., and enhancer) is a homologous (e.g., mouse promoter, polyadenylation signal, or regulatory region) promoter, polyadenylation signal, or regulatory region (e.g., and enhancer). In some embodiments, the homologous promoter, polyadenylation signal, or regulatory region (e.g., and enhancer), is a mouse GAA, or Rosa26 promoter, polyadenylation signal, or regulatory region (e.g., and enhancer).

In some embodiments, the exogenous promoter, polyadenylation signal, or regulatory region (e.g., and enhancer) is a heterologous (e.g., non-mouse eukaryotic (e.g. human), bacterial, or viral promoter and/or regulatory regions in a transgenic mouse) promoter, polyadenylation signal, or regulatory region (e.g., and enhancer). In some embodiments, the heterologous promoter, polyadenylation signal, or regulatory region (e.g., and enhancer) is a human promoter, polyadenylation signal, or regulatory region (e.g., and enhancer). In some embodiments, the human promoter, polyadenylation signal, or regulatory region (e.g., and enhancer) is a GAA human promoter, polyadenylation signal, or regulatory region (e.g., and enhancer). In some embodiments, the promoter is a GAA human promoter. In some embodiments, the polyadenylation signal is a GAA human polyadenylation signal. In some embodiments, the regulatory region (e.g., and enhancer) is a GAA human regulatory region (e.g., and enhancer).

In some embodiments, the exogenous promoter is selected from the group consisting of a CMV early enhancer/chicken β actin (CBA) promoter. CAG promoter, CMV, EFla, EF1α with a CMV enhancer, a CMV promoter with a CMV enhancer (CMVe/p), a CMV promoter with a SV40 intron. In some embodiments, the promoter is a CAG promoter. In some embodiments, the promoter comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 3. In some embodiments, the polyadenylation signal is an rGB-pA polyadenylation signal. In some embodiments, the polyadenylation signal comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 4.

In some embodiments, the recombinant nucleic acid molecules disclosed herein further comprise a Neo (neomycin) resistance gene, an Amp (ampicillin) resistance gene.

Techniques for producing transgenic animals are described in the literature. See., e.g., Houdebine, Transgenic animals-Generation and Use (Harwood Academic, 1997); Hogan et al., Manipulating the Mouse Embryo: A Laboratory Manual Cold Spring Harbor Laboratory, 2d ed., (Cold Spring Harbor Laboratory, 1994): Krimpenfort et al., Bio/Technology 1991, 9:844; Palmiter et al., Cell 1985, 41:343; Hammer et al., Nature 1985, 315:680; U.S. Pat. Nos. 5,602,299; 5,175,384; 6,066,778 and 6,037,521, which are incorporated herein in their entirety. Technologies used in generating transgenic animals include, but are not limited to, pronuclear injection (Gordon, Proc. Nat. Acad. Sci. USA 1980, 77:7380-7384; U.S. Pat. No. 4,873,191), electroporation (Lo, Mol. Cell. Biol. 1983. 3:1803-1814), homologous recombination (Thompson et al., Cell 1989, 56:313-321; Hanks et al., Science 1995, 269: 679-682), retrovirus gene transfer into germ lines (Van der Putten et al., Proc. Nat. Acad. Sci. USA 1985, 82:6148-6152), and sperm-mediated gene transfer (Lavitrano et al., Cell 1989, 57:717-723). In some embodiments, genome editing techniques, including zinc-finger nucleases (ZFNs), transcription activator-like effector nucleases (TALENs) and the RNA-guided CRISPR-Cas nuclease system, can be used to produce the transgenic animals of the disclosure. In a preferred embodiment, the RNA-guided CRISPR-Cas9 nuclease system is used to produce the transgenic animals of the disclosure.

The specificity of the Cas9 nuclease is determined by the 20-nt guide sequence within the sgRNA. For example, in the S. pyogenes system, the target sequence (e.g., 5′-GTCACCTCCAATGACTAGGG-3′) must immediately precede (i.e., be 5′ to) a 5′-NGG PAM, and the 20-nt guide sequence base pairs with the opposite strand to mediate Cas9 cleavage at −3 bp upstream of the PAM. The PAM sequence is required to immediately follow the target DNA locus, but that it is not a part of the 20-nt guide sequence within the sgRNA. Bioinformatic CRISPR Design Tools that take a genomic sequence of interest and identify suitable target sites may be used for the design of the specific sgRNAs. In a preferred embodiment, the specific sgRNAs comprise a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 18.

Off-target sites for each sgRNA can be also computationally predicted for each intended target using bioinformatics. For increased targeting specificity, an alternative strategy using the D10A nickase mutant of Cas9 (Cas9n) along with a pair of sgRNAs may be used. On occasions, certain sgRNAs may not work for reasons yet unknown, therefore, it is recommend designing at least two sgRNAs for each locus and testing their efficiencies in the intended cell type.

Depending on the desired application, sgRNAs can be delivered as either PCR amplicons containing an expression cassette or sgRNA-expressing plasmids. In addition to PCR and plasmid-based delivery methods, Cas9 and sgRNAs can be introduced into cells as mRNA and RNA, respectively. Furthermore, Cas9 may be delivered to the cells as a polypeptide.

In some aspects, the GAA splicing mutant, or the wt-GAA, optionally operably linked to any promoter, and/or regulatory element, and/or polyadenylation signal, and linked to a nucleic acid sequence comprising homology arms, is used as repair template. In some embodiments the GAA splicing mutant, or the wt-GAA, optionally operably linked to any promoter, and/or regulatory element, and/or polyadenylation signal, and linked to a nucleic acid sequence comprising homology arms is comprised in a plasmid, and such plasmid is introduced into the cell. In some embodiments the GAA splicing mutant, or the wt-GAA, optionally operably linked to any promoter, and/or regulatory element, and/or polyadenylation signal, and linked to a nucleic acid sequence comprising homology arms is delivered to the cell as PCR amplicons.

In a preferred embodiment, the Cas9 is delivered to the cell as a polypeptide, the sgRNAs are delivered to the cell as RNAs. and the repair template, comprising the GAA-IVS1-13T-G, is delivered to the cell as PCR amplicon.

In some embodiment about 1 ng/μL, about 5 ng/μL, about 10 ng/μL, about 15 ng/μL, about 20 ng/μL, about 25 ng/μL, about 30 ng/μL, about 35 ng/μL, about 40 ng/μL, about 45 ng/μL, about 50 ng/μL, about 55 ng/μL, about 60 ng/μL, about 65 ng/μL, about 70 ng/μL, about 75 ng/μL, about 80 ng/μL, about 85 ng/μL, about 90 ng/μL, about 95 ng/μL, about 100 ng/μL, of Cas9 polypeptide is delivered to the cell. In a preferred embodiment about 10 ng/mL, about 15 ng/mL, about 20 ng/mL, about 25 ng/mL, about 30 ng/mL, about 35 ng/mL, about 40 ng/mL, about 45 ng/mL, about 50 ng/mL of Cas9 polypeptide is delivered to the cell. In an even more preferred embodiment about 30 ng/mL of Cas9 polypeptide is delivered to the cell.

In some embodiment about 1 ng/μL, about 5 ng/μL, about 10 ng/μL, about 15 ng/μL, about 20 ng/μL, about 25 ng/μL, about 30 ng/μL, about 35 ng/μL, about 40 ng/μL, about 45 ng/μL, about 50 ng/μL, about 55 ng/μL, about 60 ng/μL, about 65 ng/μL, about 70 ng/μL, about 75 ng/μL, about 80 ng/μL, about 85 ng/μL, about 90 ng/μL, about 95 ng/μL, about 100 ng/μL, of the PCR amplicon corresponding to the repair template comprising the GAA splicing mutant, or the wt-GAA is delivered to the cell. In a preferred embodiment about 1 ng/mL, about 5 ng/mL, about 10 ng/mL, about 15 ng/mL, about 20 ng/mL, about 25 ng/mL, about 30 ng/mL, about 35 ng/mL, about 40 ng/mL of the PCR amplicon corresponding to the repair template comprising the GAA splicing mutant, or the wt-GAA is delivered to the cell. In an even more preferred embodiment about 15 ng/mL of the PCR amplicon corresponding to the repair template comprising the GAA splicing mutant, or the wt-GAA is delivered to the cell.

In some embodiment about 1 ng/μL, about 5 ng/μL, about 10 ng/μL, about 15 ng/μL, about 20 ng/μL, about 25 ng/μL, about 30 ng/μL, about 35 ng/μL, about 40 ng/μL, about 45 ng/μL, about 50 ng/μL, about 55 ng/μL, about 60 ng/μL, about 65 ng/μL, about 70 ng/μL, about 75 ng/μL, about 80 ng/μL, about 85 ng/μL, about 90 ng/μL, about 95 ng/μL, about 100 ng/μL, of the RNAs corresponding to the sgRNAs are delivered to the cell. In a preferred embodiment about 1 ng/mL, about 5 ng/mL, about 10 ng/mL, about 15 ng/mL, about 20 ng/mL, about 25 ng/mL, about 30 ng/mL, about 35 ng/mL, about 40 ng/mL of the RNAs corresponding to the sgRNAs are delivered to the cell. In an even more preferred embodiment about 15 ng/mL of the RNAs corresponding to the sgRNAs are delivered to the cell.

In some embodiments, CRISPR/Cas9 gene editing procedure is used in combination with transfection of ES cells to generate transgenic mice. In a preferred embodiment, CRISPR/Cas9 gene editing procedure is used in combination with pronuclear injection to generate transgenic mice. In an even more preferred embodiment, pronuclear injection is performed on one-cell stage zygotes obtained by mating C57BL/6N males (Charles River, China) with superovulated C57BL/6N females (Charles River, China).

In some embodiments, the injected embryos can be cultured in any suitable medium and subsequently transferred into the oviduct of pseudopregnant females at any stage. In a preferred embodiment, the injected embryos are cultured in KSOM medium overnight, and the injected embryos which develop to the two-cell stage are transferred into the oviduct of pseudopregnant females.

Transgenic animals can be screened for the presence and/or expression of a transgene, and for the presence and/or expression of splicing variants of a transgene, by any suitable methods described in the literature. In some embodiments, screening is accomplished by in situ hybridization. Southern blot or Northern blot analysis, using an oligonucleotide probe that is complementary to at least a portion of the DNA or RNA of the transgene. In other embodiments, screening is accomplished by Western blot analysis using an antibody specific binding to the protein encoded by the transgene. In some embodiments, a whole transgenic animal, and/or cells, tissues, organs derived from the transgenic animal are tested for the presence and expression of the transgene using in situ hybridization, PCR, Southern, Northern. or Western blot analysis. In some embodiments, DNA is prepared from a tissue (e.g., tail, ear, muscle) of the transgenic animal (e.g., transgenic mouse) and analyzed by Southern blot analysis or PCR for the transgene. In a preferred embodiment, animals can be screened for the presence of the transgene by PCR amplification. In an even more preferred embodiment, animals are screened for the presence of the transgene by PCR amplification using primers comprising a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NOs: 16, 17, 19-28.

In some embodiments, the whole transgenic animal, and/or cells, tissues, organs derived from the transgenic animal are tested for the presence and expression of splicing variants of the transgene using in situ hybridization, PCR, Southern, Northern, or Western blot analysis. In some embodiments, cDNA is prepared from a tissue (e.g., tail, ear, muscle) of the transgenic animal (e.g., transgenic mouse) and analyzed by Southern blot analysis or PCR for the expression of splicing variants of the transgene. In a preferred embodiment, animals can be screened for the presence and expression of splicing variants of the transgene by PCR amplification. In an even more preferred embodiment, animals are screened for the presence and expression of splicing variants of the transgene by PCR amplification using primers comprising a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NOs: 5-10.

In some embodiments, the sequence of the transgene can be verified by sequencing (e.g., Sanger sequencing). In some embodiments, PCR amplicons derived from DNA (e.g., genomic DNA, or cDNA) derived from the transgenic animal can be sequenced by any one of the techniques described in the literature (e.g., Sanger sequencing). In some embodiments, specific primers can be used to sequence the PCR amplicons derived from DNA (e.g., genomic DNA, or cDNA) derived from the transgenic animal. In some embodiments, the primers used to sequence the PCR amplicons derived from DNA (e.g., genomic DNA, or cDNA) derived from the transgenic animal comprise a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NOs: 29-31.

Founder animals can be bred, inbred, outbred, or crossbred to produce colonies of the desired transgenic animals. Non-limiting examples of such breeding strategies include: outbreeding of founder animals with more than one integration sites to establish separate lines; inbreeding of separate lines to produce compound transgenic that express the transgene at higher levels because of the additive effect of each transgene; crossing of heterozygous transgenic mice to increase expression of the transgene and/or to produce mice homozygous for a given integration site; crossing of separate homozygous lines to produce compound heterozygous or homozygous lines; breeding animals to different inbred genetic backgrounds to study effects of modifying alleles on expression of the transgene and the physiological effects of expression of the transgene.

In some embodiments, a GAA splicing mutant is inserted into the non-human animal model genome. In some embodiments, a wt-GAA is inserted into the non-human animal model genome. In some embodiments, a mutation (e.g., a T-G mutation, such as an IVS1-13T-G mutation) is introduced into a wild type GAA gene by any one of the techniques described in the literature. In some embodiments, the mutation is introduced into a wild type GAA gene before the introduction of the transgene into the non-human animal model. In some embodiments, the mutation is introduced into a wild type GAA gene at the same time (i.e., simultaneously) of the introduction of the transgene into the non-human animal model. In some embodiments, the mutation is introduced into a wild type GAA gene after the introduction of the transgene into the non-human animal model. In some embodiments, provided herein is a non-human animal model generated by mating a non-human animal model disclosed herein with a non-human animal model lacking all copies of a GAA gene endogenous to the non-human animal model.

For example, a mouse model, produced according to the methods disclosed herein, comprising at least one copy of a GAA splicing mutant inserted into its genome (i.e., into the genome of a germ cell of the mouse model), and comprising at least one (e.g., two copies) of the GAA gene endogenous to the mouse model can be mated to a mouse model lacking all the copies of the GAA gene endogenous to the mouse model. The progeny of such mating can be screened, according to the methods known in the published literature, to select non-human animal models comprising the GAA splicing mutant and not comprising any copy of the GAA gene endogenous to the non-human animal model in their genome. Such non-human animal models are herein also referred to as fully humanized non-human animal models.

### 4. Methods of Use of Genetically Engineered, Non-Human, Animal Models of Pompe Disease

The non-human animal models of Pompe disease of the disclosure can be used for a variety of studies.

In humans, the GAA gene is found on the long arm of chromosome 17 (17q25.2-q25.3) and consists of 20 exons. The GAA pre-RNA is subject to alternative splicing, which generates mature RNAs comprising different exons combination. The IVS1-13T-G (c.-32-13T>G) mutation weakens the splice acceptor of GAA exon 2 and leads to a splicing defect and skipping of exon 2. The splicing defect and skipping of exon 2 leads to low level of active enzyme (12% of normal) generated from the leakage of normally spliced mRNA.

There is a need in the art for splice modulating agents (e.g., antisense oligomers, antisense oligonucleotides, or small molecules) to enhance GAA exon 2 inclusion in the mature mRNA of patients with one c.-32-13T>G allele.

The genetically engineered, non-human, animal models of Pompe disease of the present invention, or cells, tissues, organs, or portions, derived therefrom, can be used to test the efficacy of such splice modulating agents.

In some embodiments, the splice modulating agent may interact or bind to one or more splicing protein in the cell. In some embodiments, the splice modulating agent can activate one or more splicing protein in the cell, and/or can inhibit one or more splicing protein in the cell. In some embodiments, the splice modulating agent may interact or bind to a protein that regulates one or more splicing protein in the cell. In some embodiments, the splice modulating agent can activate of one or more protein that regulates one or more splicing protein in the cell, or can inhibit one or more protein that regulates one or more splicing protein in the cell. In some embodiments, the splice modulating agent may interact or bind to target polynucleotide sequence of the partially processed mRNA transcripts (i.e., pre-mRNA).

In some embodiments the splice modulating agent may be an antisense oligomer (e.g., an antisense oligonucleotide) binding to a targeted portion of a pre-mRNA. The ASO may have exact sequence complementary to the target sequence or near complementarity (i.e., sufficient complementarity to bind the target). ASOs are designed so that they bind (hybridize) to a target nucleic acid (e.g., a targeted portion of a pre-mRNA transcript) under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the pre-mRNA transcript or a sufficiently similar nucleic acid sequence in other locations in the genome or cellular pre-mRNA or transcriptome, such that the likelihood the ASO will bind other sites and cause “off-target” effects is limited. Any antisense oligomers described in the literature, can be used to practice the methods described herein.

In some embodiments, ASOs “specifically hybridize”, or are “specific” to a target nucleic acid or a targeted portion of a pre-mRNA.

Oligomers, such as oligonucleotides, are “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an antisense oligomer (ASO) need not be 100% complementary to that of its target nucleic acid to hybridize. In certain embodiments, ASOs can comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleotides of the oligomeric compound are complementary to a target region, and would therefore specifically hybridize, would represent 90 percent complementarity. In this example, the remaining noncomplementary nucleotides may be clustered together or interspersed with complementary nucleotides and need not be contiguous to each other or to complementary nucleotides. Percent complementarity of an ASO with a region of a target nucleic acid can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656).

An ASO need not hybridize to all nucleotides in a target sequence and the nucleotides to which it does hybridize may be contiguous or contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleotides in a target pre-mRNA transcript. For example, an ASO can hybridize to nucleotides in a pre-mRNA transcript that are separated by one or more nucleotide(s) to which the ASO does not hybridize.

The ASOs described herein may comprise nucleotides that are complementary to nucleotides present in a target portion of a pre-mRNA. The term ASO embodies oligonucleotides and any other oligomeric molecule that comprises nucleotides capable of hybridizing to a complementary nucleotides on a target mRNA but does not comprise a sugar moiety, such as a peptide nucleic acid (PNA). The ASOs may comprise naturally-occurring nucleotides, nucleotide analogs, modified nucleotides, or any combination thereof. In some embodiments, all of the nucleotides of the ASO are naturally occurring nucleotides. In some embodiments, all of the nucleotides of the ASO are modified nucleotides. In some embodiments, some of the nucleotides of the ASO are naturally occurring nucleotides and some of the nucleotides of the ASO are modified nucleotides. Chemical modifications of ASOs or components of ASOs that are compatible with the methods and compositions described herein will be evident to one of skill in the art.

The nucleobase of an ASO may be any naturally occurring, or any synthetic or modified nucleobase.

The ASOs described herein also comprise a backbone structure that connects the components of an oligomer. The backbone structure may comprise 3′-5′ phosphodiester linkages connecting the sugar moieties of the oligomer. The backbone structure of the ASOs described herein may include (but are not limited to) phosphorothioate, phosphorodithioate, phosphoroselenoate, phosphorodiselenoate, phosphoroanilothioate, phosphoraniladate, phosphoramidate, and the like. In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphothioate linkage. In some embodiments, the backbone modification is a phosphoramidate linkage.

Any of the ASOs described herein may contain a sugar moiety that comprises ribose or deoxyribose, as present in naturally occurring nucleotides, or a modified sugar moiety or sugar analog.

In some embodiments, each monomer of the ASO is modified in the same way. Such modifications that are present on each of the monomer components of an ASO are referred to as “uniform modifications.” In some embodiments, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modification. In some embodiments, the ASO comprises one or more sugar moiety modification. In some embodiments, the ASO comprises one or more backbone modification and one or more sugar moiety modification. In some embodiments, the ASO comprises 2′MOE modifications and a phosphorothioate backbone. In some embodiments, the ASO comprises a phosphorodiamidate morpholino (PMO). In some embodiments, the ASO comprises a peptide nucleic acid (PNA). Any of the ASOs or any component of an ASO (e.g., a nucleobase, sugar moiety) described herein may be modified in order to achieve desired properties or activities of the ASO or reduce undesired properties or activities of the ASO. For example, an ASO or one or more component of any ASO may be modified to enhance binding affinity to atarget sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and modulate the half-life of the ASO.

In some embodiments, the ASOs are comprised of 2′-O-(2-methoxyethyl) (MOE) phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary et al., J Pharmacol Exp Ther. 2001; 296(3):890-7; Geary et al., J Pharmacol Exp Ther. 2001; 296(3):898-904.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some aspects, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1.” while a nucleotide that is directly adjacent and downstream of the reference point is designated “plus one,” e.g., “+1.”

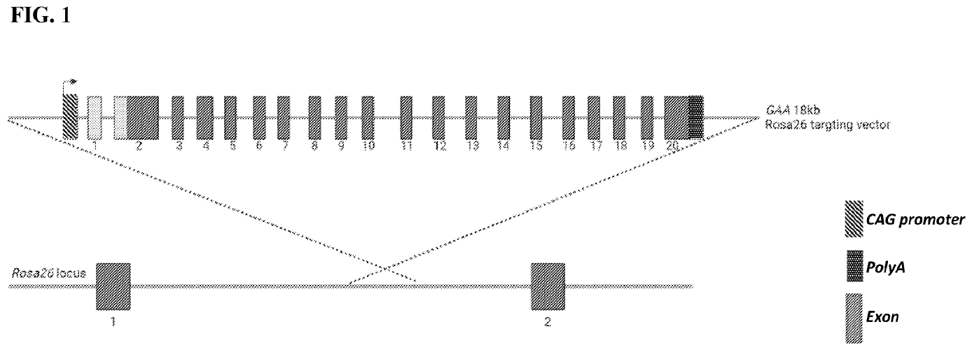

In other embodiments, the ASOs are complementary to (and bind to) a targeted portion of a pre-mRNA that is downstream (in the 3′ direction) of the 5′ splice site of the intron in a pre-mRNA (e.g., the direction designated by positive numbers relative to the 5′ splice site) (FIG. 1). In some embodiments, the ASOs are complementary to a targeted portion of the pre-mRNA that is within the region +6 to +100 relative to the 5′ splice site of the intron. In some embodiments, the ASO is not complementary to nucleotides +1 to +5 relative to the 5′ splice site (the first five nucleotides located downstream of the 5′ splice site). In some embodiments, the ASOs may be complementary to a targeted portion of a pre-mRNA that is within the region between nucleotides +6 and +50 relative to the 5′ splice site of the intron. In some aspects, the ASOs are complementary to a targeted portion that is within the region +6 to +90, +6 to +80, +6 to +70, +6 to +60, +6 to +50, +6 to +40, +6 to +30, or +6 to +20 relative to 5′ splice site of the intron.

In some embodiments, the ASOs are complementary to a targeted region of a pre-mRNA that is upstream (5′ relative) of the 3′ splice site of the intron in a pre-mRNA (e.g., in the direction designated by negative numbers) (FIG. 1). In some embodiments, the ASOs are complementary to a targeted portion of the pre-mRNA that is within the region−16 to −100 relative to the 3′ splice site of the intron. In some embodiments, the ASO is not complementary to nucleotides−1 to −15 relative to the 3′ splice site (the first 15 nucleotides located upstream of the 3′ splice site). In some embodiments, the ASOs are complementary to a targeted portion of the pre-mRNA that is within the region−16 to −50 relative to the 3′ splice site of the intron. In some aspects, the ASOs are complementary to a targeted portion that is within the region−16 to −90, −16 to −80, −16 to −70, −16 to −60, −16 to −50, −16 to −40, or −16 to −30 relative to 3′ splice site of the intron.

In embodiments, the targeted portion of the pre-mRNA is within the region +100 relative to the 5′ splice site of the intron to −100 relative to the 3′ splice site of the intron.

In some embodiments, the ASOs are complementary to a targeted portion of a pre-mRNA that is within the exon flanking the 5′ splice site (upstream) of the intron. In some embodiments, the ASOs are complementary to a targeted portion of the pre-mRNA that is within the region +2e to −4e in the exon flanking the 5′ splice site of the intron. In some embodiments, the ASOs are not complementary to nucleotides −1e to −3e relative to the 5′ splice site of the intron. In some embodiments, the ASOs are complementary to a targeted portion of the pre-mRNA that is within the region −4e to −100e, −4e to −90e, −4e to −80e, −4e to −70e. −4e to −60e, −4e to −50e, −4 to −40e, −4e to −30e, or −4e to −20e relative to the 5′ splice site of the intron.

In some embodiments, the ASOs are complementary to a targeted portion of a pre-mRNA that is within the exon flanking the 3′ splice site (downstream) of the intron (FIG. 1). In some embodiments, the ASOs are complementary to a targeted portion to the pre-mRNA that is within the region +2e to −4e in the exon flanking the 3′ splice site of the intron. In some embodiments, the ASOs are not complementary to nucleotide +1e relative to the 3′ splice site of the intron. In some embodiments, the ASOs are complementary to a targeted portion of the pre-mRNA that is within the region +2e to +100e, +2e to +90e, +2e to +80e, +2e to +70e, +2e to +60e, +2e to +50e, +2e to +40e, +2e to +30e, or +2 to +20e relative to the 3′ splice site of the intron. The ASOs may be of any length suitable for specific binding and effective enhancement of splicing. In some embodiments, the ASOs consist of 8 to 50 nucleotides. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleotides in length. In some embodiments, the ASOs consist of more than 50 nucleotides. In some embodiments, the ASO is from 8 to 50 nucleotides, 8 to 40 nucleotides, 8 to 35 nucleotides, 8 to 30 nucleotides, 8 to 25 nucleotides, 8 to 20 nucleotides, 8 to 15 nucleotides, 9 to 50 nucleotides, 9 to 40 nucleotides, 9 to 35 nucleotides, 9 to 30 nucleotides, 9 to 25 nucleotides, 9 to 20 nucleotides, 9 to 15 nucleotides, 10 to 50 nucleotides, 10 to 40 nucleotides, 10 to 35 nucleotides, 10 to 30 nucleotides, 10 to 25 nucleotides, 10 to 20 nucleotides, 10 to 15 nucleotides, 11 to 50 nucleotides, 11 to 40 nucleotides, 11 to 35 nucleotides, 11 to 30 nucleotides, 11 to 25 nucleotides, 11 to 20 nucleotides, 11 to 15 nucleotides, 12 to 50 nucleotides, 12 to 40 nucleotides, 12 to 35 nucleotides, 12 to 30 nucleotides, 12 to 25 nucleotides, 12 to 20 nucleotides, 12 to 15 nucleotides, 13 to 50 nucleotides, 13 to 40 nucleotides, 13 to 35 nucleotides, 13 to 30 nucleotides, 13 to 25 nucleotides, 13 to 20 nucleotides, 14 to 50 nucleotides, 14 to 40 nucleotides, 14 to 35 nucleotides, 14 to 30 nucleotides, 14 to 25 nucleotides, 14 to 20 nucleotides, 15 to 50 nucleotides, 15 to 40 nucleotides, 15 to 35 nucleotides, 15 to 30 nucleotides, 15 to 25 nucleotides, 15 to 20 nucleotides, 20 to 50 nucleotides, 20 to 40 nucleotides, 20 to 35 nucleotides, 20 to 30 nucleotides, 20 to 25 nucleotides. 25 to 50 nucleotides, 25 to 40 nucleotides, 25 to 35 nucleotides, or 25 to 30 nucleotides in length. In some embodiments, the ASOs are 18 nucleotides in length. In some embodiments, the ASOs are 15 nucleotides in length. In some embodiments, the ASOs are 25 nucleotides in length.

In some embodiments, two or more ASOs with different chemistries but complementary to the same targeted portion of the pre-mRNA are used. In some embodiments, two or more ASOs that are complementary to different targeted portions of the pre-mRNA are used.

In embodiments, the antisense oligonucleotides of the invention are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine or a polyethylene glycol chain, or adamantane acetic acid. Oligonucleotides comprising lipophilic moieties, and preparation methods have been described in the literature. In embodiments, the antisense oligonucleotide is conjugated with a moiety including, but not limited to, an abasic nucleotide, a polyether, a polyamine, a polyamide, a peptides, a carbohydrate, e.g., N-acetylgalactosamine (GalNAc), N-Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide at any of several positions on the sugar, base or phosphate group, as understood in the art and described in the literature, e.g., using a linker. Linkers can include a bivalent or trivalent branched linker. In embodiments, the conjugate is attached to the 3′ end of the antisense oligonucleotide. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, the nucleic acid to be targeted by an ASO is a pre-mRNA expressed in a cell, such as a eukaryotic cell. In some embodiments, the term “cell” may refer to a population of cells. In some embodiments, the cell is in a subject. In some embodiments, the cell is in vivo. In some embodiments, the cell is isolated from a subject. In some embodiments, the cell is ex vivo. In some embodiments, the cell is in vitro. In some embodiments, the cell is a condition or disease-relevant cell or a cell line. In some embodiments, the cell is in vitro (e.g., in cell culture).

In some embodiments, the therapeutic agent can be a small molecule. For example, a small molecule can be a molecule of less than 900 Daltons.

In some embodiments, the mRNA is extracted from the cells, the tissues, or the organs derived from the transgenic non-human animal model prior to administering the splice modulating agent, and after administering the splice modulating agent. The mRNA can be extracted the cells, the tissues, or the organs derived from the transgenic non-human animal model by any means described in the literature. For example, the mRNA can be extracted by organic extraction, such as phenol-Guanidine Isothiocyanate (GITC)-based solutions, silica-membrane based spin column technology, and paramagnetic particle technology.

In some embodiments, the mRNA is retrotranscribed into cDNA. The mRNA can be retrotranscribed into cDNA by any means described in the literature. For example, any reverse transcriptases can be used, such as those comprised in commercially available kits.

In some embodiments, the cDNA is processed by PCR. In some embodiments, specific pairs of primers can be used to perform PCR reactions by which the presence and/or the ratio of different splicing forms can be detected and/or measured. In some embodiments, a pair of primers is used to amplify an exon junction, which is not affected by the mutation and therefore amplifies both a GAA not comprising a splicing mutation and a GAA gene comprising a splicing mutation, and a pair of primers is used to amplify an exon junction, which is affected by the mutation and therefore amplifies only a GAA not comprising a splicing mutation. For example, in one embodiment, a pair of primers is used to amplify the 6-7 exon junction, which is not affected by the IVS1-13T-G and therefore amplifies both the wild type and the defective splicing c.-32-13T>G forms of the GAA gene, and a pair of primers is used to amplify the 1-2 exon junction, which is affected by the IVS1-13T-G and therefore amplifies only the wild type form of the GAA gene. In some embodiments, a pair of primers is used to amplify a region of the GAA gene, which has different length in the mature mRNA derived from a GAA gene comprising a splicing mutation and a GAA not comprising a splicing mutation. For example, in one embodiment, a pair of primers is used to amplify the exons 1-5, mature mRNAs derived from a GAA gene comprising the IVS1-13T-G mutation produce amplicons with a length that is different from mature mRNAs derived from a GAA gene not comprising the IVS1-13T-G mutation. In another embodiment, the primers used for the present PCR assay comprise a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NOs: 11-12. In some embodiments, the proteins products translated from a mature mRNA derived from a pre-mRNA transcribed from the mutGAA gene are extracted from the cells, the tissues, or the organs derived from the transgenic non-human animal model prior to administering the splice modulating agent, and after administering the splice modulating agent. The protein products can be extracted from the cells, the tissues, or the organs derived from the transgenic non-human animal model by any one of means described in the published literature. Methods of detecting and analyzing protein products are also known in the published literature. For example, the protein content extracted from a cell, tissue, or organ, can be assayed by Western blot, if a suitable antibody capable of recognizing a specific protein product is available. A number of antibodies capable of recognizing a protein product of a GAA gene (i.e., a GAA protein encoded by a GAA gene) are known and commercially available. Nevertheless, most, if not all, of such antibodies are not capable of discriminating between protein products of GAA genes derived from different species. For example, most, if not all, of such antibodies are capable of recognizing (i.e., bind) to both the human and the mouse GAA genes protein products (i.e., bind to both the protein encoded by the human and the protein encoded by the mouse GAA genes), this phenomenon is also known as cross-reactivity.

In some embodiments, the non-human animal models comprising a GAA splicing mutant and lacking all of the copies of the GAA gene endogenous to the non-human animal model, disclosed herein, are particularly useful for analyzing the protein products translated from a mature mRNA derived from a pre-mRNA transcribed from the mnutGAA gene prior to administering the splice modulating agent, and after administering the splice modulating agent. The absence of any copy of the GAA gene endogenous to the non-human animal model overcomes the cross-reactivity of most, if not all, the available antibodies capable of binging a protein product of a GAA gene (i.e., a GAA protein encoded by the GAA gene).

In some embodiments, a splice modulating agent alters the splicing of the pre-mRNA. In some embodiments, a splice modulating agent does not alter the splicing of the pre-mRNA. In some embodiments, the ratio of one variant of a target mRNA to another variant of the target mRNA is altered. In some embodiments, the ratio of one variant of a target mRNA to another variant of the target mRNA is not altered. In some embodiments, the ratio of one variant of a target protein to another variant of the target protein is altered. In some embodiments, the ratio of one variant of a target protein to another variant of the target protein is not altered. In some embodiments, the antisense oligomer increases the amount of functional protein (i.e., a protein having GAA activity), or of the RNA which is translated into functional protein. In some embodiments, the antisense oligomer does not increase the amount of functional protein (i.e., a protein having GAA activity), or of the RNA which is translated into functional protein.

In some embodiments, the total amount of functional protein (i.e., a protein having GAA activity), or of the RNA which is translated into functional protein produced in the cell contacted with the splice modulating agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the total amount of functional protein (i.e., a protein having GAA activity), or of the RNA which is translated into functional protein, produced in a control cell which is not contacted with the splice modulating agent.

In some embodiments, the total amount of functional protein (i.e., a protein having GAA activity), or of the RNA which is translated into functional protein produced in the cell contacted with the splice modulating agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the total amount of functional protein (i.e., a protein having GAA activity), or of the RNA which is translated into functional protein, produced in a control cell which is not contacted with the splice modulating agent.

In some embodiments, the total amount of functional protein (i.e., a protein having GAA activity), or of the RNA which is translated into functional protein produced in the cell contacted with the splice modulating agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the total amount of functional protein (i.e., a protein having GAA activity), or of the RNA which is translated into functional protein, produced in a control cell which is not contacted with the splice modulating agent.

In some embodiments, the total amount of functional protein (i.e., a protein having GAA activity), or of the RNA which is translated into functional protein produced in the cell contacted with the splice modulating agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the total amount of functional protein (i.e., a protein having GAA activity), or of the RNA which is translated into functional protein, produced in a control cell which is not contacted with the splice modulating agent.

The splice modulating agent may be delivered to the non-human animal models of Pompe disease of the present invention, or cells, tissues, organs, or portions, derived therefrom, by any means described in the literature. For example, the splice modulating agent may be administered to the non-human animal model through any suitable route. Cells, tissues, organs, or portions, can be derived from the non-human animal model by any means described in the literature. Cells, tissues, organs, or portions, derived from the non-human animal model, may be maintained and/or expanded in culture by any means described in the literature (see, for example, Parker (1961), Paul (1961), White (1963), and Merchant et. al. (1964), White (1957) and Stevenson (1962), Stewart and Kirk (1954), Waymouth (1954, 1960, 1965), Hanks (1955), Biggers et al. (1957), Geyer (1958), Morgan (1958), Swim (1959), Paul (1960), Levintow and Eagle (1961), Murray and Kopech, (1953), Murray and Kopech, (1965, 1966), Wolff(1952), Fell (1953, 1954, 1955, 1958, 1964), Gaillard (1942, 1948, 1953), Borghese (1958). Kahn (1958), Lasnitzki (1958, 1965), Trowell (1959, 1961b), and Grobstein (1962). The splice modulating agent may be administered (e.g., delivered) to the cells, the tissues, the organs, or the portions derived from the non-human animal model by any means described in the literature.

### Example 1: Generation of Pompe Disease Mouse Model

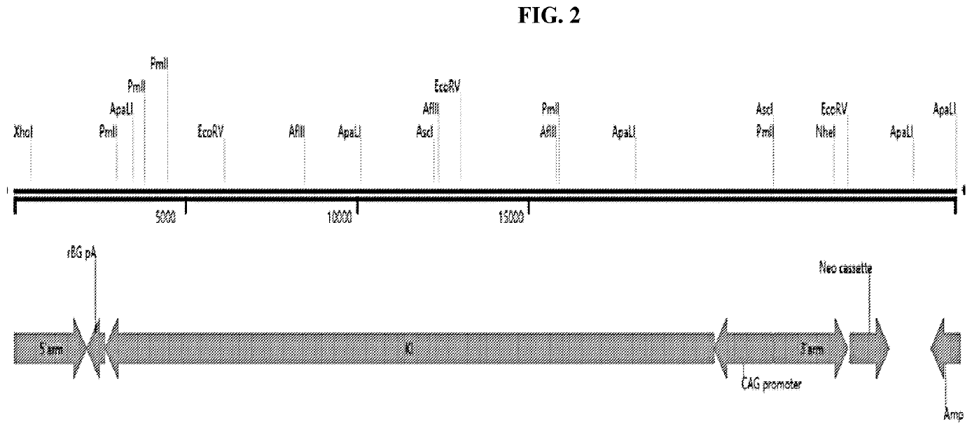

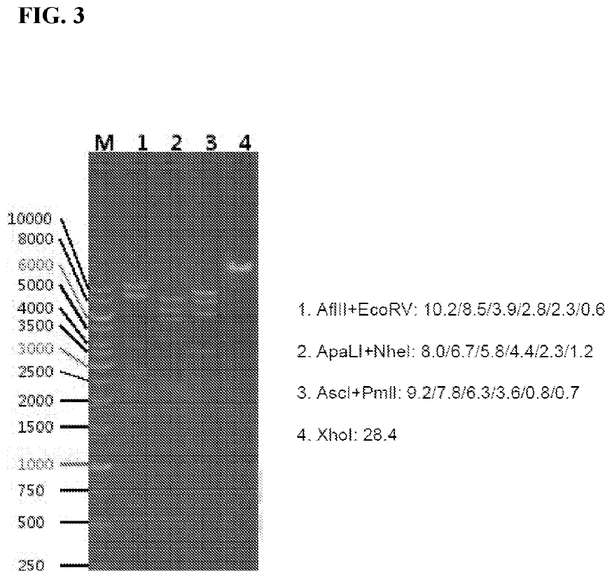

The LOPD knock-in mouse model was generated by inserting human GAA gene into the Rosa26 locus of C57BL/6 mice using CRISPR/Cas9 genome engineering (Cyagen Biosciences). First, a BAC clone containing human GAA was selected (BAC clone RP11-75G22), and the IVS1 mutation introduced by a positive/negative homologous recombination selection scheme A synthetic fragment containing a zeomycin resistance cassette and homology arms including the IVS1 mutation was introduced to the BAC containing the human GAA gene through homologous recombination. The zeomycin cassette was then removed by negative homologous recombination using a synthetic sequence not containing the zeomycin cassette. The modified gene was subcloned to introduce the CAG promoter and PolyA tail (rGB pA), into the PUC57 vector using seamless cloning enzyme C115 (Vazyme). The entire CAG-(IVS1)hGAA-polyA insert was sequenced and cloned into intron 1 of the Rosa26 locus in reverse orientation (FIG. 1) sequenced. Restriction enzyme digestions, were performed to verify the correct insertion of the insert into the cloning vector (FIG. 2 and FIG. 3). Rosa26 homology arms were amplified by PCR and the resulting targeting vector (PCR amplicon) along with Cas9 (polypeptide) and sgRNA (synthesized RNAs) were co-injected into fertilized eggs. One-cell stage zygotes were obtained by mating C57BL/6N males (Charles River, China) with C57BL/6N females (Charles River, China) superovulated by injection of pregnant mare serum gonadotropin and human chorionic gonadotropin.

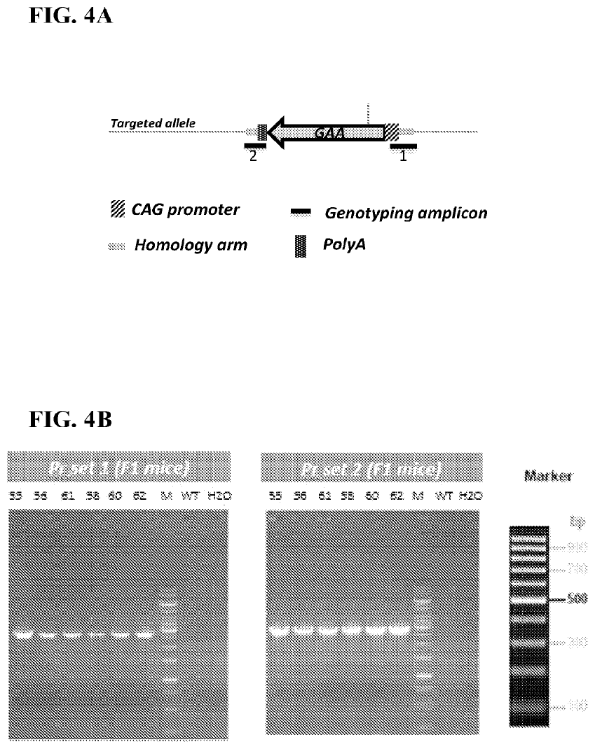

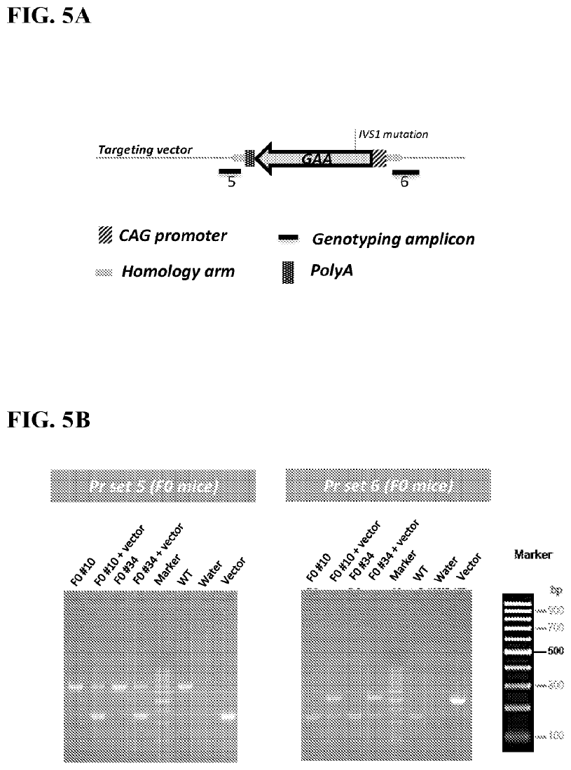

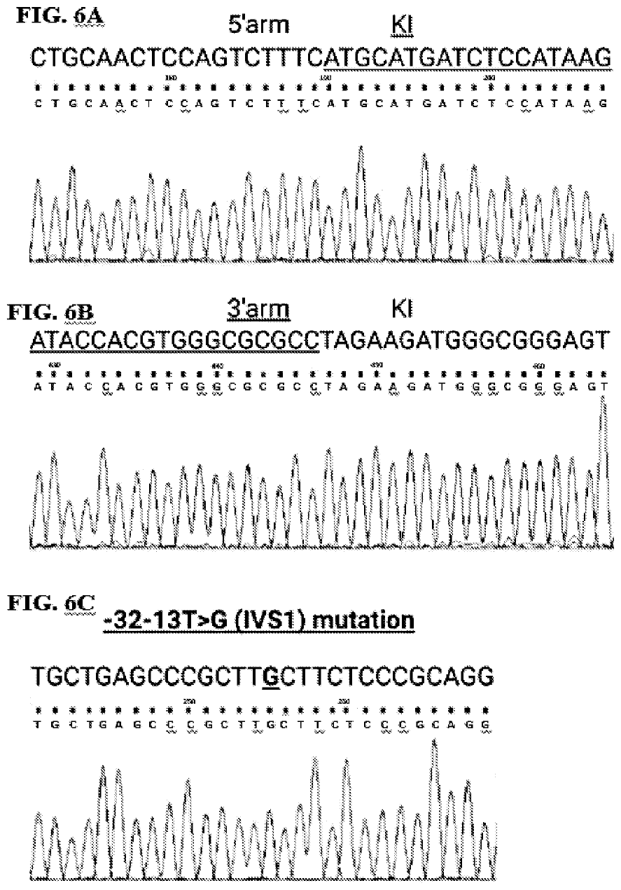

Knock-in mice were genotyped first for the presence of human GAA in the Rosa26 locus (F1 mice, FIGS. 4A-B), then for the presence of the targeting vector to assess for potential random integration (F0 mice, FIGS. 5A-B). Mice determined to contain human GAA, but no targeting vector were confirmed to harbor the GAA IVS1 mutation and correct Rosa26 insertion by Sanger sequencing (FIGS. 6A-C), then bred further to establish germline transmission. One F0 founder line was confirmed with germline transmission of the human GAA insert in the Rosa26 locus.

Genotyping PCR conditions were as described in Table 1.

TABLE 1
Genotyping PCR conditions
8.3 Short fragment PCR reaction
PCR Mixture:

| Component | x1 | |
|---|---|---|
| Mouse tail genomic DNA | 1 | μl |
| Forward primer (10 μM) | 1 | μl |
| Reverse primer (10 μM) | 1 | μl |
| Premix Taq Polymerase | 12.5 | μl |
| ddH₂O | 9.5 | μl |
| Total | 25 | μl |

Cycling Condition:

| Step | Temp. | Time | | | Cycles |
|---|---|---|---|---|---|
| Initial denaturation | 94° C. | 3 | min | | |
| Denaturation | 94° C. | 30 | s | | |
| Annealing | 60° C. | 35 | s | {close oversize bracket} | 35 x |
| Extension | 72° C. | 35 | s | | |
| Additional extension | 72° C. | 5 | min | | |

### Example 2: Determination of the Genomic Copy Number of the Human GAA Gene in the Pompe Disease Mouse Model

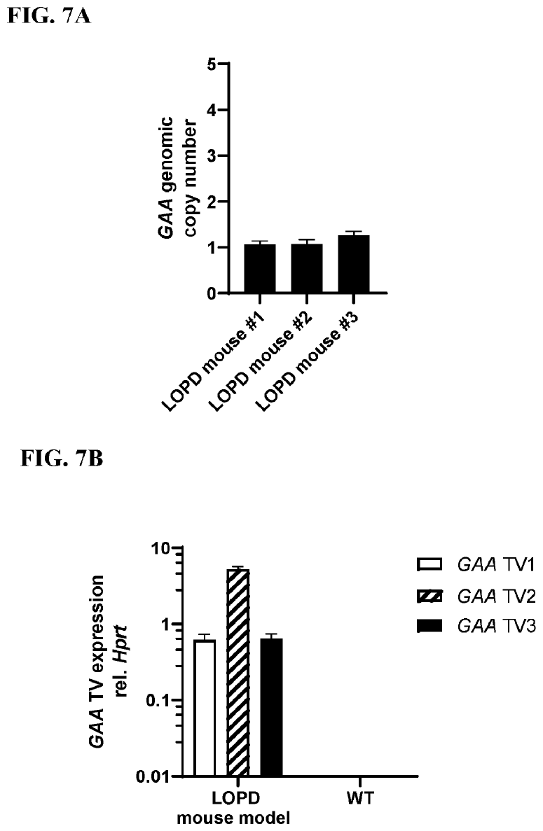

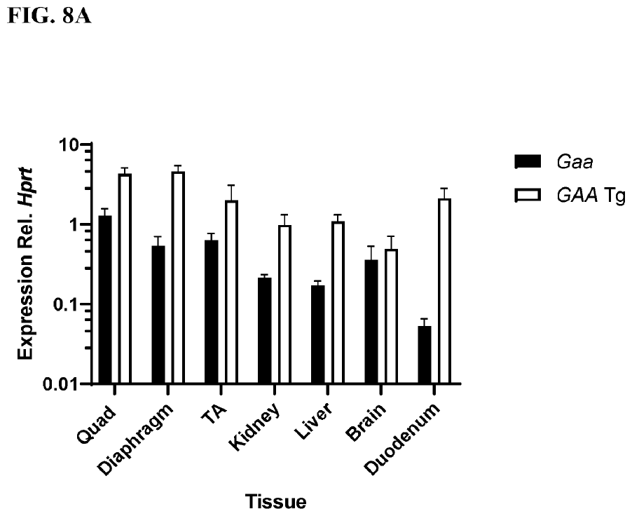

10-20 mg of fresh LOPD murine tail or ear tissue was mechanically homogenized with a metal bead beater using the Quick-DNA 96 kit (Zymo Research) lysis buffer. Total DNA was isolated following the kit protocol and diluted 5-10× before use. 1 uL of DNA was amplified in a PCR reaction containing an assay for human GAA detection on the VIC channel (Hs03961696_cn, Thermo Fisher) and mouse Tfrc (Thermo Fisher) on the VIC channel using Genotyping Master Mix (Thermo Fisher, Quantstudio 7 Pro). A relative standard curve for the duplex qPCR reaction was generated using a 1:1 mixture of mouse gDNA (Promega) and human gDNA (Promega). The relative quantity of human GAA was calculated for each sample and normalized to Tfrc (FIG. 7A). The Multiplex qPCR analysis performed on genomic DNA of three F2 mice showed insertion of one single copy of the GAA into the genome.

### Example 3: GAA TV Expression Analysis in the Pompe Disease Mouse Model

A qPCR analysis was performed on genomic DNA of F2 mice to determine the relative expression levels of the three GAA alternative splicing forms (TV1, TV2, and TV3). cDNA from LOPD mouse quadriceps muscle was amplified using PowerUp SYBR Green Master mix (Thermo Fisher) on a Bio-rad PCR thermocvcler (Bio-Rad) using GAA TV1, TV2, or TV3 primers according to the manufacturer protocol. Relative quantities of each transcript variant were normalized to mouse Hprt (Mm.PT.39a.22214828, Integrated DNA Technologies) (FIG. 7B).

### Example 4: GAA Expression Analysis in the Pompe Disease Mouse Model

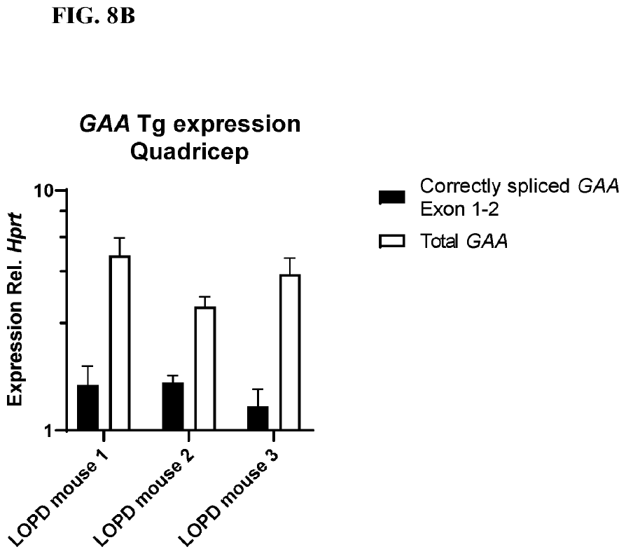

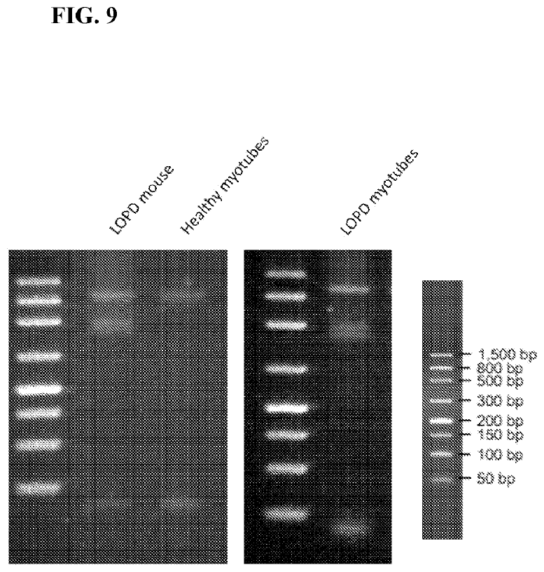

10-20 mg of snap frozen LOPD murine tissue was mechanically homogenized with a metal bead beater and RNA extracted using the Chemagic 360 RNA system (Perkin Elmer). RNA (0.5-1.0 μg) was reverse transcribed using the superscript VILO cDNA synthesis kit (Invitrogen) according to the manufacturers protocol. A multiplex qPCR assay measuring GAA expression at the exon 1-2 locus (Hs00164635_ml, Thermo Fisher) on the FAM channel, GAA at the exon 6-7 locus (AR9HMGG, Thermo Fisher) on the VIC channel, and Hprt (Mm03024075m 1_qsy, Thermo Fisher) on the JUN channel was used with Multiplex Master Mix (Thermo Fisher) on a Quantstudio 7 Pro PCR thermocycler (Thermo Fisher). The analysis was performed on genomic DNA of F2 mice to determine the relative expression levels of the human and the mouse GAA gene in different tissues (FIG. 8A), and on genomic cDNA derived from RNA extracted from LOPD mouse quadriceps muscles of three F2 mice, to determine the relative amount of GAA RNA correctly spliced (exon 1-2 junction) and total GAA RNA (exon 6-7 junction). The results are consistent with misplicing at the exon 1-2 junction (FIG. 8B). cDNA from LOPD mouse quadriceps muscle was amplified in a PCR reaction. GAA exons 1-5 were amplified using primers GAA Ex1-5 Fwd and GAA Ex1-5 Rev with LA Taq Polymerase with GC Buffer (TaKaRa) following the manufacturer's protocol using a Bio-Rad PCR thermocycler (PCR thermocycling conditions were: 95C, 5 min; (95C, 30 sec, 60C, 30 sec, 72C 2 min) X44 cycles). PCR amplification products were visualized using a 2.2% agarose gel (Flashgel System, Lonza) (FIG. 9). The results of the endpoint RT-PCR targeting the exon 1-5 region performed on RNA extracted from LOPD mice, indicated a similar mis-splicing pattern observed in LOPD patient cells.

### Example 5: GAA Splice Variant Analysis in the Pompe Disease Mouse Model

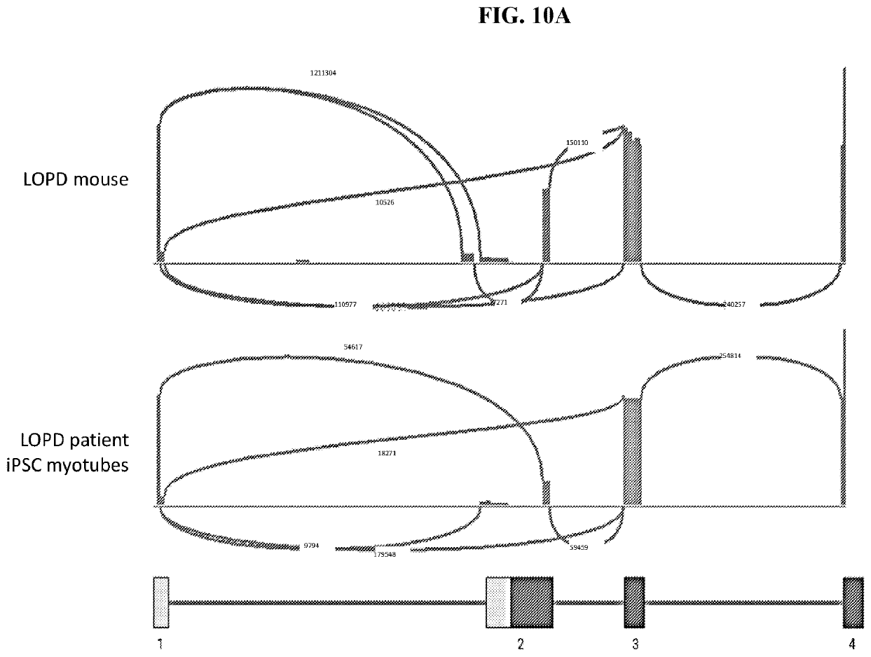

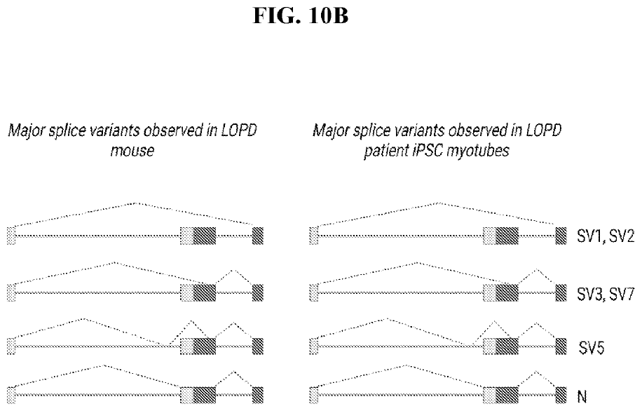

cDNA from LOPD mouse quadriceps muscle was amplified in a PCR reaction. Exons 1-5 of human GAA were PCR amplified using GAA Ex1-5 Fwd and Rev primers. PCR thermocycling conditions were: 95C, 5 min: (95C, 30 sec, 60C, 30 sec, 72C 2 min) X44 cycles using Takara LA Taq DNA polymerase with GC buffer II. The endpoint PCR reactions were purified using the QIAquick PCR Purification Kit (Qiagen), and DNA concentration was normalized to 20 ng/μL. The mixture was subjected to Amplicon-EZ MiSeq 2×150 bp sequencing (Azenta) resulting in >600,000 unfragmented primary reads per sample (>75 bp) mapped to human GAA using Spliced Transcripts Alignment to a reference (STAR) alignment (GitHub). Junctions with greater than 6000 exon-spanning reads were visualized using Integrated Genome Viewer (Broad Institute). Total RNA-seq was carried out from snap frozen LOPD mouse quadriceps tissue (Azenta). >100 M total reads obtained were similarly mapped and visualized. The MiSeq analysis of the exon 1-5 RT-PCR amplification pool, showed several major GAA slice variants in LOPD mouse quadriceps muscle (FIG. 10A). A similarity between major GAA splice variants found in the LOPD mouse quadriceps muscle and those observed in LOPD patient cells was observed (FIG. 10B). All the Splice Variants (SV) listed are deleterious and caused by the IVS1 mutation. The only correctly spliced transcript is the N (normal) variant. It appears that each Transcript Variant is affected by the IVS1 mutation in the same way.

### Example 6: Comparison of the Pompe Disease Mouse Model (GAA^{LOPD(IVS1)+/-} Gaa^{+/+}) and The Acid Alpha-Glucosidase Mouse Model (Gaa^{-/-})

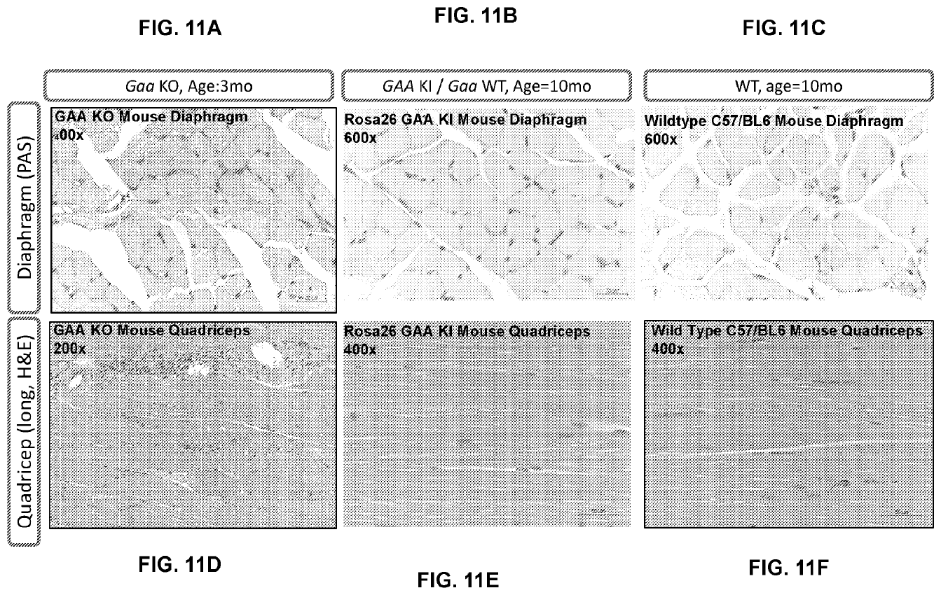

GAA^{LOPD(IVS1)+/-} Gaa^{+/+} mice, generated as described in Example 1, were aged for 10 months together with strain-matched wild type animals (i.e., C57BL/6N strain). Gaa^{-/-} mice (JAX 004154) were aged for 3 months. GAA^{LOPD(IVS1)+/-} Gaa^{+/+}, Gaa^{-/-}, and wild type animals were euthanized, quadriceps muscle were dissected and fixed in 10% neutral buffered formalin (NBF) solution. The fixed tissues (quadriceps) were sectioned and stained with hematoxylin and eosin (H&E) for viewing cellular and tissue structures (i.e., histo-cytological analysis). Additionally, the diaphragm was dissected from the euthanized mice and fixed in 10% neutral buffered formalin (NBF) solution. The fixed tissues (diaphragm) were sectioned and stained with periodic acid-Schiff (PAS) for viewing lysosomes. The stained sections (H&E and PAS) were examined for analyzing the cellular and tissue structures and for the presence of enlarged lysosomes. The quadriceps and diaphragm tissues dissected from the GAA^{LOPD(IVS1)+/-} Gaa^{+/+} (FIG. 11B and FIG. 11E) were comparable to the same tissues dissected from the wild type mice (FIG. 11C and FIG. 11F), and showed no signs of chronic inflammation, enlarged lysosomes, or myocyte degeneration. Quadriceps and diaphragm tissues dissected from the Gaa^{-/-} (FIG. 11A and FIG. 11D), instead, presented signs characteristic of chronic inflammation, enlarged lysosomes, and myocyte degeneration. These results are consistent with the presence of the Gaa gene in the GAA^{LOPD(IVS1)+/-} Gaa^{+/+} mouse model genome.

### Example 7: Assay Splice Modulating Agents in the Pompe Disease Mouse Model

(GAA^{LOPD(IVS1)+/-} Gaa^{+/+}). The GAA^{LOPD(IVS1)+/-} Gaa^{+/+} LOPD transgenic mice were injected with a single intravenous injection (IV) of PPMO (peptide-conjugated phosphorodiamidate morpholino) compounds targeting the IVS1-189 region (PPMOs 1-3, Table 2) or with a single intravenous injection of PPMO compounds targeting the IVS1-69 region (PPMOs 4-5, Table 2). Control animals were injected with a single intravenous injection of a non-targeting PPMO compound (NTC, PPMO 6, Table 2), or with a single intravenous injection of sterile saline.

TABLE 2
PPMO Compounds (“b” indicates an abasic subunit).

| | NG ID | Compound | | |
|---|---|---|---|---|
| | [TO BE | ID | | |
| | DELETED | [TO BE | | |
| Ex | ONCE | DELETED | | |
| compound | CON- | ONCE | | |
| ID | FIRMED | CONFIRMED] | Sequence | Region |
| PPMO 1 | 16-0106 | DR-0719-2 | CCAGAAGGAA | IVS1-189 |
| | | | GGCGAGAAAA | |
| | | | GC | |
| PPMO 2 | 20-0125 | DR-0701-2 | CCAGAAGGAA | IVS1-189 |
| | | | BGGCGAGAAA | |
| | | | AGC | |
| PPMO 3 | 20-0127 | DR-0718-2 | CCAGAAGGAA | IVS1-189 |
| | | | GGBCGAGAAA | |
| | | | AGC | |
| PPMO 4 | 20-0671 | DR-0702-2 | GCACTCACGG | IVS1-69 |
| | | | BGCTCTCAAA | |
| | | | GCAGC | |
| PPMO 5 | 20-0674 | DR-0720-2 | GCACTCACGB | IVS1-69 |
| | | | BGCTCTCAAA | |
| | | | GCAGC | |
| PPMO 6 | 11-0153 | NTC/eGFP | GCTATTACCT | |
| | | | TAACCCAG | |

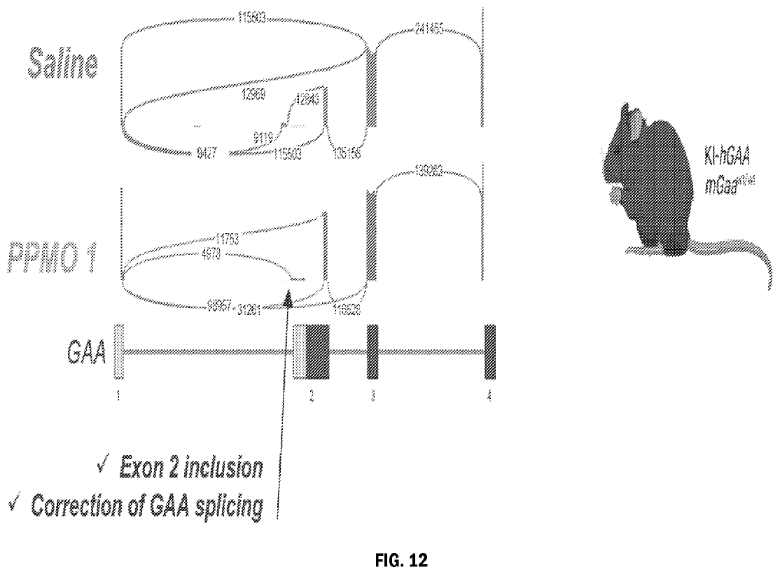

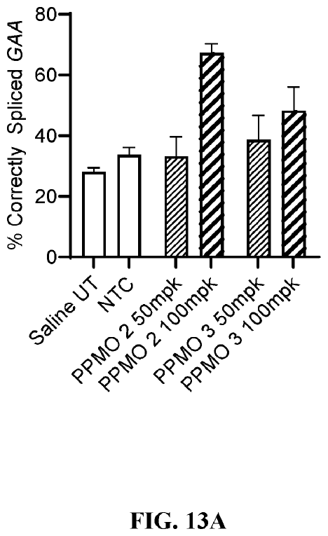

7 days later mice injected with PPMO1 and control mice were euthanized and quadriceps muscles were excised and flash frozen in liquid nitrogen. 10-20 mg of snap frozen LOPD murine tissue was mechanically homogenized with a metal bead beater and RNA extracted using the Chemagic 360 RNA system (Perkin Elmer Chemagic). RNA (0.5-1.0 μg) was reverse transcribed using the superscript VILO kit (Thermo Fisher) according to the manufacturers protocol, and subjected to PCR amplification of GAA exons 1-5. Amplicon sequencing of the amplified product pool was carried out (Azenta), and splice junctions were analyzed as described in Example 5. The results showed that PPMO1 compound corrected LOPD splicing in vivo. Amplicon sequencing of PCR-amplified GAA exons 1-5 demonstrated a qualitative improvement in correctly-spliced GAA exon 1-2 after a single IV dose of PPMO1 compound. Additionally, PPMO1 treatment did not result in the production of any new splice junction (FIG. 12).

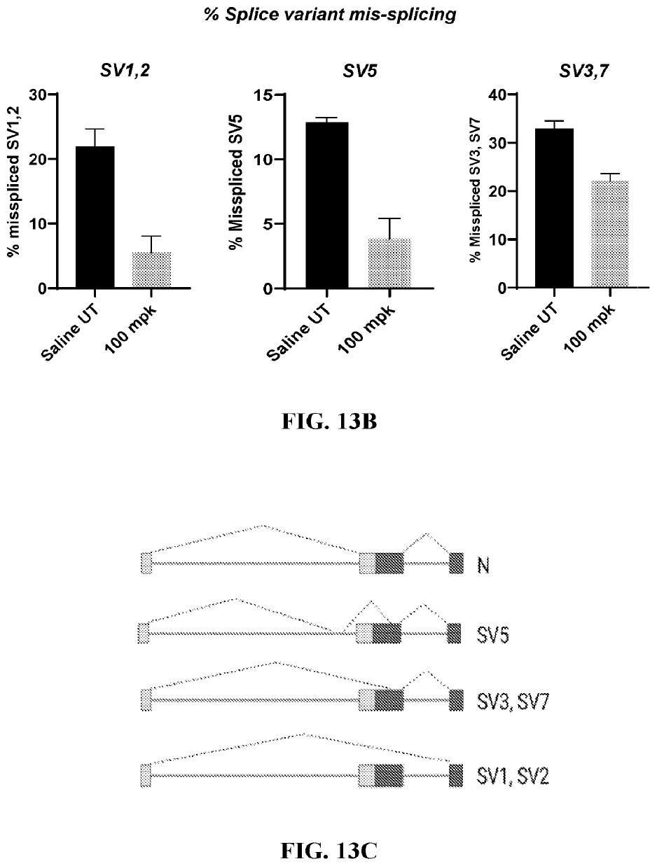

The amplicon pool prepared as described above was separated by capillary electrophoresis (Perkin Elmer LabChip), and bands corresponding to mis-spliced and correctly spliced GAA were identified and quantified. PPMO compounds increase the amount of correctly spliced GAA in vivo (FIG. 13A), and restore all known GAA splice variants (FIG. 13B-C).

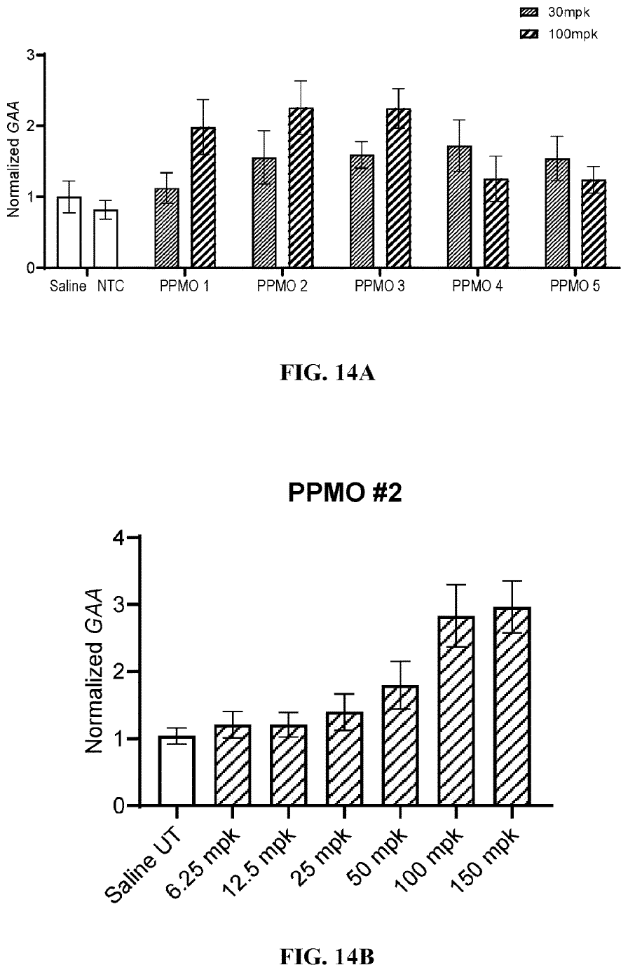

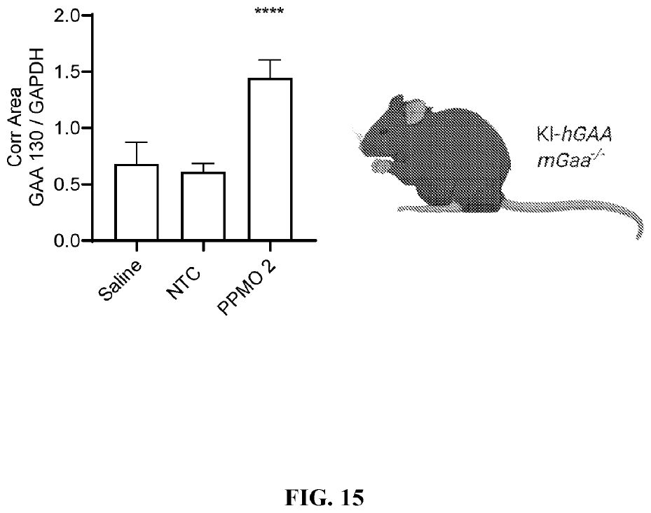

A dose-response assay for the PPMO compounds was performed. PPMO1-6 compounds were injected into GAA^{LOPD(IVS1)+/-} Gaa^{+/+} animals by IV at 30 or 100 mg/kg (n=6). Control animals were injected with a same dose of NTC, or with sterile saline. Tissues were collected 7 days later and quadriceps RNA was examined by qPCR for GAA expression as described in Example 4. All tested PPMO compounds increased the amount of correctly spliced GAA in vivo (FIG. 14A). Additionally, a dose-dependent increase of GAA in quadriceps muscle after administration of PPMO 2 at various doses was shown (FIG. 14B).

### Example 8: Generation of Fully Humanized LOPD Animals (GAA^{LOPD(IVS1)+/-} Gaa^{-/-})

Fully humanized LOPD animals (GAA^{LOPD(IVS1)+/-} Gaa^{-/-}) were generated by crossing the GAALOPD(IVS1)+/− Gaa^{+/+} animals, generated as described in Example 1, with the Gaa^{-/-} (JAX 004154) animals.

Genotyping PCR conditions were as described in Table 3.

TABLE 3
Genotyping PCR conditions

| | Concentration | |
|---|---|---|
| Component | final | |
| Kapa 2G HS buffer | 1.3x | |
| MgCl2 | 2.60 | mM |
| dNTP KAPA | 0.26 | mM |
| oIMR7076 | 0.50 | uM |
| oIMR7077 | 0.50 | uM |
| oIMR7297 | 0.50 | uM |
| Glycerol | 6.50% | |
| Dye | 1.00 | X |
| Kapa 2G HS taq polym | 0.03 | U/ul |
| DNA | 1 | uL |

| STEP | TEMP ° C. | TIME | NOTE |
|---|---|---|---|
| 1 | 94.0 | — | |
| 2 | 94.0 | — | |
| 3 | 65.0 | — | −0.5 C per cycle |
| | | | decrease |
| 4 | 68.0 | — | |
| 5 | | — | repeat steps 2-4 |
| | | | for 10 cycles |
| | | | (Touchdown) |
| 6 | 94.0 | — | |
| 7 | 60.0 | — | |
| 8 | 72.0 | — | |
| 9 | | — | repeat steps 6-8 |
| | | | for 28 cycles |
| 10 | 72.0 | — | |
| 11 | 10.0 | — | hold |

The resulting fully humanized GAA^{LOPD(IVS1)+/-} Gaa^{-/-} animals allow for protein expression analysis, for example by Western blot, because the absence of the Gaa mouse gene avoids any possible cross-reactivity issue of the anti-GAA antibody between the mouse and the human GAA proteins. The fully humanized GAA^{LOPD(IVS1)+/-} Gaa^{-/-} animals were injected (IV) with PPMO2 compound and GAA protein was analyzed in quadriceps muscles 7 days later by Western blot (ProteinSimple® Jes™ System) using a recombinant anti-GAA antibody (Abcam ab 137068) and normalized to total protein. PPMO2 compound increased GAA protein (primary translation) after a single intravenous dose of 150 mg/kg.

Sequence Table (“b” indicates an abasic subunit)

| SEQ ID | | |
|---|---|---|
| NO. | SEQUENCE | DESCRIPTION |
| SEQ ID | cgaccccggagtctccgcgggcggccagggcgcgcgtgcgcggaggtgagccgg | Sequence human |
| NO: 1 | gccggggctgcggggcttccctgagcgcgggccgggtcggtggggcggtcggctg | GAA including |
| | cccgcgcggcctctcagttgggaaagctgaggttgtcgccggggccgcgggtggag | c.-32-13T>G |
| | gtcggggatgaggcagcaggtaggacagtgacctcggtgacgcgaaggaccccgg | mutation |
| | ccacctctaggttctcctcgtccgcccgttgttcagcgagggaggctctgcgcgtgccg | |
| | cagctgacggggaaactgaggcacggagcgggtgagacacctgacgtctgccccg | |
| | cgctgccggcggtaacatcccagaagcgggtttgaacgtgcctagccgtgcccccag | |
| | cctcttcccctgagcggagcttgagccccagacctctagtcctcccggtctttatctgag | |
| | ttcagcttagagatgaacggggagccgccctcctgtgctgggcttggggctggaggct | |
| | gcatcttcccgtttctagggtttcctttccccttttgatcgacgcagtgctcagtcctggcc | |
| | gggacccgagccacctctcctgctcctgcaggacgcacatggctgggtctgaatccct | |
| | ggggtgaggagcaccgtggcctgagagggggcccctgggccagctctgaaatctga | |
| | atgtctcaatcacaaagacccccttaggccaggccaggggtgactgtctctggtctttgt | |
| | ccctggttgctggcacatagcacccgaaacccttggaaaccgagtgatgagagagcc | |
| | ttttgctcatgaggtgactgatgaccggggacaccaggtggcttcaggatggaagcag | |
| | atggccagaaagaccaaggcctgatgacgggttgggatggaaaaggggtgagggg | |
| | ctggagattgagtgaatcaccagtggcttagtcaaccatgcctgcacaatggaacccc | |
| | gtaagaaaccacagggatcagagggcttcccgccgggttgtggaacacaccaaggc | |
| | actggagggtggtgcgagcagagagcacagcatcactgcccccacctcacaccagg | |
| | ccctacgcatctcttccatacggctgtctgagttttatcctttgtaataaaccagcaactgt | |
| | aagaaacgcactttcctgagttctgtgaccctgaagagggagtcctgggaacctctgaa | |
| | tttataactagttgatcgaaagtacaagtgacaacctgggatttgccattggcctctgaag | |
| | tgaaggcagtgttgtgggactgagcccttaacctgtggagtctgtgctgactccaggta | |
| | gtgtcaagattgaattgaattgtaggacacccagccgtgtccagaaagttgcagaattg | |
| | atgggtgtgagaaaaaccctacacatttaatgtcagaagtgtgggtaaaatgtttcaccc | |
| | tccagcccagagagccctaatttaccagtggcccacggtggaacaccacgtccggcc | |
| | gggggcagagcgttcccagccaagccttctgtaacatgacatgacaggtcagactcc | |
| | ctcgggccctgagttcacttcttcctggtatgtgaccagctcccagtaccagagaaggtt | |
| | gcacagtcctctgctccaaggagcttcactggccaggggctgctttctgaaatccttgc | |
| | ctgcctctgctccaaggcccgttcctcagagacgcagacccctctgatggctgactttg | |
| | gtttgaggacctctctgcatccctcccccatggccttgctcctaggacaccttcttcctcct | |
| | ttccctggggtcagacttgcctaggtgcggtggctctcccagccttccccacgccctcc | |
| | ccatggtgtattacacacaccaaagggactcccctattgaaatccatgcatattgaatcg | |
| | catgtgggttccggctgctcctgggaggagccaggctaatagaatgtttgccataaaat | |
| | attaatgtacagagaagcgaaacaaaggtcgttggtacttgttaaccttaccagcagaat | |
| | aatgaaagcgaacccccatatctcatctgcacgcgacatccttgttgtgtctgtacccga | |
| | ggctccaggtgcagccactgttacagagactgtgtttcttccccatgtacctcgggggc | |
| | cgggaggggttctgatctgcaaagtcgccagaggttaagtcctttctctcttgtggctttg | |
| | ccacccctggagtgtcaccctcagctgcggtgcccaggattccccactgtggtatgtcc | |
| | gtgcaccagtcaataggaaagggagcaaggaaaggtactgggtccccctaaggaca | |
| | tacgagttgccagaatcacttccgctgacacccagtggaccaagccgcacctttatgca | |
| | gaagtggggctcccagccaggcgtggtcactcctgaaatcccagcacttcggaaggc | |
| | caaggggggtggatcacttgagctcaggagttcgagaccagcctgggtaacatggca | |
| | aaatcccgtctctacaaaaatacagaaaattagctgggtgcggtggtgtgtgcctacag | |
| | tcccagctactcaggaggctgaagtgggaggattgcttgagtctgggaggtggaggtt | |
| | gcagtgagccaggatctcaccacagcactctggcccaggcgacagctgtttggcctgt | |
| | ttcaagtgtctacctgccttgctggtcttcctggggacattctaagcgtgtttgatttgtaac | |
| | attttagcagactgtgcaagtgctctgcactcccctgctggagcttttctcgcccttccttc | |
| | tggccctctccccagtctagacagcagggcaacacccaccctggccaccttacccca | |
| | cctgcctgggtgctgcagtgccagccgcggttgatgtctcagagctgctttgagagcc | |
| | ccgtgagtgccgcccctcccgcctccctgctgagcccgcttgcttctcccgcaggcct | |
| | gtaggagctgtccaggccatctccaaccatgggagtgaggcacccgccctgctccca | |
| | ccggctcctggccgtctgcgccctcgtgtccttggcaaccgctgcactcctggggcac | |
| | atcctactccatgatttcctgctggttccccgagagctgagtggctcctccccagtcctg | |
| | gaggagactcacccagctcaccagcagggagccagcagaccagggccccgggat | |
| | gcccaggcacaccccggccgtcccagagcagtgcccacacagtgcgacgtccccc | |
| | ccaacagccgcttcgattgcgcccctgacaaggccatcacccaggaacagtgcgag | |
| | gcccgcggctgttgctacatccctgcaaagcaggggctgcagggagcccagatggg | |
| | gcagccctggtgcttcttcccacccagctaccccagctacaagctggagaacctgagc | |
| | tcctctgaaatgggctacacggccaccctgacccgtaccacccccaccttcttccccaa | |
| | ggacatcctgaccctgcggctggacgtgatgatggagactgagaaccgcctccacttc | |
| | acggtgggcagggcaggggcgggggcggcggccagggcagagggtgcgcgtgg | |
| | acatcgacacccacgcacctcacaagggtggggtgcatgttgcaccactgtgtgctgg | |
| | gcccttgctgggagcggaggtgtgagcagacaatggcagcgcccctcggggagca | |
| | gtggggacaccacggtgacaggtactccagaaggcagggctcggggctcattcatct | |
| | ttatgaaaaggtgggtcaggtagagtagggctgccagaggttgcgaatgaaaacagg | |
| | atgcccagtaaacccgaattgcagataccccaggcatgactttgtttttttgtgtaaggat | |
| | gcaaaatttgggatgtatttatactagaaaagctgcttgttgtttatctgaaattcagagtta | |
| | tcaggtgttctgtattttacctccatcctgggggaggcgtcctcctcctggctctgcagat | |
| | gagggagccgaggctcagagaggctgaatgtgctgcccatggtcccacatccatgtg | |
| | tggctgcaccaggacctgacctgtccttggcgtgcgggttgttctctggagagtaaggt | |
| | ggctgtggggaacatcaataaacccccatctcttctagatcaaagatccagctaacagg | |
| | cgctacgaggtgcccttggagaccccgcatgtccacagccgggcaccgtccccactc | |
| | tacagcgtggagttctccgaggagcccttcggggtgatcgtgcgccggcagctggac | |
| | ggccgcgtgctgtgagttctgggctctgtgccagcatgatggggagggcgacgcgca | |
| | tttctcacacggcagggagggccacacgcgtttgtttctcacacgatgggcagggcga | |
| | cacatgtttgtttctcacacggcggggagggcgacgggcatttctcacagggcgctcc | |
| | ctgggtcttttactcacataggtctaaatcccatgtaaacacgtgttcaggactcaccaag | |
| | cccctgcttgtcatttaactcaggaaaactctcaggaacgacagcacttggatttgcctta | |
| | atcttaagagaagttgccttcggaaatgcgtttttctttttttgctcattcatttactcagtgtc | |
| | cacgcactgaccctccgtgccgggtggtttggatcctgctcccggggacagacacac | |
| | agtgaggggaagccataagcaagtccatgcagacacagcgtcagggagtggtcatg | |
| | cagagagcacgctagaagccagctgtgcagacacggggcagggaggtcccctcta | |
| | gaagccagctgtgcagacgcaggggacagggatggcctctctggaagccagctgtg | |
| | cagatgttgggggcaggggtggcctctctggaagccagctgtgcaggagtgggggg | |
| | tggggaggccactctggaaaccagctgtgcagatgcaggggacaggggtggcctct | |
| | ctgagctgacctctgagtagagagacccaagagaagtttctcaaagcatcttatcaagc | |
| | taggtatggtggttcatgtctgcaattccagcactttgggaggccaaggcgagagggtc | |
| | acttgagcccaggagttcaagaccatcctgggcaacatagcaagaccccatctcttaa | |
| | aaaataaaaataaaaaattagctgggaattgtggcacatgcctgtggtcccagctactc | |
| | aggaggctgaggcaagaggatcccttgagcccaggggttcgaggttgcagtgaacc | |
| | atgattttgccactgcacttcagccttgctgaagaccccgtctcaaaaaacaaacaacaa | |
| | acaggcatcttatcagatctcggtcttgaaagcactcagcgtagtcttgcccaggggag | |
| | ggtgggtgcggtgtgagcccgtcctgcgaaattagctgtgctgtgttaacagaggacg | |
| | cgtcttcctgtggaccgggtttatctgcggctttcatttctcggaggtgctgtttgccttgc | |
| | acttgacccccagcaaacctcaggggtccttctcaggcatggctgggctgggatctgg | |
| | gaggactttggccacaagctcctaggcctggaaaggttctgttcagcccctgcccagc | |
| | cttgcttggggtcatgggacaggcatgtgtgccagttccggtaccagccagttcctgga | |
| | ggtcagccccttgggggcccctcaggggtggtgtgggcccagccaggcggtgcgcc | |
| | tcttctgatatgccctgagagttgatcacgctggtgccagggtgccaagggctgcagg | |
| | gctcggcacggccgcctgtcccagggtcagtgtgctgcagggctggccaggccact | |
| | ccgccctcccagggcaccagggcccgggggtgctctctgggtgctctcaggctcgtg | |
| | tggccccttgggtgtgagcaagcctggctggcctctgtcccgcaggctgaacacgac | |
| | ggtggcgcccctgttctttgcggaccagttccttcagctgtccacctcgctgccctcgca | |
| | gtatatcacaggcctcgccgagcacctcagtcccctgatgctcagcaccagctggacc | |
| | aggatcaccctgtggaaccgggaccttgcgcccacggtacagcggcgggcggcgg | |
| | gcgggggcactgagctggggagcgcaggtgctgaagcgccgtctcctgcatgtccc | |
| | agcccggtgcgaacctctacgggtctcaccctttctacctggcgctggaggacggcg | |
| | ggtcggcacacggggtgttcctgctaaacagcaatgccatgggtaagctgcccgccg | |
| | cccagcgcccgggccggggtctcctccgtgctgcctgccctggagactggaggtcc | |
| | gcatgaggggccctgggcacggtgctgggccttgtgttttctgggaaatgagtcctatg | |
| | ggctgatgcctctcccaactctggccttctgtgctcctaaggagggttctggggccctg | |
| | cctggaggtgggctggcaccacatatctttccgtcccatgccaggttcctcctgagtca | |
| | ggcttagcacggcttccccaggccactctgagctcctcgtggggagagagcctcaact | |
| | ctccgcctgtgattggcccatctgtggggtgcagagccctccaagtgaagaatctgtcc | |
| | cccaaccccagagctgcttcccttccagatgtggtcctgcagccgagccctgcccttag | |
| | ctggaggtcgacaggtgggatcctggatgtctacatcttcctgggcccagagcccaag | |
| | agcgtggtgcagcagtacctggacgttgtgggtagggcctgctccctggccgcggcc | |
| | cccgccccaaggctccctcctccctccctcatgaagtcggcgttggcctgcaggatac | |
| | ccgttcatgccgccatactggggcctgggcttccacctgtgccgctggggctactcctc | |
| | caccgctatcacccgccaggtggtggagaacatgaccagggcccacttccccctggt | |
| | gagttggggtggtggcaggggaggcaaggggctggccgggacgcgtctcctcagg | |
| | ccccagcagacggtcccgtgttgtggctgcaggacgtccagtggaacgacctggact | |
| | acatggactcccggagggacttcacgttcaacaaggatggcttccgggacttcccggc | |
| | catggtgcaggagctgcaccagggcggccggcgctacatgatgatcgtggtgtgtgc | |
| | ccccacactgtgggtctttgggaagggggccgcccggtgcccagtggctccttctctg | |
| | tgcagcgtcatcctcgtgcctgtgtggtcgccgaggatgttttctgagggtctttgtgata | |
| | tcgagggaatatcaagaagtttgcaggcttggccccagctgtccagggaggtcgggtt | |
| | tgagggtccccagaaatggccgggtgctactcagggttctgtcagatgtaggttacttg | |
| | aactgccttaaagcaaaaggccaggggcatgataaactgatgtcacctggtcctggaa | |
| | agtggagggcccggtgggcctgggcatgggtatcgctggaactgtggaggctccgt | |
| | gtgccttctggccgtgcctctccttctggccggctctgaatccctggaaaggacggcgt | |
| | gagtgagggcagcttccagccctcatgctggcaccacagagcggagacttcttcccat | |
| | cagctcccatagaaaagtcccaaagcaggactcttgagtcacccagcacaaagaggc | |
| | ccttccctgagccagtcccacagccagaaggatgcagtttgggggctggtccagccc | |
| | gagtctggtgtccggcacgatggccagaggaggaggtgggaggcagggcgagctg | |
| | aaaagatccaacagttcctgcccggaagatccacttcagcagaggaagcacagatga | |
| | gatgtggggctgtgctgatgctgcctgtttccatccctgccttctgcaggcagcaaaca | |
| | gtagtagcccttaagagcaggagtggaaacacagacttttttctttctcacatttttttaatt | |
| | ataaaagaaaagtgattactgtagaacacttgggaaactctagaggtttaaagaaaagg | |
| | taaaggtaaagcttccattccggcgcgcccctcatcagccagctggtcctgactcgccc | |
| | ggccctggctcctctccaggcaggcgtgtgcaggcatgtgcaggtacacaggcagg | |
| | catgctgtacacacgcatgatgtcatccccagcctcatcctctcactgtctcagttttccc | |
| | cgtggctggcgccagggctctgggccaccctcaccttgacaggtttccctcttcccag | |
| | gatcctgccatcagcagctcgggccctgccgggagctacaggccctacgacgaggg | |
| | tctgcggaggggggttttcatcaccaacgagaccggccagccgctgattgggaaggt | |
| | agggcgagggtccaggggacgggggttagaaagcagaggcctccagccaggggg | |
| | agccggcagctgctcaggaagacggtgggatttgaggagccatcacgcccagtggg | |
| | acagctgagaggaatgggccacagtggcccgtgacgatggtggctcctacaaggaat | |
| | ggccccgtgagttcttccatcagcaggcctttgacttcatgggcagctgggcctggccc | |
| | aggcacaagccctgcagaccctcagtgaggccttagggtcctccttgtcctcccagcc | |
| | ccccaggggcctccaggcagggcccccgctgagggagcagctagggagggtctgg | |
| | tgcggatgtgaggctgcctggcagggcttgcacggggccgtctccgctgcccttctcc | |
| | ctgacgctctctggttctgcagcccagcccctgggtggacgtgttgggggtgacccct | |
| | cgttttcccagggttgaggccccttggccccgcatcagtgccttgtggagaaagagctg | |
| | ctcattgacctccagggtgcaggtctctcagatttgcaaatgtgggcgtccactaagagt | |
| | gaggctgcccctctgctcaggctgaggctcagtggggcttccatgcaggccctgggt | |
| | ggggccgggtctccccactgcagcctctcgttgtccaggtatggcccgggtccactgc | |
| | cttccccgacttcaccaaccccacagccctggcctggtgggaggacatggtggctga | |
| | gttccatgaccaggtgcccttcgacggcatgtggattgtaagtgtggccccctcctgag | |
| | catccccaaggcctctggggactaccccaccctcctcactctgggcagagtcacctac | |
| | cagcagcgcttctcttgcaggacatgaacgagccttccaacttcatcaggggctctgag | |
| | gacggctgccccaacaatgagctggagaacccaccctacgtgcctggtcagctcgcc | |
| | ccccacctaccctggggacttaatcaaatcagagactcccttgtctggcctgggagact | |
| | tagcaccctcatctctgagaagcagatgggccagcggggaaaggggcgggggggg | |
| | gatccccaggagaaaggctcaggctgggagactcagccaagcagtgcagacaggg | |
| | tgggtgcagaggcacaggccctgccggaggagacgccgctcacaggtgcttgccag | |
| | agcacagtgaggccgactcgactcagagccgtctcgataggcgcagggaccatgca | |
| | gcggagacctacccacccgtggggagaggtcaggcccaactcgaatgcagcacgg | |
| | gcaagtggatttctagccagggagcagggtgggctcagaggcaggaattaccaaga | |
| | agaagcatgggggtcagggggattctggctgaactgacccagcaggattcttgctgaa | |
| | ggcaggccagggtgaccagacatcgcctgaggggtggtggaggttggggcttctcg | |
| | ccaaactgtcttagcaggaatggcagaaactgggttttacaaggaagtacaaggatgg | |
| | gcctgggagaaggtttgggggcctgaggctatagtttggcccagcaaagaatcagtg | |
| | agaggatggggttttgggcttaggtaaacaggcaggggagtgcttgaaatgggccaa | |
| | gagacggtggatgtgaagtctgggggtctgcagagcccaggctccagcacccgccc | |
| | agccctgtcttagaagcagtggagatgattacccaggttcccgggtaacgccagcccc | |
| | acagaggcgtggggagcggctgcaggtgcacctccagggccagcctgaagaggca | |
| | gcgacctgcacaggggctcctgggaggtggggggcagggagggcaccttggagcc | |
| | tgccgggaggaagctccctggaaaccagcccccgcctcttccaggggtggttgggg | |
| | ggaccctccaggcggccaccatctgtgcctccagccaccagtttctctccacacactac | |
| | aacctgcacaacctctacggcctgaccgaagccatcgcctcccacaggtgagggcca | |
| | cgtcccgccccactgggctctgccctcacagcctgtcctacaaggttggggcctctgc | |
| | agggcctcagggaggaggaaaagcggaggcccagaccacccggggcccgctggc | |
| | ggcccgagtgctctccccacccgctgcctgcaccccagcctgaagctggagcgctcc | |
| | ttcccacttcatgcctggggcttggagaggaaggaccctggatgctgacaggagtctg | |
| | catcagcggggacctcatgactcctgtgaggctggggggggtcctggctcacctaca | |
| | ggcatcaggtggcccagacagaggcaactgtgcccgcagacatgggcagtagcctc | |
| | gccgtcctcctccccagcctctgcctcatcccagaaagctccttgctcccagctctgcc | |
| | ctgctggtgacagggttcccgagtgaccccgctccacacagccctcacggtgtccccc | |
| | accaccccagggcgctggtgaaggctcgggggacacgcccatttgtgatctcccgct | |
| | cgacctttgctggccacggccgatacgccggccactggacgggggacgtgtggagc | |
| | tcctgggagcagctcgcctcctccgtgccaggtgagctcctaccaggaggggctgct | |
| | cagcagagtagagccgggggcctctatgggaggcttgccggggccccccacccact | |
| | tagcaggtggggctctgggtcacttggcctgagctggctctgctgcagcagcctgagg | |
| | accagcctgactctgccctcccagaaatcctgcagtttaacctgctgggggtgcctctg | |
| | gtcggggccgacgtctgcggcttcctgggcaacacctcagaggagctgtgtgtgcgc | |
| | tggacccagctgggggccttctaccccttcatgcggaaccacaacagcctgctcagtc | |
| | tggtagggtgggggtggcggcatggcaggtgggcgatcccacccacccaagactct | |
| | cccctgggaatcccacccctgctggagaagcaccccatgctgggtggctgagaagtg | |
| | cagctctcccgaggcggggactccaggggaccgcggccccagcacccaagtgcttc | |
| | ctttgcccccgcctgccctgcagccccaggagccgtacagcttcagcgagccggccc | |
| | agcaggccatgaggaaggccctcaccctgcgctacgcactcctcccccacctctaca | |
| | cactgttccaccaggcccacgtcgcgggggagaccgtggcccggcccctcttcctgg | |
| | agtgagtgacctaggcaggggcggtggcccatgtgtgccctgggggaggggcacgt | |
| | aactcccaggcagccctgtcctgctgtgggctgtgttccccaggacccagcaggttgc | |
| | cgctgagtgagacaacatttgggcctggcttaagggggaagggcagcaagaaaacc | |
| | cagtaatatcccccagacaggccgtagtacacacgaggagttcctaacaacagccctg | |
| | cacatcagtgtgttgagggaggattcccagagagtgaggtgattagttaactatttgcag | |
| | aagtcaatttatttttcttgcataccatagaaacataagttccagataaattaaatagttaat | |
| | gtcaaaaatcaagctgtggaggccaggcacggtggctaacgcctgtaatcccagaac | |
| | tttgggaggctgaggcgagtggatcacctgaggtcaagagttcgaggccagcctggc | |
| | caacatggtgaaacccatctctactaaaaatacaaaaattagccgtgcatggtggtggg | |
| | cgcctgtagtccctgctactcaggaggctgaggccagagaatcccttgaacctgggag | |
| | gaggagattgcagtgagccgagatcgcgccactgtactccagcctgtgtgactccatc | |
| | tcaaaaaaaaaaaaccaagctgtgaaagactccacaggaaaagataagtgaatgtgta | |
| | tctctggggaaaggtttcatttagagaaaaaaaaaaaaaaaaggtgaactttatttgacc | |
| | atagcagaaaaaaaaatgtaaatctctgaatataaacacaaaaagattaaaactgctctg | |
| | aacatggactgtaacagcagagaaggcattgtcaataaaacagcaaatagctactcttc | |
| | ctaataggtagagaactcatacgttggtaagaccaagactaagaaccaaattggagga | |
| | acactgtttgcaaaacaggagatacgaatggtatccacacattcttcacccggaaatcc | |
| | aagaaacgcaatttgtgttttaattactattttaatgaccttgttgtttctgtcactacactttttt | |
| | ttttttttttttagtggttgccctagggattacaattaacatcttaattttagcctcgttggaact | |
| | gatgccaacttgctttcaatagcataaaaaagctttgcttctatgtggtttccattgcctctc | |
| | gttcctctgtgctgttacacgtctgtatccatgatgagcccatccacgcagatttataatga | |
| | ccgccttattcaattatcttttaagtcaaataggagggaaaaatgggttacaaacaaaaa | |
| | gtacatacacactgtctgccttttatatttactgatgtagttacctttacccatggttttatttct | |
| | tcatgtggctttgacttcttgtctaacatctttcatttacagcagaaggactccctttagtattt | |
| | attgtagggcagttctgctagtgatgaattctctcagtttttgttgacttcagagtctcttaat | |
| | ttctccttcattttttgaaagttctgctcaacgtagggttcttgactggcggtcgtttctctga | |
| | acactttgaacttgtcagcccatggcctccgtggtttctgctgagtagcttctgtcttgcct | |
| | cttcccagattctctgtctttggcctttgaagtcccgatggtgtgtctaggtgtagatctctg | |
| | agtttatcttactgggagtttgttgagcttcttggacgtatgcattgatgtttttcatcaaactt | |
| | gggcggtttttcagccattacttcataaaatattttttctgcccctttctgtcttctactctggg | |
| | acttccgttccacacattggtatgcttgacggtgcccaggggtctttgcagctctgttgac | |
| | tccttcctcgttgtgttttctttctgttcttcagactgcttagtctgactgccctgtcttcaagg | |
| | tcactgattcttttttccaccagttctcatctgctgttgagcccctctagtgaacttctcatttt | |
| | agttgctctcttttcaactccagaatttctgtttcattcctttacgtttctctcgttaaggatatt | |
| | ccctatctggtgagtcatgttctcacactttcctttagttcttcagacacggtttcgctgagc | |
| | tctttgaacacatttaaagtagctgacgtaaactctttgtctagtaagtccaatgtctgggc | |
| | ttctctagggacaatttttattaactactgtccccccatctgtgggccatactttcttgtttctt | |
| | tgcgtgtctcatacatttttgtttcagactggacattttaaacgcagctgctgtggtcatcag | |
| | attccccatccccctcaggtctcgttgctattgctgattgttttgtgactttcttgagctaatt | |
| | ccataaggtctgtgttcttcattgtgtgtggccaccaaagtctctgcttgactagcttagtg | |
| | gacagccaataattggtcagatatccttcacagatggcatccgtgagtctcccagccttt | |
| | gctggggcgaggtggggagcaccgtcaacacttagctaggccaaggattcttgctgtt | |
| | cttacaaagattcagccattttgttaaatacatgctccccagatggctgcaaacccttggtt | |
| | agtttcaagagtcctaaaaatgtcaactctgaccatttttgccgatgttcctgttttcacaga | |
| | ggagaggatcttgagaggggcttactccaccatcttcactgacatcactccaagaaatg | |
| | tattttgcaagaaatattttgaagcagaaagacaccatttattttgcccatgaattagaata | |
| | cattagagaaaataagactatcccttgctggcaagaacacagtgacacagtagggtgg | |
| | aatataaattggcacatttgtggaaagcaacagtacatatcagtcaatgtttttgagattca | |
| | cattgatgcgttaattttacaaatagaaatagggcctaaagaagtcacctcaaaaggcat | |
| | gaacgctccatgaatgtatccatgcagggatcatagctgagcactggatgccccctgc | |
| | acgtccgggggcaggaaacaggacagggcagagctgcgtcacagggcaggacag | |
| | tctccgttagacggagaatcctccgtagagctgcttgcacatgtacattcatctttttgtca | |
| | gatgttaattcaagttgcctttggttgtgggactgggaggatcttttctctttgttgatactttt | |
| | tcgtactttccaaatacttgactgatgagcacatgctgccttggttaccggaggataagtg | |
| | agcgagcaaagtgaggccagtgctgtgtccatcctggtgcctcaagcacaagcccct | |
| | attcctgccctgagcccagctgccggcatgtccggggagaaggcttctcccagctccg | |
| | gcattgacttctatctgctggaatcatccctgcccgtctgacctgagtcctccaagtcctc | |
| | cggcaccttgagctccagagagcagaattcagcctcttcctgtgcctccccagggtgg | |
| | gcatatgagccagccccatcccattcatcacccgtatgcctgtgtgcccatcccccttgc | |
| | aggttccccaaggactctagcacctggactgtggaccaccagctcctgtggggggag | |
| | gccctgctcatcaccccagtgctccaggccgggaaggccgaagtgactggctacttc | |
| | cccttgggcacatggtacgacctgcagacggtgagtctggggaccctaagccctggg | |
| | gagacgggagaccagagcagccctcccacctgccccctccacccagttggtgtgac | |
| | caggtggcggaaagaggaacgtatgtgttgagtcccggccatgtgccaggcccccac | |
| | ccggctgctccgcacccatcagcctctccgctcctcacaccatccccatttcccagatg | |
| | agcagactgaggcctgcttgcagaacctggccaagtcccacggccatcacaggctgt | |
| | gcctgtgctgagctggcatacccaggcctctcaggcactgtccccactcagtagccag | |
| | gagggtccctacctacagtgagccctgagtctgcgcctgaagtcacagttcagcccgt | |
| | ctgtgccaggcctcctaggcctccacgtggagccccgggagatggagagcgtggttc | |
| | ctgaggacagcatgggggcctcggcacggcccagaatcctcaaagcaacatctccct | |
| | ccaggtgccagtagaggcccttggcagcctcccacccccacctgcagctccccgtga | |
| | gccagccatccacagcgaggggcagtgggtgacgctgccggcccccctggacacc | |
| | atcaacgtccacctccgggctgggtacatcatccccctgcaggtacctgggccaggc | |
| | ggctatggtgggggtgtggacagcacactgcagagctgggggaggcacagggaga | |
| | tggtgggggagaggcccaggtggggcttctgaggggccgccccccgcagtgtaggt | |
| | tatcaaggagccagccaggccagtgaggtggggagggcacagccccacaaaggcg | |
| | tggagcatggccggcaggagctcagtggtctgcatggtggaggttctgccgggcccg | |
| | gcctcgggcagccgtgggatagcacttgaggtggggaaggtcttgggtcatcaccac | |
| | ggggttccagcccctgcggccgcaggtgttcctgcagatcctagttactggcagcctg | |
| | gtgctgtaccagcctagcattcccgggccctggaggcctccacctccaccagggtgg | |
| | ggatgatgacatcacgtgtccttccctttccagggccctggcctcacaaccacagagtc | |
| | ccgccagcagcccatggccctggctgtggccctgaccaagggtggggaggcccga | |
| | ggggagctgttctgggacgatggagagagcctggaagtgctggagcgaggggccta | |
| | cacacaggtcatcttcctggccaggaatgtgagtcctggggctgctcaggctggtggg | |
| | caggggccggctcggggttgagaaggggtgaggggacctgggcttgggggtccca | |
| | cgatggctacctgccactaggacactctagcaggtggcctggggtcctagagtgagc | |
| | agtggggccgtgcactctgccctttcgtgtacacagagggaggtcacctccctgatgc | |
| | catcatgagtccctgttctcatgggtgttcctgccccagctgtctgctgacacctccacat | |
| | tctctgccttttcatctctctctgctcggcccagaacacgatcgtgaatgagctggtacgt | |
| | gtgaccagtgagggagctggcctgcagctgcagaaggtgactgtcctgggcgtggc | |
| | cacggcgccccagcaggtcctctccaacggtgtccctgtctccaacttcacctacagc | |
| | cccgacaccaaggcaagagggcccagagtggcacagggatcgcgtcccccagccg | |
| | tggtgcagggggcagaaggtgctgggcgtcctggtgaccgatgccaggaacagag | |
| | gatgctgggacctcccaagggggtctttggggaggagtgggaagggtcaggccaca | |
| | caggctgtgcctttcctcctcctgtgtctacacgtgggtgatggggccacaatgacgac | |
| | ctctgagccgtgttgaagcagcaccgcgtttctggcgtgcgttaaggtgacccgcactg | |
| | agagccggggtccccctgcgcctgccggggaggaaccgggtgcgaagcatcccag | |
| | ggccagacggagctgccccctgagcgccgggcctcgctgctgctgggatctcgggg | |
| | ccagatggagccgccttctgagcgctggggtctcactgctgctgggatctcgggctgc | |
| | tccatttgtgctctctcttttccaggtcctggacatctgtgtctcgctgttgatgggagagc | |
| | agtttctcgtcagctggtgttagccgggcggagtgtgttagtctctccagagggaggct | |
| | ggttccccagggaagcagagcctgtgtgcgggcagcagctgtgtgcgggcctgggg | |
| | gttgcatgtgtcacctggagctgggcactaaccattccaagccgccgcatcgcttgtttc | |
| | cacctcctgggccggggctctggcccccaacgtgtctaggagagctttctccctagatc | |
| | gcactgtgggccggggccctggagggctgctctgtgttaataagattgtaaggtttgcc | |
| | ctcctcacctgttgccggcatgcgggtagtattagccacccccctccatctgttcccagc | |
| | accggagaagggggtgctcaggtggaggtgtggggtatgcacctgagctcctgcttc | |
| | gcgcctgctgctctgccccaacgcgaccgctgcccggctgcccagagggctggatg | |
| | cctgccggtccccgagcaagcctgggaactcaggaaaattcacaggacttgggagat | |
| | tctaaatcttaagtgcaattatttttaataaaaggggcatttggaatcag | |
| SEQ ID | ctaacaccagctgacgagaaactgctctccc | KnockIn |
| NO: 2 | ATCAACAGCGAGACACAGATGTCCAGGACCTGGAAA | |
| | AGAGAGAGCACAAATGGAGCAGCCCGAGATCCCAGC | |
| | AGCAGTGA | |
| | GACCCCAGCGCTCAGAAGGCGGCTCCATCTGGCCCC | |
| | GAGATCCCAGCAGCAGCGAGGCCCGGCGCTCAGGGG | |
| | GCAGCTCC | |
| | GTCTGGCCCTGGGATGCTTCGCACCCGGTTCCTCCC | |
| | CGGCAGGCGCAGGGGGACCCCGGCTCTCAGTGCGGG | |
| | TCACCTTA | |
| | ACGCACGCCAGAAACGCGGTGCTGCTTCAACACGGC | |
| | TCAGAGGTCGTCATTGTGGCCCCATCACCCACGTGT | |
| | AGACACAG | |
| | GAGGAGGAAAGGCACAGCCTGTGTGGCCTGACCCTT | |
| | CCCACTCCTCCCCAAAGACCCCCTTGGGAGGTCCCA | |
| | GCATCCTC | |
| | TGTTCCTGGCATCGGTCACCAGGACGCCCAGCACCT | |
| | TCTGCCCCCTGCACCACGGCTGGGGGACGCGATCCC | |
| | TGTGCCAC | |
| | TCTGGGCCCTCTTGCCTTGGTGTCGGGGCTGTAGGT | |
| | GAAGTTGGAGACAGGGACACCGTTGGAGAGGACCTG | |
| | CTGGGGCG | |
| | CCGTGGCCACGCCCAGGACAGTCACCTTCTGCAGCT | |
| | GCAGGCCAGCTCCCTCACTGGTCACACGTACCAGCT | |
| | CATTCACG | |
| | ATCGTGTTCTGGGCCGAGCAGAGAGAGATGAAAAGG | |
| | CAGAGAATGTGGAGGTGTCAGCAGACAGCTGGGGCA | |
| | GGAACACC | |
| | CATGAGAACAGGGACTCATGATGGCATCAGGGAGGT | |
| | GACCTCCCTCTGTGTACACGAAAGGGCAGAGTGCAC | |
| | GGCCCCAC | |
| | TGCTCACTCTAGGACCCCAGGCCACCTGCTAGAGTG | |
| | TCCTAGTGGCAGGTAGCCATCGTGGGACCCCCAAGC | |
| | CCAGGTCC | |
| | CCTCACCCCTTCTCAACCCCGAGCCGGCCCCTGCCC | |
| | ACCAGCCTGAGCAGCCCCAGGACTCACATTCCTGGC | |
| | CAGGAAGA | |
| | TGACCTGTGTGTAGGCCCCTCGCTCCAGCACTTCCA | |
| | GGCTCTCTCCATCGTCCCAGAACAGCTCCCCTCGGG | |
| | CCTCCCCA | |
| | CCCTTGGTCAGGGCCACAGCCAGGGCCATGGGCTGC | |
| | TGGCGGGACTCTGTGGTTGTGAGGCCAGGGCCCTGG | |
| | AAAGGGAA | |
| | GGACACGTGATGTCATCATCCCCACCCTGGTGGAGG | |
| | TGGAGGCCTCCAGGGCCCGGGAATGCTAGGCTGGTA | |
| | CAGCACCA | |
| | GGCTGCCAGTAACTAGGATCTGCAGGAACACCTGCG | |
| | GCCGCAGGGGCTGGAACCCCGTGGTGATGACCCAAG | |
| | ACCTTCCC | |
| | CACCTCAAGTGCTATCCCACGGCTGCCCGAGGCCGG | |
| | GCCCGGCAGAACCTCCACCATGCAGACCACTGAGCT | |
| | CCTGCCGG | |
| | CCATGCTCCACGCCTTTGTGGGGCTGTGCCCTCCCC | |
| | ACCTCACTGGCCTGGCTGGCTCCTTGATAACCTACA | |
| | CTGCGGGG | |
| | GGCGGCCCCTCAGAAGCCCCACCTGGGCCTCTCCCC | |
| | CACCATCTCCCTGTGCCTCCCCCAGCTCTGCAGTGT | |
| | GCTGTCCA | |
| | CACCCCCACCATAGCCGCCTGGCCCAGGTACCTGCA | |
| | GGGGGATGATGTACCCAGCCCGGAGGTGGACGTTGA | |
| | TGGTGTCC | |
| | AGGGGGGCCGGCAGCGTCACCCACTGCCCCTCGCTG | |
| | TGGATGGCTGGCTCACGGGGAGCTGCAGGTGGGGGT | |
| | GGGAGGCT | |
| | GCCAAGGGCCTCTACTGGCACCTGGAGGGAGATGTT | |
| | GCTTTGAGGATTCTGGGCCGTGCCGAGGCCCCCATG | |
| | CTGTCCTC | |
| | AGGAACCACGCTCTCCATCTCCCGGGGCTCCACGTG | |
| | GAGGCCTAGGAGGCCTGGCACAGACGGGCTGAACTG | |
| | TGACTTCA | |
| | GGCGCAGACTCAGGGCTCACTGTAGGTAGGGACCCT | |
| | CCTGGCTACTGAGTGGGGACAGTGCCTGAGAGGCCT | |
| | GGGTATGC | |
| | CAGCTCAGCACAGGCACAGCCTGTGATGGCCGTGGG | |
| | ACTTGGCCAGGTTCTGCAAGCAGGCCTCAGTCTGCT | |
| | CATCTGGG | |
| | AAATGGGGATGGTGTGAGGAGCGGAGAGGCTGATGG | |
| | GTGCGGAGCAGCCGGGTGGGGGCCTGGCACATGGCC | |
| | GGGACTCA | |
| | ACACATACGTTCCTCTTTCCGCCACCTGGTCACACC | |
| | AACTGGGTGGAGGGGGCAGGTGGGAGGGCTGCTCTG | |
| | GTCTCCCG | |
| | TCTCCCCAGGGCTTAGGGTCCCCAGACTCACCGTCT | |
| | GCAGGTCGTACCATGTGCCCAAGGGGAAGTAGCCAG | |
| | TCACTTCG | |
| | GCCTTCCCGGCCTGGAGCACTGGGGTGATGAGCAGG | |
| | GCCTCCCCCCACAGGAGCTGGTGGTCCACAGTCCAG | |
| | GTGCTAGA | |
| | GTCCTTGGGGAACCTGCAAGGGGGATGGGCACACAG | |
| | GCATACGGGTGATGAATGGGATGGGGCTGGCTCATA | |
| | TGCCCACC | |
| | CTGGGGAGGCACAGGAAGAGGCTGAATTCTGCTCTC | |
| | TGGAGCTCAAGGTGCCGGAGGACTTGGAGGACTCAG | |
| | GTCAGACG | |
| | GGCAGGGATGATTCCAGCAGATAGAAGTCAATGCCG | |
| | GAGCTGGGAGAAGCCTTCTCCCCGGACATGCCGGCA | |
| | GCTGGGCT | |
| | CAGGGCAGGAATAGGGGCTTGTGCTTGAGGCACCAG | |
| | GATGGACACAGCACTGGCCTCACTTTGCTCGCTCAC | |
| | TTATCCTC | |
| | CGGTAACCAAGGCAGCATGTGCTCATCAGTCAAGTA | |
| | TTTGGAAAGTACGAAAAAGTATCAACAAAGAGAAAA | |
| | GATCCTCC | |
| | CAGTCCCACAACCAAAGGCAACTTGAATTAACATCT | |
| | GACAAAAAGATGAATGTACATGTGCAAGCAGCTCTA | |
| | CGGAGGAT | |
| | TCTCCGTCTAACGGAGACTGTCCTGCCCTGTGACGC | |
| | AGCTCTGCCCTGTCCTGTTTCCTGCCCCCGGACGTG | |
| | CAGGGGGC | |
| | ATCCAGTGCTCAGCTATGATCCCTGCATGGATACAT | |
| | TCATGGAGCGTTCATGCCTTTTGAGGTGACTTCTTT | |
| | AGGCCCTA | |
| | TTTCTATTTGTAAAATTAACGCATCAATGTGAATCT | |
| | CAAAAACATTGACTGATATGTACTGTTGCTTTCCAC | |
| | AAATGTGC | |
| | CAATTTATATTCCACCCTACTGTGTCACTGTGTTCT | |
| | TGCCAGCAAGGGATAGTCTTATTTTCTCTAATGTAT | |
| | TCTAATTC | |
| | ATGGGCAAAATAAATGGTGTCTTTCTGCTTCAAAAT | |
| | ATTTCTTGCAAAATACATTTCTTGGAGTGATGTCAG | |
| | TGAAGATG | |
| | GTGGAGTAAGCCCCTCTCAAGATCCTCTCCTCTGTG | |
| | AAAACAGGAACATCGGCAAAAATGGTCAGAGTTGAC | |
| | ATTTTTAG | |
| | GACTCTTGAAACTAACCAAGGGTTTGCAGCCATCTG | |
| | GGGAGCATGTATTTAACAAAATGGCTGAATCTTTGT | |
| | AAGAACAG | |
| | CAAGAATCCTTGGCCTAGCTAAGTGTTGACGGTGCT | |
| | CCCCACCTCGCCCCAGCAAAGGCTGGGAGACTCACG | |
| | GATGCCAT | |
| | CTGTGAAGGATATCTGACCAATTATTGGCTGTCCAC | |
| | TAAGCTAGTCAAGCAGAGACTTTGGTGGCCACACAC | |
| | AATGAAGA | |
| | ACACAGACCTTATGGAATTAGCTCAAGAAAGTCACA | |
| | AAACAATCAGCAATAGCAACGAGACCTGAGGGGGAT | |
| | GGGGAATC | |
| | TGATGACCACAGCAGCTGCGTTTAAAATGTCCAGTC | |
| | TGAAACAAAAATGTATGAGACACGCAAAGAAACAAG | |
| | AAAGTATG | |
| | GCCCACAGATGGGGGGACAGTAGTTAATAAAAATTG | |
| | TCCCTAGAGAAGCCCAGACATTGGACTTACTAGACA | |
| | AAGAGTTT | |
| | ACGTCAGCTACTTTAAATGTGTTCAAAGAGCTCAGC | |
| | GAAACCGTGTCTGAAGAACTAAAGGAAAGTGTGAGA | |
| | ACATGACT | |
| | CACCAGATAGGGAATATCCTTAACGAGAGAAACGTA | |
| | AAGGAATGAAACAGAAATTCTGGAGTTGAAAAGAGA | |
| | GCAACTAA | |
| | AATGAGAAGTTCACTAGAGGGGCTCAACAGCAGATG | |
| | AGAACTGGTGGAAAAAAGAATCAGTGACCTTGAAGA | |
| | CAGGGCAG | |
| | TCAGACTAAGCAGTCTGAAGAACAGAAAGAAAACAC | |
| | AACGAGGAAGGAGTCAACAGAGCTGCAAAGACCCCT | |
| | GGGCACCG | |
| | TCAAGCATACCAATGTGTGGAACGGAAGTCCCAGAG | |
| | TAGAAGACAGAAAGGGGCAGAAAAAATATTTTATGA | |
| | AGTAATGG | |
| | CTGAAAAACCGCCCAAGTTTGATGAAAAACATCAAT | |
| | GCATACGTCCAAGAAGCTCAACAAACTCCCAGTAAG | |
| | ATAAACTC | |
| | AGAGATCTACACCTAGACACACCATCGGGACTTCAA | |
| | AGGCCAAAGACAGAGAATCTGGGAAGAGGCAAGACA | |
| | GAAGCTAC | |
| | TCAGCAGAAACCACGGAGGCCATGGGCTGACAAGTT | |
| | CAAAGTGTTCAGAGAAACGACCGCCAGTCAAGAACC | |
| | CTACGTTG | |
| | AGCAGAACTTTCAAAAAATGAAGGAGAAATTAAGAG | |
| | ACTCTGAAGTCAACAAAAACTGAGAGAATTCATCAC | |
| | TAGCAGAA | |
| | CTGCCCTACAATAAATACTAAAGGGAGTCCTTCTGC | |
| | TGTAAATGAAAGATGTTAGACAAGAAGTCAAAGCCA | |
| | CATGAAGA | |
| | AATAAAACCATGGGTAAAGGTAACTACATCAGTAAA | |
| | TATAAAAGGCAGACAGTGTGTATGTACTTTTTGTTT | |
| | GTAACCCA | |
| | TTTTTCCCTCCTATTTGACTTAAAAGATAATTGAAT | |
| | AAGGCGGTCATTATAAATCTGCGTGGATGGGCTCAT | |
| | CATGGATA | |
| | CAGACGTGTAACAGCACAGAGGAACGAGAGGCAATG | |
| | GAAACCACATAGAAGCAAAGCTTTTTTATGCTATTG | |
| | AAAGCAAG | |
| | TTGGCATCAGTTCCAACGAGGCTAAAATTAAGATGT | |
| | TAATTGTAATCCCTAGGGCAACCACTAAAAAAAAAA | |
| | AAAAAAAA | |
| | GTGTAGTGACAGAAACAACAAGGTCATTAAAATAGT | |
| | AATTAAAACACAAATTGCGTTTCTTGGATTTCCGGG | |
| | TGAAGAAT | |
| | GTGTGGATACCATTCGTATCTCCTGTTTTGCAAACA | |
| | GTGTTCCTCCAATTTGGTTCTTAGTCTTGGTCTTAC | |
| | CAACGTAT | |
| | GAGTTCTCTACCTATTAGGAAGAGTAGCTATTTGCT | |
| | GTTTTATTGACAATGCCTTCTCTGCTGTTACAGTCC | |
| | ATGTTCAG | |
| | AGCAGTTTTAATCTTTTTGTGTTTATATTCAGAGAT | |
| | TTACATTTTTTTTTCTGCTATGGTCAAATAAAGTTC | |
| | ACCTTTTT | |
| | TTTTTTTTTTTCTCTAAATGAAACCTTTCCCCAGAG | |
| | ATACACATTCACTTATCTTTTCCTGTGGAGTCTTTC | |
| | ACAGCTTG | |
| | GTTTTTTTTTTTTGAGATGGAGTCACACAGGCTGGA | |
| | GTACAGTGGCGCGATCTCGGCTCACTGCAATCTCCT | |
| | CCTCCCAG | |
| | GTTCAAGGGATTCTCTGGCCTCAGCCTCCTGAGTAG | |
| | CAGGGACTACAGGCGCCCACCACCATGCACGGCTAA | |
| | TTTTTGTA | |
| | TTTTTAGTAGAGATGGGTTTCACCATGTTGGCCAGG | |
| | CTGGCCTCGAACTCTTGACCTCAGGTGATCCACTCG | |
| | CCTCAGCC | |
| | TCCCAAAGTTCTGGGATTACAGGCGTTAGCCACCGT | |
| | GCCTGGCCTCCACAGCTTGATTTTTGACATTAACTA | |
| | TTTAATTT | |
| | ATCTGGAACTTATGTTTCTATGGTATGCAAGAAAAA | |
| | TAAATTGACTTCTGCAAATAGTTAACTAATCACCTC | |
| | ACTCTCTG | |
| | GGAATCCTCCCTCAACACACTGATGTGCAGGGCTGT | |
| | TGTTAGGAACTCCTCGTGTGTACTACGGCCTGTCTG | |
| | GGGGATAT | |
| | TACTGGGTTTTCTTGCTGCCCTTCCCCCTTAAGCCA | |
| | GGCCCAAATGTTGTCTCACTCAGCGGCAACCTGCTG | |
| | GGTCCTGG | |
| | GGAACACAGCCCACAGCAGGACAGGGCTGCCTGGGA | |
| | GTTACGTGCCCCTCCCCCAGGGCACACATGGGCCAC | |
| | CGCCCCTG | |
| | CCTAGGTCACTCACTCCAGGAAGAGGGGCCGGGCCA | |
| | CGGTCTCCCCCGCGACGTGGGCCTGGTGGAACAGTG | |
| | TGTAGAGG | |
| | TGGGGGAGGAGTGCGTAGCGCAGGGTGAGGGCCTTC | |
| | CTCATGGCCTGCTGGGCCGGCTCGCTGAAGCTGTAC | |
| | GGCTCCTG | |
| | GGGCTGCAGGGCAGGCGGGGGCAAAGGAAGCACTTG | |
| | GGTGCTGGGGCCGCGGTCCCCTGGAGTCCCCGCCTC | |
| | GGGAGAGC | |
| | TGCACTTCTCAGCCACCCAGCATGGGGTGCTTCTCC | |
| | AGCAGGGGTGGGATTCCCAGGGGAGAGTCTTGGGTG | |
| | GGTGGGAT | |
| | CGCCCACCTGCCATGCCGCCACCCCCACCCTACCAG | |
| | ACTGAGCAGGCTGTTGTGGTTCCGCATGAAGGGGTA | |
| | GAAGGCCC | |
| | CCAGCTGGGTCCAGCGCACACACAGCTCCTCTGAGG | |
| | TGTTGCCCAGGAAGCCGCAGACGTCGGCCCCGACCA | |
| | GAGGCACC | |
| | CCCAGCAGGTTAAACTGCAGGATTTCTGGGAGGGCA | |
| | GAGTCAGGCTGGTCCTCAGGCTGCTGCAGCAGAGCC | |
| | AGCTCAGG | |
| | CCAAGTGACCCAGAGCCCCACCTGCTAAGTGGGTGG | |
| | GGGGCCCCGGCAAGCCTCCCATAGAGGCCCCCGGCT | |
| | CTACTCTG | |
| | CTGAGCAGCCCCTCCTGGTAGGAGCTCACCTGGCAC | |
| | GGAGGAGGCGAGCTGCTCCCAGGAGCTCCACACGTC | |
| | CCCCGTCC | |
| | AGTGGCCGGCGTATCGGCCGTGGCCAGCAAAGGTCG | |
| | AGCGGGAGATCACAAATGGGCGTGTCCCCCGAGCCT | |
| | TCACCAGC | |
| | GCCCTGGGGTGGTGGGGGACACCGTGAGGGCTGTGT | |
| | GGAGCGGGGTCACTCGGGAACCCTGTCACCAGCAGG | |
| | GCAGAGCT | |
| | GGGAGCAAGGAGCTTTCTGGGATGAGGCAGAGGCTG | |
| | GGGAGGAGGACGGCGAGGCTACTGCCCATGTCTGCG | |
| | GGCACAGT | |
| | TGCCTCTGTCTGGGCCACCTGATGCCTGTAGGTGAG | |
| | CCAGGACCCCCCCCAGCCTCACAGGAGTCATGAGGT | |
| | CCCCGCTG | |
| | ATGCAGACTCCTGTCAGCATCCAGGGTCCTTCCTCT | |
| | CCAAGCCCCAGGCATGAAGTGGGAAGGAGCGCTCCA | |
| | GCTTCAGG | |
| | CTGGGGTGCAGGCAGCGGGTGGGGAGAGCACTCGGG | |
| | CCGCCAGCGGGCCCCGGGTGGTCTGGGCCTCCGCTT | |
| | TTCCTCCT | |
| | CCCTGAGGCCCTGCAGAGGCCCCAACCTTGTAGGAC | |
| | AGGCTGTGAGGGCAGAGCCCAGTGGGGGGGGACGTG | |
| | GCCCTCAC | |
| | CTGTGGGAGGCGATGGCTTCGGTCAGGCCGTAGAGG | |
| | TTGTGCAGGTTGTAGTGTGTGGAGAGAAACTGGTGG | |
| | CTGGAGGC | |
| | ACAGATGGTGGCCGCCTGGAGGGTCCCCCCAACCAC | |
| | CCCTGGAAGAGGCGGGGGCTGGTTTCCAGGGAGCTT | |
| | CCTCCCGG | |
| | CAGGCTCCAAGGTGCCCTCCCTGCCCCCCACCTCCC | |
| | AGGAGCCCCTGTGCAGGTCGCTGCCTCTTCAGGCTG | |
| | GCCCTGGA | |
| | GGTGCACCTGCAGCCGCTCCCCACGCCTCTGTGGGG | |
| | CTGGCGTTACCCGGGAACCTGGGTAATCATCTCCAC | |
| | TGCTTCTA | |
| | AGACAGGGCTGGGCGGGTGCTGGAGCCTGGGCTCTG | |
| | CAGACCCCCAGACTTCACATCCACCGTCTCTTGGCC | |
| | CATTTCAA | |
| | GCACTCCCCTGCCTGTTTACCTAAGCCCAAAACCCC | |
| | ATCCTCTCACTGATTCTTTGCTGGGCCAAACTATAG | |
| | CCTCAGGC | |
| | CCCCAAACCTTCTCCCAGGCCCATCCTTGTACTTCC | |
| | TTGTAAAACCCAGTTTCTGCCATTCCTGCTAAGACA | |
| | GTTTGGCG | |
| | AGAAGCCCCAACCTCCACCACCCCTCAGGCGATGTC | |
| | TGGTCACCCTGGCCTGCCTTCAGCAAGAATCCTGCT | |
| | GGGTCAGT | |
| | TCAGCCAGAATCCCCCTGACCCCCATGCTTCTTCTT | |
| | GGTAATTCCTGCCTCTGAGCCCACCCTGCTCCCTGG | |
| | CTAGAAAT | |
| | CCACTTGCCCGTGCTGCATTCGAGTTGGGCCTGACC | |
| | TCTCCCCACGGGTGGGTAGGTCTCCGCTGCATGGTC | |
| | CCTGCGCC | |
| | TATCGAGACGGCTCTGAGTCGAGTCGGCCTCACTGT | |
| | GCTCTGGCAAGCACCTGTGAGCGGCGTCTCCTCCGG | |
| | CAGGGCCT | |
| | GTGCCTCTGCACCCACCCTGTCTGCACTGCTTGGCT | |
| | GAGTCTCCCAGCCTGAGCCTTTCTCCTGGGGATCCC | |
| | CCCCCCGC | |
| | CCCTTTCCCCGCTGGCCCATCTGCTTCTCAGAGATG | |
| | AGGGTGCTAAGTCTCCCAGGCCAGACAAGGGAGTCT | |
| | CTGATTTG | |
| | ATTAAGTCCCCAGGGTAGGTGGGGGGCGAGCTGACC | |
| | AGGCACGTAGGGTGGGTTCTCCAGCTCATTGTTGGG | |
| | GCAGCCGT | |
| | CCTCAGAGCCCCTGATGAAGTTGGAAGGCTCGTTCA | |
| | TGTCCTGCAAGAGAAGCGCTGCTGGTAGGTGACTCT | |
| | GCCCAGAG | |
| | TGAGGAGGGTGGGGTAGTCCCCAGAGGCCTTGGGGA | |
| | TGCTCAGGAGGGGGCCACACTTACAATCCACATGCC | |
| | GTCGAAGG | |
| | GCACCTGGTCATGGAACTCAGCCACCATGTCCTCCC | |
| | ACCAGGCCAGGGCTGTGGGGTTGGTGAAGTCGGGGA | |
| | AGGCAGTG | |
| | GACCCGGGCCATACCTGGACAACGAGAGGCTGCAGT | |
| | GGGGAGACCCGGCCCCACCCAGGGCCTGCATGGAAG | |
| | CCCCACTG | |
| | AGCCTCAGCCTGAGCAGAGGGGCAGCCTCACTCTTA | |
| | GTGGACGCCCACATTTGCAAATCTGAGAGACCTGCA | |
| | CCCTGGAG | |
| | GTCAATGAGCAGCTCTTTCTCCACAAGGCACTGATG | |
| | CGGGGCCAAGGGGCCTCAACCCTGGGAAAACGAGGG | |
| | GTCACCCC | |
| | CAACACGTCCACCCAGGGGCTGGGCTGCAGAACCAG | |
| | AGAGCGTCAGGGAGAAGGGCAGCGGAGACGGCCCCG | |
| | TGCAAGCC | |
| | CTGCCAGGCAGCCTCACATCCGCACCAGACCCTCCC | |
| | TAGCTGCTCCCTCAGCGGGGGCCCTGCCTGGAGGCC | |
| | CCTGGGGG | |
| | GCTGGGAGGACAAGGAGGACCCTAAGGCCTCACTGA | |
| | GGGTCTGCAGGGCTTGTGCCTGGGCCAGGCCCAGCT | |
| | GCCCATGA | |
| | AGTCAAAGGCCTGCTGATGGAAGAACTCACGGGGCC | |
| | ATTCCTTGTAGGAGCCACCATCGTCACGGGCCACTG | |
| | TGGCCCAT | |
| | TCCTCTCAGCTGTCCCACTGGGCGTGATGGCTCCTC | |
| | AAATCCCACCGTCTTCCTGAGCAGCTGCCGGCTCCC | |
| | CCTGGCTG | |
| | GAGGCCTCTGCTTTCTAACCCCCGTCCCCTGGACCC | |
| | TCGCCCTACCTTCCCAATCAGCGGCTGGCCGGTCTC | |
| | GTTGGTGA | |
| | TGAAAACCCCCCTCCGCAGACCCTCGTCGTAGGGCC | |
| | TGTAGCTCCCGGCAGGGCCCGAGCTGCTGATGGCAG | |
| | GATCCTGG | |
| | GAAGAGGGAAACCTGTCAAGGTGAGGGTGGCCCAGA | |
| | GCCCTGGCGCCAGCCACGGGGAAAACTGAGACAGTG | |
| | AGAGGATG | |
| | AGGCTGGGGATGACATCATGCGTGTGTACAGCATGC | |
| | CTGCCTGTGTACCTGCACATGCCTGCACACGCCTGC | |
| | CTGGAGAG | |
| | GAGCCAGGGCCGGGCGAGTCAGGACCAGCTGGCTGA | |
| | TGAGGGGCGCGCCGGAATGGAAGCTTTACCTTTACC | |
| | TTTTCTTT | |
| | AAACCTCTAGAGTTTCCCAAGTGTTCTACAGTAATC | |
| | ACTTTTCTTTTATAATTAAAAAAATGTGAGAAAGAA | |
| | AAAAGTCT | |
| | GTGTTTCCACTCCTGCTCTTAAGGGCTACTACTGTT | |
| | TGCTGCCTGCAGAAGGCAGGGATGGAAACAGGCAGC | |
| | ATCAGCAC | |
| | AGCCCCACATCTCATCTGTGCTTCCTCTGCTGAAGT | |
| | GGATCTTCCGGGCAGGAACTGTTGGATCTTTTCAGC | |
| | TCGCCCTG | |
| | CCTCCCACCTCCTCCTCTGGCCATCGTGCCGGACAC | |
| | CAGACTCGGGCTGGACCAGCCCCCAAACTGCATCCT | |
| | TCTGGCTG | |
| | TGGGACTGGCTCAGGGAAGGGCCTCTTTGTGCTGGG | |
| | TGACTCAAGAGTCCTGCTTTGGGACTTTTCTATGGG | |
| | AGCTGATG | |
| | GGAAGAAGTCTCCGCTCTGTGGTGCCAGCATGAGGG | |
| | CTGGAAGCTGCCCTCACTCACGCCGTCCTTTCCAGG | |
| | GATTCAGA | |
| | GCCGGCCAGAAGGAGAGGCACGGCCAGAAGGCACAC | |
| | GGAGCCTCCACAGTTCCAGCGATACCCATGCCCAGG | |
| | CCCACCGG | |
| | GCCCTCCACTTTCCAGGACCAGGTGACATCAGTTTA | |
| | TCATGCCCCTGGCCTTTTGCTTTAAGGCAGTTCAAG | |
| | TAACCTAC | |
| | ATCTGACAGAACCCTGAGTAGCACCCGGCCATTTCT | |
| | GGGGACCCTCAAACCCGACCTCCCTGGACAGCTGGG | |
| | GCCAAGCC | |
| | TGCAAACTTCTTGATATTCCCTCGATATCACAAAGA | |
| | CCCTCAGAAAACATCCTCGGCGACCACACAGGCACG | |
| | AGGATGAC | |
| | GCTGCACAGAGAAGGAGCCACTGGGCACCGGGCGGC | |
| | CCCCTTCCCAAAGACCCACAGTGTGGGGGCACACAC | |
| | CACGATCA | |
| | TCATGTAGCGCCGGCCGCCCTGGTGCAGCTCCTGCA | |
| | CCATGGCCGGGAAGTCCCGGAAGCCATCCTTGTTGA | |
| | ACGTGAAG | |
| | TCCCTCCGGGAGTCCATGTAGTCCAGGTCGTTCCAC | |
| | TGGACGTCCTGCAGCCACAACACGGGACCGTCTGCT | |
| | GGGGCCTG | |
| | AGGAGACGCGTCCCGGCCAGCCCCTTGCCTCCCCTG | |
| | CCACCACCCCAACTCACCAGGGGGAAGTGGGCCCTG | |
| | GTCATGTT | |
| | CTCCACCACCTGGCGGGTGATAGCGGTGGAGGAGTA | |
| | GCCCCAGCGGCACAGGTGGAAGCCCAGGCCCCAGTA | |
| | TGGCGGCA | |
| | TGAACGGGTATCCTGCAGGCCAACGCCGACTTCATG | |
| | AGGGAGGGAGGAGGGAGCCTTGGGGCGGGGGCCGCG | |
| | GCCAGGGA | |
| | GCAGGCCCTACCCACAACGTCCAGGTACTGCTGCAC | |
| | CACGCTCTTGGGCTCTGGGCCCAGGAAGATGTAGAC | |
| | ATCCAGGA | |
| | TCCCACCTGTCGACCTCCAGCTAAGGGCAGGGCTCG | |
| | GCTGCAGGACCACATCTGGAAGGGAAGCAGCTCTGG | |
| | GGTTGGGG | |
| | GACAGATTCTTCACTTGGAGGGCTCTGCACCCCACA | |
| | GATGGGCCAATCACAGGCGGAGAGTTGAGGCTCTCT | |
| | CCCCACGA | |
| | GGAGCTCAGAGTGGCCTGGGGAAGCCGTGCTAAGCC | |
| | TGACTCAGGAGGAACCTGGCATGGGACGGAAAGATA | |
| | TGTGGTGC | |
| | CAGCCCACCTCCAGGCAGGGCCCCAGAACCCTCCTT | |
| | AGGAGCACAGAAGGCCAGAGTTGGGAGAGGCATCAG | |
| | CCCATAGG | |
| | ACTCATTTCCCAGAAAACACAAGGCCCAGCACCGTG | |
| | CCCAGGGCCCCTCATGCGGACCTCCAGTCTCCAGGG | |
| | CAGGCAGC | |
| | ACGGAGGAGACCCCGGCCCGGGCGCTGGGCGGCGGG | |
| | CAGCTTACCCATGGCATTGCTGTTTAGCAGGAACAC | |
| | CCCGTGTG | |
| | CCGACCCGCCGTCCTCCAGCGCCAGGTAGAAAGGGT | |
| | GAGACCCGTAGAGGTTCGCACCGGGCTGGGACATGC | |
| | AGGAGACG | |
| | GCGCTTCAGCACCTGCGCTCCCCAGCTCAGTGCCCC | |
| | CGCCCGCCGCCCGCCGCTGTACCGTGGGCGCAAGGT | |
| | CCCGGTTC | |
| | CACAGGGTGATCCTGGTCCAGCTGGTGCTGAGCATC | |
| | AGGGGACTGAGGTGCTCGGCGAGGCCTGTGATATAC | |
| | TGCGAGGG | |
| | CAGCGAGGTGGACAGCTGAAGGAACTGGTCCGCAAA | |
| | GAACAGGGGCGCCACCGTCGTGTTCAGCCTGCGGGA | |
| | CAGAGGCC | |
| | AGCCAGGCTTGCTCACACCCAAGGGGCCACACGAGC | |
| | CTGAGAGCACCCAGAGAGCACCCCCGGGCCCTGGTG | |
| | CCCTGGGA | |
| | GGGCGGAGTGGCCTGGCCAGCCCTGCAGCACACTGA | |
| | CCCTGGGACAGGCGGCCGTGCCGAGCCCTGCAGCCC | |
| | TTGGCACC | |
| | CTGGCACCAGCGTGATCAACTCTCAGGGCATATCAG | |
| | AAGAGGCGCACCGCCTGGCTGGGCCCACACCACCCC | |
| | TGAGGGGC | |
| | CCCCAAGGGGCTGACCTCCAGGAACTGGCTGGTACC | |
| | GGAACTGGCACACATGCCTGTCCCATGACCCCAAGC | |
| | AAGGCTGG | |
| | GCAGGGGCTGAACAGAACCTTTCCAGGCCTAGGAGC | |
| | TTGTGGCCAAAGTCCTCCCAGATCCCAGCCCAGCCA | |
| | TGCCTGAG | |
| | AAGGACCCCTGAGGTTTGCTGGGGGTCAAGTGCAAG | |
| | GCAAACAGCACCTCCGAGAAATGAAAGCCGCAGATA | |
| | AACCCGGT | |
| | CCACAGGAAGACGCGTCCTCTGTTAACACAGCACAG | |
| | CTAATTTCGCAGGACGGGCTCACACCGCACCCACCC | |
| | TCCCCTGG | |
| | GCAAGACTACGCTGAGTGCTTTCAAGACCGAGATCT | |
| | GATAAGATGCCTGTTTGTTGTTTGTTTTTTGAGACG | |
| | GGGTCTTC | |
| | AGCAAGGCTGAAGTGCAGTGGCAAAATCATGGTTCA | |
| | CTGCAACCTCGAACCCCTGGGCTCAAGGGATCCTCT | |
| | TGCCTCAG | |
| | CCTCCTGAGTAGCTGGGACCACAGGCATGTGCCACA | |
| | ATTCCCAGCTAATTTTTTATTTTTATTTTTTAAGAG | |
| | ATGGGGTC | |
| | TTGCTATGTTGCCCAGGATGGTCTTGAACTCCTGGG | |
| | CTCAAGTGACCCTCTCGCCTTGGCCTCCCAAAGTGC | |
| | TGGAATTG | |
| | CAGACATGAACCACCATACCTAGCTTGATAAGATGC | |
| | TTTGAGAAACTTCTCTTGGGTCTCTCTACTCAGAGG | |
| | TCAGCTCA | |
| | GAGAGGCCACCCCTGTCCCCTGCATCTGCACAGCTG | |
| | GTTTCCAGAGTGGCCTCCCCACCCCCCACTCCTGCA | |
| | CAGCTGGC | |
| | TTCCAGAGAGGCCACCCCTGCCCCCAACATCTGCAC | |
| | AGCTGGCTTCCAGAGAGGCCATCCCTGTCCCCTGCG | |
| | TCTGCACA | |
| | GCTGGCTTCTAGAGGGGACCTCCCTGCCCCGTGTCT | |
| | GCACAGCTGGCTTCTAGCGTGCTCTCTGCATGACCA | |
| | CTCCCTGA | |
| | CGCTGTGTCTGCATGGACTTGCTTATGGCTTCCCCT | |
| | CACTGTGTGTCTGTCCCCGGGAGCAGGATCCAAACC | |
| | ACCCGGCA | |
| | CGGAGGGTCAGTGCGTGGACACTGAGTAAATGAATG | |
| | AGCAAAAAAAGAAAAACGCATTTCCGAAGGCAACTT | |
| | CTCTTAAG | |
| | ATTAAGGCAAATCCAAGTGCTGTCGTTCCTGAGAGT | |
| | TTTCCTGAGTTAAATGACAAGCAGGGGCTTGGTGAG | |
| | TCCTGAAC | |
| | ACGTGTTTACATGGGATTTAGACCTATGTGAGTAAA | |
| | AGACCCAGGGAGCGCCCTGTGAGAAATGCCCGTCGC | |
| | CCTCCCCG | |
| | CCGTGTGAGAAACAAACATGTGTCGCCCTGCCCATC | |
| | GTGTGAGAAACAAACGCGTGTGGCCCTCCCTGCCGT | |
| | GTGAGAAA | |
| | TGCGCGTCGCCCTCCCCATCATGCTGGCACAGAGCC | |
| | CAGAACTCACAGCACGCGGCCGTCCAGCTGCCGGCG | |
| | CACGATCA | |
| | CCCCGAAGGGCTCCTCGGAGAACTCCACGCTGTAGA | |
| | GTGGGGACGGTGCCCGGCTGTGGACATGCGGGGTCT | |
| | CCAAGGGC | |
| | ACCTCGTAGCGCCTGTTAGCTGGATCTTTGATCTAG | |
| | AAGAGATGGGGGTTTATTGATGTTCCCCACAGCCAC | |
| | CTTACTCT | |
| | CCAGAGAACAACCCGCACGCCAAGGACAGGTCAGGT | |
| | CCTGGTGCAGCCACACATGGATGTGGGACCATGGGC | |
| | AGCACATT | |
| | CAGCCTCTCTGAGCCTCGGCTCCCTCATCTGCAGAG | |
| | CCAGGAGGAGGACGCCTCCCCCAGGATGGAGGTAAA | |
| | ATACAGAA | |
| | CACCTGATAACTCTGAATTTCAGATAAACAACAAGC | |
| | AGCTTTTCTAGTATAAATACATCCCAAATTTTGCAT | |
| | CCTTACAC | |
| | AAAAAAACAAAGTCATGCCTGGGGTATCTGCAATTC | |
| | GGGTTTACTGGGCATCCTGTTTTCATTCGCAACCTC | |
| | TGGCAGCC | |
| | CTACTCTACCTGACCCACCTTTTCATAAAGATGAAT | |
| | GAGCCCCGAGCCCTGCCTTCTGGAGTACCTGTCACC | |
| | GTGGTGTC | |
| | CCCACTGCTCCCCGAGGGGCGCTGCCATTGTCTGCT | |
| | CACACCTCCGCTCCCAGCAAGGGCCCAGCACACAGT | |
| | GGTGCAAC | |
| | ATGCACCCCACCCTTGTGAGGTGCGTGGGTGTCGAT | |
| | GTCCACGCGCACCCTCTGCCCTGGCCGCCGCCCCCG | |
| | CCCCTGCC | |
| | CTGCCCACCGTGAAGTGGAGGCGGTTCTCAGTCTCC | |
| | ATCATCACGTCCAGCCGCAGGGTCAGGATGTCCTTG | |
| | GGGAAGAA | |
| | GGTGGGGGTGGTACGGGTCAGGGTGGCCGTGTAGCC | |
| | CATTTCAGAGGAGCTCAGGTTCTCCAGCTTGTAGCT | |
| | GGGGTAGC | |
| | TGGGTGGGAAGAAGCACCAGGGCTGCCCCATCTGGG | |
| | CTCCCTGCAGCCCCTGCTTTGCAGGGATGTAGCAAC | |
| | AGCCGCGG | |
| | GCCTCGCACTGTTCCTGGGTGATGGCCTTGTCAGGG | |
| | GCGCAATCGAAGCGGCTGTTGGGGGGGACGTCGCAC | |
| | TGTGTGGG | |
| | CACTGCTCTGGGACGGCCGGGGTGTGCCTGGGCATC | |
| | CCGGGGCCCTGGTCTGCTGGCTCCCTGCTGGTGAGC | |
| | TGGGTGAG | |
| | TCTCCTCCAGGACTGGGGAGGAGCCACTCAGCTCTC | |
| | GGGGAACCAGCAGGAAATCATGGAGTAGGATGTGCC | |
| | CCAGGAGT | |
| | GCAGCGGTTGCCAAGGACACGAGGGCGCAGACGGCC | |
| | AGGAGCCGGTGGGAGCAGGGCGGGTGCCTCACTCCC | |
| | ATGGTTGG | |
| | AGATGGCCTGGACAGCTCCTACAGGCCTGCGGGAGA | |
| | AGCAAGCGGGCTCAGCAGGGAGGCGGGAGGGGCGGC | |
| | ACTCACGG | |
| | GGCTCTCAAAGCAGCTCTGAGACATCAACCGCGGCT | |
| | GGCACTGCAGCACCCAGGCAGGTGGGGTAAGGTGGC | |
| | CAGGGTGG | |
| | GTGTTGCCCTGCTGTCTAGACTGGGGAGAGGGCCAG | |
| | AAGGAAGGGCGAGAAAAGCTCCAGCAGGGGAGTGCA | |
| | GAGCACTT | |
| | GCACAGTCTGCTAAAATGTTACAAATCAAACACGCT | |
| | TAGAATGTCCCCAGGAAGACCAGCAAGGCAGGTAGA | |
| | CACTTGAA | |
| | ACAGGCCAAACAGCTGTCGCCTGGGCCAGAGTGCTG | |
| | TGGTGAGATCCTGGCTCACTGCAACCTCCACCTCCC | |
| | AGACTCAA | |
| | GCAATCCTCCCACTTCAGCCTCCTGAGTAGCTGGGA | |
| | CTGTAGGCACACACCACCGCACCCAGCTAATTTTCT | |
| | GTATTTTT | |
| | GTAGAGACGGGATTTTGCCATGTTACCCAGGCTGGT | |
| | CTCGAACTCCTGAGCTCAAGTGATCCACCCCCCTTG | |
| | GCCTTCCG | |
| | AAGTGCTGGGATTTCAGGAGTGACCACGCCTGGCTG | |
| | GGAGCCCCACTTCTGCATAAAGGTGCGGCTTGGTCC | |
| | ACTGGGTG | |
| | TCAGCGGAAGTGATTCTGGCAACTCGTATGTCCTTA | |
| | GGGGGACCCAGTACCTTTCCTTGCTCCCTTTCCTAT | |
| | TGACTGGT | |
| | GCACGGACATACCACAGTGGGGAATCCTGGGCACCG | |
| | CAGCTGAGGGTGACACTCCAGGGGTGGCAAAGCCAC | |
| | AAGAGAGA | |
| | AAGGACTTAACCTCTGGCGACTTTGCAGATCAGAAC | |
| | CCCTCCCGGCCCCCGAGGTACATGGGGAAGAAACAC | |
| | AGTCTCTG | |
| | TAACAGTGGCTGCACCTGGAGCCTCGGGTACAGACA | |
| | CAACAAGGATGTCGCGTGCAGATGAGATATGGGGGT | |
| | TCGCTTTC | |
| | ATTATTCTGCTGGTAAGGTTAACAAGTACCAACGAC | |
| | CTTTGTTTCGCTTCTCTGTACATTAATATTTTATGG | |
| | CAAACATT | |
| | CTATTAGCCTGGCTCCTCCCAGGAGCAGCCGGAACC | |
| | CACATGCGATTCAATATGCATGGATTTCAATAGGGG | |
| | AGTCCCTT | |
| | TGGTGTGTGTAATACACCATGGGGAGGGCGTGGGGA | |
| | AGGCTGGGAGAGCCACCGCACCTAGGCAAGTCTGAC | |
| | CCCAGGGA | |
| | AAGGAGGAAGAAGGTGTCCTAGGAGCAAGGCCATGG | |
| | GGGAGGGATGCAGAGAGGTCCTCAAACCAAAGTCAG | |
| | CCATCAGA | |
| | GGGGTCTGCGTCTCTGAGGAACGGGCCTTGGAGCAG | |
| | AGGCAGGCAAGGATTTCAGAAAGCAGCCCCTGGCCA | |
| | GTGAAGCT | |
| | CCTTGGAGCAGAGGACTGTGCAACCTTCTCTGGTAC | |
| | TGGGAGCTGGTCACATACCAGGAAGAAGTGAACTCA | |
| | GGGCCCGA | |
| | GGGAGTCTGACCTGTCATGTCATGTTACAGAAGGCT | |
| | TGGCTGGGAACGCTCTGCCCCCGGCCGGACGTGGTG | |
| | TTCCACCG | |
| | TGGGCCACTGGTAAATTAGGGCTCTCTGGGCTGGAG | |
| | GGTGAAACATTTTACCCACACTTCTGACATTAAATG | |
| | TGTAGGGT | |
| | TTTTCTCACACCCATCAATTCTGCAACTTTCTGGAC | |
| | ACGGCTGGGTGTCCTACAATTCAATTCAATCTTGAC | |
| | ACTACCTG | |
| | GAGTCAGCACAGACTCCACAGGTTAAGGGCTCAGTC | |
| | CCACAACACTGCCTTCACTTCAGAGGCCAATGGCAA | |
| | ATCCCAGG | |
| | TTGTCACTTGTACTTTCGATCAACTAGTTATAAATT | |
| | CAGAGGTTCCCAGGACTCCCTCTTCAGGGTCACAGA | |
| | ACTCAGGA | |
| | AAGTGCGTTTCTTACAGTTGCTGGTTTATTACAAAG | |
| | GATAAAACTCAGACAGCCGTATGGAAGAGATGCGTA | |
| | GGGCCTGG | |
| | TGTGAGGTGGGGGCAGTGATGCTGTGCTCTCTGCTC | |
| | GCACCACCCTCCAGTGCCTTGGTGTGTTCCACAACC | |
| | CGGCGGGA | |
| | AGCCCTCTGATCCCTGTGGTTTCTTACGGGGTTCCA | |
| | TTGTGCAGGCATGGTTGACTAAGCCACTGGTGATTC | |
| | ACTCAATC | |
| | TCCAGCCCCTCACCCCTTTTCCATCCCAACCCGTCA | |
| | TCAGGCCTTGGTCTTTCTGGCCATCTGCTTCCATCC | |
| | TGAAGCCA | |
| | CCTGGTGTCCCCGGTCATCAGTCACCTCATGAGCAA | |
| | AAGGCTCTCTCATCACTCGGTTTCCAAGGGTTTCGG | |
| | GTGCTATG | |
| | TGCCAGCAACCAGGGACAAAGACCAGAGACAGTCAC | |
| | CCCTGGCCTGGCCTAAGGGGGTCTTTGTGATTGAGA | |
| | CATTCAGA | |
| | TTTCAGAGCTGGCCCAGGGGCCCCCTCTCAGGCCAC | |
| | GGTGCTCCTCACCCCAGGGATTCAGACCCAGCCATG | |
| | TGCGTCCT | |
| | GCAGGAGCAGGAGAGGTGGCTCGGGTCCCGGCCAGG | |
| | ACTGAGCACTGCGTCGATCAAAAGGGGAAAGGAAAC | |
| | CCTAGAAA | |
| | CGGGAAGATGCAGCCTCCAGCCCCAAGCCCAGCACA | |
| | GGAGGGCGGCTCCCCGTTCATCTCTAAGCTGAACTC | |
| | AGATAAAG | |
| | ACCGGGAGGACTAGAGGTCTGGGGCTCAAGCTCCGC | |
| | TCAGGGGAAGAGGCTGGGGGCACGGCTAGGCACGTT | |
| | CAAACCCG | |
| | CTTCTGGGATGTTACCGCCGGCAGCGCGGGGCAGAC | |
| | GTCAGGTGTCTCACCCGCTCCGTGCCTCAGTTTCCC | |
| | CGTCAGCT | |
| | GCGGCACGCGCAGAGCCTCCCTCGCTGAACAACGGG | |
| | CGGACGAGGAGAACCTAGAGGTGGCCGGGGTCCTTC | |
| | GCGTCACC | |
| | GAGGTCACTGTCCTACCTGCTGCCTCATCCCCGACC | |
| | TCCACCCGCGGCCCCGGCGACAACCTCAGCTTTCCC | |
| | AACTGAGA | |
| | GGCCGCGCGGGCAGCCGACCGCCCCACCGACCCGGC | |
| | CCGCGCTCAGGGAAGCCCCGCAGCCCCGGCCCGGCT | |
| | CACCTCCG | |
| | CGCACGCGCGCCCTGGCCGCCCGCGGAGACTCCGGG | |
| | GTCG | |
| SEQ ID | AATTCTTTGCCAAAATGATGAGACAGCACAATAACC | CAG promoter |
| NO: 3 | AGCACGTTGCCCAGGAGCTGTAGGAAAAAGAAGAAG | |
| | GCATGAAC | |
| | ATGGTTAGCAGAGGCTCTAGAGCCGCCGGTCACACG | |
| | CCAGAAGCCGAACCCCGCCCTGCCCCGTCCCCCCCG | |
| | AAGGCAGC | |
| | CGTCCCCCCGCGGACAGCCCCGAGGCTGGAGAGGGA | |
| | GAAGGGGACGGCGGCGCGGCGACGCACGAAGGCCCT | |
| | CCCCGCCC | |
| | ATTTCCTTCCTGCCGGCGCCGCACCGCTTCGCCCCG | |
| | CGCCCGCTAGAGGGGGTGCGGCGGCGCCTCCCAGAT | |
| | TTCGGCTC | |
| | CGCACAGATTTGGGACAAAGGAAGTCCCTGCGCCCT | |
| | CTCGCACGATTACCATAAAAGGCAATGGCTGCGGCT | |
| | CGCCGCGC | |
| | CTCGACAGCCGCCGGCGCTCCGGGGGCCGCCGCGCC | |
| | CCTCCCCCGAGCCCTCCCCGGCCCGAGGCGGCCCCG | |
| | CCCCGCCC | |
| | GGCACCCCCACCTGCCGCCACCCCCCGCCCGGCACG | |
| | GCGAGCCCCGCGCCACGCCCCGTACGGAGCCCCGCA | |
| | CCCGAAGC | |
| | CGGGCCGTGCTCAGCAACTCGGGGAGGGGGGTGCAG | |
| | GGGGGGTTGCAGCCCGACCGACGCGCCCACACCCCC | |
| | TGCTCACC | |
| | CCCCCACGCACACACCCCGCACGCAGCCTTTGTTCC | |
| | CCTCGCAGCCCCCCCCGCACCGCGGGGCACCGCCCC | |
| | CGGCCGCG | |
| | CTCCCCTCGCGCACACTGCGGAGCGCACAAAGCCCC | |
| | GCGCCGCGCCCGCAGCGCTCACAGCCGCCGGGCAGC | |
| | GCGGAGCC | |
| | GCACGCGGCGCTCCCCACGCACACACACACGCACGC | |
| | ACCCCCCGAGCCGCTCCCCCCGCACAAAGGGCCCTC | |
| | CCGGAGCC | |
| | CCTCAAGGCTTTCACGCAGCCACAGAAAAGAAACAA | |
| | GCCGTCATTAAACCAAGCGCTAATTACAGCCCGGAG | |
| | GAGAAGGG | |
| | CCGTCCCGCCCGCTCACCTGTGGGAGTAACGCGGTC | |
| | AGTCAGAGCCGGGGGGGGCGGCGCGAGGCGGCGGCG | |
| | GAGCGGGG | |
| | CACGGGGCGAAGGCAGCGCGCAGCGACTCCCGCCCG | |
| | CCGCGCGCTTCGCTTTTTATAGGGCCGCCGCCGCCG | |
| | CCGCCTCG | |
| | CCATAAAAGGAAACTTTCGGAGCGCGCCGCTCTGAT | |
| | TGGCTGCCGCCGCACCTCTCCGCCTCGCCCCGCCCC | |
| | GCCCCTCG | |
| | CCCCGCCCCGCCCCGCCTGGCGCGCGCCCCCCCCCC | |
| | CCCCCCGCCCCCATCGCTGCACAAAATAATTAAAAA | |
| | ATAAATAA | |
| | ATACAAAATTGGGGGTGGGGAGGGGGGGGAGATGGG | |
| | GAGAGTGAAGCAGAACGTGGGGCTCACCTCGACCAT | |
| | GGTAATAG | |
| | CGATGACTAATACGTAGATGTACTGCCAAGTAGGAA | |
| | AGTCCCATAAGGTCATGTACTGGGCATAATGCCAGG | |
| | CGGGCCAT | |
| | TTACCGTCATTGACGTCAATAGGGGGCGTACTTGGC | |
| | ATATGATACACTTGATGTACTGCCAAGTGGGCAGTT | |
| | TACCGTAA | |
| | ATACTCCACCCATTGACGTCAATGGAAAGTCCCTAT | |
| | TGGCGTTACTATGGGAACATACGTCATTATTGACGT | |
| | CAATGGGC | |
| | GGGGGTCGTTGGGCGGTCAGCCAGGCGGGCCATTTA | |
| | CCGTAAGTTATGTAACGCGGAACTCCATATATGGGC | |
| | TATGAACT | |
| | AATGACCCCGTAATTGATTACTATTAATAACTAGTC | |
| | AATAATCAAT | |
| SEQ ID | GATCTCCATAAGAGAAGAGGGACAGCTATGACTGGG | rBG pA |
| NO: 4 | AGTAGTCAGGAGAGGAGGAAAAATCTGGCTAGTAAA | |
| | ACAT | |
| | GTAAGGAAAATTTTAGGGATGTTAAAGAAAAAAATA | |
| | ACACAAAACAAAATATAAAAAAAATCTAACCTCAAG | |
| | TCAAGGCT | |
| | TTTCTATGGAATAAGGAATGGACAGCAGGGGGCTGT | |
| | TTCATATACTGATGACCTCTTTATAGCCAACCTTTG | |
| | TTCATGGC | |
| | AGCCAGCATATGGGCATATGTTGCCAAACTCTAAAC | |
| | CAAATACTCATTCTGATGTTTTAAATGATTTGCCCT | |
| | CCCATATG | |
| | TCCTTCCGAGTGAGAGACACAAAAAATTCCAACACA | |
| | CTATTGCAATGAAAATAAATTTCCTTTATTAGCCAG | |
| | AAGTCAGA | |
| | TGCTCAAGGGGCTTCATGATGTCCCCATAATTTTTG | |
| | GCAGAGGGAAAAAGATCTCAGTGGTATTTGTGAGCC | |
| | AGGGCATT | |
| | GGCCACACCAGCCACCACCTTCTGATAGGCAGCCTG | |
| | CACCTGAGGA | |
| SEQ ID | AAGGACCCCGGCCACCTCTAG | GAA TV1 Fwd |
| NO: 5 | | |
| SEQ ID | CCTACAGGCCCGCTCCGTG | GAA TV1 Rev |
| NO: 6 | | |
| SEQ ID | CGCGGAGGTTCTCCTCGTC | GAA TV2 Fwd |
| NO: 7 | | |
| SEQ ID | AGCTCCTACAGGCCCGC | GAA TV2 Rev |
| NO: 8 | | |
| SEQ ID | CGCGGAGGCCTGTAGGA | GAA TV3 Fwd |
| NO: 9 | | |
| SEQ ID | AGGACACGAGGGCGCA | GAA TV3 Rev |
| NO: 10 | | |
| SEQ ID | GGAAACTGAGGCACGGAGCG | GAA Ex1-5 Fwd |
| NO: 11 | | |
| SEQ ID | GGACCACATCCATGGCATTGC | GAA Ex1-5 Rev |
| NO: 12 | | |
| SEQ ID | CTAAATTGTAAGCGTTAATATTTTGTTAAAATTCGC | Sequence of the |
| NO: 13 | GTTAAATTTTTGTTAAATCAGCTCATTTTTTAACCA | final targeting |
| | ATAGGCCG | vector |
| | AAATCGGCAAAATCCCTTATAAATCAAAAGAATAGA | |
| | CCGAGATAGGGTTGAGTGTTGTTCCAGTTTGGAACA | |
| | AGAGTCCA | |
| | CTATTAAAGAACGTGGACTCCAACGTCAAAGGGCGA | |
| | AAAACCGTCTATCAGGGCGATGGCCCACTACGTGAA | |
| | CCATCACC | |
| | CTAATCAAGTTTTTTGGGGTCGAGGTGCCGTAAAGC | |
| | ACTAAATCGGAACCCTAAAGGGAGCCCCCGATTTAG | |
| | AGCTTGAC | |
| | GGGGAAAGCCGGCGAACGTGGCGAGAAAGGAAGGGA | |
| | AGAAAGCGAAAGGAGCGGGCGCTAGGGCGCTGGCAA | |
| | GTGTAGCG | |
| | GTCACGCTGCGCGTAACCACCACACCCGCCGCGCTT | |
| | AATGCGCCGCTACAGGGCGCGTCCCATTCGCCATTC | |
| | AGGCTGCG | |
| | CAACTGTTGGGAAGGGCGATCGGTGCGGGCCTCTTC | |
| | GCTATTACGCCAGCTGGCGAAAGGGGGATGTGCTGC | |
| | AAGGCGAT | |
| | TAAGTTGGGTAACGCCAGGGTTTTCCCAGTCACGAC | |
| | GTTGTAAAACGACGGCCAGTGAATTGTAATACGACT | |
| | CACTATAG | |
| | GGCGAATTGGGTACGCGGCCGCATTCTGGTACCACG | |
| | AGCGACCAGAGTTGTCACAAGGCCGCAAGAACAGGG | |
| | GAGGTGGG | |
| | GGGCTCAGGGACAGAAAAAAAAGTATGTGTATTTTG | |
| | AGAGCAGGGTTGGGAGGCCTCTCCTGAAAAGGGTAT | |
| | AAACGTGG | |
| | AGTAGGCAATACCCAGGCAAAAAGGGGAGACCAGAG | |
| | TAGGGGGAGGGGAAGAGTCCTGACCCAGGGAAGACA | |
| | TTAAAAAG | |
| | GTAGTGGGGTCGACTAGATGAAGGAGAGCCTTTCTC | |
| | TCTGGGCAAGAGCGGTGCAATGGTGTGTAAAGGTAG | |
| | CTGAGAAG | |
| | ACGAAAAGGGCAAGCATCTTCCTGCTACCAGGCTGG | |
| | GGAGGCCCAGGCCCACGACCCCGAGGAGAGGGAACG | |
| | CAGGGAGA | |
| | CTGAGGTGACCCTTCTTTCCCCCGGGGCCCGGTCGT | |
| | GTGGTTCGGTGTCTCTTTTCTGTTGGACCCTTACCT | |
| | TGACCCAG | |
| | GCGCTGCCGGGGCCTGGGCCCGGGCTGCGGCGCACG | |
| | GCACTCCCGGGAGGCAGCGAGACTCGAGTTAGGCCC | |
| | AACGCGGC | |
| | GCCACGGCGTTTCCTGGCCGGGAATGGCCCGTACCC | |
| | GTGAGGTGGGGGTGGGGGGCAGAAAAGGCGGAGCGA | |
| | GCCCGAGG | |
| | CGGGGAGGGGGAGGGCCAGGGGCGGAGGGGGCCGGC | |
| | ACTACTGTGTTGGCGGACTGGCGGGACTAGGGCTGC | |
| | GTGAGTCT | |
| | CTGAGCGCAGGCGGGCGGCGGCCGCCCCTCCCCCGG | |
| | CGGCGGCAGCGGCGGCAGCGGCGGCAGCTCACTCAG | |
| | CCCGCTGC | |
| | CCGAGCGGAAACGCCACTGACCGCACGGGGATTCCC | |
| | AGTGCCGGCGCCAGGGGCACGCGGGACACGCCCCCT | |
| | CCCGCCGC | |
| | GCCATTGGCCTCTCCGCCCACCGCCCCACACTTATT | |
| | GGCCGGTGCGCCGCCAATCAGCGGAGGCTGCCGGGG | |
| | CCGCCTAA | |
| | AGAAGAGGCTGTGCTTTGGGGCTCCGGCTCCTCAGA | |
| | GAGCCTCGGCTAGGTAGGGGATCGGGACTCTGGCGG | |
| | GAGGGCGG | |
| | CTTGGTGCGTTTGCGGGGATGGGCGGCCGCGGCAGG | |
| | CCCTCCGAGCGTGGTGGAGCCGTTCTGTGAGACAGC | |
| | CGGGTACG | |
| | AGTCGTGACGCTGGAAGGGGCAAGCGGGTGGTGGGC | |
| | AGGAATGCGGTCCGCCCTGCAGCAACCGGAGGGGGA | |
| | GGGAGAAG | |
| | GGAGCGGAAAAGTCTCCACCGGACGCGGCCATGGCT | |
| | CGGGGGGGGGGGGGCAGCGGAGGAGCGCTTCCGGCC | |
| | GACGTCTC | |
| | GTCGCTGATTGGCTTCTTTTCCTCCCGCCGTGTGTG | |
| | AAAACACAAATGGCGTGTTTTGGTTGGCGTAAGGCG | |
| | CCTGTCAG | |
| | TTAACGGCAGCCGGAGTGCGCAGCCGCCGGCAGCCT | |
| | CGCTCTGCCCACTGGGTGGGGGGGGAGGTAGGTGGG | |
| | GTGAGGCG | |
| | AGCTGGACGTGCGGGCGCGGTCGGCCTCTGGCGGGG | |
| | CGGGGGAGGGGAGGGAGGGTCAGCGAAAGTAGCTCG | |
| | CGCGCGAG | |
| | CGGCCGCCCACCCTCCCCTTCCTCTGGGGGAGTCGT | |
| | TTTACCCGCCGCCGGCCGGGCCTCGTCGTCTGATTG | |
| | GCTCTCGG | |
| | GGCCCAGAAAACTGGCCCTTGCCATTGGCTCGTGTT | |
| | CGTGCAAGTTGAGTCCATCCGCCGGCCAGCGGGGGC | |
| | GGCGAGGA | |
| | GGCGCTCCCAGGTTCCGGCCCTCCCCTCGGCCCCGC | |
| | GCCGCAGAGTCTGGCCGCGCGCCCCTGCGCAACGTG | |
| | GCAGGAAG | |
| | CGCGCGCTGGGGGCGGGGACGGGCAGTAGGGCTGAG | |
| | CGGCTGCGGGGGGGGTGCAAGCACGTTTCCGACTTG | |
| | AGTTGCCT | |
| | CAAGAGGGGCGTGCTGAGCCAGACCTCCATCGCGCA | |
| | CTCCGGGGAGTGGAGGGAAGGAGCGAGGGCTCAGTT | |
| | GGGCTGTT | |
| | TTGGAGGCAGGAAGCACTTGCTCTCCCAAAGTCGCT | |
| | CTGAGTTGTTATCAGTAAGGGAGCTGCAGTGGAGTA | |
| | GGCGGGGA | |
| | GAAGGCCGCACCCTTCTCCGGAGGGGGGAGGGGAGT | |
| | GTTGCAATACCTTTCTGGGAGTTCTCTGCTGCCTCC | |
| | TGGCTTCT | |
| | GAGGACCGCCCTGGGCCTGGGAGAATCCCTTCCCCC | |
| | TCTTCCCTCGTGATCTGCAACTCCAGTCTTTCATGC | |
| | ATGATCTC | |
| | CATAAGAGAAGAGGGACAGCTATGACTGGGAGTAGT | |
| | CAGGAGAGGAGGAAAAATCTGGCTAGTAAAACATGT | |
| | AAGGAAAA | |
| | TTTTAGGGATGTTAAAGAAAAAAATAACACAAAACA | |
| | AAATATAAAAAAAATCTAACCTCAAGTCAAGGCTTT | |
| | TCTATGGA | |
| | ATAAGGAATGGACAGCAGGGGGCTGTTTCATATACT | |
| | GATGACCTCTTTATAGCCAACCTTTGTTCATGGCAG | |
| | CCAGCATA | |
| | TGGGCATATGTTGCCAAACTCTAAACCAAATACTCA | |
| | TTCTGATGTTTTAAATGATTTGCCCTCCCATATGTC | |
| | CTTCCGAG | |
| | TGAGAGACACAAAAAATTCCAACACACTATTGCAAT | |
| | GAAAATAAATTTCCTTTATTAGCCAGAAGTCAGATG | |
| | CTCAAGGG | |
| | GCTTCATGATGTCCCCATAATTTTTGGCAGAGGGAA | |
| | AAAGATCTCAGTGGTATTTGTGAGCCAGGGCATTGG | |
| | CCACACCA | |
| | GCCACCACCTTCTGATAGGCAGCCTGCACCTGAGGA | |
| | CAATTTTAATTAACTAACACCAGCTGACGAGAAACT | |
| | GCTCTCCC | |
| | ATCAACAGCGAGACACAGATGTCCAGGACCTGGAAA | |
| | AGAGAGAGCACAAATGGAGCAGCCCGAGATCCCAGC | |
| | AGCAGTGA | |
| | GACCCCAGCGCTCAGAAGGCGGCTCCATCTGGCCCC | |
| | GAGATCCCAGCAGCAGCGAGGCCCGGCGCTCAGGGG | |
| | GCAGCTCC | |
| | GTCTGGCCCTGGGATGCTTCGCACCCGGTTCCTCCC | |
| | CGGCAGGCGCAGGGGGACCCCGGCTCTCAGTGCGGG | |
| | TCACCTTA | |
| | ACGCACGCCAGAAACGCGGTGCTGCTTCAACACGGC | |
| | TCAGAGGTCGTCATTGTGGCCCCATCACCCACGTGT | |
| | AGACACAG | |
| | GAGGAGGAAAGGCACAGCCTGTGTGGCCTGACCCTT | |
| | CCCACTCCTCCCCAAAGACCCCCTTGGGAGGTCCCA | |
| | GCATCCTC | |
| | TGTTCCTGGCATCGGTCACCAGGACGCCCAGCACCT | |
| | TCTGCCCCCTGCACCACGGCTGGGGGACGCGATCCC | |
| | TGTGCCAC | |
| | TCTGGGCCCTCTTGCCTTGGTGTCGGGGCTGTAGGT | |
| | GAAGTTGGAGACAGGGACACCGTTGGAGAGGACCTG | |
| | CTGGGGCG | |
| | CCGTGGCCACGCCCAGGACAGTCACCTTCTGCAGCT | |
| | GCAGGCCAGCTCCCTCACTGGTCACACGTACCAGCT | |
| | CATTCACG | |
| | ATCGTGTTCTGGGCCGAGCAGAGAGAGATGAAAAGG | |
| | CAGAGAATGTGGAGGTGTCAGCAGACAGCTGGGGCA | |
| | GGAACACC | |
| | CATGAGAACAGGGACTCATGATGGCATCAGGGAGGT | |
| | GACCTCCCTCTGTGTACACGAAAGGGCAGAGTGCAC | |
| | GGCCCCAC | |
| | TGCTCACTCTAGGACCCCAGGCCACCTGCTAGAGTG | |
| | TCCTAGTGGCAGGTAGCCATCGTGGGACCCCCAAGC | |
| | CCAGGTCC | |
| | CCTCACCCCTTCTCAACCCCGAGCCGGCCCCTGCCC | |
| | ACCAGCCTGAGCAGCCCCAGGACTCACATTCCTGGC | |
| | CAGGAAGA | |
| | TGACCTGTGTGTAGGCCCCTCGCTCCAGCACTTCCA | |
| | GGCTCTCTCCATCGTCCCAGAACAGCTCCCCTCGGG | |
| | CCTCCCCA | |
| | CCCTTGGTCAGGGCCACAGCCAGGGCCATGGGCTGC | |
| | TGGCGGGACTCTGTGGTTGTGAGGCCAGGGCCCTGG | |
| | AAAGGGAA | |
| | GGACACGTGATGTCATCATCCCCACCCTGGTGGAGG | |
| | TGGAGGCCTCCAGGGCCCGGGAATGCTAGGCTGGTA | |
| | CAGCACCA | |
| | GGCTGCCAGTAACTAGGATCTGCAGGAACACCTGCG | |
| | GCCGCAGGGGCTGGAACCCCGTGGTGATGACCCAAG | |
| | ACCTTCCC | |
| | CACCTCAAGTGCTATCCCACGGCTGCCCGAGGCCGG | |
| | GCCCGGCAGAACCTCCACCATGCAGACCACTGAGCT | |
| | CCTGCCGG | |
| | CCATGCTCCACGCCTTTGTGGGGCTGTGCCCTCCCC | |
| | ACCTCACTGGCCTGGCTGGCTCCTTGATAACCTACA | |
| | CTGCGGGG | |
| | GGCGGCCCCTCAGAAGCCCCACCTGGGCCTCTCCCC | |
| | CACCATCTCCCTGTGCCTCCCCCAGCTCTGCAGTGT | |
| | GCTGTCCA | |
| | CACCCCCACCATAGCCGCCTGGCCCAGGTACCTGCA | |
| | GGGGGATGATGTACCCAGCCCGGAGGTGGACGTTGA | |
| | TGGTGTCC | |
| | AGGGGGGCCGGCAGCGTCACCCACTGCCCCTCGCTG | |
| | TGGATGGCTGGCTCACGGGGAGCTGCAGGTGGGGGT | |
| | GGGAGGCT | |
| | GCCAAGGGCCTCTACTGGCACCTGGAGGGAGATGTT | |
| | GCTTTGAGGATTCTGGGCCGTGCCGAGGCCCCCATG | |
| | CTGTCCTC | |
| | AGGAACCACGCTCTCCATCTCCCGGGGCTCCACGTG | |
| | GAGGCCTAGGAGGCCTGGCACAGACGGGCTGAACTG | |
| | TGACTTCA | |
| | GGCGCAGACTCAGGGCTCACTGTAGGTAGGGACCCT | |
| | CCTGGCTACTGAGTGGGGACAGTGCCTGAGAGGCCT | |
| | GGGTATGC | |
| | CAGCTCAGCACAGGCACAGCCTGTGATGGCCGTGGG | |
| | ACTTGGCCAGGTTCTGCAAGCAGGCCTCAGTCTGCT | |
| | CATCTGGG | |
| | AAATGGGGATGGTGTGAGGAGCGGAGAGGCTGATGG | |
| | GTGCGGAGCAGCCGGGTGGGGGCCTGGCACATGGCC | |
| | GGGACTCA | |
| | ACACATACGTTCCTCTTTCCGCCACCTGGTCACACC | |
| | AACTGGGTGGAGGGGGCAGGTGGGAGGGCTGCTCTG | |
| | GTCTCCCG | |
| | TCTCCCCAGGGCTTAGGGTCCCCAGACTCACCGTCT | |
| | GCAGGTCGTACCATGTGCCCAAGGGGAAGTAGCCAG | |
| | TCACTTCG | |
| | GCCTTCCCGGCCTGGAGCACTGGGGTGATGAGCAGG | |
| | GCCTCCCCCCACAGGAGCTGGTGGTCCACAGTCCAG | |
| | GTGCTAGA | |
| | GTCCTTGGGGAACCTGCAAGGGGGATGGGCACACAG | |
| | GCATACGGGTGATGAATGGGATGGGGCTGGCTCATA | |
| | TGCCCACC | |
| | CTGGGGAGGCACAGGAAGAGGCTGAATTCTGCTCTC | |
| | TGGAGCTCAAGGTGCCGGAGGACTTGGAGGACTCAG | |
| | GTCAGACG | |
| | GGCAGGGATGATTCCAGCAGATAGAAGTCAATGCCG | |
| | GAGCTGGGAGAAGCCTTCTCCCCGGACATGCCGGCA | |
| | GCTGGGCT | |
| | CAGGGCAGGAATAGGGGCTTGTGCTTGAGGCACCAG | |
| | GATGGACACAGCACTGGCCTCACTTTGCTCGCTCAC | |
| | TTATCCTC | |
| | CGGTAACCAAGGCAGCATGTGCTCATCAGTCAAGTA | |
| | TTTGGAAAGTACGAAAAAGTATCAACAAAGAGAAAA | |
| | GATCCTCC | |
| | CAGTCCCACAACCAAAGGCAACTTGAATTAACATCT | |
| | GACAAAAAGATGAATGTACATGTGCAAGCAGCTCTA | |
| | CGGAGGAT | |
| | TCTCCGTCTAACGGAGACTGTCCTGCCCTGTGACGC | |
| | AGCTCTGCCCTGTCCTGTTTCCTGCCCCCGGACGTG | |
| | CAGGGGGC | |
| | ATCCAGTGCTCAGCTATGATCCCTGCATGGATACAT | |
| | TCATGGAGCGTTCATGCCTTTTGAGGTGACTTCTTT | |
| | AGGCCCTA | |
| | TTTCTATTTGTAAAATTAACGCATCAATGTGAATCT | |
| | CAAAAACATTGACTGATATGTACTGTTGCTTTCCAC | |
| | AAATGTGC | |
| | CAATTTATATTCCACCCTACTGTGTCACTGTGTTCT | |
| | TGCCAGCAAGGGATAGTCTTATTTTCTCTAATGTAT | |
| | TCTAATTC | |
| | ATGGGCAAAATAAATGGTGTCTTTCTGCTTCAAAAT | |
| | ATTTCTTGCAAAATACATTTCTTGGAGTGATGTCAG | |
| | TGAAGATG | |
| | GTGGAGTAAGCCCCTCTCAAGATCCTCTCCTCTGTG | |
| | AAAACAGGAACATCGGCAAAAATGGTCAGAGTTGAC | |
| | ATTTTTAG | |
| | GACTCTTGAAACTAACCAAGGGTTTGCAGCCATCTG | |
| | GGGAGCATGTATTTAACAAAATGGCTGAATCTTTGT | |
| | AAGAACAG | |
| | CAAGAATCCTTGGCCTAGCTAAGTGTTGACGGTGCT | |
| | CCCCACCTCGCCCCAGCAAAGGCTGGGAGACTCACG | |
| | GATGCCAT | |
| | CTGTGAAGGATATCTGACCAATTATTGGCTGTCCAC | |
| | TAAGCTAGTCAAGCAGAGACTTTGGTGGCCACACAC | |
| | AATGAAGA | |
| | ACACAGACCTTATGGAATTAGCTCAAGAAAGTCACA | |
| | AAACAATCAGCAATAGCAACGAGACCTGAGGGGGAT | |
| | GGGGAATC | |
| | TGATGACCACAGCAGCTGCGTTTAAAATGTCCAGTC | |
| | TGAAACAAAAATGTATGAGACACGCAAAGAAACAAG | |
| | AAAGTATG | |
| | GCCCACAGATGGGGGGACAGTAGTTAATAAAAATTG | |
| | TCCCTAGAGAAGCCCAGACATTGGACTTACTAGACA | |
| | AAGAGTTT | |
| | ACGTCAGCTACTTTAAATGTGTTCAAAGAGCTCAGC | |
| | GAAACCGTGTCTGAAGAACTAAAGGAAAGTGTGAGA | |
| | ACATGACT | |
| | CACCAGATAGGGAATATCCTTAACGAGAGAAACGTA | |
| | AAGGAATGAAACAGAAATTCTGGAGTTGAAAAGAGA | |
| | GCAACTAA | |
| | AATGAGAAGTTCACTAGAGGGGCTCAACAGCAGATG | |
| | AGAACTGGTGGAAAAAAGAATCAGTGACCTTGAAGA | |
| | CAGGGCAG | |
| | TCAGACTAAGCAGTCTGAAGAACAGAAAGAAAACAC | |
| | AACGAGGAAGGAGTCAACAGAGCTGCAAAGACCCCT | |
| | GGGCACCG | |
| | TCAAGCATACCAATGTGTGGAACGGAAGTCCCAGAG | |
| | TAGAAGACAGAAAGGGGCAGAAAAAATATTTTATGA | |
| | AGTAATGG | |
| | CTGAAAAACCGCCCAAGTTTGATGAAAAACATCAAT | |
| | GCATACGTCCAAGAAGCTCAACAAACTCCCAGTAAG | |
| | ATAAACTC | |
| | AGAGATCTACACCTAGACACACCATCGGGACTTCAA | |
| | AGGCCAAAGACAGAGAATCTGGGAAGAGGCAAGACA | |
| | GAAGCTAC | |
| | TCAGCAGAAACCACGGAGGCCATGGGCTGACAAGTT | |
| | CAAAGTGTTCAGAGAAACGACCGCCAGTCAAGAACC | |
| | CTACGTTG | |
| | AGCAGAACTTTCAAAAAATGAAGGAGAAATTAAGAG | |
| | ACTCTGAAGTCAACAAAAACTGAGAGAATTCATCAC | |
| | TAGCAGAA | |
| | CTGCCCTACAATAAATACTAAAGGGAGTCCTTCTGC | |
| | TGTAAATGAAAGATGTTAGACAAGAAGTCAAAGCCA | |
| | CATGAAGA | |
| | AATAAAACCATGGGTAAAGGTAACTACATCAGTAAA | |
| | TATAAAAGGCAGACAGTGTGTATGTACTTTTTGTTT | |
| | GTAACCCA | |
| | TTTTTCCCTCCTATTTGACTTAAAAGATAATTGAAT | |
| | AAGGCGGTCATTATAAATCTGCGTGGATGGGCTCAT | |
| | CATGGATA | |
| | CAGACGTGTAACAGCACAGAGGAACGAGAGGCAATG | |
| | GAAACCACATAGAAGCAAAGCTTTTTTATGCTATTG | |
| | AAAGCAAG | |
| | TTGGCATCAGTTCCAACGAGGCTAAAATTAAGATGT | |
| | TAATTGTAATCCCTAGGGCAACCACTAAAAAAAAAA | |
| | AAAAAAAA | |
| | GTGTAGTGACAGAAACAACAAGGTCATTAAAATAGT | |
| | AATTAAAACACAAATTGCGTTTCTTGGATTTCCGGG | |
| | TGAAGAAT | |
| | GTGTGGATACCATTCGTATCTCCTGTTTTGCAAACA | |
| | GTGTTCCTCCAATTTGGTTCTTAGTCTTGGTCTTAC | |
| | CAACGTAT | |
| | GAGTTCTCTACCTATTAGGAAGAGTAGCTATTTGCT | |
| | GTTTTATTGACAATGCCTTCTCTGCTGTTACAGTCC | |
| | ATGTTCAG | |
| | AGCAGTTTTAATCTTTTTGTGTTTATATTCAGAGAT | |
| | TTACATTTTTTTTTCTGCTATGGTCAAATAAAGTTC | |
| | ACCTTTTT | |
| | TTTTTTTTTTTCTCTAAATGAAACCTTTCCCCAGAG | |
| | ATACACATTCACTTATCTTTTCCTGTGGAGTCTTTC | |
| | ACAGCTTG | |
| | GTTTTTTTTTTTTGAGATGGAGTCACACAGGCTGGA | |
| | GTACAGTGGCGCGATCTCGGCTCACTGCAATCTCCT | |
| | CCTCCCAG | |
| | GTTCAAGGGATTCTCTGGCCTCAGCCTCCTGAGTAG | |
| | CAGGGACTACAGGCGCCCACCACCATGCACGGCTAA | |
| | TTTTTGTA | |
| | TTTTTAGTAGAGATGGGTTTCACCATGTTGGCCAGG | |
| | CTGGCCTCGAACTCTTGACCTCAGGTGATCCACTCG | |
| | CCTCAGCC | |
| | TCCCAAAGTTCTGGGATTACAGGCGTTAGCCACCGT | |
| | GCCTGGCCTCCACAGCTTGATTTTTGACATTAACTA | |
| | TTTAATTT | |
| | ATCTGGAACTTATGTTTCTATGGTATGCAAGAAAAA | |
| | TAAATTGACTTCTGCAAATAGTTAACTAATCACCTC | |
| | ACTCTCTG | |
| | GGAATCCTCCCTCAACACACTGATGTGCAGGGCTGT | |
| | TGTTAGGAACTCCTCGTGTGTACTACGGCCTGTCTG | |
| | GGGGATAT | |
| | TACTGGGTTTTCTTGCTGCCCTTCCCCCTTAAGCCA | |
| | GGCCCAAATGTTGTCTCACTCAGCGGCAACCTGCTG | |
| | GGTCCTGG | |
| | GGAACACAGCCCACAGCAGGACAGGGCTGCCTGGGA | |
| | GTTACGTGCCCCTCCCCCAGGGCACACATGGGCCAC | |
| | CGCCCCTG | |
| | CCTAGGTCACTCACTCCAGGAAGAGGGGCCGGGCCA | |
| | CGGTCTCCCCCGCGACGTGGGCCTGGTGGAACAGTG | |
| | TGTAGAGG | |
| | TGGGGGAGGAGTGCGTAGCGCAGGGTGAGGGCCTTC | |
| | CTCATGGCCTGCTGGGCCGGCTCGCTGAAGCTGTAC | |
| | GGCTCCTG | |
| | GGGCTGCAGGGCAGGCGGGGGCAAAGGAAGCACTTG | |
| | GGTGCTGGGGCCGCGGTCCCCTGGAGTCCCCGCCTC | |
| | GGGAGAGC | |
| | TGCACTTCTCAGCCACCCAGCATGGGGTGCTTCTCC | |
| | AGCAGGGGTGGGATTCCCAGGGGAGAGTCTTGGGTG | |
| | GGTGGGAT | |
| | CGCCCACCTGCCATGCCGCCACCCCCACCCTACCAG | |
| | ACTGAGCAGGCTGTTGTGGTTCCGCATGAAGGGGTA | |
| | GAAGGCCC | |
| | CCAGCTGGGTCCAGCGCACACACAGCTCCTCTGAGG | |
| | TGTTGCCCAGGAAGCCGCAGACGTCGGCCCCGACCA | |
| | GAGGCACC | |
| | CCCAGCAGGTTAAACTGCAGGATTTCTGGGAGGGCA | |
| | GAGTCAGGCTGGTCCTCAGGCTGCTGCAGCAGAGCC | |
| | AGCTCAGG | |
| | CCAAGTGACCCAGAGCCCCACCTGCTAAGTGGGTGG | |
| | GGGGCCCCGGCAAGCCTCCCATAGAGGCCCCCGGCT | |
| | CTACTCTG | |
| | CTGAGCAGCCCCTCCTGGTAGGAGCTCACCTGGCAC | |
| | GGAGGAGGCGAGCTGCTCCCAGGAGCTCCACACGTC | |
| | CCCCGTCC | |
| | AGTGGCCGGCGTATCGGCCGTGGCCAGCAAAGGTCG | |
| | AGCGGGAGATCACAAATGGGCGTGTCCCCCGAGCCT | |
| | TCACCAGC | |
| | GCCCTGGGGTGGTGGGGGACACCGTGAGGGCTGTGT | |
| | GGAGCGGGGTCACTCGGGAACCCTGTCACCAGCAGG | |
| | GCAGAGCT | |
| | GGGAGCAAGGAGCTTTCTGGGATGAGGCAGAGGCTG | |
| | GGGAGGAGGACGGCGAGGCTACTGCCCATGTCTGCG | |
| | GGCACAGT | |
| | TGCCTCTGTCTGGGCCACCTGATGCCTGTAGGTGAG | |
| | CCAGGACCCCCCCCAGCCTCACAGGAGTCATGAGGT | |
| | CCCCGCTG | |
| | ATGCAGACTCCTGTCAGCATCCAGGGTCCTTCCTCT | |
| | CCAAGCCCCAGGCATGAAGTGGGAAGGAGCGCTCCA | |
| | GCTTCAGG | |
| | CTGGGGTGCAGGCAGCGGGTGGGGAGAGCACTCGGG | |
| | CCGCCAGCGGGCCCCGGGTGGTCTGGGCCTCCGCTT | |
| | TTCCTCCT | |
| | CCCTGAGGCCCTGCAGAGGCCCCAACCTTGTAGGAC | |
| | AGGCTGTGAGGGCAGAGCCCAGTGGGGGGGGACGTG | |
| | GCCCTCAC | |
| | CTGTGGGAGGCGATGGCTTCGGTCAGGCCGTAGAGG | |
| | TTGTGCAGGTTGTAGTGTGTGGAGAGAAACTGGTGG | |
| | CTGGAGGC | |
| | ACAGATGGTGGCCGCCTGGAGGGTCCCCCCAACCAC | |
| | CCCTGGAAGAGGCGGGGGCTGGTTTCCAGGGAGCTT | |
| | CCTCCCGG | |
| | CAGGCTCCAAGGTGCCCTCCCTGCCCCCCACCTCCC | |
| | AGGAGCCCCTGTGCAGGTCGCTGCCTCTTCAGGCTG | |
| | GCCCTGGA | |
| | GGTGCACCTGCAGCCGCTCCCCACGCCTCTGTGGGG | |
| | CTGGCGTTACCCGGGAACCTGGGTAATCATCTCCAC | |
| | TGCTTCTA | |
| | AGACAGGGCTGGGCGGGTGCTGGAGCCTGGGCTCTG | |
| | CAGACCCCCAGACTTCACATCCACCGTCTCTTGGCC | |
| | CATTTCAA | |
| | GCACTCCCCTGCCTGTTTACCTAAGCCCAAAACCCC | |
| | ATCCTCTCACTGATTCTTTGCTGGGCCAAACTATAG | |
| | CCTCAGGC | |
| | CCCCAAACCTTCTCCCAGGCCCATCCTTGTACTTCC | |
| | TTGTAAAACCCAGTTTCTGCCATTCCTGCTAAGACA | |
| | GTTTGGCG | |
| | AGAAGCCCCAACCTCCACCACCCCTCAGGCGATGTC | |
| | TGGTCACCCTGGCCTGCCTTCAGCAAGAATCCTGCT | |
| | GGGTCAGT | |
| | TCAGCCAGAATCCCCCTGACCCCCATGCTTCTTCTT | |
| | GGTAATTCCTGCCTCTGAGCCCACCCTGCTCCCTGG | |
| | CTAGAAAT | |
| | CCACTTGCCCGTGCTGCATTCGAGTTGGGCCTGACC | |
| | TCTCCCCACGGGTGGGTAGGTCTCCGCTGCATGGTC | |
| | CCTGCGCC | |
| | TATCGAGACGGCTCTGAGTCGAGTCGGCCTCACTGT | |
| | GCTCTGGCAAGCACCTGTGAGCGGCGTCTCCTCCGG | |
| | CAGGGCCT | |
| | GTGCCTCTGCACCCACCCTGTCTGCACTGCTTGGCT | |
| | GAGTCTCCCAGCCTGAGCCTTTCTCCTGGGGATCCC | |
| | CCCCCCGC | |
| | CCCTTTCCCCGCTGGCCCATCTGCTTCTCAGAGATG | |
| | AGGGTGCTAAGTCTCCCAGGCCAGACAAGGGAGTCT | |
| | CTGATTTG | |
| | ATTAAGTCCCCAGGGTAGGTGGGGGGCGAGCTGACC | |
| | AGGCACGTAGGGTGGGTTCTCCAGCTCATTGTTGGG | |
| | GCAGCCGT | |
| | CCTCAGAGCCCCTGATGAAGTTGGAAGGCTCGTTCA | |
| | TGTCCTGCAAGAGAAGCGCTGCTGGTAGGTGACTCT | |
| | GCCCAGAG | |
| | TGAGGAGGGTGGGGTAGTCCCCAGAGGCCTTGGGGA | |
| | TGCTCAGGAGGGGGCCACACTTACAATCCACATGCC | |
| | GTCGAAGG | |
| | GCACCTGGTCATGGAACTCAGCCACCATGTCCTCCC | |
| | ACCAGGCCAGGGCTGTGGGGTTGGTGAAGTCGGGGA | |
| | AGGCAGTG | |
| | GACCCGGGCCATACCTGGACAACGAGAGGCTGCAGT | |
| | GGGGAGACCCGGCCCCACCCAGGGCCTGCATGGAAG | |
| | CCCCACTG | |
| | AGCCTCAGCCTGAGCAGAGGGGCAGCCTCACTCTTA | |
| | GTGGACGCCCACATTTGCAAATCTGAGAGACCTGCA | |
| | CCCTGGAG | |
| | GTCAATGAGCAGCTCTTTCTCCACAAGGCACTGATG | |
| | CGGGGCCAAGGGGCCTCAACCCTGGGAAAACGAGGG | |
| | GTCACCCC | |
| | CAACACGTCCACCCAGGGGCTGGGCTGCAGAACCAG | |
| | AGAGCGTCAGGGAGAAGGGCAGCGGAGACGGCCCCG | |
| | TGCAAGCC | |
| | CTGCCAGGCAGCCTCACATCCGCACCAGACCCTCCC | |
| | TAGCTGCTCCCTCAGCGGGGGCCCTGCCTGGAGGCC | |
| | CCTGGGGG | |
| | GCTGGGAGGACAAGGAGGACCCTAAGGCCTCACTGA | |
| | GGGTCTGCAGGGCTTGTGCCTGGGCCAGGCCCAGCT | |
| | GCCCATGA | |
| | AGTCAAAGGCCTGCTGATGGAAGAACTCACGGGGCC | |
| | ATTCCTTGTAGGAGCCACCATCGTCACGGGCCACTG | |
| | TGGCCCAT | |
| | TCCTCTCAGCTGTCCCACTGGGCGTGATGGCTCCTC | |
| | AAATCCCACCGTCTTCCTGAGCAGCTGCCGGCTCCC | |
| | CCTGGCTG | |
| | GAGGCCTCTGCTTTCTAACCCCCGTCCCCTGGACCC | |
| | TCGCCCTACCTTCCCAATCAGCGGCTGGCCGGTCTC | |
| | GTTGGTGA | |
| | TGAAAACCCCCCTCCGCAGACCCTCGTCGTAGGGCC | |
| | TGTAGCTCCCGGCAGGGCCCGAGCTGCTGATGGCAG | |
| | GATCCTGG | |
| | GAAGAGGGAAACCTGTCAAGGTGAGGGTGGCCCAGA | |
| | GCCCTGGCGCCAGCCACGGGGAAAACTGAGACAGTG | |
| | AGAGGATG | |
| | AGGCTGGGGATGACATCATGCGTGTGTACAGCATGC | |
| | CTGCCTGTGTACCTGCACATGCCTGCACACGCCTGC | |
| | CTGGAGAG | |
| | GAGCCAGGGCCGGGCGAGTCAGGACCAGCTGGCTGA | |
| | TGAGGGGCGCGCCGGAATGGAAGCTTTACCTTTACC | |
| | TTTTCTTT | |
| | AAACCTCTAGAGTTTCCCAAGTGTTCTACAGTAATC | |
| | ACTTTTCTTTTATAATTAAAAAAATGTGAGAAAGAA | |
| | AAAAGTCT | |
| | GTGTTTCCACTCCTGCTCTTAAGGGCTACTACTGTT | |
| | TGCTGCCTGCAGAAGGCAGGGATGGAAACAGGCAGC | |
| | ATCAGCAC | |
| | AGCCCCACATCTCATCTGTGCTTCCTCTGCTGAAGT | |
| | GGATCTTCCGGGCAGGAACTGTTGGATCTTTTCAGC | |
| | TCGCCCTG | |
| | CCTCCCACCTCCTCCTCTGGCCATCGTGCCGGACAC | |
| | CAGACTCGGGCTGGACCAGCCCCCAAACTGCATCCT | |
| | TCTGGCTG | |
| | TGGGACTGGCTCAGGGAAGGGCCTCTTTGTGCTGGG | |
| | TGACTCAAGAGTCCTGCTTTGGGACTTTTCTATGGG | |
| | AGCTGATG | |
| | GGAAGAAGTCTCCGCTCTGTGGTGCCAGCATGAGGG | |
| | CTGGAAGCTGCCCTCACTCACGCCGTCCTTTCCAGG | |
| | GATTCAGA | |
| | GCCGGCCAGAAGGAGAGGCACGGCCAGAAGGCACAC | |
| | GGAGCCTCCACAGTTCCAGCGATACCCATGCCCAGG | |
| | CCCACCGG | |
| | GCCCTCCACTTTCCAGGACCAGGTGACATCAGTTTA | |
| | TCATGCCCCTGGCCTTTTGCTTTAAGGCAGTTCAAG | |
| | TAACCTAC | |
| | ATCTGACAGAACCCTGAGTAGCACCCGGCCATTTCT | |
| | GGGGACCCTCAAACCCGACCTCCCTGGACAGCTGGG | |
| | GCCAAGCC | |
| | TGCAAACTTCTTGATATTCCCTCGATATCACAAAGA | |
| | CCCTCAGAAAACATCCTCGGCGACCACACAGGCACG | |
| | AGGATGAC | |
| | GCTGCACAGAGAAGGAGCCACTGGGCACCGGGCGGC | |
| | CCCCTTCCCAAAGACCCACAGTGTGGGGGCACACAC | |
| | CACGATCA | |
| | TCATGTAGCGCCGGCCGCCCTGGTGCAGCTCCTGCA | |
| | CCATGGCCGGGAAGTCCCGGAAGCCATCCTTGTTGA | |
| | ACGTGAAG | |
| | TCCCTCCGGGAGTCCATGTAGTCCAGGTCGTTCCAC | |
| | TGGACGTCCTGCAGCCACAACACGGGACCGTCTGCT | |
| | GGGGCCTG | |
| | AGGAGACGCGTCCCGGCCAGCCCCTTGCCTCCCCTG | |
| | CCACCACCCCAACTCACCAGGGGGAAGTGGGCCCTG | |
| | GTCATGTT | |
| | CTCCACCACCTGGCGGGTGATAGCGGTGGAGGAGTA | |
| | GCCCCAGCGGCACAGGTGGAAGCCCAGGCCCCAGTA | |
| | TGGCGGCA | |
| | TGAACGGGTATCCTGCAGGCCAACGCCGACTTCATG | |
| | AGGGAGGGAGGAGGGAGCCTTGGGGCGGGGGCCGCG | |
| | GCCAGGGA | |
| | GCAGGCCCTACCCACAACGTCCAGGTACTGCTGCAC | |
| | CACGCTCTTGGGCTCTGGGCCCAGGAAGATGTAGAC | |
| | ATCCAGGA | |
| | TCCCACCTGTCGACCTCCAGCTAAGGGCAGGGCTCG | |
| | GCTGCAGGACCACATCTGGAAGGGAAGCAGCTCTGG | |
| | GGTTGGGG | |
| | GACAGATTCTTCACTTGGAGGGCTCTGCACCCCACA | |
| | GATGGGCCAATCACAGGCGGAGAGTTGAGGCTCTCT | |
| | CCCCACGA | |
| | GGAGCTCAGAGTGGCCTGGGGAAGCCGTGCTAAGCC | |
| | TGACTCAGGAGGAACCTGGCATGGGACGGAAAGATA | |
| | TGTGGTGC | |
| | CAGCCCACCTCCAGGCAGGGCCCCAGAACCCTCCTT | |
| | AGGAGCACAGAAGGCCAGAGTTGGGAGAGGCATCAG | |
| | CCCATAGG | |
| | ACTCATTTCCCAGAAAACACAAGGCCCAGCACCGTG | |
| | CCCAGGGCCCCTCATGCGGACCTCCAGTCTCCAGGG | |
| | CAGGCAGC | |
| | ACGGAGGAGACCCCGGCCCGGGCGCTGGGCGGCGGG | |
| | CAGCTTACCCATGGCATTGCTGTTTAGCAGGAACAC | |
| | CCCGTGTG | |
| | CCGACCCGCCGTCCTCCAGCGCCAGGTAGAAAGGGT | |
| | GAGACCCGTAGAGGTTCGCACCGGGCTGGGACATGC | |
| | AGGAGACG | |
| | GCGCTTCAGCACCTGCGCTCCCCAGCTCAGTGCCCC | |
| | CGCCCGCCGCCCGCCGCTGTACCGTGGGCGCAAGGT | |
| | CCCGGTTC | |
| | CACAGGGTGATCCTGGTCCAGCTGGTGCTGAGCATC | |
| | AGGGGACTGAGGTGCTCGGCGAGGCCTGTGATATAC | |
| | TGCGAGGG | |
| | CAGCGAGGTGGACAGCTGAAGGAACTGGTCCGCAAA | |
| | GAACAGGGGCGCCACCGTCGTGTTCAGCCTGCGGGA | |
| | CAGAGGCC | |
| | AGCCAGGCTTGCTCACACCCAAGGGGCCACACGAGC | |
| | CTGAGAGCACCCAGAGAGCACCCCCGGGCCCTGGTG | |
| | CCCTGGGA | |
| | GGGCGGAGTGGCCTGGCCAGCCCTGCAGCACACTGA | |
| | CCCTGGGACAGGCGGCCGTGCCGAGCCCTGCAGCCC | |
| | TTGGCACC | |
| | CTGGCACCAGCGTGATCAACTCTCAGGGCATATCAG | |
| | AAGAGGCGCACCGCCTGGCTGGGCCCACACCACCCC | |
| | TGAGGGGC | |
| | CCCCAAGGGGCTGACCTCCAGGAACTGGCTGGTACC | |
| | GGAACTGGCACACATGCCTGTCCCATGACCCCAAGC | |
| | AAGGCTGG | |
| | GCAGGGGCTGAACAGAACCTTTCCAGGCCTAGGAGC | |
| | TTGTGGCCAAAGTCCTCCCAGATCCCAGCCCAGCCA | |
| | TGCCTGAG | |
| | AAGGACCCCTGAGGTTTGCTGGGGGTCAAGTGCAAG | |
| | GCAAACAGCACCTCCGAGAAATGAAAGCCGCAGATA | |
| | AACCCGGT | |
| | CCACAGGAAGACGCGTCCTCTGTTAACACAGCACAG | |
| | CTAATTTCGCAGGACGGGCTCACACCGCACCCACCC | |
| | TCCCCTGG | |
| | GCAAGACTACGCTGAGTGCTTTCAAGACCGAGATCT | |
| | GATAAGATGCCTGTTTGTTGTTTGTTTTTTGAGACG | |
| | GGGTCTTC | |
| | AGCAAGGCTGAAGTGCAGTGGCAAAATCATGGTTCA | |
| | CTGCAACCTCGAACCCCTGGGCTCAAGGGATCCTCT | |
| | TGCCTCAG | |
| | CCTCCTGAGTAGCTGGGACCACAGGCATGTGCCACA | |
| | ATTCCCAGCTAATTTTTTATTTTTATTTTTTAAGAG | |
| | ATGGGGTC | |
| | TTGCTATGTTGCCCAGGATGGTCTTGAACTCCTGGG | |
| | CTCAAGTGACCCTCTCGCCTTGGCCTCCCAAAGTGC | |
| | TGGAATTG | |
| | CAGACATGAACCACCATACCTAGCTTGATAAGATGC | |
| | TTTGAGAAACTTCTCTTGGGTCTCTCTACTCAGAGG | |
| | TCAGCTCA | |
| | GAGAGGCCACCCCTGTCCCCTGCATCTGCACAGCTG | |
| | GTTTCCAGAGTGGCCTCCCCACCCCCCACTCCTGCA | |
| | CAGCTGGC | |
| | TTCCAGAGAGGCCACCCCTGCCCCCAACATCTGCAC | |
| | AGCTGGCTTCCAGAGAGGCCATCCCTGTCCCCTGCG | |
| | TCTGCACA | |
| | GCTGGCTTCTAGAGGGGACCTCCCTGCCCCGTGTCT | |
| | GCACAGCTGGCTTCTAGCGTGCTCTCTGCATGACCA | |
| | CTCCCTGA | |
| | CGCTGTGTCTGCATGGACTTGCTTATGGCTTCCCCT | |
| | CACTGTGTGTCTGTCCCCGGGAGCAGGATCCAAACC | |
| | ACCCGGCA | |
| | CGGAGGGTCAGTGCGTGGACACTGAGTAAATGAATG | |
| | AGCAAAAAAAGAAAAACGCATTTCCGAAGGCAACTT | |
| | CTCTTAAG | |
| | ATTAAGGCAAATCCAAGTGCTGTCGTTCCTGAGAGT | |
| | TTTCCTGAGTTAAATGACAAGCAGGGGCTTGGTGAG | |
| | TCCTGAAC | |
| | ACGTGTTTACATGGGATTTAGACCTATGTGAGTAAA | |
| | AGACCCAGGGAGCGCCCTGTGAGAAATGCCCGTCGC | |
| | CCTCCCCG | |
| | CCGTGTGAGAAACAAACATGTGTCGCCCTGCCCATC | |
| | GTGTGAGAAACAAACGCGTGTGGCCCTCCCTGCCGT | |
| | GTGAGAAA | |
| | TGCGCGTCGCCCTCCCCATCATGCTGGCACAGAGCC | |
| | CAGAACTCACAGCACGCGGCCGTCCAGCTGCCGGCG | |
| | CACGATCA | |
| | CCCCGAAGGGCTCCTCGGAGAACTCCACGCTGTAGA | |
| | GTGGGGACGGTGCCCGGCTGTGGACATGCGGGGTCT | |
| | CCAAGGGC | |
| | ACCTCGTAGCGCCTGTTAGCTGGATCTTTGATCTAG | |
| | AAGAGATGGGGGTTTATTGATGTTCCCCACAGCCAC | |
| | CTTACTCT | |
| | CCAGAGAACAACCCGCACGCCAAGGACAGGTCAGGT | |
| | CCTGGTGCAGCCACACATGGATGTGGGACCATGGGC | |
| | AGCACATT | |
| | CAGCCTCTCTGAGCCTCGGCTCCCTCATCTGCAGAG | |
| | CCAGGAGGAGGACGCCTCCCCCAGGATGGAGGTAAA | |
| | ATACAGAA | |
| | CACCTGATAACTCTGAATTTCAGATAAACAACAAGC | |
| | AGCTTTTCTAGTATAAATACATCCCAAATTTTGCAT | |
| | CCTTACAC | |
| | AAAAAAACAAAGTCATGCCTGGGGTATCTGCAATTC | |
| | GGGTTTACTGGGCATCCTGTTTTCATTCGCAACCTC | |
| | TGGCAGCC | |
| | CTACTCTACCTGACCCACCTTTTCATAAAGATGAAT | |
| | GAGCCCCGAGCCCTGCCTTCTGGAGTACCTGTCACC | |
| | GTGGTGTC | |
| | CCCACTGCTCCCCGAGGGGCGCTGCCATTGTCTGCT | |
| | CACACCTCCGCTCCCAGCAAGGGCCCAGCACACAGT | |
| | GGTGCAAC | |
| | ATGCACCCCACCCTTGTGAGGTGCGTGGGTGTCGAT | |
| | GTCCACGCGCACCCTCTGCCCTGGCCGCCGCCCCCG | |
| | CCCCTGCC | |
| | CTGCCCACCGTGAAGTGGAGGCGGTTCTCAGTCTCC | |
| | ATCATCACGTCCAGCCGCAGGGTCAGGATGTCCTTG | |
| | GGGAAGAA | |
| | GGTGGGGGTGGTACGGGTCAGGGTGGCCGTGTAGCC | |
| | CATTTCAGAGGAGCTCAGGTTCTCCAGCTTGTAGCT | |
| | GGGGTAGC | |
| | TGGGTGGGAAGAAGCACCAGGGCTGCCCCATCTGGG | |
| | CTCCCTGCAGCCCCTGCTTTGCAGGGATGTAGCAAC | |
| | AGCCGCGG | |
| | GCCTCGCACTGTTCCTGGGTGATGGCCTTGTCAGGG | |
| | GCGCAATCGAAGCGGCTGTTGGGGGGGACGTCGCAC | |
| | TGTGTGGG | |
| | CACTGCTCTGGGACGGCCGGGGTGTGCCTGGGCATC | |
| | CCGGGGCCCTGGTCTGCTGGCTCCCTGCTGGTGAGC | |
| | TGGGTGAG | |
| | TCTCCTCCAGGACTGGGGAGGAGCCACTCAGCTCTC | |
| | GGGGAACCAGCAGGAAATCATGGAGTAGGATGTGCC | |
| | CCAGGAGT | |
| | GCAGCGGTTGCCAAGGACACGAGGGCGCAGACGGCC | |
| | AGGAGCCGGTGGGAGCAGGGCGGGTGCCTCACTCCC | |
| | ATGGTTGG | |
| | AGATGGCCTGGACAGCTCCTACAGGCCTGCGGGAGA | |
| | AGCAAGCGGGCTCAGCAGGGAGGCGGGAGGGGCGGC | |
| | ACTCACGG | |
| | GGCTCTCAAAGCAGCTCTGAGACATCAACCGCGGCT | |
| | GGCACTGCAGCACCCAGGCAGGTGGGGTAAGGTGGC | |
| | CAGGGTGG | |
| | GTGTTGCCCTGCTGTCTAGACTGGGGAGAGGGCCAG | |
| | AAGGAAGGGCGAGAAAAGCTCCAGCAGGGGAGTGCA | |
| | GAGCACTT | |
| | GCACAGTCTGCTAAAATGTTACAAATCAAACACGCT | |
| | TAGAATGTCCCCAGGAAGACCAGCAAGGCAGGTAGA | |
| | CACTTGAA | |
| | ACAGGCCAAACAGCTGTCGCCTGGGCCAGAGTGCTG | |
| | TGGTGAGATCCTGGCTCACTGCAACCTCCACCTCCC | |
| | AGACTCAA | |
| | GCAATCCTCCCACTTCAGCCTCCTGAGTAGCTGGGA | |
| | CTGTAGGCACACACCACCGCACCCAGCTAATTTTCT | |
| | GTATTTTT | |
| | GTAGAGACGGGATTTTGCCATGTTACCCAGGCTGGT | |
| | CTCGAACTCCTGAGCTCAAGTGATCCACCCCCCTTG | |
| | GCCTTCCG | |
| | AAGTGCTGGGATTTCAGGAGTGACCACGCCTGGCTG | |
| | GGAGCCCCACTTCTGCATAAAGGTGCGGCTTGGTCC | |
| | ACTGGGTG | |
| | TCAGCGGAAGTGATTCTGGCAACTCGTATGTCCTTA | |
| | GGGGGACCCAGTACCTTTCCTTGCTCCCTTTCCTAT | |
| | TGACTGGT | |
| | GCACGGACATACCACAGTGGGGAATCCTGGGCACCG | |
| | CAGCTGAGGGTGACACTCCAGGGGTGGCAAAGCCAC | |
| | AAGAGAGA | |
| | AAGGACTTAACCTCTGGCGACTTTGCAGATCAGAAC | |
| | CCCTCCCGGCCCCCGAGGTACATGGGGAAGAAACAC | |
| | AGTCTCTG | |
| | TAACAGTGGCTGCACCTGGAGCCTCGGGTACAGACA | |
| | CAACAAGGATGTCGCGTGCAGATGAGATATGGGGGT | |
| | TCGCTTTC | |
| | ATTATTCTGCTGGTAAGGTTAACAAGTACCAACGAC | |
| | CTTTGTTTCGCTTCTCTGTACATTAATATTTTATGG | |
| | CAAACATT | |
| | CTATTAGCCTGGCTCCTCCCAGGAGCAGCCGGAACC | |
| | CACATGCGATTCAATATGCATGGATTTCAATAGGGG | |
| | AGTCCCTT | |
| | TGGTGTGTGTAATACACCATGGGGAGGGCGTGGGGA | |
| | AGGCTGGGAGAGCCACCGCACCTAGGCAAGTCTGAC | |
| | CCCAGGGA | |
| | AAGGAGGAAGAAGGTGTCCTAGGAGCAAGGCCATGG | |
| | GGGAGGGATGCAGAGAGGTCCTCAAACCAAAGTCAG | |
| | CCATCAGA | |
| | GGGGTCTGCGTCTCTGAGGAACGGGCCTTGGAGCAG | |
| | AGGCAGGCAAGGATTTCAGAAAGCAGCCCCTGGCCA | |
| | GTGAAGCT | |
| | CCTTGGAGCAGAGGACTGTGCAACCTTCTCTGGTAC | |
| | TGGGAGCTGGTCACATACCAGGAAGAAGTGAACTCA | |
| | GGGCCCGA | |
| | GGGAGTCTGACCTGTCATGTCATGTTACAGAAGGCT | |
| | TGGCTGGGAACGCTCTGCCCCCGGCCGGACGTGGTG | |
| | TTCCACCG | |
| | TGGGCCACTGGTAAATTAGGGCTCTCTGGGCTGGAG | |
| | GGTGAAACATTTTACCCACACTTCTGACATTAAATG | |
| | TGTAGGGT | |
| | TTTTCTCACACCCATCAATTCTGCAACTTTCTGGAC | |
| | ACGGCTGGGTGTCCTACAATTCAATTCAATCTTGAC | |
| | ACTACCTG | |
| | GAGTCAGCACAGACTCCACAGGTTAAGGGCTCAGTC | |
| | CCACAACACTGCCTTCACTTCAGAGGCCAATGGCAA | |
| | ATCCCAGG | |
| | TTGTCACTTGTACTTTCGATCAACTAGTTATAAATT | |
| | CAGAGGTTCCCAGGACTCCCTCTTCAGGGTCACAGA | |
| | ACTCAGGA | |
| | AAGTGCGTTTCTTACAGTTGCTGGTTTATTACAAAG | |
| | GATAAAACTCAGACAGCCGTATGGAAGAGATGCGTA | |
| | GGGCCTGG | |
| | TGTGAGGTGGGGGCAGTGATGCTGTGCTCTCTGCTC | |
| | GCACCACCCTCCAGTGCCTTGGTGTGTTCCACAACC | |
| | CGGCGGGA | |
| | AGCCCTCTGATCCCTGTGGTTTCTTACGGGGTTCCA | |
| | TTGTGCAGGCATGGTTGACTAAGCCACTGGTGATTC | |
| | ACTCAATC | |
| | TCCAGCCCCTCACCCCTTTTCCATCCCAACCCGTCA | |
| | TCAGGCCTTGGTCTTTCTGGCCATCTGCTTCCATCC | |
| | TGAAGCCA | |
| | CCTGGTGTCCCCGGTCATCAGTCACCTCATGAGCAA | |
| | AAGGCTCTCTCATCACTCGGTTTCCAAGGGTTTCGG | |
| | GTGCTATG | |
| | TGCCAGCAACCAGGGACAAAGACCAGAGACAGTCAC | |
| | CCCTGGCCTGGCCTAAGGGGGTCTTTGTGATTGAGA | |
| | CATTCAGA | |
| | TTTCAGAGCTGGCCCAGGGGCCCCCTCTCAGGCCAC | |
| | GGTGCTCCTCACCCCAGGGATTCAGACCCAGCCATG | |
| | TGCGTCCT | |
| | GCAGGAGCAGGAGAGGTGGCTCGGGTCCCGGCCAGG | |
| | ACTGAGCACTGCGTCGATCAAAAGGGGAAAGGAAAC | |
| | CCTAGAAA | |
| | CGGGAAGATGCAGCCTCCAGCCCCAAGCCCAGCACA | |
| | GGAGGGCGGCTCCCCGTTCATCTCTAAGCTGAACTC | |
| | AGATAAAG | |
| | ACCGGGAGGACTAGAGGTCTGGGGCTCAAGCTCCGC | |
| | TCAGGGGAAGAGGCTGGGGGCACGGCTAGGCACGTT | |
| | CAAACCCG | |
| | CTTCTGGGATGTTACCGCCGGCAGCGCGGGGCAGAC | |
| | GTCAGGTGTCTCACCCGCTCCGTGCCTCAGTTTCCC | |
| | CGTCAGCT | |
| | GCGGCACGCGCAGAGCCTCCCTCGCTGAACAACGGG | |
| | CGGACGAGGAGAACCTAGAGGTGGCCGGGGTCCTTC | |
| | GCGTCACC | |
| | GAGGTCACTGTCCTACCTGCTGCCTCATCCCCGACC | |
| | TCCACCCGCGGCCCCGGCGACAACCTCAGCTTTCCC | |
| | AACTGAGA | |
| | GGCCGCGCGGGCAGCCGACCGCCCCACCGACCCGGC | |
| | CCGCGCTCAGGGAAGCCCCGCAGCCCCGGCCCGGCT | |
| | CACCTCCG | |
| | CGCACGCGCGCCCTGGCCGCCCGCGGAGACTCCGGG | |
| | GTCGGAATTCTTTGCCAAAATGATGAGACAGCACAA | |
| | TAACCAGC | |
| | ACGTTGCCCAGGAGCTGTAGGAAAAAGAAGAAGGCA | |
| | TGAACATGGTTAGCAGAGGCTCTAGAGCCGCCGGTC | |
| | ACACGCCA | |
| | GAAGCCGAACCCCGCCCTGCCCCGTCCCCCCCGAAG | |
| | GCAGCCGTCCCCCCGCGGACAGCCCCGAGGCTGGAG | |
| | AGGGAGAA | |
| | GGGGACGGCGGCGCGGCGACGCACGAAGGCCCTCCC | |
| | CGCCCATTTCCTTCCTGCCGGCGCCGCACCGCTTCG | |
| | CCCCGCGC | |
| | CCGCTAGAGGGGGTGCGGCGGCGCCTCCCAGATTTC | |
| | GGCTCCGCACAGATTTGGGACAAAGGAAGTCCCTGC | |
| | GCCCTCTC | |
| | GCACGATTACCATAAAAGGCAATGGCTGCGGCTCGC | |
| | CGCGCCTCGACAGCCGCCGGCGCTCCGGGGGCCGCC | |
| | GCGCCCCT | |
| | CCCCCGAGCCCTCCCCGGCCCGAGGCGGCCCCGCCC | |
| | CGCCCGGCACCCCCACCTGCCGCCACCCCCCGCCCG | |
| | GCACGGCG | |
| | AGCCCCGCGCCACGCCCCGTACGGAGCCCCGCACCC | |
| | GAAGCCGGGCCGTGCTCAGCAACTCGGGGAGGGGGG | |
| | TGCAGGGG | |
| | GGGTTGCAGCCCGACCGACGCGCCCACACCCCCTGC | |
| | TCACCCCCCCACGCACACACCCCGCACGCAGCCTTT | |
| | GTTCCCCT | |
| | CGCAGCCCCCCCCGCACCGCGGGGCACCGCCCCCGG | |
| | CCGCGCTCCCCTCGCGCACACTGCGGAGCGCACAAA | |
| | GCCCCGCG | |
| | CCGCGCCCGCAGCGCTCACAGCCGCCGGGCAGCGCG | |
| | GAGCCGCACGCGGCGCTCCCCACGCACACACACACG | |
| | CACGCACC | |
| | CCCCGAGCCGCTCCCCCCGCACAAAGGGCCCTCCCG | |
| | GAGCCCCTCAAGGCTTTCACGCAGCCACAGAAAAGA | |
| | AACAAGCC | |
| | GTCATTAAACCAAGCGCTAATTACAGCCCGGAGGAG | |
| | AAGGGCCGTCCCGCCCGCTCACCTGTGGGAGTAACG | |
| | CGGTCAGT | |
| | CAGAGCCGGGGCGGGCGGCGCGAGGCGGCGGCGGAG | |
| | CGGGGCACGGGGCGAAGGCAGCGCGCAGCGACTCCC | |
| | GCCCGCCG | |
| | CGCGCTTCGCTTTTTATAGGGCCGCCGCCGCCGCCG | |
| | CCTCGCCATAAAAGGAAACTTTCGGAGCGCGCCGCT | |
| | CTGATTGG | |
| | CTGCCGCCGCACCTCTCCGCCTCGCCCCGCCCCGCC | |
| | CCTCGCCCCGCCCCGCCCCGCCTGGCGCGCGCCCCC | |
| | CCCCCCCC | |
| | CCCGCCCCCATCGCTGCACAAAATAATTAAAAAATA | |
| | AATAAATACAAAATTGGGGGTGGGGAGGGGGGGGAG | |
| | ATGGGGAG | |
| | AGTGAAGCAGAACGTGGGGCTCACCTCGACCATGGT | |
| | AATAGCGATGACTAATACGTAGATGTACTGCCAAGT | |
| | AGGAAAGT | |
| | CCCATAAGGTCATGTACTGGGCATAATGCCAGGCGG | |
| | GCCATTTACCGTCATTGACGTCAATAGGGGGCGTAC | |
| | TTGGCATA | |
| | TGATACACTTGATGTACTGCCAAGTGGGCAGTTTAC | |
| | CGTAAATACTCCACCCATTGACGTCAATGGAAAGTC | |
| | CCTATTGG | |
| | CGTTACTATGGGAACATACGTCATTATTGACGTCAA | |
| | TGGGCGGGGGTCGTTGGGCGGTCAGCCAGGCGGGCC | |
| | ATTTACCG | |
| | TAAGTTATGTAACGCGGAACTCCATATATGGGCTAT | |
| | GAACTAATGACCCCGTAATTGATTACTATTAATAAC | |
| | TAGTCAAT | |
| | AATCAATGTCGACGGATACCACGTGGGCGCGCCTAG | |
| | AAGATGGGCGGGAGTCTTCTGGGCAGGCTTAAAGGC | |
| | TAACCTGG | |
| | TGTGTGGGCGTTGTCCTGCAGGGGAATTGAACAGGT | |
| | GTAAAATTGGAGGGACAAGACTTCCCACAGATTTTC | |
| | GGTTTTGT | |
| | CGGGAAGTTTTTTAATAGGGGCAAATAAGGAAAATG | |
| | GGAGGATAGGTAGTCATCTGGGGTTTTATGCAGCAA | |
| | AACTACAG | |
| | GTTATTATTGCTTGTGATCCGCCTCGGAGTATTTTC | |
| | CATCGAGGTAGATTAAAGACATGCTCACCCGAGTTT | |
| | TATACTCT | |
| | CCTGCTTGAGATCCTTACTACAGTATGAAATTACAG | |
| | TGTCGCGAGTTAGACTATGTAAGCAGAATTTTAATC | |
| | ATTTTTAA | |
| | AGAGCCCAGTACTTCATATCCATTTCTCCCGCTCCT | |
| | TCTGCAGCCTTATCAAAAGGTATTTTAGAACACTCA | |
| | TTTTAGCC | |
| | CCATTTTCATTTATTATACTGGCTTATCCAACCCCT | |
| | AGACAGAGCATTGGCATTTTCCCTTTCCTGATCTTA | |
| | GAAGTCTG | |
| | ATGACTCATGAAACCAGACAGATTAGTTACATACAC | |
| | CACAAATCGAGGCTGTAGCTGGGGCCTCAACACTGC | |
| | AGTTCTTT | |
| | TATAACTCCTTAGTACACTTTTTGTTGATCCTTTGC | |
| | CTTGATCCTTAATTTTCAGTGTCTATCACCTCTCCC | |
| | GTCAGGTG | |
| | GTGTTCCACATTTGGGCCTATTCTCAGTCCAGGGAG | |
| | TTTTACAACAATAGATGTATTGAGAATCCAACCTAA | |
| | AGCTTAAC | |
| | TTTCCACTCCCATGAATGCCTCTCTCCTTTTTCTCC | |
| | ATTTATAAACTGAGCTATTAACCATTAATGGTTTCC | |
| | AGGTGGAT | |
| | GTCTCCTCCCCCAATATTACCTGATGTATCTTACAT | |
| | ATTGCCAGGCTGATATTTTAAGACATTAAAAGGTAT | |
| | ATTTCATT | |
| | ATTGAGCCACATGGTATTGATTACTGCTTACTAAAA | |
| | TTTTGTCATTGTACACATCTGTAAAAGGTGGTTCCT | |
| | TTTGGAAT | |
| | GCAAAGTTCAGGTGTTTGTTGTCTTTCCTGACCTAA | |
| | GGTCTTGTGAGCTTGTATTTTTTCTATTTAAGCAGT | |
| | GCTTTCTC | |
| | TTGGACTGGCTTGACTCATGGCATTCTACACGTTAT | |
| | TGCTGGTCTAAATGTGATTTTGCCAAGCTTCTTCAG | |
| | GACCTATA | |
| | ATTTTGCTTGACTTGTAGCCAAACACAAGTAAAATG | |
| | ATTAAGCAACAAATGTATTTGTGAAGCTTGGTTTTT | |
| | AGGTTGTT | |
| | GTGTTGTGTGTGCTTGTGCTCTATAATAATACTATC | |
| | CAGGGGCTGGAGAGGTGGCTCGGAGTTCAAGAGCAC | |
| | AGACTGCT | |
| | CTTCCAGAAGTCCTGAGTTCAATTCCCAGCAACCAC | |
| | ATGGTGGCTCACAACCATCTGTAATGGGATCTGATG | |
| | CCCTCTTC | |
| | TGGTGTGTCTGAAGACCACAAGTGTATTCACATTAA | |
| | ATAAATAAATCCTCCTTCTTCTTCTTTTTTTTTTTT | |
| | TTAAAGAG | |
| | AATACTGTCTCCAGTAGAATTTACTGAAGTAATGAA | |
| | ATACTTTGTGTTTGTTCCAATATGGTAGCCAATAAT | |
| | CAAATTAC | |
| | TCTTTAAGCACTGGAAATGTTACCAAGGAACTAATT | |
| | TTTATTTGAAGTGTAACTGTGGACAGAGGAGCCATA | |
| | ACTGCAGA | |
| | CTTGTGGGATACAGAAGACCAATGCAGACTTTAATG | |
| | TCTTTTCTCTTACACTAAGCAATAAAGAAATAAAAA | |
| | TTGAACTT | |
| | CTAGTATCCTATTTGTTTAAACTGCTAGCTTTACTT | |
| | AACTTTTGTGCTTCATCTATACAAAGCTGAAAGCTA | |
| | AGTCTGCA | |
| | GCCATTACTAAACATGAAAGCAAGTAATGATAATTT | |
| | TGGATTTCAAAAATGTAGGGCCAGAGTTTAGCCAGC | |
| | CAGTGGTG | |
| | GTGCTTGCCTTTATGCCTTTAATCCCAGCACTCTGG | |
| | AGGCAGAGACAGGCAGATCTCTGAGTTTGAGCCCAG | |
| | CCTGGTCT | |
| | ACACATCAAGTTCTATCTAGGATAGCCAGGAATACA | |
| | CACAGAAACCCTGTTGGGGAGGGGGGCTCTGAGATT | |
| | TCATAAAA | |
| | TTATAATTGAAGCATTCCCTAATGAGCCACTATGGA | |
| | TGTGGCTAAATCCGTCTACCTTTCTGATGAGATTTG | |
| | GGTATTAT | |
| | TTTTTCTGTCTCTGCTGTTGGTTGGGGATATCCACC | |
| | CCTAGGAGGTATCGCGACGGATGGATCCAAGAACCA | |
| | GCCCGGGC | |
| | GGTGGAGCTCGTTGACAATTAATCATCGGCATAGTA | |
| | TATCGGCATAGTATAATACGACAAGGTGAGGAACTA | |
| | AACCATGG | |
| | GATCGGCCATTGAACAAGATGGATTGCACGCAGGTT | |
| | CTCCGGCCGCTTGGGTGGAGAGGCTATTCGGCTATG | |
| | ACTGGGCA | |
| | CAACAGACAATCGGCTGCTCTGATGCCGCCGTGTTC | |
| | CGGCTGTCAGCGCAGGGGCGCCCGGTTCTTTTTGTC | |
| | AAGACCGA | |
| | CCTGTCCGGTGCCCTGAATGAACTGCAGGACGAGGC | |
| | AGCGCGGCTATCGTGGCTGGCCACGACGGGCGTTCC | |
| | TTGCGCAG | |
| | CTGTGCTCGACGTTGTCACTGAAGCGGGAAGGGACT | |
| | GGCTGCTATTGGGCGAAGTGCCGGGGCAGGATCTCC | |
| | TGTCATCT | |
| | CACCTTGCTCCTGCCGAGAAAGTATCCATCATGGCT | |
| | GATGCAATGCGGCGGCTGCATACGCTTGATCCGGCT | |
| | ACCTGCCC | |
| | ATTCGACCACCAAGCGAAACATCGCATCGAGCGAGC | |
| | ACGTACTCGGATGGAAGCCGGTCTTGTCGATCAGGA | |
| | TGATCTGG | |
| | ACGAAGAGCATCAGGGGCTCGCGCCAGCCGAACTGT | |
| | TCGCCAGGCTCAAGGCGCGCATGCCCGACGGCGATG | |
| | ATCTCGTC | |
| | GTGACCCATGGCGATGCCTGCTTGCCGAATATCATG | |
| | GTGGAAAATGGCCGCTTTTCTGGATTCATCGACTGT | |
| | GGCCGGCT | |
| | GGGTGTGGCGGACCGCTATCAGGACATAGCGTTGGC | |
| | TACCCGTGATATTGCTGAAGAGCTTGGCGGCGAATG | |
| | GGCTGACC | |
| | GCTTCCTCGTGCTTTACGGTATCGCCGCTCCCGATT | |
| | CGCAGCGCATCGCCTTCTATCGCCTTCTTGACGAGT | |
| | TCTTCTGA | |
| | GGGGATCAATTCTCTAGAGCTCGCTGATCAGCCTCG | |
| | ACTGTGCCTTCTAGTTGCCAGCCATCTGTTGTTTGC | |
| | CCCTCCCC | |
| | CGTGCCTTCCTTGACCCTGGAAGGTGCCACTCCCAC | |
| | TGTCCTTTCCTAATAAAATGAGGAAATTGCATCGCA | |
| | TTGTCTGA | |
| | GTAGGTGTCATTCTATTCTGGGGGGTGGGGTGGGGC | |
| | AGGACAGCAAGGGGGAGGATTGGGAAGACAATAGCA | |
| | GGCATGCT | |
| | GGGGATGCGGTGGGCTCTATGGCTTCTGAGGCGGAC | |
| | AGCTTTTGTTCCCTTTAGTGAGGGTTAATTTCGAGC | |
| | TTGGCGTA | |
| | ATCATGGTCATAGCTGTTTCCTGTGTGAAATTGTTA | |
| | TCCGCTCACAATTCCACACAACATACGAGCCGGAAG | |
| | CATAAAGT | |
| | GTAAAGCCTGGGGTGCCTAATGAGTGAGCTAACTCA | |
| | CATTAATTGCGTTGCGCTCACTGCCCGCTTTCCAGT | |
| | CGGGAAAC | |
| | CTGTCGTGCCAGCTGCATTAATGAATCGGCCAACGC | |
| | GCGGGGAGAGGCGGTTTGCGTATTGGGCGCTCTTCC | |
| | GCTTCCTC | |
| | GCTCACTGACTCGCTGCGCTCGGTCGTTCGGCTGCG | |
| | GCGAGCGGTATCAGCTCACTCAAAGGCGGTAATACG | |
| | GTTATCCA | |
| | CAGAATCAGGGGATAACGCAGGAAAGAACATGTGAG | |
| | CAAAAGGCCAGCAAAAGGCCAGGAACCGTAAAAAGG | |
| | CCGCGTTG | |
| | CTGGCGTTTTTCCATAGGCTCCGCCCCCCTGACGAG | |
| | CATCACAAAAATCGACGCTCAAGTCAGAGGTGGCGA | |
| | AACCCGAC | |
| | AGGACTATAAAGATACCAGGCGTTTCCCCCTGGAAG | |
| | CTCCCTCGTGCGCTCTCCTGTTCCGACCCTGCCGCT | |
| | TACCGGAT | |
| | ACCTGTCCGCCTTTCTCCCTTCGGGAAGCGTGGCGC | |
| | TTTCTCATAGCTCACGCTGTAGGTATCTCAGTTCGG | |
| | TGTAGGTC | |
| | GTTCGCTCCAAGCTGGGCTGTGTGCACGAACCCCCC | |
| | GTTCAGCCCGACCGCTGCGCCTTATCCGGTAACTAT | |
| | CGTCTTGA | |
| | GTCCAACCCGGTAAGACACGACTTATCGCCACTGGC | |
| | AGCAGCCACTGGTAACAGGATTAGCAGAGCGAGGTA | |
| | TGTAGGCG | |
| | GTGCTACAGAGTTCTTGAAGTGGTGGCCTAACTACG | |
| | GCTACACTAGAAGAACAGTATTTGGTATCTGCGCTC | |
| | TGCTGAAG | |
| | CCAGTTACCTTCGGAAAAAGAGTTGGTAGCTCTTGA | |
| | TCCGGCAAACAAACCACCGCTGGTAGCGGTGGTTTT | |
| | TTTGTTTG | |
| | CAAGCAGCAGATTACGCGCAGAAAAAAAGGATCTCA | |
| | AGAAGATCCTTTGATCTTTTCTACGGGGTCTGACGC | |
| | TCAGTGGA | |
| | ACGAAAACTCACGTTAAGGGATTTTGGTCATGAGAT | |
| | TATCAAAAAGGATCTTCACCTAGATCCTTTTAAATT | |
| | AAAAATGA | |
| | AGTTTTAAATCAATCTAAAGTATATATGAGTAAACT | |
| | TGGTCTGACAGTTACCAATGCTTAATCAGTGAGGCA | |
| | CCTATCTC | |
| | AGCGATCTGTCTATTTCGTTCATCCATAGTTGCCTG | |
| | ACTCCCCGTCGTGTAGATAACTACGATACGGGAGGG | |
| | CTTACCAT | |
| | CTGGCCCCAGTGCTGCAATGATACCGCGAGACCCAC | |
| | GCTCACCGGCTCCAGATTTATCAGCAATAAACCAGC | |
| | CAGCCGGA | |
| | AGGGCCGAGCGCAGAAGTGGTCCTGCAACTTTATCC | |
| | GCCTCCATCCAGTCTATTAATTGTTGCCGGGAAGCT | |
| | AGAGTAAG | |
| | TAGTTCGCCAGTTAATAGTTTGCGCAACGTTGTTGC | |
| | CATTGCTACAGGCATCGTGGTGTCACGCTCGTCGTT | |
| | TGGTATGG | |
| | CTTCATTCAGCTCCGGTTCCCAACGATCAAGGCGAG | |
| | TTACATGATCCCCCATGTTGTGCAAAAAAGCGGTTA | |
| | GCTCCTTC | |
| | GGTCCTCCGATCGTTGTCAGAAGTAAGTTGGCCGCA | |
| | GTGTTATCACTCATGGTTATGGCAGCACTGCATAAT | |
| | TCTCTTAC | |
| | TGTCATGCCATCCGTAAGATGCTTTTCTGTGACTGG | |
| | TGAGTACTCAACCAAGTCATTCTGAGAATAGTGTAT | |
| | GCGGCGAC | |
| | CGAGTTGCTCTTGCCCGGCGTCAATACGGGATAATA | |
| | CCGCGCCACATAGCAGAACTTTAAAAGTGCTCATCA | |
| | TTGGAAAA | |
| | CGTTCTTCGGGGCGAAAACTCTCAAGGATCTTACCG | |
| | CTGTTGAGATCCAGTTCGATGTAACCCACTCGTGCA | |
| | CCCAACTG | |
| | ATCTTCAGCATCTTTTACTTTCACCAGCGTTTCTGG | |
| | GTGAGCAAAAACAGGAAGGCAAAATGCCGCAAAAAA | |
| | GGGAATAA | |
| | GGGCGACACGGAAATGTTGAATACTCATACTCTTCC | |
| | TTTTTCAATATTATTGAAGCATTTATCAGGGTTATT | |
| | GTCTCATG | |
| | AGCGGATACATATTTGAATGTATTTAGAAAAATAAA | |
| | CAAATAGGGGTTCCGCGCACATTTCCCCGAAAAGTG | |
| | CCAC | |
| SEQ ID | ACGAGCGACCAGAGTTGTCACAAGGCCGCAAGAAC | Homology arm 1 |
| NO: 14 | AGGGGAGGTGGGGGGCTCAGGGACAGAAAAAAAAG | |
| | TATGTGTATT | |
| | TTGAGAGCAGGGTTGGGAGGCCTCTCCTGAAAAGG | |
| | GTATAAACGTGGAGTAGGCAATACCCAGGCAAAAA | |
| | GGGGAGACCA | |
| | GAGTAGGGGGAGGGGAAGAGTCCTGACCCAGGGAA | |
| | GACATTAAAAAGGTAGTGGGGTCGACTAGATGAAG | |
| | GAGAGCCTTT | |
| | CTCTCTGGGCAAGAGCGGTGCAATGGTGTGTAAAG | |
| | GTAGCTGAGAAGACGAAAAGGGCAAGCATCTTCCT | |
| | GCTACCAGGC | |
| | TGGGGAGGCCCAGGCCCACGACCCCGAGGAGAGGG | |
| | AACGCAGGGAGACTGAGGTGACCCTTCTTTCCCCC | |
| | GGGGCCCGGT | |
| | CGTGTGGTTCGGTGTCTCTTTTCTGTTGGACCCTT | |
| | ACCTTGACCCAGGCGCTGCCGGGGCCTGGGCCCGG | |
| | GCTGCGGCGC | |
| | ACGGCACTCCCGGGAGGCAGCGAGACTCGAGTTAG | |
| | GCCCAACGCGGCGCCACGGCGTTTCCTGGCCGGGA | |
| | ATGGCCCGTA | |
| | CCCGTGAGGTGGGGGTGGGGGGCAGAAAAGGCGGA | |
| | GCGAGCCCGAGGCGGGGAGGGGGAGGGCCAGGGGC | |
| | GGAGGGGGCC | |
| | GGCACTACTGTGTTGGCGGACTGGCGGGACTAGGG | |
| | CTGCGTGAGTCTCTGAGCGCAGGCGGGCGGCGGCC | |
| | GCCCCTCCCC | |
| | CGGCGGCGGCAGCGGCGGCAGCGGCGGCAGCTCAC | |
| | TCAGCCCGCTGCCCGAGCGGAAACGCCACTGACCG | |
| | CACGGGGATT | |
| | CCCAGTGCCGGCGCCAGGGGCACGCGGGACACGCC | |
| | CCCTCCCGCCGCGCCATTGGCCTCTCCGCCCACCG | |
| | CCCCACACTT | |
| | ATTGGCCGGTGCGCCGCCAATCAGCGGAGGCTGCC | |
| | GGGGCCGCCTAAAGAAGAGGCTGTGCTTTGGGGCT | |
| | CCGGCTCCTC | |
| | AGAGAGCCTCGGCTAGGTAGGGGATCGGGACTCTG | |
| | GCGGGAGGGCGGCTTGGTGCGTTTGCGGGGATGGG | |
| | CGGCCGCGGC | |
| | AGGCCCTCCGAGCGTGGTGGAGCCGTTCTGTGAGA | |
| | CAGCCGGGTACGAGTCGTGACGCTGGAAGGGGCAA | |
| | GCGGGTGGTG | |
| | GGCAGGAATGCGGTCCGCCCTGCAGCAACCGGAGG | |
| | GGGAGGGAGAAGGGAGCGGAAAAGTCTCCACCGGA | |
| | CGCGGCCATG | |
| | GCTCGGGGGGGGGGGGGCAGCGGAGGAGCGCTTCC | |
| | GGCCGACGTCTCGTCGCTGATTGGCTTCTTTTCCT | |
| | CCCGCCGTGT | |
| | GTGAAAACACAAATGGCGTGTTTTGGTTGGCGTAA | |
| | GGCGCCTGTCAGTTAACGGCAGCCGGAGTGCGCAG | |
| | CCGCCGGCAG | |
| | CCTCGCTCTGCCCACTGGGTGGGGGGGGAGGTAGG | |
| | TGGGGTGAGGCGAGCTGGACGTGCGGGCGCGGTCG | |
| | GCCTCTGGCG | |
| | GGGCGGGGGAGGGGAGGGAGGGTCAGCGAAAGTAG | |
| | CTCGCGCGCGAGCGGCCGCCCACCCTCCCCTTCCT | |
| | CTGGGGGAGT | |
| | CGTTTTACCCGCCGCCGGCCGGGCCTCGTCGTCTG | |
| | ATTGGCTCTCGGGGCCCAGAAAACTGGCCCTTGCC | |
| | ATTGGCTCGT | |
| | GTTCGTGCAAGTTGAGTCCATCCGCCGGCCAGCGG | |
| | GGGCGGCGAGGAGGCGCTCCCAGGTTCCGGCCCTC | |
| | CCCTCGGCCC | |
| | CGCGCCGCAGAGTCTGGCCGCGCGCCCCTGCGCAA | |
| | CGTGGCAGGAAGCGCGCGCTGGGGGCGGGGACGGG | |
| | CAGTAGGGCT | |
| | GAGCGGCTGCGGGGGGGGTGCAAGCACGTTTCCGA | |
| | CTTGAGTTGCCTCAAGAGGGGCGTGCTGAGCCAGA | |
| | CCTCCATCGC | |
| | GCACTCCGGGGAGTGGAGGGAAGGAGCGAGGGCTC | |
| | AGTTGGGCTGTTTTGGAGGCAGGAAGCACTTGCTC | |
| | TCCCAAAGTC | |
| | GCTCTGAGTTGTTATCAGTAAGGGAGCTGCAGTGG | |
| | AGTAGGCGGGGAGAAGGCCGCACCCTTCTCCGGAG | |
| | GGGGGAGGGG | |
| | AGTGTTGCAATACCTTTCTGGGAGTTCTCTGCTGC | |
| | CTCCTGGCTTCTGAGGACCGCCCTGGGCCTGGGAG | |
| | AATCCCTTCC | |
| | CCCTCTTCCCTCGTGATCTGCAACTCCAGTCTTTC | |
| SEQ ID | TAGAAGATGGGCGGGAGTCTTCTGGGCAGGCTTAAA | Homology arm 2 |
| NO: 15 | GGCTAACCTGGTGTGTGGGCGTTGTCCTGCAGGGGA | |
| | ATTGAACA | |
| | GGTGTAAAATTGGAGGGACAAGACTTCCCACAGATT | |
| | TTCGGTTTTGTCGGGAAGTTTTTTAATAGGGGCAAA | |
| | TAAGGAAA | |
| | ATGGGAGGATAGGTAGTCATCTGGGGTTTTATGCAG | |
| | CAAAACTACAGGTTATTATTGCTTGTGATCCGCCTC | |
| | GGAGTATT | |
| | TTCCATCGAGGTAGATTAAAGACATGCTCACCCGAG | |
| | TTTTATACTCTCCTGCTTGAGATCCTTACTACAGTA | |
| | TGAAATTA | |
| | CAGTGTCGCGAGTTAGACTATGTAAGCAGAATTTTA | |
| | ATCATTTTTAAAGAGCCCAGTACTTCATATCCATTT | |
| | CTCCCGCT | |
| | CCTTCTGCAGCCTTATCAAAAGGTATTTTAGAACAC | |
| | TCATTTTAGCCCCATTTTCATTTATTATACTGGCTT | |
| | ATCCAACC | |
| | CCTAGACAGAGCATTGGCATTTTCCCTTTCCTGATC | |
| | TTAGAAGTCTGATGACTCATGAAACCAGACAGATTA | |
| | GTTACATA | |
| | CACCACAAATCGAGGCTGTAGCTGGGGCCTCAACAC | |
| | TGCAGTTCTTTTATAACTCCTTAGTACACTTTTTGT | |
| | TGATCCTT | |
| | TGCCTTGATCCTTAATTTTCAGTGTCTATCACCTCT | |
| | CCCGTCAGGTGGTGTTCCACATTTGGGCCTATTCTC | |
| | AGTCCAGG | |
| | GAGTTTTACAACAATAGATGTATTGAGAATCCAACC | |
| | TAAAGCTTAACTTTCCACTCCCATGAATGCCTCTCT | |
| | CCTTTTTC | |
| | TCCATTTATAAACTGAGCTATTAACCATTAATGGTT | |
| | TCCAGGTGGATGTCTCCTCCCCCAATATTACCTGAT | |
| | GTATCTTA | |
| | CATATTGCCAGGCTGATATTTTAAGACATTAAAAGG | |
| | TATATTTCATTATTGAGCCACATGGTATTGATTACT | |
| | GCTTACTA | |
| | AAATTTTGTCATTGTACACATCTGTAAAAGGTGGTT | |
| | CCTTTTGGAATGCAAAGTTCAGGTGTTTGTTGTCTT | |
| | TCCTGACC | |
| | TAAGGTCTTGTGAGCTTGTATTTTTTCTATTTAAGC | |
| | AGTGCTTTCTCTTGGACTGGCTTGACTCATGGCATT | |
| | CTACACGT | |
| | TATTGCTGGTCTAAATGTGATTTTGCCAAGCTTCTT | |
| | CAGGACCTATAATTTTGCTTGACTTGTAGCCAAACA | |
| | CAAGTAAA | |
| | ATGATTAAGCAACAAATGTATTTGTGAAGCTTGGTT | |
| | TTTAGGTTGTTGTGTTGTGTGTGCTTGTGCTCTATA | |
| | ATAATACT | |
| | ATCCAGGGGCTGGAGAGGTGGCTCGGAGTTCAAGAG | |
| | CACAGACTGCTCTTCCAGAAGTCCTGAGTTCAATTC | |
| | CCAGCAAC | |
| | CACATGGTGGCTCACAACCATCTGTAATGGGATCTG | |
| | ATGCCCTCTTCTGGTGTGTCTGAAGACCACAAGTGT | |
| | ATTCACAT | |
| | TAAATAAATAAATCCTCCTTCTTCTTCTTTTTTTTT | |
| | TTTTTAAAGAGAATACTGTCTCCAGTAGAATTTACT | |
| | GAAGTAAT | |
| | GAAATACTTTGTGTTTGTTCCAATATGGTAGCCAAT | |
| | AATCAAATTACTCTTTAAGCACTGGAAATGTTACCA | |
| | AGGAACTA | |
| | ATTTTTATTTGAAGTGTAACTGTGGACAGAGGAGCC | |
| | ATAACTGCAGACTTGTGGGATACAGAAGACCAATGC | |
| | AGACTTTA | |
| | ATGTCTTTTCTCTTACACTAAGCAATAAAGAAATAA | |
| | AAATTGAACTTCTAGTATCCTATTTGTTTAAACTGC | |
| | TAGCTTTA | |
| | CTTAACTTTTGTGCTTCATCTATACAAAGCTGAAAG | |
| | CTAAGTCTGCAGCCATTACTAAACATGAAAGCAAGT | |
| | AATGATAA | |
| | TTTTGGATTTCAAAAATGTAGGGCCAGAGTTTAGCC | |
| | AGCCAGTGGTGGTGCTTGCCTTTATGCCTTTAATCC | |
| | CAGCACTC | |
| | TGGAGGCAGAGACAGGCAGATCTCTGAGTTTGAGCC | |
| | CAGCCTGGTCTACACATCAAGTTCTATCTAGGATAG | |
| | CCAGGAAT | |
| | ACACACAGAAACCCTGTTGGGGAGGGGGGCTCTGAG | |
| | ATTTCATAAAATTATAATTGAAGCATTCCCTAATGA | |
| | GCCACTAT | |
| | GGATGTGGCTAAATCCGTCTACCTTTCTGATGAGAT | |
| | TTGGGTATTATTTTTTCTGTCTCTGCTGTTGGTTGG | |
| | G | |
| SEQ ID | TACGCCACAGGGAGTCCAAGAATG | 5′arm forward |
| NO: 16 | | primer (F2) |
| SEQ ID | CTGGAAATCAGGCTGCAAATCTC | 3′arm reverse |
| NO: 17 | | primer (R1) |
| SEQ ID | CTCCAGTCTTTCTAGAAGATGGG | Underlined = |
| NO: 18 | | PAM |
| SEQ ID | GCATCTGACTTCTGGCTAATAAAG | 3′KI reverse |
| NO: 19 | | primer (R2) |
| SEQ ID | GATGGGGAGAGTGAAGCAGAACG | 5′KI forward |
| NO: 20 | | primer (F1) |
| SEQ ID | ATTCAGGCTGCGCAACTGTTG | Forward primer |
| NO: 21 | | (F5): |
| SEQ ID | CTTTTTGCCTGGGTATTGCCTAC | Reverse primer |
| NO: 22 | | (R5): |
| SEQ ID | GCAGAAGAGGACAGATACATTCAT | Internal control |
| NO: 23 | | PCR primer F1 |
| SEQ ID | CCTACTGAAGAATCTATCCCACAG | Internal control |
| NO: 24 | | PCR primer R1: |
| SEQ ID | TGGTGCTTGCCTTTATGCCTTTAATC | Forward primer |
| NO: 25 | | (F6) |
| SEQ ID | GCATCAGAGCAGCCGATTGTC | Reverse primer |
| NO: 26 | | (R6): |
| SEQ ID | CATGCCAATGGTTCACTCTAAGGT | Internal control |
| NO: 27 | | PCR primer F2: |
| SEQ ID | TCTCTATGTCCCAAAGTGCAGACAC | Internal control |
| NO: 28 | | PCR primer R2: |
| SEQ ID | CACTTGCTCTCCCAAAGTCGCTC | LOPD seq 1 |
| NO: 29 | | |
| SEQ ID | ATACTCCGAGGCGGATCACAA | LOPD seq 2 |
| NO: 30 | | |
| SEQ ID | TCCTGGGGACATTCTAAGCGTG | LOPD seq 3 |
| NO: 31 | | |
| SEQ ID | CCAGAAGGAAGGCGAGAAAAGC | PPMO 1 |
| NO: 32 | | |
| SEQ ID | CCAGAAGGAABGGCGAGAAAAGC | PPMO 2 |
| NO: 33 | | |
| SEQ ID | CCAGAAGGAAGGBCGAGAAAAGC | PPMO 3 |
| NO: 34 | | |
| SEQ ID | GCACTCACGGBGCTCTCAAAGCAGC | PPMO 4 |
| NO: 35 | | |
| SEQ ID | GCACTCACGBBGCTCTCAAAGCAGC | PPMO 5 |
| NO: 36 | | |
| SEQ ID | GCTATTACCTTAACCCAG | PPMO 6 |
| NO: 37 | | |
| SEQ ID | TCC TGA GCC CAA ACA CTT CT | Fully Humanized |
| NO: 38 | | Mice-Wildtype |
| | | Forward |
| SEQ ID | ATT GTT GCA CAA CGC TCT TG | Fully Humanized |
| NO: 39 | | Mice-Common |
| SEQ ID | CGT TGG CTA CCC GTG ATA TT | Fully Humanized |
| NO: 40 | | Mice-Mutant |
| | | Forward |

What is claimed is:

1. A transgenic non-human animal model, comprising a nucleic acid sequence of an acid alpha-glucosidase (GAA) gene, or fragment thereof, comprising a mutation, wherein the mutation causes a defective splicing of the pre-mRNA transcribed from the nucleic acid sequence, and wherein the nucleic acid sequence comprising the mutation would have encoded a polypeptide having a GAA activity, if the nucleic acid sequence would have not comprised the mutation.

2. The transgenic non-human animal model of claim 1, wherein the mutation is a T-G mutation.

3. The transgenic non-human animal model of any one of claim 1 or 2, wherein the mutation is an IVS1-13T-G mutation.

4. The transgenic non-human animal model of any one of claims 1-3, wherein the GAA gene, or fragment thereof, comprising the mutation is transcribed into pre-mRNA.

5. The transgenic non-human animal model of claim 4, wherein the pre-mRNA transcribed from the GAA gene, or fragment thereof, comprising the mutation is processed by splicing into mature mRNA.

6. The transgenic non-human animal model of claim 5, wherein the mature mRNA derived from the pre-mRNA transcribed from the GAA gene, or fragment thereof, comprising the mutation is different from the mature mRNA derived from a pre-mRNA transcribed from a GAA gene, or fragment thereof, not comprising the mutation.

7. The transgenic non-human animal model of claim 6, wherein the mutation weakens the splice acceptor of GAA exon 2.

8. The transgenic non-human animal model of claim 7, wherein the mutation leads to skipping of exon 2.

9. The transgenic non-human animal model of claim 8, wherein the mature mRNA derived from the pre-mRNA transcribed from the GAA gene, or fragment thereof, comprising the mutation does not comprise exon 2.

10. The transgenic non-human animal model of any one of claims 5-9, wherein the mature mRNA derived from the pre-mRNA transcribed from the GAA gene, or fragment thereof, comprising the mutation is translated into a polypeptide having reduced GAA activity compared to a polypeptide translated from the mature mRNA transcribed from the a GAA gene, or fragment thereof, not comprising the mutation.

11. The transgenic non-human animal model of any one of claims 5-9, wherein the mature mRNA derived from the pre-mRNA transcribed from the GAA gene, or fragment thereof, comprising the mutation is translated into a polypeptide not having GAA activity.

12. The transgenic non-human animal model of any one of claims 1-11, wherein the non-human animal model is a model of Pompe disease.

13. The transgenic non-human animal model of claim 12, wherein the non-human animal model is a model of late onset Pompe disease.

14. The transgenic non-human animal model of any one of claims 1-13, wherein the nucleic acid sequence of the GAA gene, or fragment thereof, comprising the mutation comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NOs: 1 or 2.

15. The transgenic non-human animal model of any one of claims 1-14, wherein the nucleic acid sequence of the GAA gene, or fragment thereof, comprising the mutation is inserted in a Rosa26 locus.

16. The transgenic non-human animal model of any one of claims 1-14, wherein the nucleic acid sequence of the GAA gene, or fragment thereof, comprising the mutation is inserted in an endogenous GAA locus.

17. The transgenic non-human animal model of any one of claims 1-16, wherein the nucleic acid sequence of the GAA gene, or fragment thereof, comprising the mutation is operably linked to a heterologous promoter.

18. The transgenic non-human animal model of any one of claims 1-17, wherein the heterologous promoter is selected from the group consisting of a CV early enhancer/chicken β actin (CBA) promoter, CAGpromoter, CMV, EF1α, EF1α with a CMV enhancer, a CMV promoter with a CMV enhancer (CMVe/p), a CMV promoter with a SV40 intron.

19. The transgenic non-human animal model of claim 18, wherein the heterologous promoter is a CAG promoter.

20. The transgenic non-human animal model of any one of claim 18 or 19, wherein the heterologous promoter comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 3.

21. The transgenic non-human animal model of any one of claims 1-20, wherein the nucleic acid sequence of the GAA gene, or fragment thereof, comprising the mutation is operably linked to a heterologous polyadenylation signal.

22. The transgenic non-human animal model of claim 21, wherein the heterologous polyadenylation signal is an rGB-pA polyadenylation signal.

23. The transgenic non-human animal model of any one of claim 21 or 22, wherein the polyadenylation signal comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 4.

24. The transgenic non-human animal model of any one of claims 1-23, wherein the transgenic non-human animal model is generated by RNA-guided CRISPR-Cas nuclease system.

25. The transgenic non-human animal model of any one of claims 1-24 wherein the non-human animal model is a mouse

26. The transgenic non-human animal model of claim 25, wherein the mouse is a C57BL/6 mouse.

27. A recombinant nucleic acid molecule, comprising a 5′ homology arm, a polyadenylation signal, a nucleic acid sequence of an acid alpha-glucosidase (GAA) gene, or fragment thereof, comprising a mutation, wherein the mutation causes a defective splicing of the pre-mRNA transcribed from the nucleic acid sequence, and wherein the nucleic acid sequence comprising the mutation would have encoded a polypeptide having a GAA activity, if the nucleic acid sequence would have not comprised the mutation, a promoter, a 3′ homology arm.

28. The recombinant nucleic acid molecule of claim 27, further comprising a Neo (neomycin) resistance gene, an Amp (ampicillin) resistance gene.

29. The recombinant nucleic acid molecule of any one of claim 27 or 28, wherein the nucleic acid sequence of an acid alpha-glucosidase (GAA) gene, or fragment thereof, comprising the mutation, comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1 or 2.

30. The recombinant nucleic acid molecule of any one of claims 27-29, wherein the promoter is selected from the group consisting of a CMV early enhancer/chicken R actin (CBA) promoter, CAG promoter, CMV, EF1α, EF1α with a CMV enhancer, a CMV promoter with a CMV enhancer (CMVe/p), a CMV promoter with a SV40 intron.

31. The recombinant nucleic acid molecule of claim 30, wherein the promoter is a CAG promoter.

32. The recombinant nucleic acid molecule of any one of claim 29 or 30, wherein the promoter comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 3.

33. The recombinant nucleic acid molecule of any one of claims 27-32, wherein the polyadenylation signal is an rGB-pA polyadenylation signal.

34. The recombinant nucleic acid molecule of any one of claims 27-33, wherein the polyadenylation signal comprises a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 4.

35. The recombinant nucleic acid molecule of any one of claims 27-34, wherein the homology arms comprise a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to a region of the Rosa26 locus in a mouse genome.

36. The recombinant nucleic acid molecule of any one of claims 27-34, wherein the homology arms comprise a nucleic acid sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to a region of the GAA locus in a mouse genome.

37. A method of generating a transgenic mouse, comprising delivering to a cell the recombinant nucleic acid molecule of any one of claims 27-36.

38. The method of claim 37, wherein the cell is a mouse embryonic stem cell or a one-cell mouse embryo.

39. The method of any one of claim 37 or 38, further comprising delivering to the cell a sgRNA and a Cas9 nuclease.

40. The method of claim 39, wherein the delivered sgRNA target a locus in the mouse cell genome.

41. The method of any one of claims 37-41, wherein the polyadenylation signal, the nucleic acid sequence of an acid alpha-glucosidase (GAA) gene, or fragment thereof, comprising the mutation, and the promoter are stably integrated in a locus in the mouse genome.

42. The method of any one of claims 38-41 wherein the polyadenylation signal, the nucleic acid sequence of an acid alpha-glucosidase (GAA) gene, or fragment thereof, comprising the mutation, the promoter, the Neo (neomycin) resistance gene, and the Amp (ampicillin) resistance gene are stably integrated in a locus in the mouse genome.

43. The method of any one of claims 40-42, wherein the locus is a Rosa26 locus.

44. The method of any one of claims 40-42, wherein the locus is a GAA locus.

45. A method of testing a splice modulating agent comprising (a) administering a splice modulating agent to the transgenic non-human animal model of any one of claims 6-26, (b) obtaining a testing sample from the non-human animal model (c) and assaying for the presence of (i) mature mRNA derived from pre-mRNA transcribed from the GAA gene, or fragment thereof, comprising the mutation, and/or (ii) mature mRNA derived from pre-mRNA transcribed from a GAA gene, or fragment thereof, not comprising the mutation.

46. The method of claim 45, wherein the splice modulating agent is a small molecule.

47. The method of claim 45, wherein the splice modulating agent is an antisense oligonucleotide.

48. The method of any one of claims 45-47, wherein the splice modulating agent is administered to the transgenic non-human animal model.

49. The method of any one of claims 45-48, wherein the splice modulating agent is administered to cells, tissues, or organs derived from the transgenic non-human animal model.

50. The method of any one of claims 45-49, wherein the mature mRNA derived from the pre-mRNA transcribed from the GAA gene, or fragment thereof, comprising the mutation, is not different from the mature mRNA derived from a pre-mRNA transcribed from a GAA gene, or fragment thereof, not comprising the mutation, after administration of the splice modulating agent.

51. The method of any one of claims 45-50, wherein the mature mRNA derived from the pre-mRNA transcribed from the GAA gene, or fragment thereof, comprising the mutation, comprises exon 2, after administration of the splice modulating agent.

52. The method of any one of claims 45-51, wherein the mature mRNA derived from the pre-mRNA transcribed from the GAA gene, or fragment thereof, comprising the mutation is translated into a polypeptide having a GAA activity of a polypeptide translated from the mature mRNA transcribed from a GAA gene, or fragment thereof, not comprising the mutation, after administration of the splice modulating agent.

53. The method of any one of claims 45-49, wherein the mature mRNA derived from the pre-mRNA transcribed from the GAA gene, or fragment thereof, comprising the mutation is different from the mature mRNA derived from a pre-mRNA transcribed from a GAA gene, or fragment thereof, not comprising the mutation, after administration of the splice modulating agent.

54. The method of any one of claims 45-49, or 53 wherein the mature mRNA derived from the pre-mRNA transcribed from the GAA gene, or fragment thereof, comprising the mutation does not comprise exon 2, after administration of the splice modulating agent.

55. The method of any one of claims 45-49, 53 or 54, wherein the mature mRNA derived from the pre-mRNA transcribed from the GAA gene, or fragment thereof, comprising the mutation is translated into a polypeptide having reduced GAA activity compared to a polypeptide translated from the mature mRNA transcribed from a GAA gene, or fragment thereof, not comprising the mutation, after administration of the splice modulating agent.

56. The method of any one of claims 45-55, comprising extracting the mRNA from the cells, the tissues, or the organs derived from the transgenic non-human animal model prior to administering the splice modulating agent, and after administering the splice modulating agent.

57. The method of claim 56, comprising retrotranscribing the extracted mRNA into cDNA.

58. The method of claim 57, wherein the cDNA is amplified by a PCR comprising a first pair of primers capable of amplifying an exon junction which is not affected by the mutation, and a second pair of primers capable of amplifying an exon junction which is affected by the mutation.

59. The method of any one of claim 57 or 58, wherein the cDNA is amplified by a PCR comprising a pair of primers capable of amplifying cDNA derived from mRNA comprising exon junctions not affected by the mutation, and cDNA derived from mRNA comprising exon junctions affected by the mutation

60. The method of any one of claim 58 or 59, wherein the primers comprise a sequence which is at least 70%, at least 80%, at least 85%, at least 90%, at least 91%, at least 92%, at least 93%, at least 94%, at least 95%, at least 96%, at least 97%, at least 98%, at least 99%, or 100% identical to SEQ ID NOs:11-12.

61. The transgenic non-human animal model of any one of claims 1-26, wherein the acid alpha-glucosidase (GAA) gene is a human acid alpha-glucosidase (GAA) gene.

62. The transgenic non-human animal model of any one of claims 1-26, wherein at least one copy of an acid alpha-glucosidase gene endogenous to the non-human animal model is present in the genome of the transgenic non-human animal model.

63. The transgenic non-human animal model of claim 62, wherein all copies of the acid alpha-glucosidase gene endogenous to the non-human animal model are present in the genome of the transgenic non-human animal model.

64. The transgenic non-human animal model of claims 1-26, wherein at least one copy of an acid alpha-glucosidase gene endogenous to the non-human animal model is absent from the genome of the transgenic non-human animal mode.

65. The transgenic non-human animal model of claim 64, wherein all copies of the acid alpha-glucosidase gene endogenous to the non-human animal model are absent from the genome of the transgenic non-human animal mode.

66. The recombinant nucleic acid molecule of any one of claims 27-36, wherein the acid alpha-glucosidase (GAA) gene is a human acid alpha-glucosidase (GAA) gene.

67. The method of any one of claims 37-44, wherein the cell comprises at least one copy of an acid alpha-glucosidase gene endogenous to the cell genome.

68. The method of claim 67, wherein the cell comprises all copies of the acid alpha-glucosidase gene endogenous to the cell genome.

69. The method of any one of claims 37-44, wherein the cell lacks at least one copy of an acid alpha-glucosidase gene endogenous to the cell genome.

70. The method of claim 69, wherein the cell lacks all copies of the acid alpha-glucosidase gene endogenous to the cell genome.

71. A method of generating a transgenic mouse, comprising mating a first transgenic mouse generated by the method of claims 67-68 with a second transgenic mouse lacking all copies of a mouse GAA gene.

72. A method of testing a splice modulating agent comprising (a) administering a splice modulating agent to the transgenic non-human animal model of claim 65 (b) obtaining a testing sample from the non-human animal model (c) and assaying for the presence of (i) a protein product translated from a mature mRNA derived from a pre-mRNA transcribed from the GAA gene, or fragment thereof, comprising the mutation, and/or (ii) a protein product translated from a mature mRNA derived from a pre-mRNA transcribed from a GAA gene, or fragment thereof, not comprising the mutation.

73. The method of claim 72, wherein the splice modulating agent is a small molecule.

74. The method of claim 73, wherein the splice modulating agent is an antisense oligonucleotide.

75. The method of any one of claims 72-74, wherein the splice modulating agent is administered to the transgenic non-human animal model.

76. The method of any one of claims 72-75, wherein the splice modulating agent is administered to cells, tissues, or organs derived from the transgenic non-human animal model.

77. The method of any one of claims 72-76, wherein the protein product translated from a mature mRNA derived from a pre-mRNA transcribed from the GAA gene, or fragment thereof, comprising the mutation, is not different from a protein product translated from a mature mRNA derived from a pre-mRNA transcribed from a GAA gene, or fragment thereof, not comprising the mutation, after administration of the splice modulating agent.

78. The method of any one of claims 72-77, wherein the protein product translated from a mature mRNA derived from a pre-mRNA transcribed from the GAA gene, or fragment thereof, comprising the mutation, comprises an amino acid sequence encoded by exon 2, after administration of the splice modulating agent.

79. The method of any one of claims 72-78, wherein the protein product translated from a mature mRNA derived from a pre-mRNA transcribed from the GAA gene, or fragment thereof, comprising the mutation, has a GAA activity of a polypeptide translated from a mature mRNA transcribed from a GAA gene, or fragment thereof, not comprising the mutation, after administration of the splice modulating agent.

80. The method of any one of claims 72-76, wherein the protein product translated from a mature mRNA derived from a pre-mRNA transcribed from the GAA gene, or fragment thereof, comprising the mutation, is different from a protein product translated from a mature mRNA derived from a pre-mRNA transcribed from a GAA gene, or fragment thereof, not comprising the mutation, after administration of the splice modulating agent.

81. The method of any one of claims 72-76, or 80 wherein the protein product translated from a mature mRNA derived from a pre-mRNA transcribed from the GAA gene, or fragment thereof, comprising the mutation, does not comprise an amino acid sequence encoded by exon 2, after administration of the splice modulating agent.

82. The method of any one of claims 72-76, 80, or 81 wherein the protein product translated from a mature mRNA derived from a pre-mRNA transcribed from the GAA gene, or fragment thereof, comprising the mutation, has a reduced GAA activity compared to a polypeptide translated from a mature mRNA transcribed from a GAA gene, or fragment thereof not comprising the mutation, after administration of the splice modulating agent.

83. The method of any one of claims 72-82, comprising extracting the protein content from the cells, the tissues, or the organs derived from the transgenic non-human animal model prior to administering the splice modulating agent, and after administering the splice modulating agent.

84. The method of claim 83, wherein the protein content is analyzed by a Western blot assay.

85. The method of claim 84, wherein the Western blot assay comprises an anti-GAA antibody.
